SEQUENCE LISTING (20 sequences)

SEQ ID NO: 1 - DNA, 20 bp, Artificial Sequence
  GAPDH FW
ctccaaaatcaagtggggcg

SEQ ID NO: 2 - DNA, 20 bp, Artificial Sequence
  GAPDH RV
atgacgaacatgggggcatc

SEQ ID NO: 3 - DNA, 20 bp, Artificial Sequence
  MYCN FW
gcacagactgtagccatccg

SEQ ID NO: 4 - DNA, 21 bp, Artificial Sequence
  MYCN RV
tttaataccgggggtgcttcc

SEQ ID NO: 5 - DNA, 20 bp, Artificial Sequence
  cMYC FW
tcaagaggtgccacgtctcc

SEQ ID NO: 6 - DNA, 22 bp, Artificial Sequence
  cMYC RV
tcttggcagcaggatagtcctt

SEQ ID NO: 7 - DNA, 21 bp, Artificial Sequence
  PLK1 FW
tatatccctgcccgtctcccc

SEQ ID NO: 8 - DNA, 20 bp, Artificial Sequence
  PLK1 RV
cctcacctgtctctcgaacc

SEQ ID NO: 9 - DNA, 18 bp, Artificial Sequence
  LIN28b FW
ccttgagtcaatacgggt

SEQ ID NO: 10 - DNA, 18 bp, Artificial Sequence
  LIN28b RV
gctctgacagtaatggca

SEQ ID NO: 11 - DNA, 20 bp, Artificial Sequence
  RCOR2 FW
tctgggatcctgtcccgtag

SEQ ID NO: 12 - DNA, 20 bp, Artificial Sequence
  RCOR2 RV
ggtaattggttccaacgcgg

SEQ ID NO: 13 - DNA, 20 bp, Artificial Sequence
  BMI1 FW
tggctcgcattcattttctg

SEQ ID NO: 14 - DNA, 20 bp, Artificial Sequence
  BMI1 RV
agtagtggtctggtcttgtg

SEQ ID NO: 15 - DNA, 24 bp, Artificial Sequence
  CCL5 FW
gagtatttctacaccagtggcaag

SEQ ID NO: 16 - DNA, 21 bp, Artificial Sequence
  CCL5 RV
tcccgaacccatttcttctct

SEQ ID NO: 17 - DNA, 19 bp, Artificial Sequence
  CSNK2A1 FW
cttctcaggggaggcagga

SEQ ID NO: 18 - DNA, 21 bp, Artificial Sequence
  CSNK2A1 RV
cacacttccacaagagccact

SEQ ID NO: 19 - DNA, 24 bp, Artificial Sequence
  CSNK2A2 FW
cacttttccataagcagaacaaga

SEQ ID NO: 20 - DNA, 24 bp, Artificial Sequence
  CSNK2A2 RV
tacattcggaagtgaggtttgata

(19) United States
(12) Patent Application Publication
(10) Pub. No.: US 2022/0119399 A1
(43) Pub. Date: Apr. 21, 2022

(54) COMPOUNDS AND COMPOSITIONS THAT CAUSE MYCN AND/OR CMYC DEGRADATION AND METHODS OF USE THEREOF

(71) Applicant: THE TRUSTEES OF COLUMBIA UNIVERSITY IN THE CITY OF NEW YORK, New York, NY (US)

(72) Inventors: Brent R. Stockwell, New York, NY (US); Michael Stokes, New York, NY (US); Andrea Califano, New York, NY (US); Arie Zask, New York, NY (US)

(21) Appl. No.: 17/345,688

(22) Filed: Jun. 11, 2021

Related U.S. Application Data
(63) Continuation-in-part of application No. PCT/US19/65785, filed on Dec. 11, 2019.
(60) Provisional application No. 62/778,223, filed on Dec. 11, 2018.

(51) Int. Cl. C07D 491/052; C07D 493/04; C07D 493/14; C07D 311/36; A61P 35/00

(52) U.S. Cl. CPC C07D 491/052; C07D 493/04; A61P 35/00; C07D 311/36; C07D 493/14

(57) ABSTRACT

The present disclosure provides, inter alia, scaffolds and compounds having the structure:

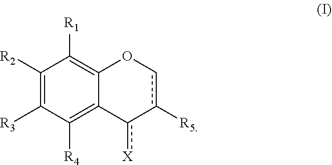

Also provided are compositions containing a pharmaceutically acceptable carrier and one or more compounds according to the present disclosure. Further provided are methods for treating or ameliorating the effects of a cancer in a subject, methods for selectively killing a cancer cell, methods of modulating mTORC1/2 signaling activity in a cell, methods of modulating the activity of a Master Regulator for MycN in a subject having MycN-amplified neuroblastoma (MycN^{AMP} NBL), methods of selectively treating or ameliorating effects of a cancer in a subject in need thereof, and platforms and methods for identifying a compound that induces degradation of a cancer-related protein. Also provided are kits comprising a compound or a pharmaceutical composition according to the present disclosure. Methods for treating cancers and methods for modulating MYC Master Regulators using other compounds are also provided.

### CROSS REFERENCE TO RELATED APPLICATIONS

The present application is a continuation-in-part of PCT international application no. PCT/US2019/065785, filed on Dec. 11, 2019, which claims benefit of U.S. Provisional Patent Application Ser. No. 62/778,223, filed on Dec. 11, 2018, which applications are incorporated by reference herein in their entireties.

### GOVERNMENT FUNDING

This invention was made with government support under grant no. CA217858, awarded by the National Institutes of Health. The government has certain rights in the invention.

### FIELD OF THE DISCLOSURE

The present disclosure provides, inter alia, scaffolds and compounds having the structure:

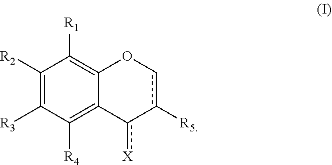

Platforms and methods for identifying such compounds are also provided. Pharmaceutical compositions containing the compounds of the present disclosure, as well as methods of using such compounds and compositions are also provided.

### INCORPORATION BY REFERENCE OF SEQUENCE LISTING

This application contains references to amino acids and/or nucleic acid sequences that have been filed concurrently herewith as sequence listing text file “CU19121-seq.txt”, file size of 4 KB, created on Dec. 8, 2019. The aforementioned sequence listing is hereby incorporated by reference in its entirety pursuant to 37 C.F.R. § 1.52(e)(5).

### BACKGROUND OF THE DISCLOSURE

MYCN-amplified neuroblastoma (MycN^{AMP}) is an aggressive pediatric tumor associated with poor prognosis and increased mortality (Huang and Weiss 2013). There are ˜700 new cases diagnosed in the U.S. every year; approximately 30% of neuroblastoma (NBL) tumors are “high-risk” MycN^{AMP} subtype, which has a 5-year survival rate of only ˜40% (Shimada et al. 2001). Patients with MycN^{AMP} NBL are easily identifiable through tumor biopsy samples, providing a robust method of identifying patients that would benefit from this therapeutic and enabling patient stratification in clinical studies. However, there are currently no targeted small molecule therapies approved for MycN^{AMP} NBL, making it an orphan disease with high unmet medical need.

In addition to NBL, there is a small but well defined patient subpopulation from a variety of cancers that are driven by MycN and would benefit from a novel MycN-suppressing therapy. For example, 2-3% of lung cancers and 2-3% of liver cancers are dysregulated in MycN expression, while ˜12% of Wilms' tumors have activated MycN.

MycN is a transcription factor of the Myc family that drives a number of processes, including cell proliferation (Koppen et al. 2007; Huang et al. 2011), metastasis (Ma et al. 2010) and immune evasion (Brandetti et al. 2017). Despite being a desirable target, targeting MycN directly has been impossible because it is a transcription factor that lacks binding pockets necessary to dock small molecule ligands. Inhibition of MycN is further challenged by sophisticated feedback mechanisms that stabilize MycN, and enables tumor cells to overcome therapeutic intervention (Koppen et al. 2007; Huang et al. 2011).

Recent network analysis revealed a complex regulatory module that centers on a MycN-TEAD4 interaction in MycN^{AMP} tumors (Rajbhandari et al. 2018). This regulatory module consists of transcription factors with dysregulated activity in MycN^{AMP} tumors that work coordinately to establish and maintain an aggressive phenotype (Rajbhandari et al. 2018; Califano and Alvarez 2017). Gene knock-down studies revealed that the regulatory module can compensate for chemical perturbation through a series of sophisticated feedback mechanisms (Rajbhandari et al. 2018; Califano and Alvarez 2017), suggesting that a successful drug candidate must disrupt the entire module to sustain tumor inhibition in patients (Califano and Alvarez 2017).

The current standard of care for MycN^{AMP} NBL is particularly grueling for pediatric patients, and can have long-lasting implications for growth and development (Cohen et al. 2014; Laverdiere et al. 2005; Laverdiere et al. 2009). Children that receive high-dose radiotherapy and chemotherapy experience reduced growth rates throughput adolescence and higher incidence of hypothyroidism, ovarian failure, hearing loss and dental issues as adults (Cohen et al. 2014). There are currently no targeted therapies approved for MycN^{AMP} NBL, despite significant effort by multiple research groups.

### SUMMARY OF THE DISCLOSURE

The identification of relevant targets that can form the basis of novel small molecule therapeutics is an ongoing challenge in drug discovery. It is hypothesized that cell line-selective chemical screening, followed by high-throughput gene expression profiling of drug perturbations could identify targeted agents that disrupt key drivers of disease. The present disclosure identified compounds that suppress MycN, an “undruggable” driver of aggressive high-risk pediatric neuroblastoma. Over 5500 bioactive molecules were screened across a panel of cell lines representing the MYCNA or mesenchymal (MES) NBL subtype, in search of subtype-selective compounds that disrupt a regulatory module mechanistically linked to tumor initiation and MycN stability. High-throughput expression profiling (PLATE-Seq) followed by Virtual Inference of Protein activity by Enriched Regulon analysis (VIPER) revealed a collection compounds that collapse the regulatory module and depleting MycN abundance in cell models of MYCNA NBL. The present disclosure identified a prenylated isoflavonoid molecule, named isopomiferin, which induced MycN degradation in cell models and tumor xenografts. An integrative analysis identified the pleiotropic kinase Casein Kinase 2 (CK2) as a direct target of isopomiferin and an essential regulator of the MYCNA tumor checkpoint module. Isopomiferin and its structural analogs were effective MYC suppressors in both NBL and lung cancers. The present disclosure provides a promising new precision-oncology framework to reveal actionable targets across aggressive cancer subtypes.

Accordingly, one embodiment of the present disclosure is a compound having the formula (I):

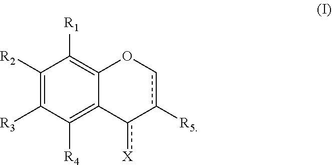

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof, or an N-oxide, crystalline form, hydrate thereof, or a pharmaceutically acceptable salt thereof,

with the proviso that the compound is not

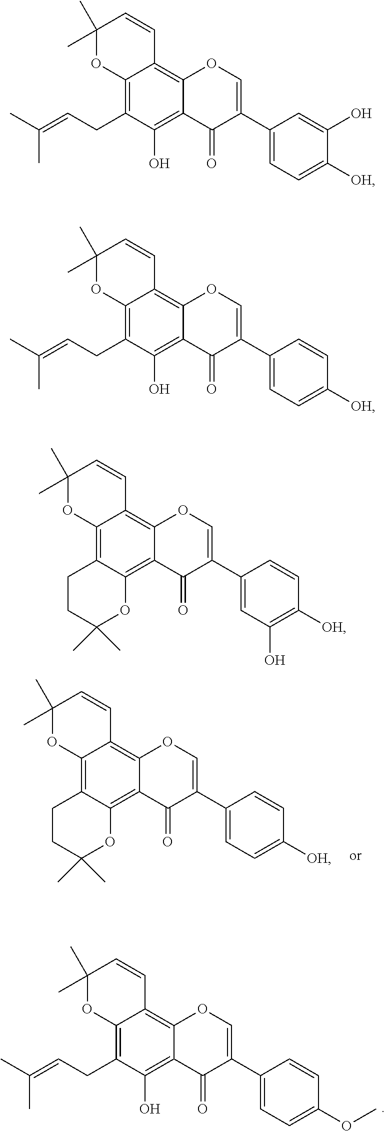

Another embodiment of the present disclosure is a pharmaceutical composition comprising a pharmaceutically acceptable carrier or diluent and a compound according to formula (I):

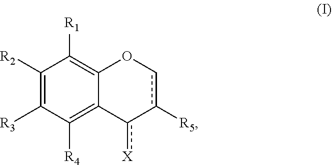

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof, or an N-oxide, crystalline form, hydrate thereof, or a pharmaceutically acceptable salt thereof,

with the proviso that the compound is not

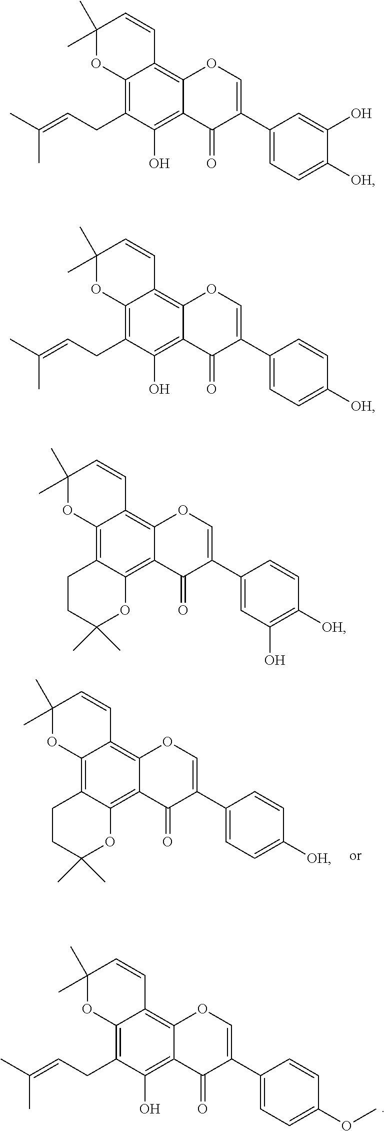

A further embodiment of the present disclosure is a kit. This kit comprises a compound or a pharmaceutical composition according to the present disclosure with instructions for the use of the compound or the pharmaceutical composition, respectively.

Another embodiment of the present disclosure is a method for treating or ameliorating the effects of a cancer in a subject. This method comprises administering to the subject a therapeutically effective amount of a compound having the structure of formula (I):

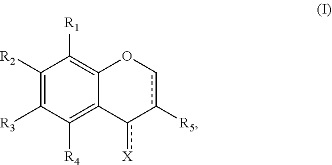

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof, or an N-oxide, crystalline form, hydrate thereof, or a pharmaceutically acceptable salt thereof.

Another embodiment of the present disclosure is a method for selectively killing a cancer cell. This method comprises contacting the cancer cell with an effective amount of a compound having the structure of formula (I):

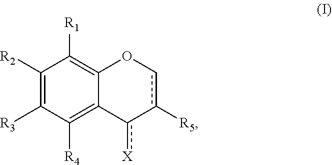

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof, or an N-oxide, crystalline form, hydrate thereof, or a pharmaceutically acceptable salt thereof.

Another embodiment of the present disclosure is a method of modulating mTORC1/2 signaling activity in a cell. The method comprises contacting the cell with an effective amount of a compound having the structure of formula (I):

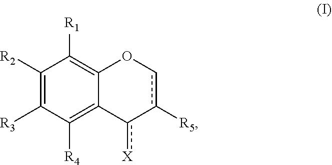

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄alkyl, CF₃, and combinations thereof, or an N-oxide, crystalline form, hydrate thereof, or a pharmaceutically acceptable salt thereof.

Another embodiment of the present disclosure is a method of modulating the activity of a master regulator for MycN in a subject having MycN-amplified neuroblastoma (MycN^{AMP} NBL). This method comprises administering to the subject a therapeutically effective amount of a compound having the structure of formula (I):

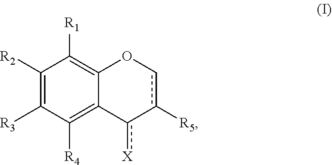

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof, or an N-oxide, crystalline form, hydrate thereof, or a pharmaceutically acceptable salt thereof.

Yet another embodiment of the present disclosure is a method of selectively treating or ameliorating effects of a cancer in a subject in need thereof. This method comprises the steps of: (a) obtaining a biological sample from the subject; (b) determining the expression level of MycN in the sample and comparing it with a predetermined reference; (c) identifying the subject as a MycN^{AMP} subtype if MycN in the sample is determined to be overexpressed in step (b); and (d) treating the MycN^{AMP} subtype subject with a therapeutically effective amount of a compound or a pharmaceutical composition disclosed herein.

Another embodiment of the present disclosure is a method of selectively treating or ameliorating effects of a cancer in a subject in need thereof. This method comprises the steps of: (a) obtaining a biological sample from the subject; (b) determining the expression level of cMyc in the sample and comparing it with a predetermined reference; (c) identifying the subject as a MycN^{AMP} subtype if cMyc in the sample is determined to be overexpressed in step (b); and (d) treating the MycN^{AMP} subtype subject with a therapeutically effective amount of a compound or a pharmaceutical composition disclosed herein.

Still another embodiment of the present disclosure is a method for identifying a compound that induces degradation of a cancer-related protein. This method comprises the steps of: (a) obtaining cancer cell lines that express the protein (AMP cell lines) and cancer cell lines that do not express the protein (NULL cell lines); (b) identifying compounds that are lethal to at least one of the cell lines; (c) identifying compounds that are selective for AMP cell lines from those identified in step (b) based on cell line subtype selectivity; (d) determining the expression level of the protein in AMP cell lines for each selective compound identified in step (c) by performing a high-throughput gene expression profiling; and (e) identifying a candidate compound that induces degradation of the cancer-related protein based on the result of step (d).

Another embodiment of the present disclosure is a method for treating or ameliorating the effects of a cancer in a subject. This method comprises administering to the subject a therapeutically effective amount of a compound selected from the group consisting of mycophenolate, NSC 80997, podofilox, cloxyquin, NSC 305798, NSC 255109, narasin, methylene blue, azure A, azure B, rapamycin, NSC 3905, and combinations thereof, or an N-oxide, crystalline form, hydrate thereof, or a pharmaceutically acceptable salt thereof.

Another embodiment of the present disclosure is a method of modulating the activity of a Master Regulator for MycN in a subject having MycN-amplified neuroblastoma (MycN^{AMP} NBL). This method comprises administering to the subject a therapeutically effective amount of a compound selected from the group consisting of mycophenolate, NSC 80997, podofilox, cloxyquin, NSC 305798, NSC 255109, narasin, methylene blue, azure A, azure B, rapamycin, NSC 3905, and combinations thereof, or an N-oxide, crystalline form, hydrate thereof, or a pharmaceutically acceptable salt thereof.

Another embodiment of the present disclosure is a compound having the structure of

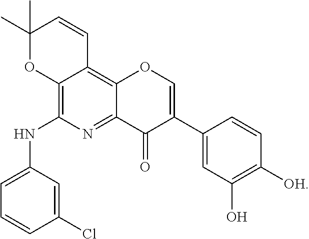 [SMILES: CC1(C)C=Cc2c(c(Nc3cccc(Cl)c3)nc3c(=O)c(-c4ccc(O)c(O)c4)coc23)O1]

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1D show that isopomiferin disrupts the regulatory drivers of MycN^{AMP} NBL.

FIG. 1A shows that >5500 compounds were screened across four NBL cell lines in a dose gradient. Heatmap of row-normalized IC₅₀ values identified subtype-selective compounds. Each row represents compound IC₅₀ values across four cell lines.

FIG. 1B shows the cell viability of two MycN^{AMP} cell lines (SK-N-Be2; IMR-32) and two MESN cell lines (SK-N-AS; NLF) treated with isopomiferin for 72 hrs.

FIG. 1C shows VIPER GSEA plots of SK-N-Be2 cells treated with the IC₂₀ of isopomiferin and doxorubicin for 24 hrs. Ranked along x-axis are 1479 transcription factor activities, from high to low. Isopomiferin induces coordinated reversion of regulatory module; doxorubicin induces spurious effects.

FIG. 1D shows the radar plots of PLATESeq GSEA analysis using Hallmarks of Cancer gene sets. Data points inside green hashed circle are significantly suppressed; points outside red circle are enriched. Two time-points (6 hrs; 24 hrs) and two concentrations of isopomiferin tested (3.3 μM and 10 μM).

FIGS. 2A-2G show that isopomiferin suppresses MycN expression and disrupts signaling through mTORC1/2.

FIG. 2A shows SK-N-Be2 cells treated with isopomiferin across a series of doses for 24 hrs.

FIG. 2B shows SK-N-Be2 cells treated with 15 μM isopomiferin and sampled across time.

FIG. 2C shows MYCN transcript abundance from cells treated with 15 μM isopomiferin and sampled across time.

FIG. 2D shows mouse xenografts of SK-N-Be2 cells treated with isopomiferin by intraperitoneal injection for 24 hrs. Two treated mice and one non-treated control mouse are shown.

FIG. 2E shows SK-N-Be2 cells treated with isopomiferin for 2 hrs and treated with IGF to induce pAKT (Ser473) activation; NVP-BEZ 235 at 100 nM used as a positive control.

FIG. 2F shows SK-N-Be2 cells assayed for p70^{S6K} phosphorylation following treatment with isopomiferin for 24 hrs.

FIG. 2G shows A549 cells assayed for p70^{S6K} phosphorylation following treatment with isopomiferin for 24 hrs.

FIGS. 3A-3E show that isopomiferin suppresses MYC proteins in lung cancer cell lines.

FIG. 3A shows that treatment with isopomiferin for 24 hrs suppresses MycN in two MycN-expressing SCLC lines.

FIG. 3B shows that treatment with isopomiferin for 72 hrs shows selective activity against MycN-expressing SCLC cell lines.

FIG. 3C shows the MYCN expression from cell line expression profiles from CCLE. Blue font indicates NBL cell models.

FIG. 3D shows the MycN abundance in A549 cells treated with isopomiferin for 24 hrs.

FIG. 3E shows qPCR analysis of CCL5 transcript abundance from A549 NSCLC cell lines treated with isopomiferin for 48 hrs.

FIGS. 4A-4C show that structural analogs vary in cell potency and activity.

FIG. 4A shows the structures of isopomiferin and four structural analogs. Potency varies greatly between compounds, gleaning insight into the SAR of the molecule. IC₅₀ at cell viability in SK-N-Be2 cells for 48 hrs.

FIG. 4B shows the dose-response curves of SK-N-Be2 treated with isopomiferin and four structural analogs.

FIG. 4C shows that isopomiferin contains four sites for optimization: i) Break or remove ring, replace 0 with N. ii) Remove double bond, break or remove ring, replace O with N. iii) Saturate ring or remove carbonyl. iv) Modify hydroxy groups or replace ring with heterocycles.

FIGS. 5A-5B show that TCM-reverting compounds destabilize MycN.

FIG. 5A shows the VIPER GSEA plots of control (DMSO) and 13 TCM-reverting compounds.

FIG. 5B shows that eleven TCM-reverting compounds were tested at the IC₂₀ and IC₅₀ for 24 hrs, and blotted for MycN and aurora kinase A (AurKA). Actin was used as a loading control.

FIGS. 6A-6E show that phenothiazine derivatives disrupt the MycN^{AMP} regulatory module and suppress MycN in NBL cells.

FIG. 6A shows the phenothiazine derivatives, their chemical structures and effect on cell viability after 48 hrs. Blue lines indicate MESN cells, and magenta lines indicate MycN^{AMP} cells.

FIG. 6B shows the VIPER GSEA plots of phenothiazine and derivatives at IC₂₀ treatments. Phenothiazing does not revert the signature while three derivates do have an effect.

FIG. 6C shows that methylene blue, azure A, and alisertib inhibit Histone H3 phosphorylation. Total histone H3 was shown as loading control.

FIG. 6D shows the treatment with methylene blue, azure A, and alisertib for 24 hrs following blotting for MycN abundance.

FIG. 6E shows the combination treatments of alisertib and azure A at indicated concentrations for 24 hrs, followed by blotting for MycN abundance.

FIGS. 7A-7D show that chemical screen identifies subtype-selective compounds.

FIG. 7A shows a heatmap of z-scores from row-normalized IC₅₀ values. Compounds ranked on ratio of average IC₅₀ of MycN^{AMP} cells over MESN cell line models.

FIG. 7B shows Western blot of MycN protein expression in MycN^{AMP} and MESN cell lines.

FIG. 7C show dose-response curves from four NBL cell lines treated for 48 hrs.

FIG. 7D shows chemical structures of four subtype-selective compounds.

FIGS. 8A-8D show that VIPER identifies lethal compounds that revert the MYCNA tumor checkpoint module (TCM).

FIG. 8A is schematic of PLATE-Seq workflow.

FIG. 8B shows that evaluating the effect of 90 compounds on the MYCNA TCM revealed hit molecules predicted to collapse the TCM and suppress MycN activity.

FIG. 8C shows VIPER GSEA plots rank 1473 transcription factors from high-to-low activity along x-axis. Highlighted are 25 Master Regulators (MRs) that are upregulated (red) or suppressed (blue) in MYCNA tumors. Arrow head indicates VIPER-inferred measurement of MycN activity, which is suppressed by compounds that collapse the TCM.

FIG. 8D shows Western blot of MycN protein abundance in SK-N-Be2 cells treated with TCM-reverting compounds for 24 hrs.

FIGS. 9A-9I show that isopomiferin induces degradation of MycN.

FIG. 9A shows radar plots of GSEA results of cancer hallmarks gene sets.

FIG. 9B shows MycN abundance measured in SK-N-Be2 cells treated with isopomiferin for 24 hrs, at indicated concentrations.

FIG. 9C shows MycN accumulation in SK-N-Be2 tumor xenografts 24 hrs following administration of isopomiferin by i.p. injection.

FIG. 9D shows Western blot analysis of MycN abundance in SK-N-Be2 cells treated with 5 μM MG132 and 15 μM isopomiferin for 6 hrs.

FIG. 9E shows measurement of P70^{S6K} phosphorylation levels following treatment with isopomiferin for 24 hrs. Total P70^{S6K} used as a loading control.

FIG. 9F shows measurement of AKT phosphorylation (Ser473) levels following treatment with isopomiferin for 24 hrs. B: treatment with 250 nM BEZ-235 used as a positive control. Total AKT used as loading control.

FIG. 9G shows cell-free mTOR kinase assay comparing pomiferin to the mTOR inhibitor PI-103.

FIG. 9H shows VIPER GSEA plots of 10 μM isopomiferin or 1 μM rapamycin for 24 hrs.

FIG. 9I shows Western blot analysis of Cleaved Caspase3 and Cleaved PARP. SK-N-Be2 cells treated at indicated concentration of isopomiferin for 48 hrs.

FIGS. 10A-10I show that structural analogs reveal a potent MycN inhibitor and preliminary SAR data.

FIG. 10A shows chemical structure of isopomiferin and five analogs.

FIG. 10B shows dose-response curves of SK-N-Be2 cells treated with isopomiferin analogs for 72 hrs.

FIG. 10C shows MycN abundance in SK-N-Be2 cells treated with isopomiferin, pomiferin, pomiferin dimethyl ether, or hydrogenated pomiferin for 24.

FIG. 10D shows Western blot analysis of MycN and TEAD4 abundance in SK-N-Be2 cells following treatment with pomiferin for 24 hrs.

FIG. 10E shows Western blot analysis of MycN abundance in SK-N-Be2 cells following treatment with 10 μM pomiferin across time.

FIG. 10F shows transcript abundance of three direct targets of MycN associated with poor patient outcome. SK-N-Be2 cells treated with 10 μM pomiferin for indicated time point.

FIG. 10G shows metabolic stability of isopomiferin and pomiferin in presence of mouse liver microsomes, 7-ethoxycoumarin used as positive control.

FIG. 10H shows metabolic stability of isopomiferin and pomiferin in mouse plasma. Ferrostatin-1 used as positive control.

FIG. 10I shows pharmacodynamic effect of isopomiferin and pomiferin in SK-N-Be2 flank tumor xenografts. Western blot of MycN abundance following daily i.p. injections at 20 mg/kg for 72 hrs.

FIGS. 11A-11F show that prenylated isoflavonoids bind and inhibit CK2 alpha subunits.

FIG. 11A shows VIKING output of predicted kinases targeted by isopomiferin.

FIG. 11B shows the effect of pomiferin on two casein kinase 2 alpha isoforms and mTOR kinase activity in cell-free assays.

FIG. 11C shows Western blot analysis of PTEN phosphorylation (Thr366) following treatment with either pomiferin or CX-4945 for 24 hrs.

FIG. 11D shows the crystal structure of Casein Kinase 2a1 dimer. E) Docking of pomiferin in kinase domain of CK2a1.

FIG. 11F shows the ligand interaction diagram of pomiferin in the crystal structure of CK2a1, corresponding to the docking pose shown in FIG. 11 E; arrows indicate hydrogen bonds.

FIGS. 12A-12I show the genetic and pharmacological validation of CK2a as a direct target of pomiferin.

FIG. 12A shows Western blot analysis of MycN and two CK2a isoforms following siRNA-mediated knock-down CK2a.

FIG. 12B shows cell viability measured following expression of mutant isoform with impaired binding to inhibitor.

FIG. 12C shows SK-N-Be2 cell viability following treatment with pomiferin, isopomiferin or CX-4945 for 72 hrs.

FIG. 12D shows Western blot analysis of hallmarks of apoptosis, cleaved PARP and cleaved caspase 3, following treatment with pomiferin or CX-4945 for 48 hrs.

FIG. 12E shows MycN protein abundance following treatment with pomiferin or CX-4945 for 24 hrs.

FIG. 12F shows cell free CK2a1 kinase activity in presence of pomiferin, isopomiferin or CX-4945.

FIG. 12G shows cellular accumulation of pomiferin or CX-4945 for indicated time points, and associated chemical structures.

FIG. 12H shows quantification of cellular abundance following treatment with prenylated isoflavonoids and CX-4945. Cell samples treated with 10 μM for 3 h, followed by LC-MS analysis.

FIG. 12I shows cell-free kinase inhibition assays of five kinases treated with pomiferin, isopomiferin, or a null analog (pomiferin dimethyl ether). Data points represent mean value of three technical replicates ±S.D.

FIGS. 13A-13F show that prenylated isoflavones are active across MYC-driven cancers.

FIG. 13A shows cell viability assays from four lung cancer lines treated with pomiferin for 72 hrs.

FIG. 13B shows Western blot analysis of cMyc protein abundance Sy5Y NBL cells treated with pomiferin for 24 hrs.

FIG. 13C shows MycN abundance measured in SCLC NCI-H69 cells treated with pomiferin for 24 hrs.

FIG. 13D shows SCLC NCI-H209 treated with isopomiferin for 24 hrs; MycL abundance measured by western blot.

FIG. 13E shows cMyc abundance in NSCLC A549 cells treated with isopomiferin for 24 hrs.

FIG. 13F shows qPCR measurement of CCL5 transcript in A549 cells following isopomiferin treatment.

FIG. 14 shows VIPER GSEA plots of all compounds that revert the MR signature (p<0.001).

FIGS. 15A-15D show that pomiferin disrupts oncogenic signaling through the MYC regulatory axis.

FIGS. 15A and 15B show the effect of 10 μM pomiferin on MYCN and cMYC transcript abundance. Despite induction of cMYC transcript, pomiferin blocks protein abundance of both MycN and cMyc. Sy5Y lysate included as control for cMYC western blot detection as shown in FIG. 15B.

FIG. 15C shows Kaplan Meyer curves of three direct targets of MycN; patient samples grouped by median expression value.

FIG. 15D shows transcript abundance of RCOR2 following treatment with 10 μM pomiferin for indicated time.

FIGS. 16A-16C show the isolation of prenylated isoflavonoids and their effect on NBL cell viability.

FIG. 16A shows liquid chromatograph of Osage orange extracts.

FIG. 16B shows cell viability of SK-N-Be2 cells treated with five prenylated isoflavonoids isolated from Osage oranges for 72 hrs.

FIG. 16C shows cell viability of SK-N-Be2 cells treated with isoflavonoids and flavonoid structures for 72 hrs.

FIGS. 17A-17C show the genetic and chemical interrogation of CK2 alpha subunits.

FIG. 17A shows qPCR validation of siRNA knockdown of CK2a1 or CK2a2.

FIG. 17B shows Western blot analysis of CK2a1 abundance following treatment with either pomiferin or CX-4945 for 24 hrs.

FIG. 17C shows LC-MS detection of pomiferin and CX-4945, as a standard and from cellular extracts from SK-N-Be2 cells treated with 10 μM of pomiferin or CX-4945 for 3 hrs.

FIG. 18 shows the ranking CCLE expression profiles reveals cell models with high MycN expression.

FIGS. 19A-19F show that pomiferin delays NBL tumor growth in mouse xenografts.

FIG. 19A shows stability of pomiferin isopomiferin and ferrostatin-1 (positive control) in mouse plasma across 4 h. Data represent mean±S.D. of three replicates.

FIG. 19B shows stability of isopomiferin, pomiferin and 7-ethoxycoumarin (positive control) following 2 h incubation in mouse liver microsomes at 0.5 mg/mL. Data represent mean±S.D. of three replicates.

FIG. 19C shows tumor growth across three-week treatment with 20 mg/kg pomiferin or vehicle control. (* indicates statistical significance at p<0.05).

FIG. 19D shows tumor volume from mice treated with either isopomiferin (20 mg/kg), pomiferin (20 mg/kg), or vehicle control for 21 days (* indicates p<0.05).

FIG. 19E shows western blot analysis of MYCN abundance in tumor xenografts on day 14 of the treatment regimen.

FIG. 19F shows mouse body weight across time, following daily treatment with isopomiferin (20 mg/kg), pomiferin (20 mg/kg), or vehicle control for 21 d.

FIG. 20A shows transcriptome analysis of pomiferin and isopomiferin, compared to dimethyl pomiferin.

FIG. 20B shows VIPER analysis of individual master regulators that comprise the signature. Most MRs exhibited decreases in protein activity across all three compounds tested. Only six MRs were suppressed by pomiferin and isopomiferin, while being unaffected by the null analog dimethyl pomiferin.

FIG. 20C shows gene set enrichment analysis revealed that pomiferin and isopomiferin both suppressed transcripts associated with G2/M checkpoint and E2F pathways, consistent with previous results generated using PLATESeq.

FIG. 20D shows that VIPER analysis of expression profiles revealed that pomiferin had the strongest effect on the MYCN MR signature, whereas dimethyl pomiferin did not.

FIG. 20E shows protein activity profiles for pomiferin, isopomiferin and dimethyl pomiferin.

### DETAILED DESCRIPTION OF THE DISCLOSURE

Neuroblastoma is the most common extracranial solid tumors affecting children, responsible for ˜15% of all pediatric cancer deaths each year (Huang and Weiss, 2013; Louis and Shohet, 2015). Neuroblastoma derives from the neural crest, an embryonic structure that gives rise to the sympathetic nervous system (Marshall et al. 2014; Cheung and Dyer, 2013). As neural crest cells proliferate and differentiate, genetic alterations can occur that result in tumor development; the severity of disease determined by the specific combinations of genetic lesions (Marshall et al. 2014; Cheung and Dyer, 2013; Brodeur and Bagatell, 2014). For example, tumors driven by amplification of the MYCN locus (MYCNA subtype) are associated with an aggressive phenotype and poor prognosis for patients (Brodeur et al. 1984; Seeger et al. 1985). Tumor stratification based on clinical, pathologic, and genetic factors places patients into risk categories, with high risk NBL carrying 40-50% chance of survival (Huang and Weiss, 2013; Irwin and Park, 2015; Ora and Eggert, 2011).

MycN is considered an “undruggable” protein, due to the lack of potential binding sites on its surface amenable to small molecule docking. To circumvent the challenge of targeting MycN directly, strategies that disrupt MycN protein regulation could indirectly inhibit MycN activity. MycN abundance is a determined by the relative rates of synthesis and degradation; modulation of these processes alters MycN activity in cells. Small molecule approaches have had some success at indirect MycN suppression. Inhibition of aurora kinase A by MLN8237 (Alisertib) induced MycN destabilization in both cell and animal models of MYCNA NBL (Richards et al. 2016; Gustafson et al. 2014; Otto et al. 2009). Targeting MycN indirectly with small molecules may be a viable strategy to disrupt MycN in cells.

MycN expression is driven by sophisticated feedback system that stabilizes MycN protein and supports drug resistance. Recently, a systems-level understanding of this regulatory architecture was elucidated using network-based analysis of NBL primary tumor expression profiles (Rajbhandari et al. 2018). This analysis revealed a core set of ten proteins that comprise a tumor checkpoint module that converges on a MycN-TEAD4 regulatory interaction (Rajbhandari et al. 2018). Genetic disruption of this module suppresses MycN in vivo, suggesting that targeting the module with small molecule inhibitors may be an effective strategy to ameliorate MycN in cells.

It is hypothesized that collapse of the tumor checkpoint module is sufficient for MycN suppression, and developed a methodology to identify compounds that disrupt a regulatory signature associated with MYCNA tumors. This approach relies on a novel high-throughput expression profiling tool that evaluates drug perturbation in a 96-well format, called PLATE-seq (Bush et al. 2017). When used in conjunction with network-based algorithms that infer the activity of regulatory proteins (Alvarez et al. 2016; Wang et al. 2009), this technology enables us to screen lethal molecules for the ability to collapse the 10-protein MYCNA checkpoint module.

This screening methodology revealed a suite of compounds that disrupt the MYCNA signature in cell models of MYCNA NBL, suppressing MycN protein expression. The top ranked molecule was a prenylated isoflavonoid, named isopomiferin, which collapsed the TCM and suppressed MycN in cells. Informatic analysis of network dysregulation of isopomiferin-treated cells identified Casein Kinase 2a (CK2) as a direct functional target of isopomiferin and structurally-related isoflavonoid molecules. CK2 is a pleiotropic kinase that regulates a variety of cellular processes, including cellular proliferation (Turowec et al. 2010; Trembley et al. 2009). The present disclosure characterizes the mechanism of this unique class of inhibitors that act through CK2 to disrupt the MYCNA tumor checkpoint module and suppress MycN in cells. This methodology can now be expanded across cancers to identify selective compounds that suppress key regulatory architecture driving aggressive tumors.

Isopomiferin is known as being tailored for MycN tumors, it can also suppress cMyc activity, likely through shared upstream regulatory factors. cMyc is amplified in ˜10% of breast and 10% of lung cancers (www.cBioportal.org), among many others, which greatly expands the potential target market. It is believed that developing isopomiferin into a therapeutic compound that can suppress cMyc in tumor cells would be a great advance for the treatment of recalcitrant tumors.

As to the regulatory module, in the present disclosure, an alternate approach is developed to inhibit the signaling pathways that drive and stabilize MycN protein expression. Given the complex feedback mechanisms that maintain elevated MycN levels, it is believed that a successful therapeutic would need to suppress the entire feedback regulatory module to sustain prolonged MycN suppression in cells.

Moreover, in the present disclosure, there is provided a novel targeted therapy that disrupts core regulatory drivers of MycN^{AMP} NBL, from a scaffold that should be well-tolerated in humans. The goal is to introduce a novel small molecule therapy that improves clinical outcomes for patients, and reduces the long-term health effects caused by current treatment modalities.

Accordingly, one embodiment of the present disclosure is a compound having the formula (I):

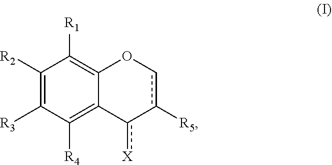

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄alkyl, CF₃, and combinations thereof, or an N-oxide, crystalline form, hydrate thereof, or a pharmaceutically acceptable salt thereof,

with the proviso that the compound is not

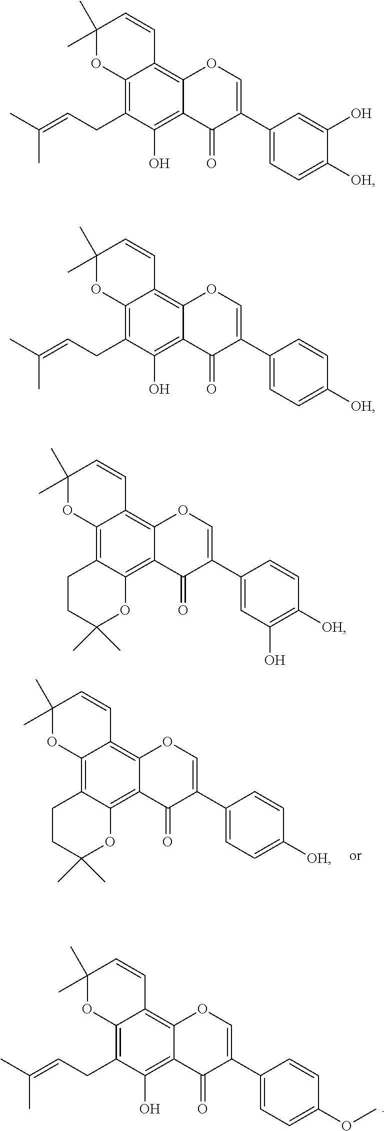

In some embodiments, the compound having the structure of formula (I) does not have —OH group at R₂ position.

In some embodiments, the compound is selected from the group consisting of:

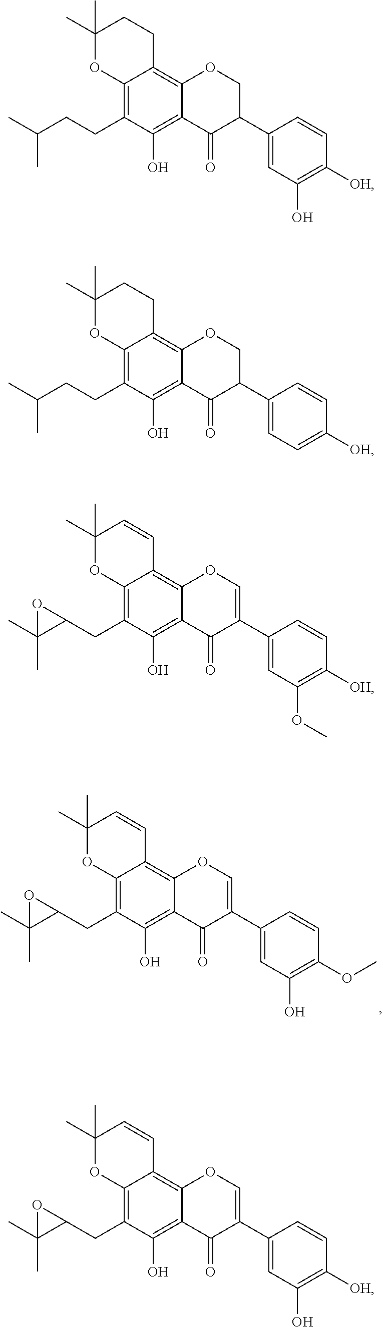

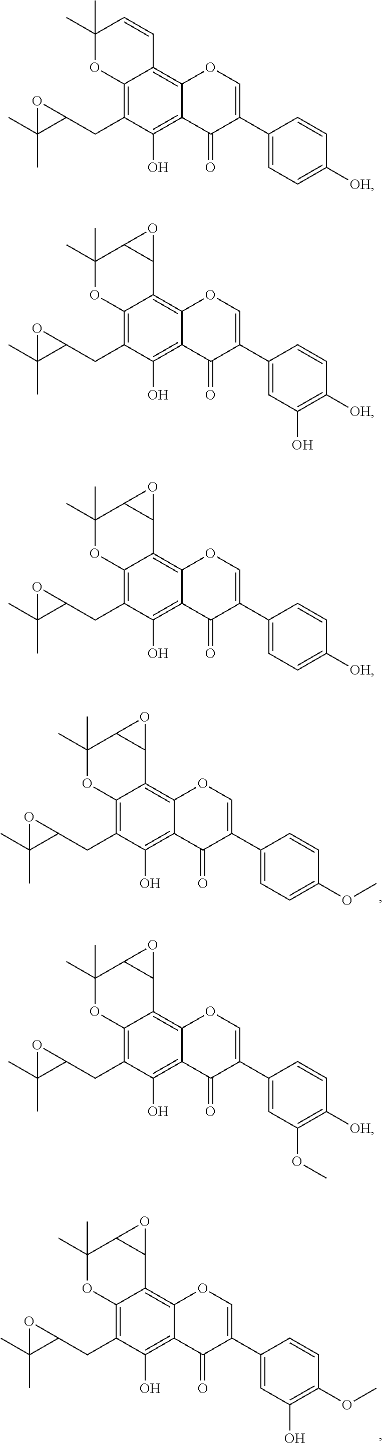

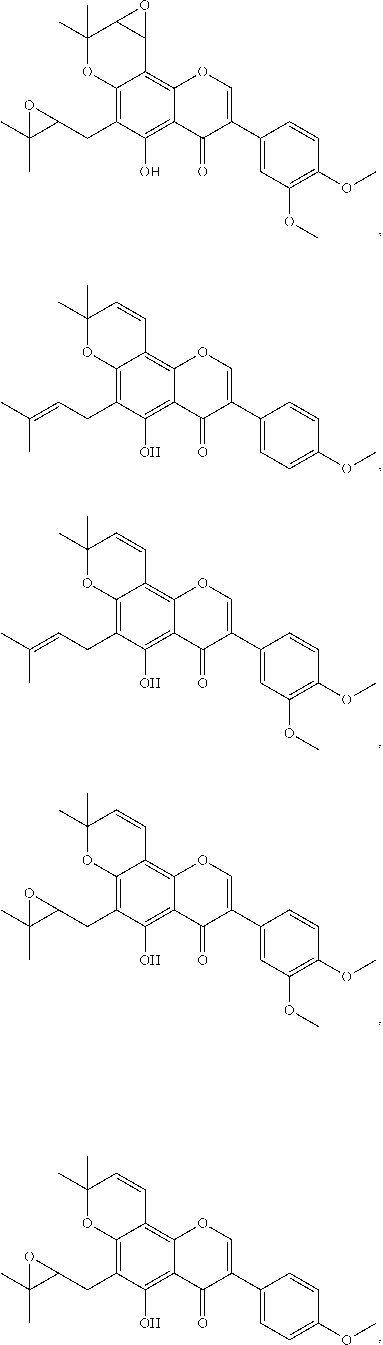

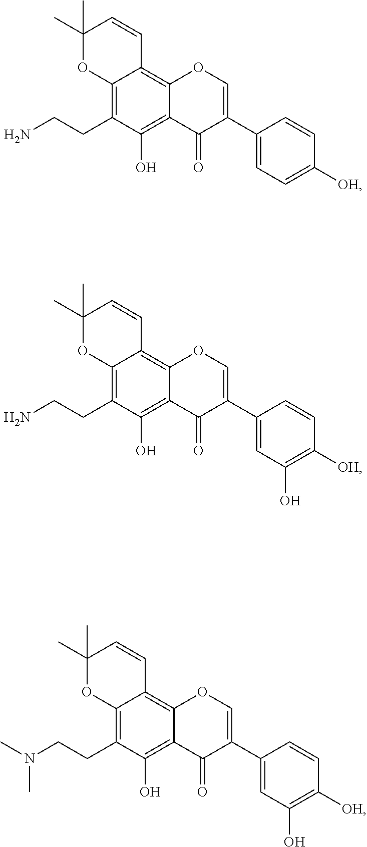 [SMILES: CC1(C)C=Cc2c(c(CCN)c(O)c3c(=O)c(-c4ccc(O)cc4)coc23)O1 ; CC1(C)C=Cc2c(c(CCN)c(O)c3c(=O)c(-c4ccc(O)c(O)c4)coc23)O1 ; CN(C)CCc1c2c(c3occ(-c4ccc(O)c(O)c4)c(=O)c3c1O)C=CC(C)(C)O2]

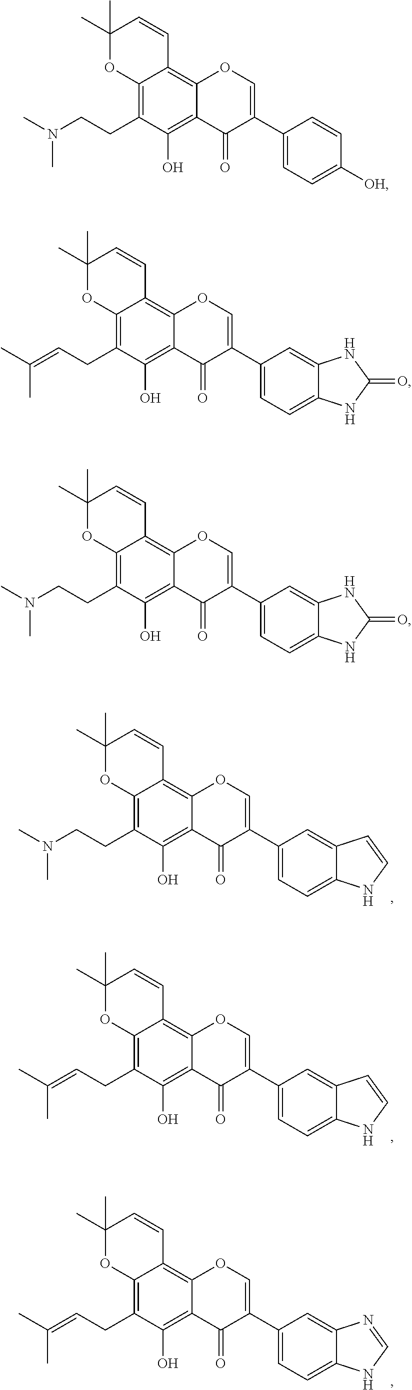 [SMILES: CN(C)CCc1c2c(c3occ(-c4ccc(O)cc4)c(=O)c3c1O)C=CC(C)(C)O2 ; CC(C)=CCc1c2c(c3occ(-c4ccc5[nH]c(=O)[nH]c5c4)c(=O)c3c1O)C=CC(C)(C)O2 ; CN(C)CCc1c2c(c3occ(-c4ccc5[nH]c(=O)[nH]c5c4)c(=O)c3c1O)C=CC(C)(C)O2 ; CN(C)CCc1c2c(c3occ(-c4ccc5[nH]ccc5c4)c(=O)c3c1O)C=CC(C)(C)O2 ; CC(C)=CCc1c2c(c3occ(-c4ccc5[nH]ccc5c4)c(=O)c3c1O)C=CC(C)(C)O2 ; CC(C)=CCc1c2c(c3occ(-c4ccc5[nH]cnc5c4)c(=O)c3c1O)C=CC(C)(C)O2]

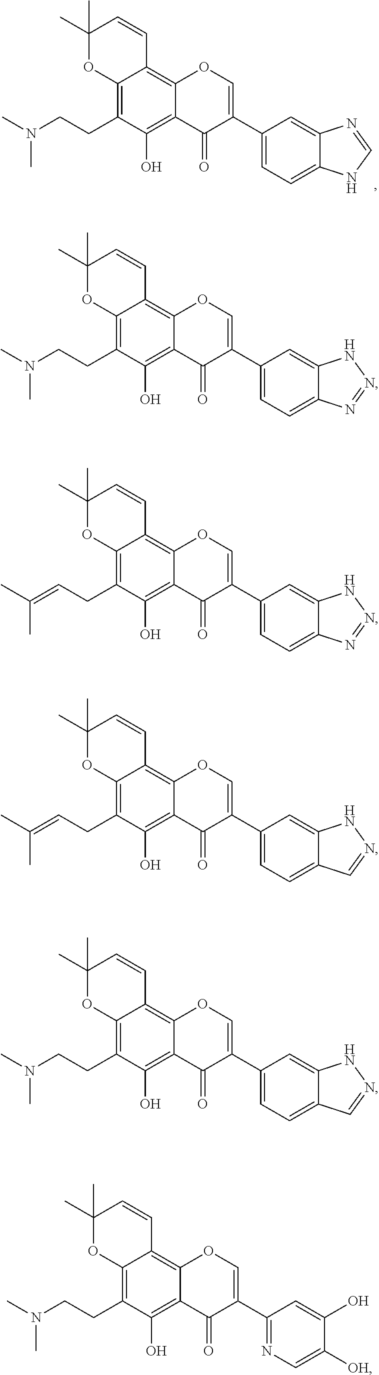 [SMILES: CN(C)CCc1c2c(c3occ(-c4ccc5[nH]cnc5c4)c(=O)c3c1O)C=CC(C)(C)O2 ; CN(C)CCc1c2c(c3occ(-c4ccc5nn[nH]c5c4)c(=O)c3c1O)C=CC(C)(C)O2 ; CC(C)=CCc1c2c(c3occ(-c4ccc5nn[nH]c5c4)c(=O)c3c1O)C=CC(C)(C)O2 ; CC(C)=CCc1c2c(c3occ(-c4ccc5cn[nH]c5c4)c(=O)c3c1O)C=CC(C)(C)O2 ; CN(C)CCc1c2c(c3occ(-c4ccc5cn[nH]c5c4)c(=O)c3c1O)C=CC(C)(C)O2 ; CN(C)CCc1c2c(c3occ(-c4cc(O)c(O)cn4)c(=O)c3c1O)C=CC(C)(C)O2]

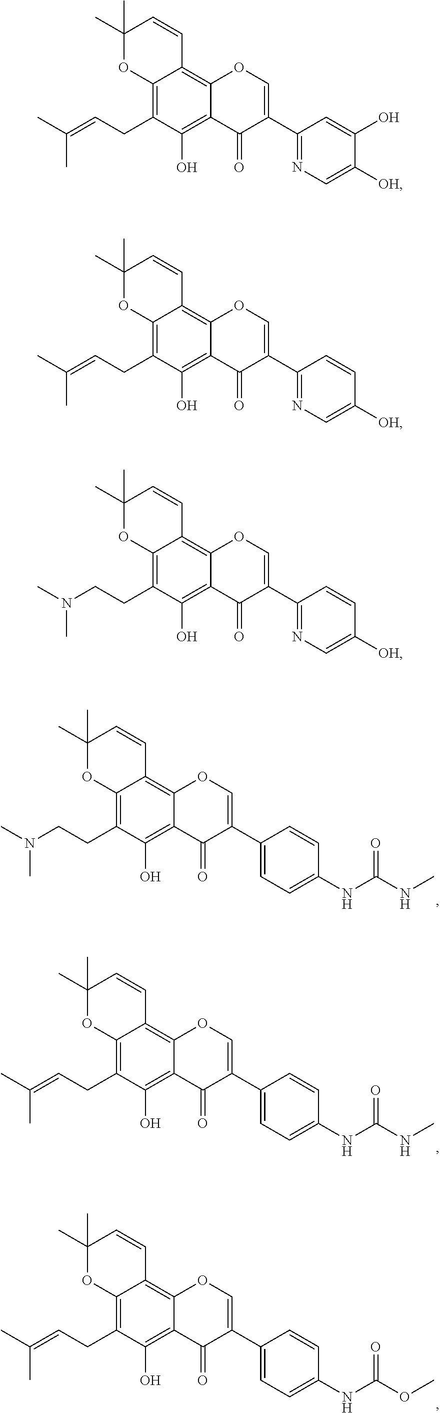

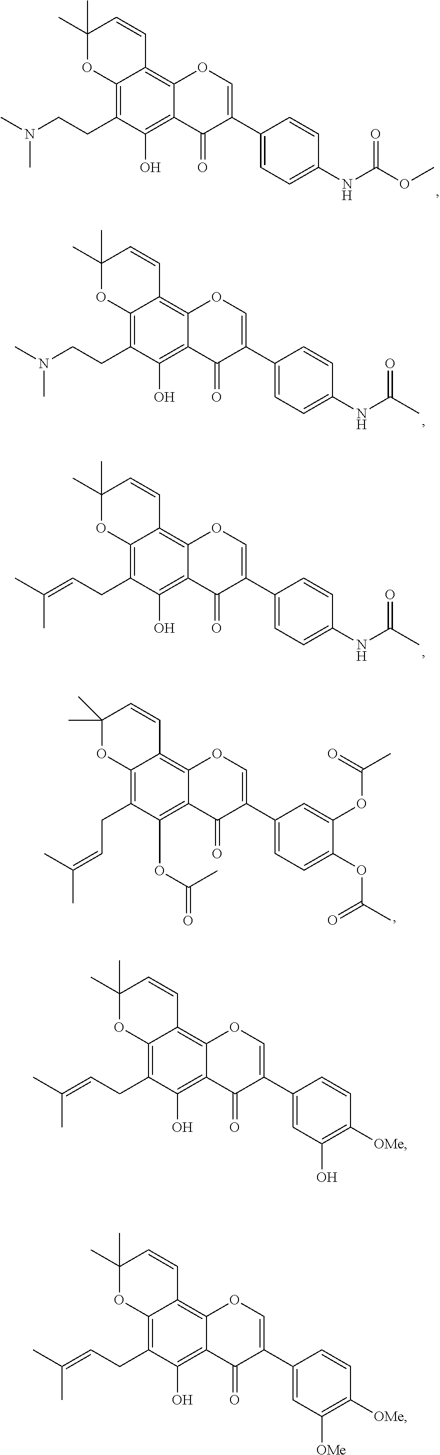

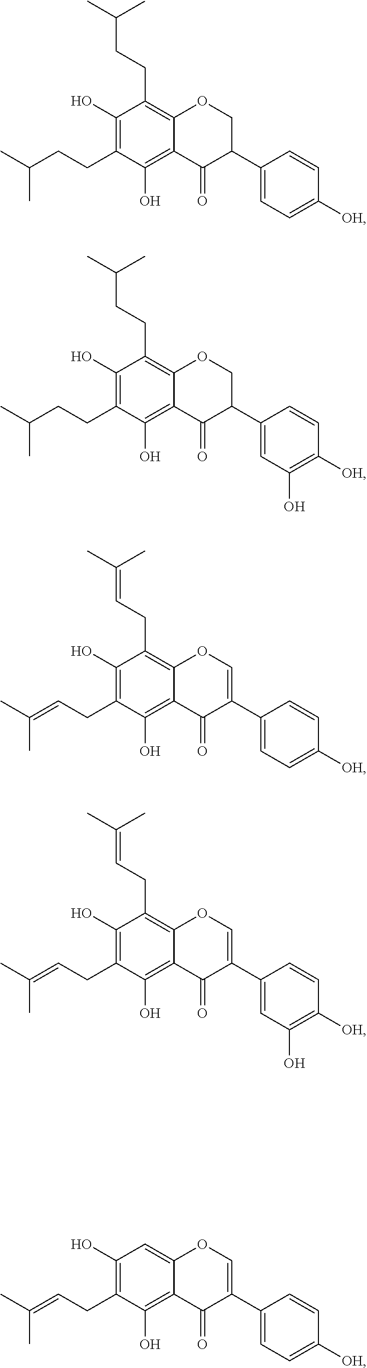 [SMILES: CC(C)CCc1c(O)c(CCC(C)C)c2c(c1O)C(=O)C(c1ccc(O)cc1)CO2 ; CC(C)CCc1c(O)c(CCC(C)C)c2c(c1O)C(=O)C(c1ccc(O)c(O)c1)CO2 ; CC(C)=CCc1c(O)c(CC=C(C)C)c2occ(-c3ccc(O)cc3)c(=O)c2c1O ; CC(C)=CCc1c(O)c(CC=C(C)C)c2occ(-c3ccc(O)c(O)c3)c(=O)c2c1O ; CC(C)=CCc1c(O)cc2occ(-c3ccc(O)cc3)c(=O)c2c1O]

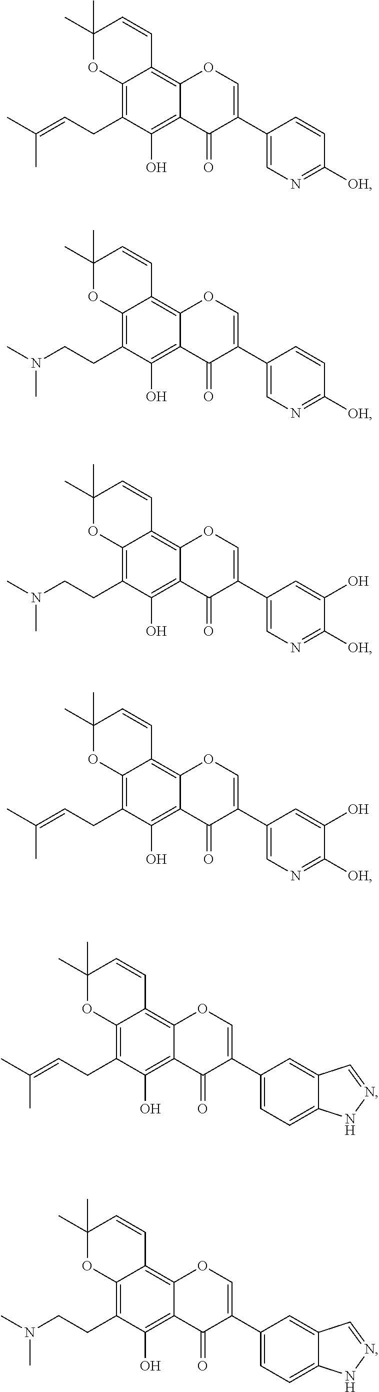 [SMILES: CC(C)=CCc1c2c(c3occ(-c4ccc(O)nc4)c(=O)c3c1O)C=CC(C)(C)O2 ; CN(C)CCc1c2c(c3occ(-c4ccc(O)nc4)c(=O)c3c1O)C=CC(C)(C)O2 ; CN(C)CCc1c2c(c3occ(-c4cnc(O)c(O)c4)c(=O)c3c1O)C=CC(C)(C)O2 ; CC(C)=CCc1c2c(c3occ(-c4cnc(O)c(O)c4)c(=O)c3c1O)C=CC(C)(C)O2 ; CC(C)=CCc1c2c(c3occ(-c4ccc5[nH]ncc5c4)c(=O)c3c1O)C=CC(C)(C)O2 ; CN(C)CCc1c2c(c3occ(-c4ccc5[nH]ncc5c4)c(=O)c3c1O)C=CC(C)(C)O2]

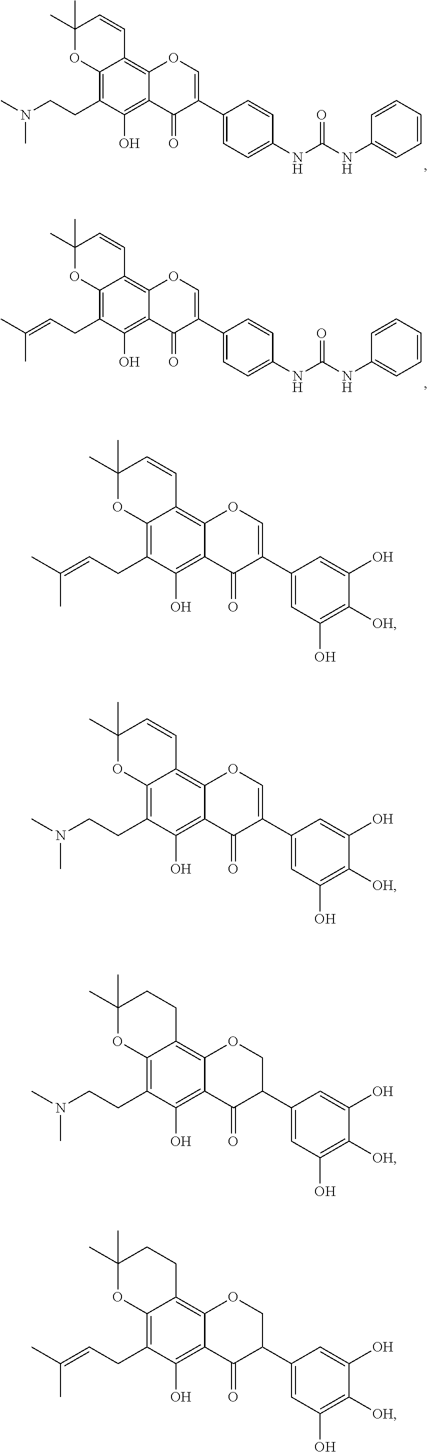 [SMILES: CN(C)CCc1c2c(c3occ(-c4ccc(NC(=O)Nc5ccccc5)cc4)c(=O)c3c1O)C=CC(C)(C)O2 ; CC(C)=CCc1c2c(c3occ(-c4ccc(NC(=O)Nc5ccccc5)cc4)c(=O)c3c1O)C=CC(C)(C)O2 ; CC(C)=CCc1c2c(c3occ(-c4cc(O)c(O)c(O)c4)c(=O)c3c1O)C=CC(C)(C)O2 ; CN(C)CCc1c2c(c3occ(-c4cc(O)c(O)c(O)c4)c(=O)c3c1O)C=CC(C)(C)O2 ; CN(C)CCc1c(O)c2c(c3c1OC(C)(C)CC3)OCC(c1cc(O)c(O)c(O)c1)C2=O ; CC(C)=CCc1c(O)c2c(c3c1OC(C)(C)CC3)OCC(c1cc(O)c(O)c(O)c1)C2=O]

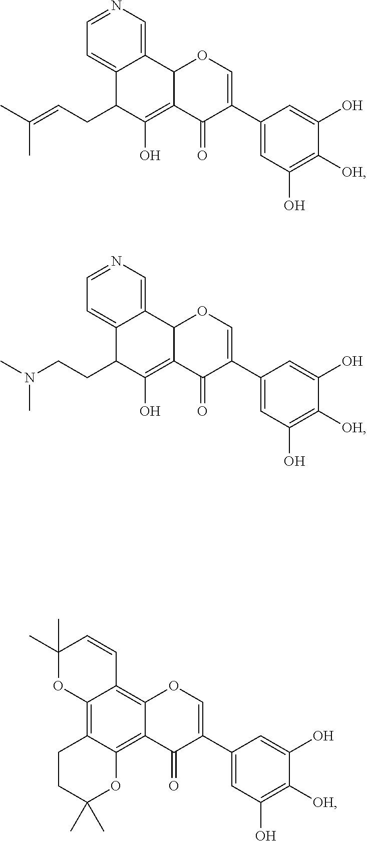 [SMILES: CC(C)=CCC1C(O)=C2C(=O)C(c3cc(O)c(O)c(O)c3)=COC2c2cnccc21 ; CN(C)CCC1C(O)=C2C(=O)C(c3cc(O)c(O)c(O)c3)=COC2c2cnccc21 ; CC1(C)C=Cc2c(c3c(c4c(=O)c(-c5cc(O)c(O)c(O)c5)coc24)OC(C)(C)CC3)O1]

and combinations thereof,
or an N-oxide, crystalline form, hydrate thereof, or a pharmaceutically acceptable salt thereof.

Another embodiment of the present disclosure is a pharmaceutical composition comprising a pharmaceutically acceptable carrier or diluent and a compound according to formula (I):

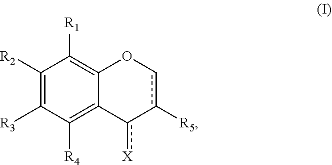

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof, or an N-oxide, crystalline form, hydrate thereof, or a pharmaceutically acceptable salt thereof,

with the proviso that the compound is not

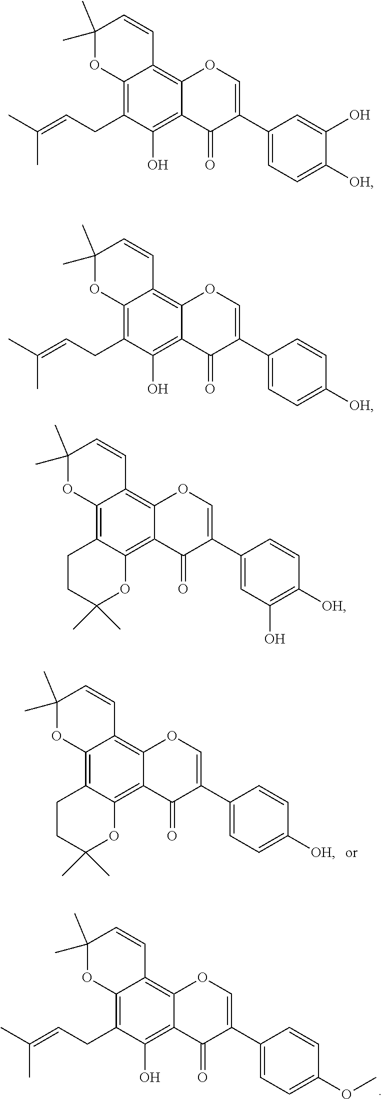

In some embodiments, the compound according to formula (I) does not have —OH group at R₂ position.

In some embodiments, the pharmaceutical composition comprises a compound that is selected from the group consisting of:

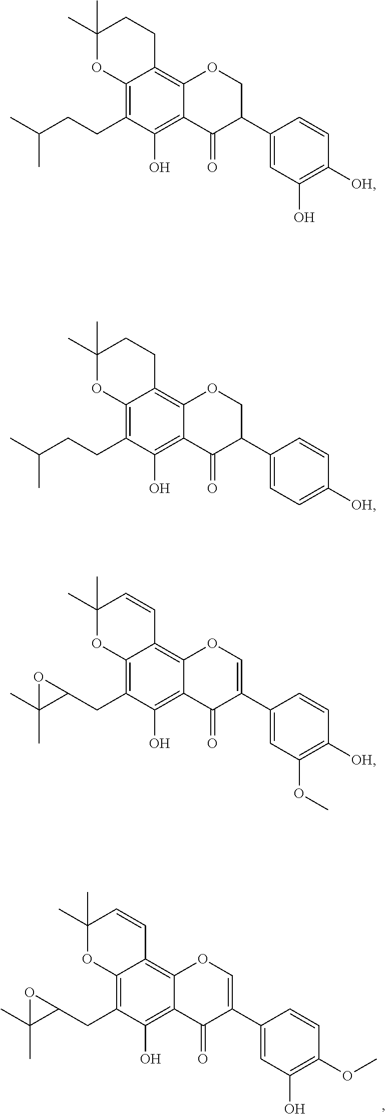

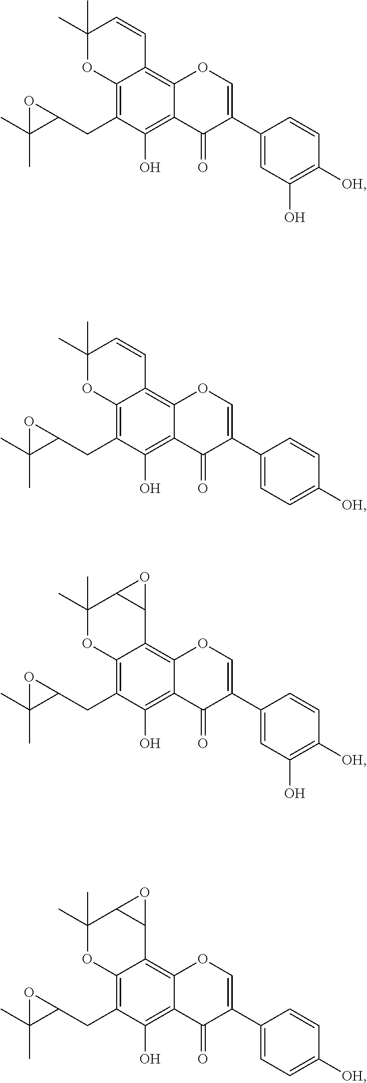 [SMILES: CC1(C)C=Cc2c(c(CC3OC3(C)C)c(O)c3c(=O)c(-c4ccc(O)c(O)c4)coc23)O1 ; CC1(C)C=Cc2c(c(CC3OC3(C)C)c(O)c3c(=O)c(-c4ccc(O)cc4)coc23)O1 ; CC1(C)OC1Cc1c2c(c3occ(-c4ccc(O)c(O)c4)c(=O)c3c1O)C1OC1C(C)(C)O2 ; CC1(C)OC1Cc1c2c(c3occ(-c4ccc(O)cc4)c(=O)c3c1O)C1OC1C(C)(C)O2]

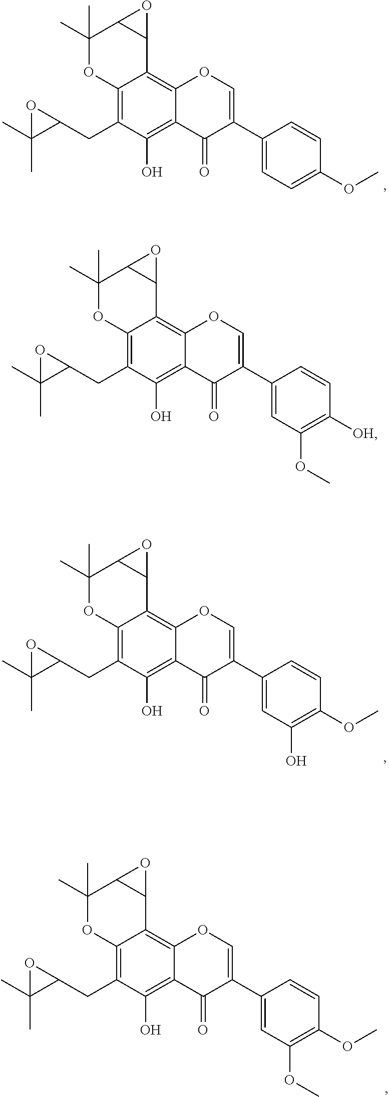

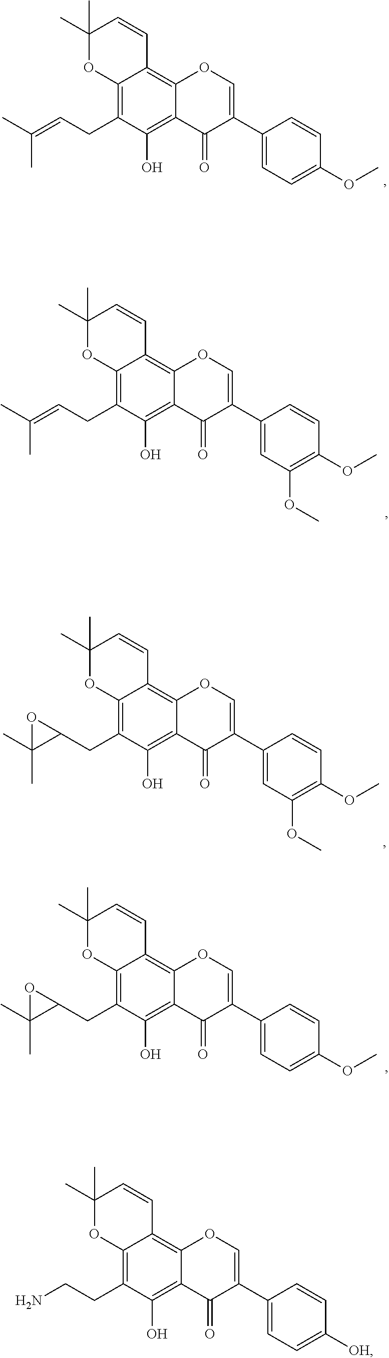

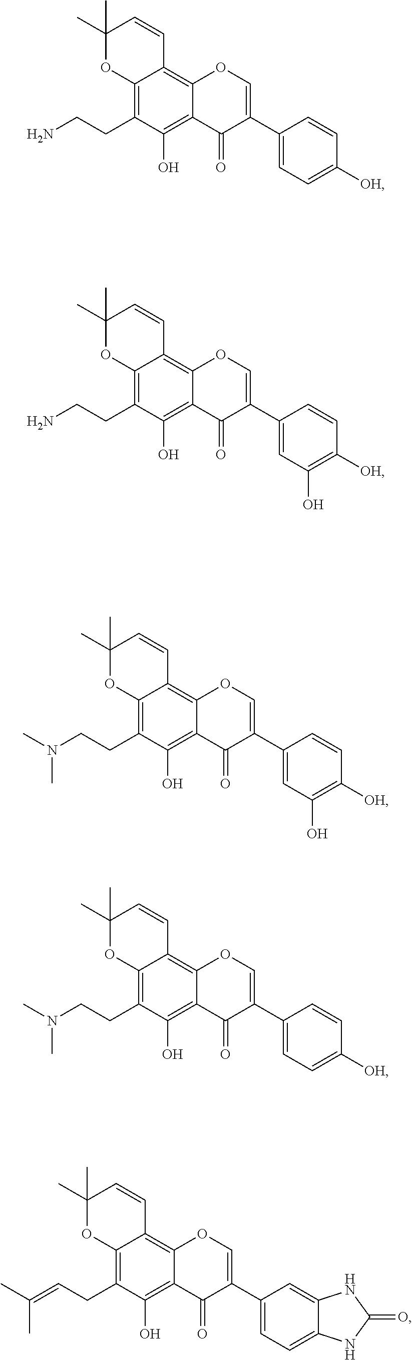 [SMILES: CC1(C)C=Cc2c(c(CCN)c(O)c3c(=O)c(-c4ccc(O)cc4)coc23)O1 ; CC1(C)C=Cc2c(c(CCN)c(O)c3c(=O)c(-c4ccc(O)c(O)c4)coc23)O1 ; CN(C)CCc1c2c(c3occ(-c4ccc(O)c(O)c4)c(=O)c3c1O)C=CC(C)(C)O2 ; CN(C)CCc1c2c(c3occ(-c4ccc(O)cc4)c(=O)c3c1O)C=CC(C)(C)O2 ; CC(C)=CCc1c2c(c3occ(-c4ccc5[nH]c(=O)[nH]c5c4)c(=O)c3c1O)C=CC(C)(C)O2]

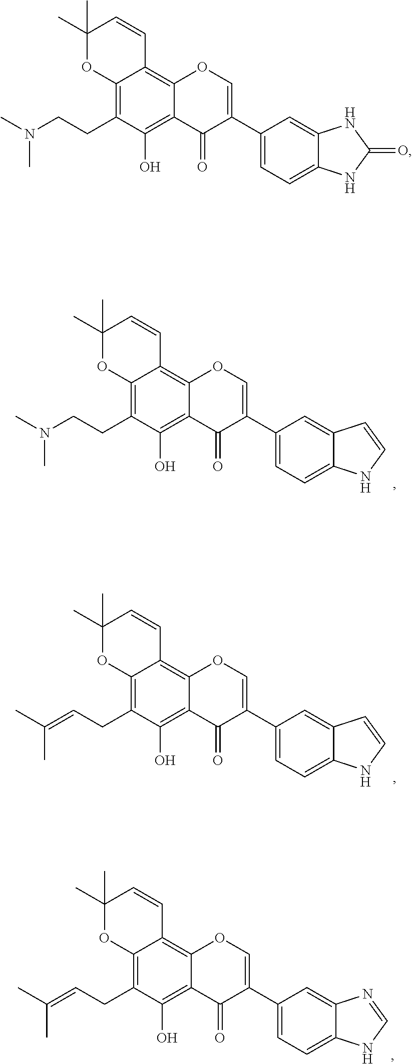 [SMILES: CN(C)CCc1c2c(c3occ(-c4ccc5[nH]c(=O)[nH]c5c4)c(=O)c3c1O)C=CC(C)(C)O2 ; CN(C)CCc1c2c(c3occ(-c4ccc5[nH]ccc5c4)c(=O)c3c1O)C=CC(C)(C)O2 ; CC(C)=CCc1c2c(c3occ(-c4ccc5[nH]ccc5c4)c(=O)c3c1O)C=CC(C)(C)O2 ; CC(C)=CCc1c2c(c3occ(-c4ccc5[nH]cnc5c4)c(=O)c3c1O)C=CC(C)(C)O2]

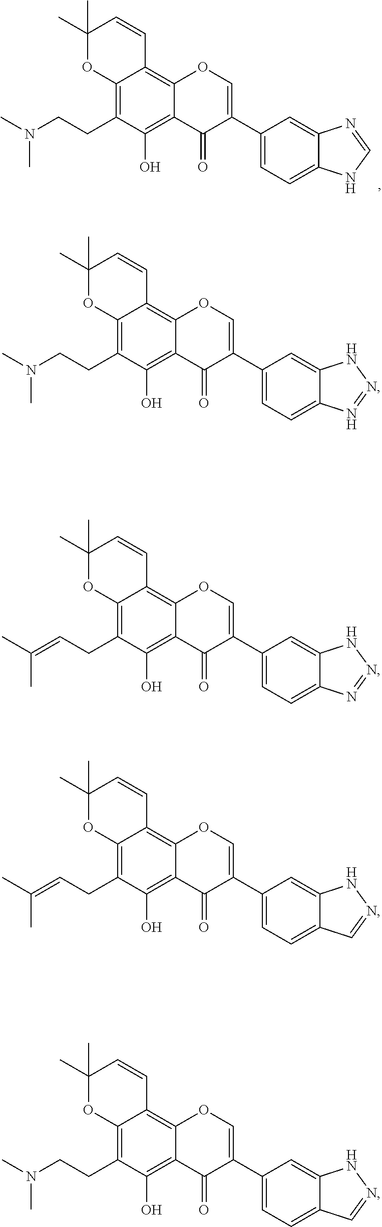 [SMILES: CN(C)CCc1c2c(c3occ(-c4ccc5[nH]cnc5c4)c(=O)c3c1O)C=CC(C)(C)O2 ; CN(C)CCc1c2c(c3occ(-c4ccc5nn[nH]c5c4)c(=O)c3c1O)C=CC(C)(C)O2 ; CC(C)=CCc1c2c(c3occ(-c4ccc5nn[nH]c5c4)c(=O)c3c1O)C=CC(C)(C)O2 ; CC(C)=CCc1c2c(c3occ(-c4ccc5cn[nH]c5c4)c(=O)c3c1O)C=CC(C)(C)O2 ; CN(C)CCc1c2c(c3occ(-c4ccc5cn[nH]c5c4)c(=O)c3c1O)C=CC(C)(C)O2]

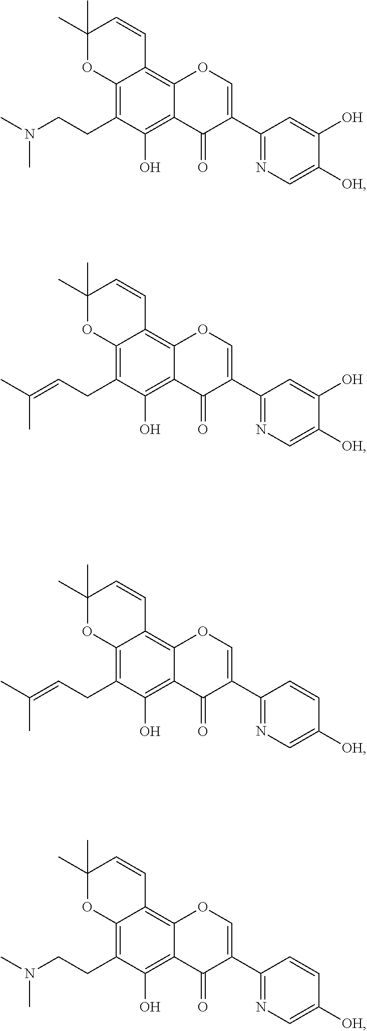 [SMILES: CN(C)CCc1c2c(c3occ(-c4cc(O)c(O)cn4)c(=O)c3c1O)C=CC(C)(C)O2 ; CC(C)=CCc1c2c(c3occ(-c4cc(O)c(O)cn4)c(=O)c3c1O)C=CC(C)(C)O2 ; CC(C)=CCc1c2c(c3occ(-c4ccc(O)cn4)c(=O)c3c1O)C=CC(C)(C)O2 ; CN(C)CCc1c2c(c3occ(-c4ccc(O)cn4)c(=O)c3c1O)C=CC(C)(C)O2]

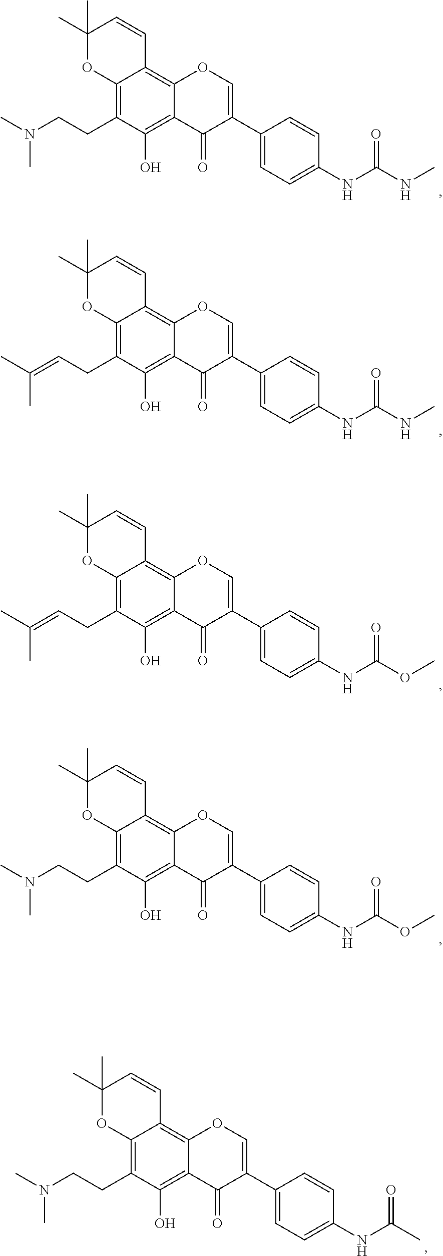

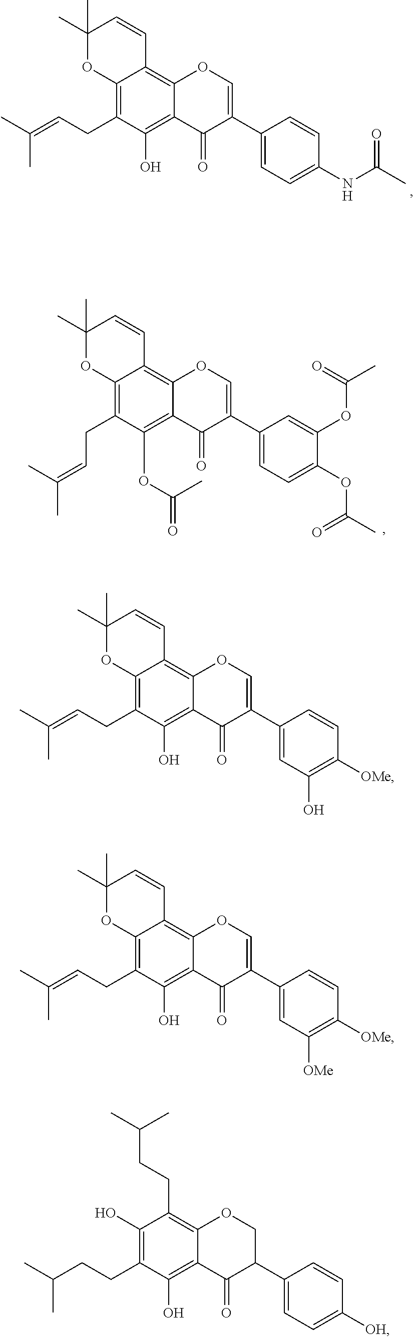

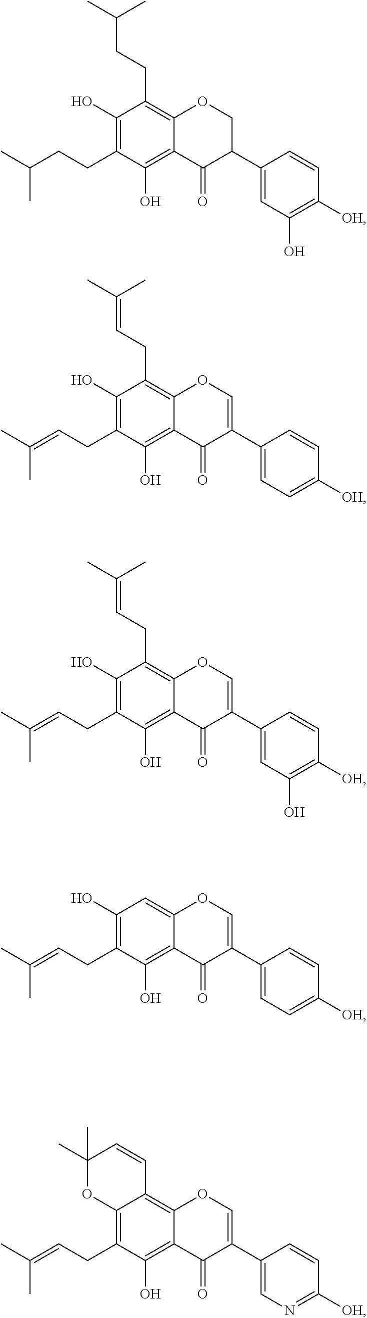 [SMILES: CC(C)CCc1c(O)c(CCC(C)C)c2c(c1O)C(=O)C(c1ccc(O)c(O)c1)CO2 ; CC(C)=CCc1c(O)c(CC=C(C)C)c2occ(-c3ccc(O)cc3)c(=O)c2c1O ; CC(C)=CCc1c(O)c(CC=C(C)C)c2occ(-c3ccc(O)c(O)c3)c(=O)c2c1O ; CC(C)=CCc1c(O)cc2occ(-c3ccc(O)cc3)c(=O)c2c1O ; CC(C)=CCc1c2c(c3occ(-c4ccc(O)nc4)c(=O)c3c1O)C=CC(C)(C)O2]

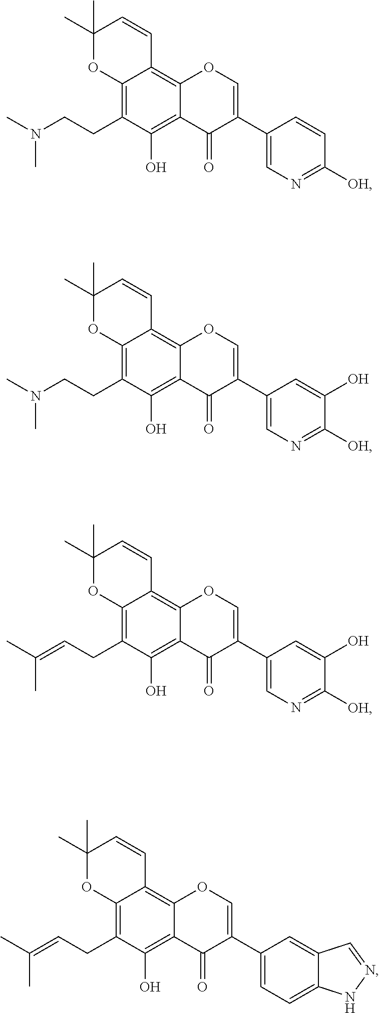 [SMILES: CN(C)CCc1c2c(c3occ(-c4ccc(O)nc4)c(=O)c3c1O)C=CC(C)(C)O2 ; CN(C)CCc1c2c(c3occ(-c4cnc(O)c(O)c4)c(=O)c3c1O)C=CC(C)(C)O2 ; CC(C)=CCc1c2c(c3occ(-c4cnc(O)c(O)c4)c(=O)c3c1O)C=CC(C)(C)O2 ; CC(C)=CCc1c2c(c3occ(-c4ccc5[nH]ncc5c4)c(=O)c3c1O)C=CC(C)(C)O2]

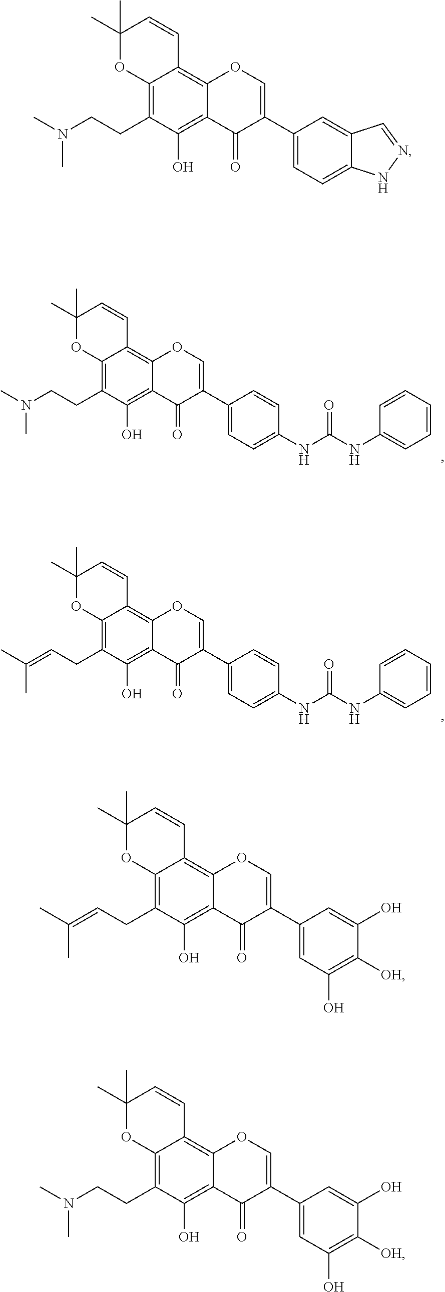 [SMILES: CN(C)CCc1c2c(c3occ(-c4ccc5[nH]ncc5c4)c(=O)c3c1O)C=CC(C)(C)O2 ; CN(C)CCc1c2c(c3occ(-c4ccc(NC(=O)Nc5ccccc5)cc4)c(=O)c3c1O)C=CC(C)(C)O2 ; CC(C)=CCc1c2c(c3occ(-c4ccc(NC(=O)Nc5ccccc5)cc4)c(=O)c3c1O)C=CC(C)(C)O2 ; CC(C)=CCc1c2c(c3occ(-c4cc(O)c(O)c(O)c4)c(=O)c3c1O)C=CC(C)(C)O2 ; CN(C)CCc1c2c(c3occ(-c4cc(O)c(O)c(O)c4)c(=O)c3c1O)C=CC(C)(C)O2]

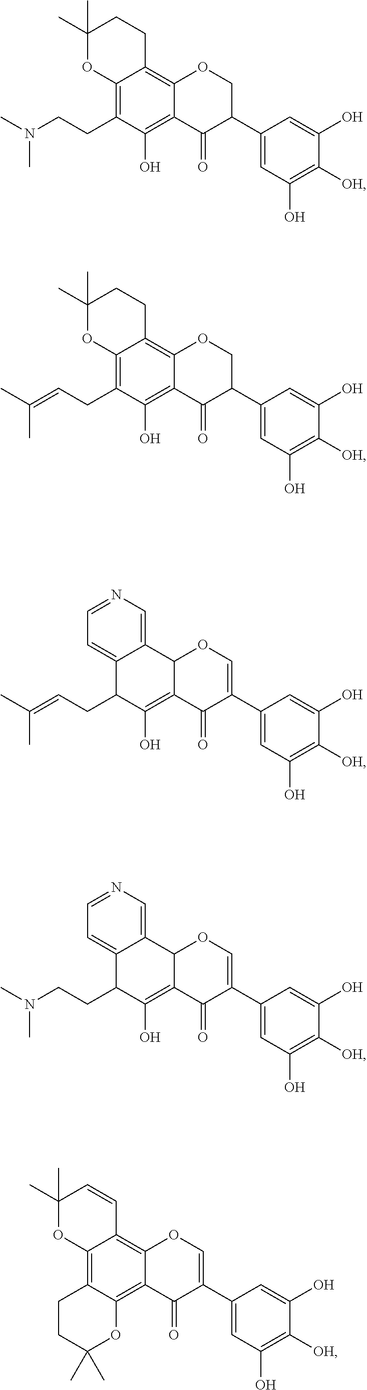 [SMILES: CN(C)CCc1c(O)c2c(c3c1OC(C)(C)CC3)OCC(c1cc(O)c(O)c(O)c1)C2=O ; CC(C)=CCc1c(O)c2c(c3c1OC(C)(C)CC3)OCC(c1cc(O)c(O)c(O)c1)C2=O ; CC(C)=CCC1C(O)=C2C(=O)C(c3cc(O)c(O)c(O)c3)=COC2c2cnccc21 ; CN(C)CCC1C(O)=C2C(=O)C(c3cc(O)c(O)c(O)c3)=COC2c2cnccc21 ; CC1(C)C=Cc2c(c3c(c4c(=O)c(-c5cc(O)c(O)c(O)c5)coc24)OC(C)(C)CC3)O1]

and combinations thereof,
or an N-oxide, crystalline form, hydrate thereof, or a pharmaceutically acceptable salt thereof.

As used herein, a “pharmaceutically acceptable salt” means a salt of the compounds of the present disclosure which are pharmaceutically acceptable, as defined herein, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

In the present disclosure, an “effective amount” or “therapeutically effective amount” of a compound or pharmaceutical composition is an amount of such a compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a compound or pharmaceutical composition according to the disclosure will be that amount of the compound or composition, which is the lowest dose effective to produce the desired effect with no or minimal side effects. The effective dose of a compound or pharmaceutical composition according to the present disclosure may be administered as two, three, four, five, six or more sub-doses, administered separately at appropriate intervals throughout the day.

Pharmaceutically acceptable carriers and diluents are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, salicylate, etc. Each pharmaceutically acceptable carrier or diluent used in a composition of the disclosure must be “acceptable” in the sense of being compatible with the other ingredients of the formulation and not injurious to the subject. Carriers or diluents suitable for a selected dosage form and intended route of administration are well known in the art, and acceptable carriers or diluents for a chosen dosage form and method of administration can be determined using ordinary skill in the art.

A further embodiment of the present disclosure is a kit. This kit comprises a compound or a pharmaceutical composition according to the present disclosure with instructions for the use of the compound or the pharmaceutical composition, respectively.

Another embodiment of the present disclosure is a method for treating or ameliorating the effects of a cancer in a subject. This method comprises administering to the subject a therapeutically effective amount of a compound having the structure of formula (I):

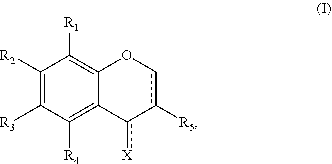

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄alkyl, CF₃, and combinations thereof, or an N-oxide, crystalline form, hydrate thereof, or a pharmaceutically acceptable salt thereof.

In some embodiments, the compound used in the methods disclosed herein is selected from the group consisting of:

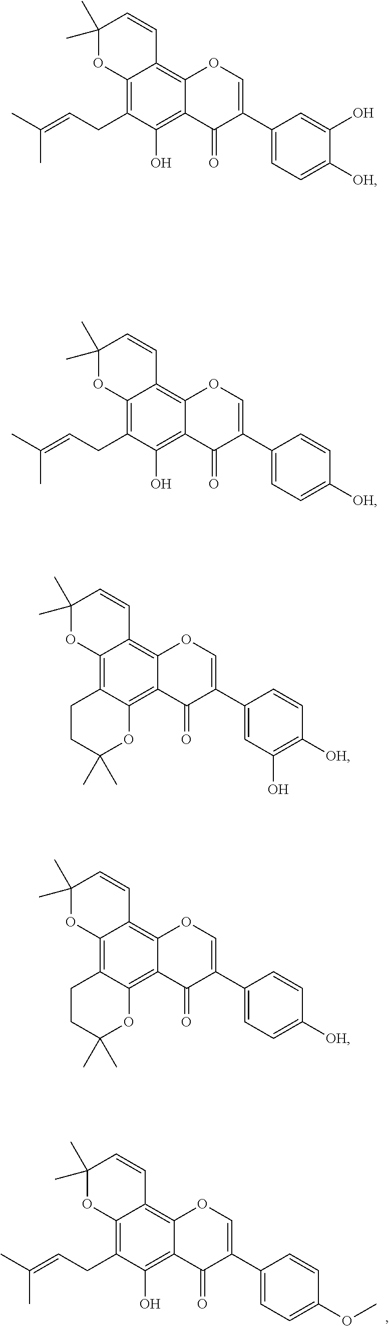 [SMILES: CC(C)=CCc1c2c(c3occ(-c4ccc(O)c(O)c4)c(=O)c3c1O)C=CC(C)(C)O2 ; CC(C)=CCc1c2c(c3occ(-c4ccc(O)cc4)c(=O)c3c1O)C=CC(C)(C)O2 ; CC1(C)C=Cc2c(c3c(c4c(=O)c(-c5ccc(O)c(O)c5)coc24)OC(C)(C)CC3)O1 ; CC1(C)C=Cc2c(c3c(c4c(=O)c(-c5ccc(O)cc5)coc24)OC(C)(C)CC3)O1 ; COc1ccc(-c2coc3c4c(c(CC=C(C)C)c(O)c3c2=O)OC(C)(C)C=C4)cc1]

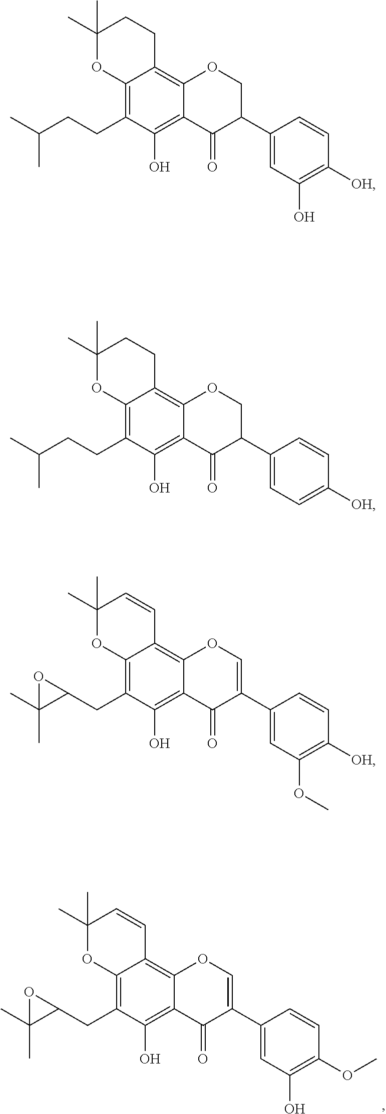

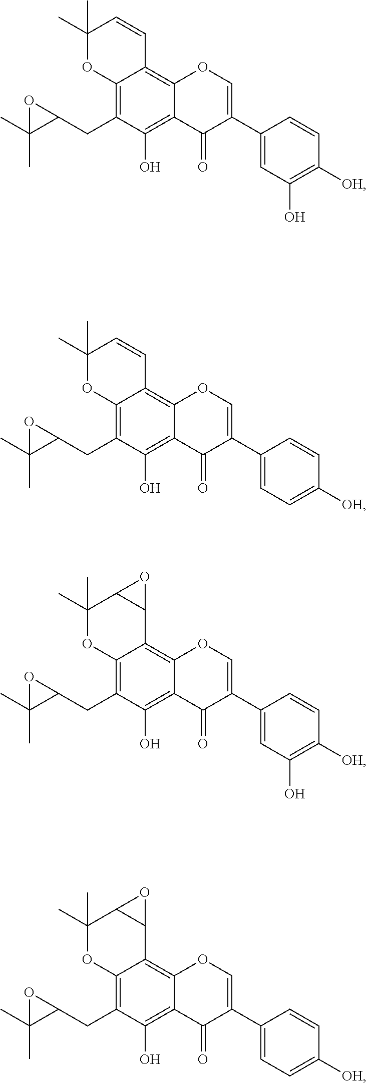 [SMILES: CC1(C)C=Cc2c(c(CC3OC3(C)C)c(O)c3c(=O)c(-c4ccc(O)c(O)c4)coc23)O1 ; CC1(C)C=Cc2c(c(CC3OC3(C)C)c(O)c3c(=O)c(-c4ccc(O)cc4)coc23)O1 ; CC1(C)OC1Cc1c2c(c3occ(-c4ccc(O)c(O)c4)c(=O)c3c1O)C1OC1C(C)(C)O2 ; CC1(C)OC1Cc1c2c(c3occ(-c4ccc(O)cc4)c(=O)c3c1O)C1OC1C(C)(C)O2]

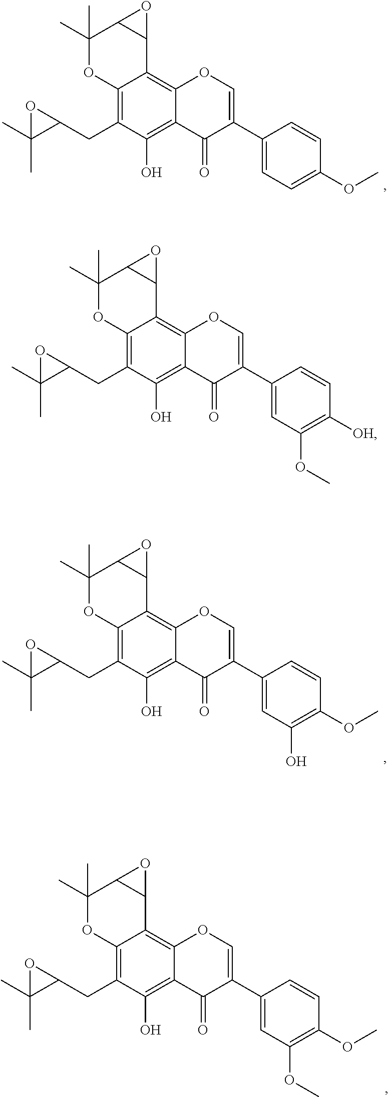

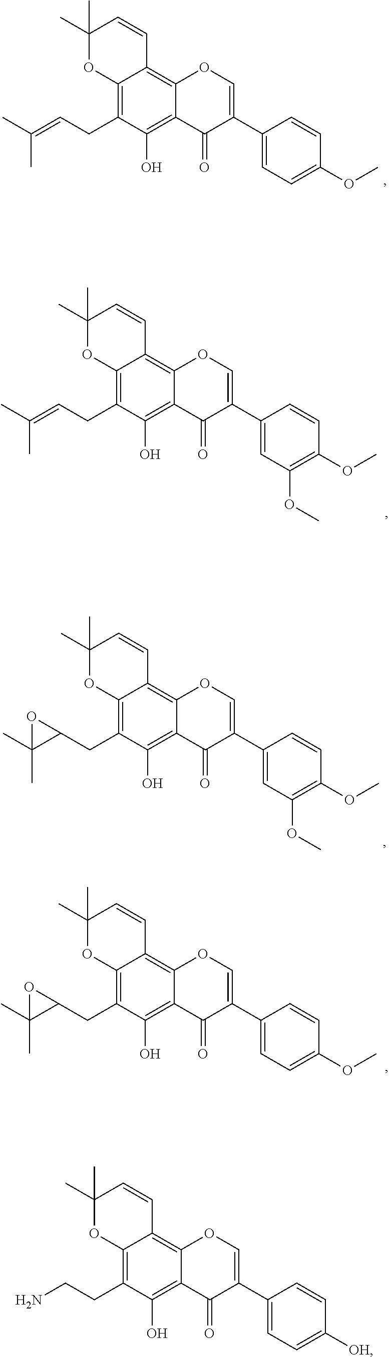

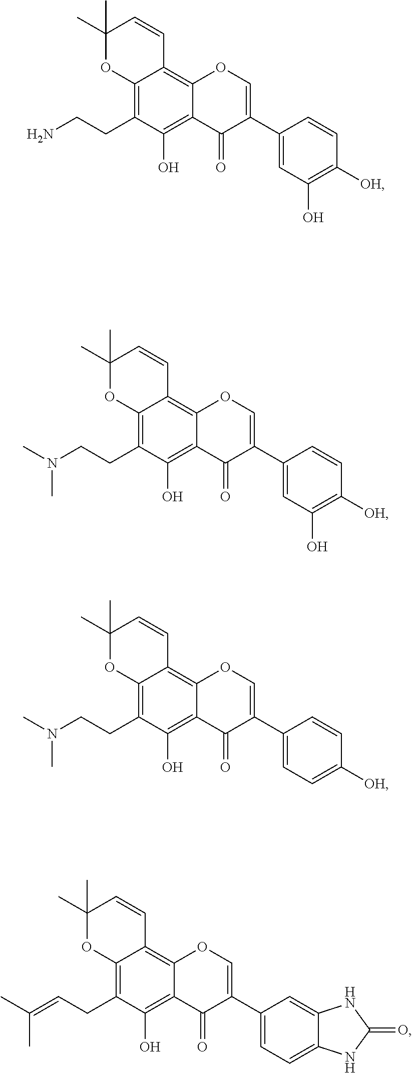 [SMILES: CC1(C)C=Cc2c(c(CCN)c(O)c3c(=O)c(-c4ccc(O)c(O)c4)coc23)O1 ; CN(C)CCc1c2c(c3occ(-c4ccc(O)c(O)c4)c(=O)c3c1O)C=CC(C)(C)O2 ; CN(C)CCc1c2c(c3occ(-c4ccc(O)cc4)c(=O)c3c1O)C=CC(C)(C)O2 ; CC(C)=CCc1c2c(c3occ(-c4ccc5[nH]c(=O)[nH]c5c4)c(=O)c3c1O)C=CC(C)(C)O2]

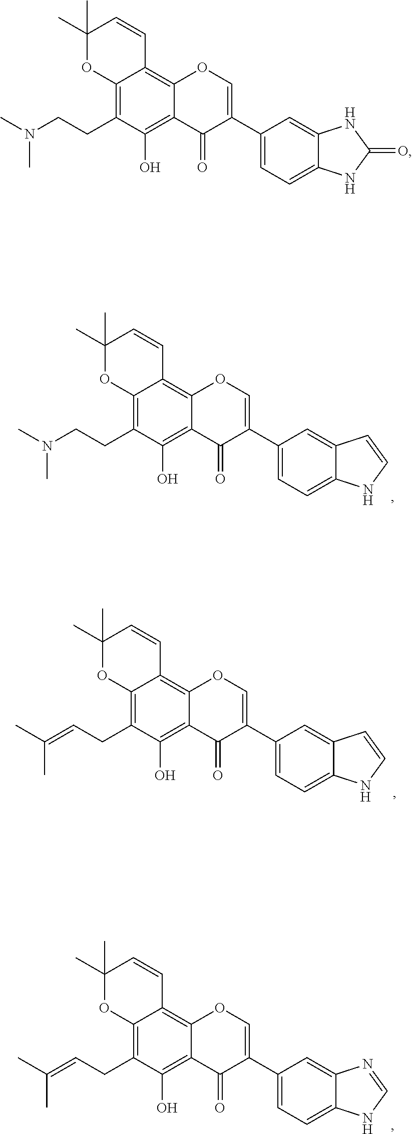 [SMILES: CN(C)CCc1c2c(c3occ(-c4ccc5[nH]c(=O)[nH]c5c4)c(=O)c3c1O)C=CC(C)(C)O2 ; CN(C)CCc1c2c(c3occ(-c4ccc5[nH]ccc5c4)c(=O)c3c1O)C=CC(C)(C)O2 ; CC(C)=CCc1c2c(c3occ(-c4ccc5[nH]ccc5c4)c(=O)c3c1O)C=CC(C)(C)O2 ; CC(C)=CCc1c2c(c3occ(-c4ccc5[nH]cnc5c4)c(=O)c3c1O)C=CC(C)(C)O2]

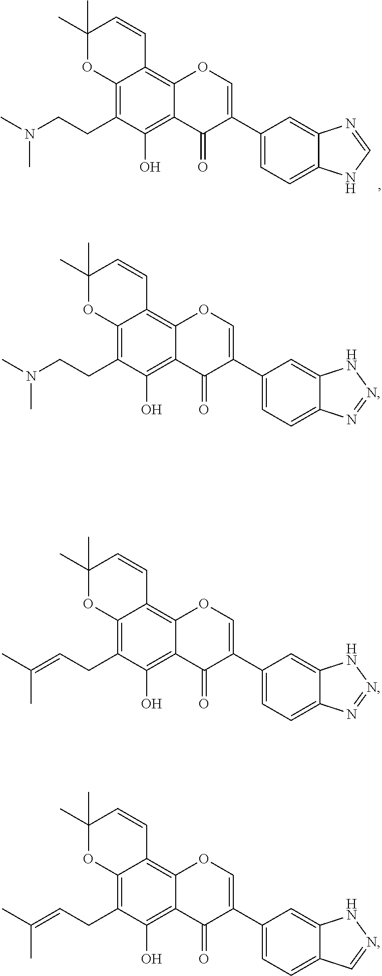 [SMILES: CN(C)CCc1c2c(c3occ(-c4ccc5[nH]cnc5c4)c(=O)c3c1O)C=CC(C)(C)O2 ; CN(C)CCc1c2c(c3occ(-c4ccc5nn[nH]c5c4)c(=O)c3c1O)C=CC(C)(C)O2 ; CC(C)=CCc1c2c(c3occ(-c4ccc5nn[nH]c5c4)c(=O)c3c1O)C=CC(C)(C)O2 ; CC(C)=CCc1c2c(c3occ(-c4ccc5cn[nH]c5c4)c(=O)c3c1O)C=CC(C)(C)O2]

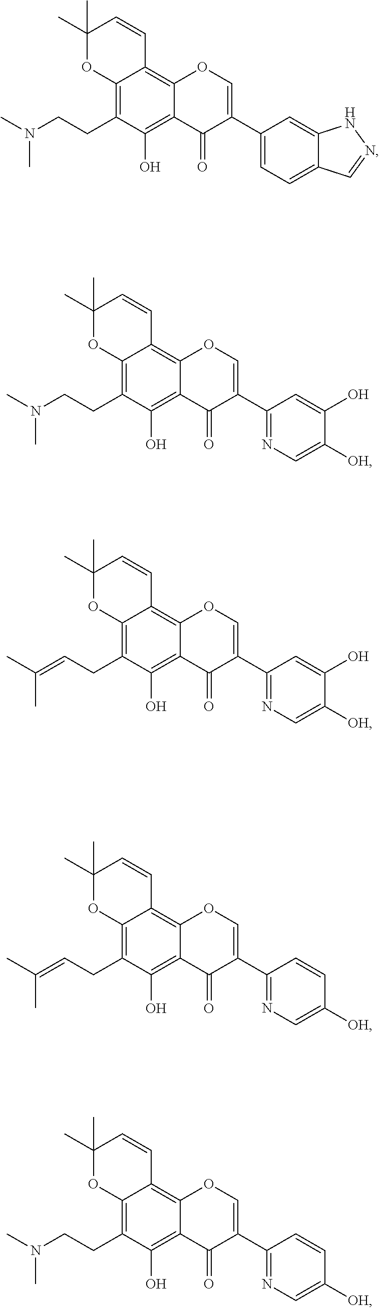 [SMILES: CN(C)CCc1c2c(c3occ(-c4ccc5cn[nH]c5c4)c(=O)c3c1O)C=CC(C)(C)O2 ; CN(C)CCc1c2c(c3occ(-c4cc(O)c(O)cn4)c(=O)c3c1O)C=CC(C)(C)O2 ; CC(C)=CCc1c2c(c3occ(-c4cc(O)c(O)cn4)c(=O)c3c1O)C=CC(C)(C)O2 ; CC(C)=CCc1c2c(c3occ(-c4ccc(O)cn4)c(=O)c3c1O)C=CC(C)(C)O2 ; CN(C)CCc1c2c(c3occ(-c4ccc(O)cn4)c(=O)c3c1O)C=CC(C)(C)O2]

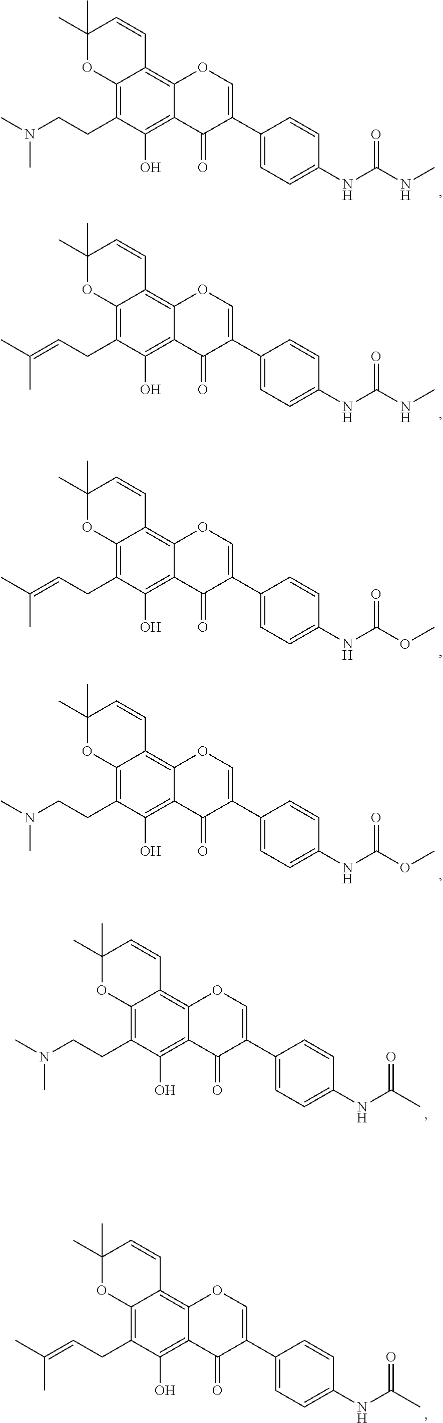

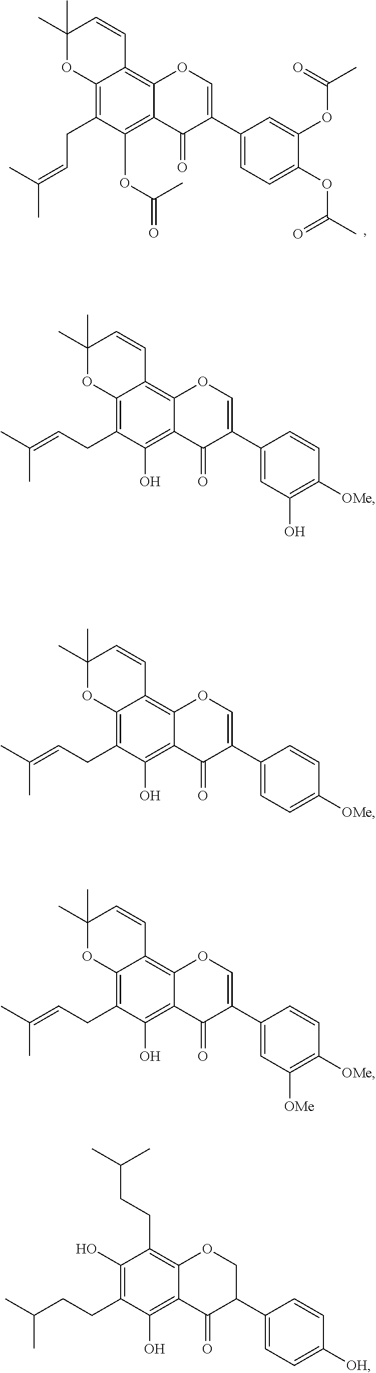

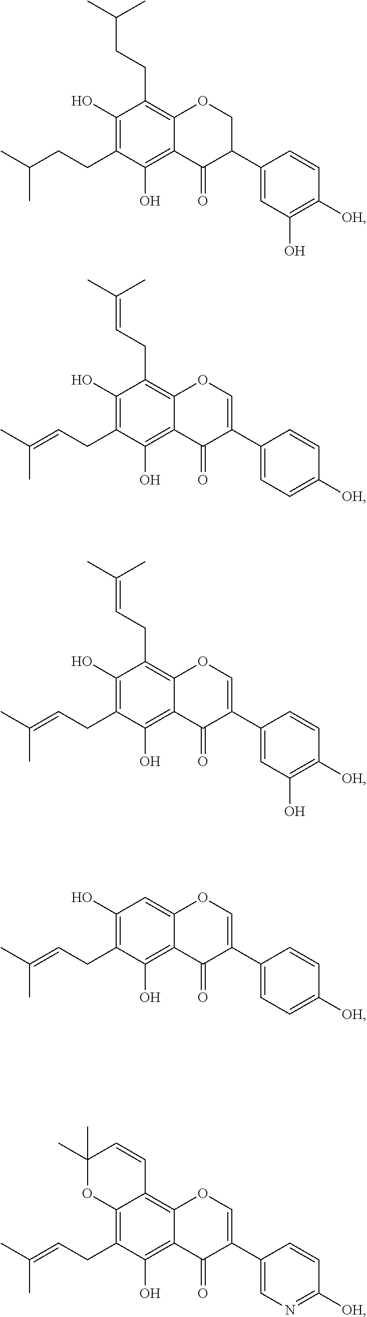 [SMILES: CC(C)CCc1c(O)c(CCC(C)C)c2c(c1O)C(=O)C(c1ccc(O)c(O)c1)CO2 ; CC(C)=CCc1c(O)c(CC=C(C)C)c2occ(-c3ccc(O)cc3)c(=O)c2c1O ; CC(C)=CCc1c(O)c(CC=C(C)C)c2occ(-c3ccc(O)c(O)c3)c(=O)c2c1O ; CC(C)=CCc1c(O)cc2occ(-c3ccc(O)cc3)c(=O)c2c1O ; CC(C)=CCc1c2c(c3occ(-c4ccc(O)nc4)c(=O)c3c1O)C=CC(C)(C)O2]

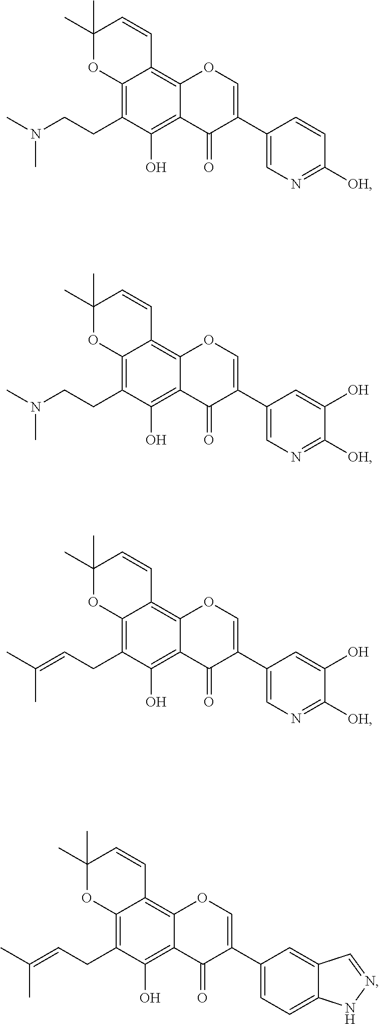 [SMILES: CN(C)CCc1c2c(c3occ(-c4ccc(O)nc4)c(=O)c3c1O)C=CC(C)(C)O2 ; CN(C)CCc1c2c(c3occ(-c4cnc(O)c(O)c4)c(=O)c3c1O)C=CC(C)(C)O2 ; CC(C)=CCc1c2c(c3occ(-c4cnc(O)c(O)c4)c(=O)c3c1O)C=CC(C)(C)O2 ; CC(C)=CCc1c2c(c3occ(-c4ccc5[nH]ncc5c4)c(=O)c3c1O)C=CC(C)(C)O2]

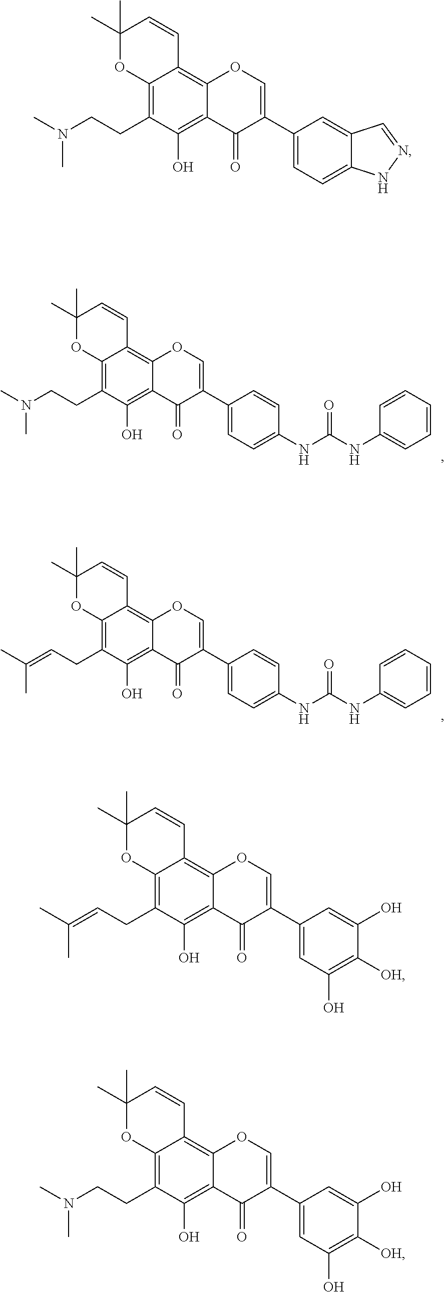 [SMILES: CN(C)CCc1c2c(c3occ(-c4ccc5[nH]ncc5c4)c(=O)c3c1O)C=CC(C)(C)O2 ; CN(C)CCc1c2c(c3occ(-c4ccc(NC(=O)Nc5ccccc5)cc4)c(=O)c3c1O)C=CC(C)(C)O2 ; CC(C)=CCc1c2c(c3occ(-c4ccc(NC(=O)Nc5ccccc5)cc4)c(=O)c3c1O)C=CC(C)(C)O2 ; CC(C)=CCc1c2c(c3occ(-c4cc(O)c(O)c(O)c4)c(=O)c3c1O)C=CC(C)(C)O2 ; CN(C)CCc1c2c(c3occ(-c4cc(O)c(O)c(O)c4)c(=O)c3c1O)C=CC(C)(C)O2]

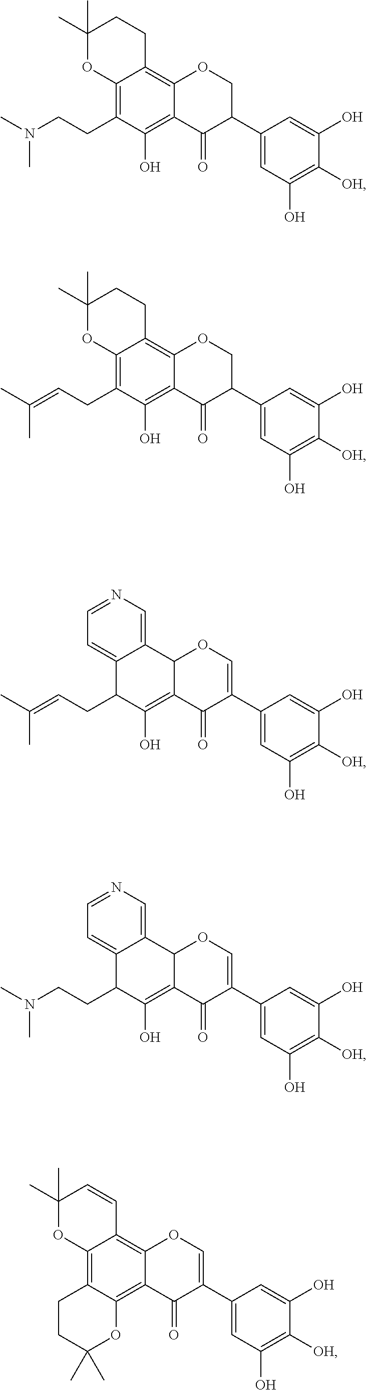 [SMILES: CN(C)CCc1c(O)c2c(c3c1OC(C)(C)CC3)OCC(c1cc(O)c(O)c(O)c1)C2=O ; CC(C)=CCc1c(O)c2c(c3c1OC(C)(C)CC3)OCC(c1cc(O)c(O)c(O)c1)C2=O ; CC(C)=CCC1C(O)=C2C(=O)C(c3cc(O)c(O)c(O)c3)=COC2c2cnccc21 ; CN(C)CCC1C(O)=C2C(=O)C(c3cc(O)c(O)c(O)c3)=COC2c2cnccc21 ; CC1(C)C=Cc2c(c3c(c4c(=O)c(-c5cc(O)c(O)c(O)c5)coc24)OC(C)(C)CC3)O1]

and combinations thereof,
or an N-oxide, crystalline form, hydrate thereof, or a pharmaceutically acceptable salt thereof.

In some embodiments, the compound used in the methods disclosed herein is selected from the group consisting of:

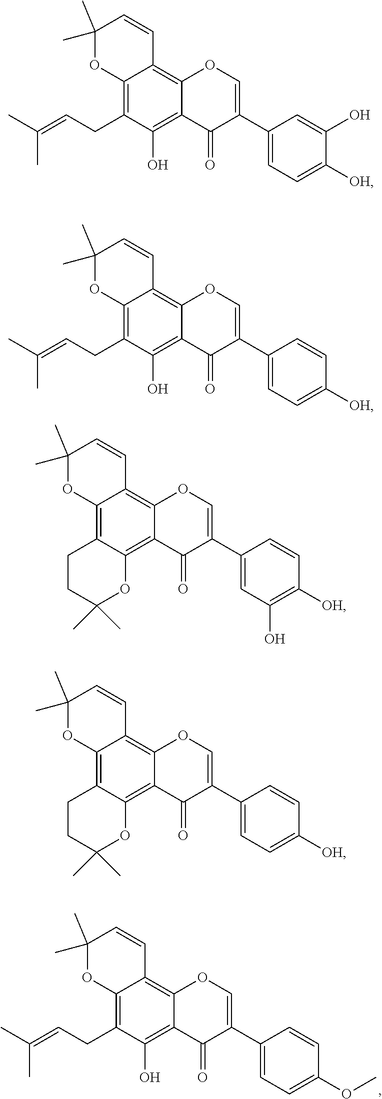

and combinations thereof,
or an N-oxide, crystalline form, hydrate thereof, or a pharmaceutically acceptable salt thereof.

In some embodiments, the compound used in the methods disclosed herein is:

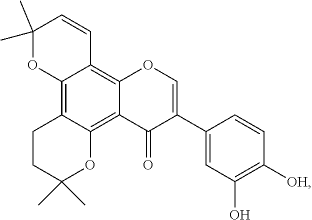 [SMILES: CC1(C)C=Cc2c(c3c(c4c(=O)c(-c5ccc(O)c(O)c5)coc24)OC(C)(C)CC3)O1]

or an N-oxide, crystalline form, hydrate thereof, or a pharmaceutically acceptable salt thereof.

Another embodiment of the present disclosure is a method for treating or ameliorating the effects of a cancer in a subject. This method comprises administering to the subject a therapeutically effective amount of a compound selected from the group consisting of mycophenolate, NSC 80997, podofilox, cloxyquin, NSC 305798, NSC 255109, narasin, methylene blue, azure A, azure B, rapamycin, NSC 3905, and combinations thereof, or an N-oxide, crystalline form, hydrate thereof, or a pharmaceutically acceptable salt thereof. In some embodiments, the method disclosed herein further comprises co-administering to the subject an effective amount of an aurora A kinase inhibitor such as, for example, alisertib (MLN8237).

Non-limiting examples of cancers according to the present disclosure include glioma, thyroid cancer, lung cancer, liver cancer, pancreatic cancer, head and neck cancer, stomach cancer, colorectal cancer, urothelial cancer, renal cancer, prostate cancer, testis cancer, breast cancer, cervical cancer, ovarian cancer, endometrial cancer, melanoma, lymphoma, acute myeloid leukemia (AML), neuroblastoma, medulloblastoma, retinoblastoma, astrocytoma, glioblastoma multiforme, castration-resistant prostate cancer (CRPC), neuroendocrine prostate cancer (NEPC), hematologic malignancies, rhabdomyosarcoma, Wilms tumors, non-small cell lung cancer (NSCLC) and small cell lung cancer (SCLC). In some embodiments, the cancer is driven by MycN and/or cMyc. In some embodiments, the cancer is MycN-amplified neuroblastoma (MycN^{AMP} NBL).

In some embodiments, the methods disclosed herein further comprise co-administering to the subject a chemotherapy drug selected from the group consisting of cisplatin, temozolomide, doxorubicin, cyclophosphamide, methotrexate, 5-fluorouracil, vinorelbine, docetaxel, bleomycin, vinblastine, dacarbazine, mustine, vincristine, procarbazine, prednisolone, etoposide, epirubicin, capecitabine, methotrexate, folinic acid, oxaliplatin, and combinations thereof. In some embodiments, the methods disclosed above further comprising co-administering radiotherapy to the subject.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments, the subject is a pediatric patient.

Another embodiment of the present disclosure is a method for selectively killing a cancer cell. This method comprises contacting the cancer cell with an effective amount of a compound having the structure of formula (I):

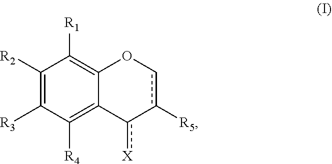

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof, or an N-oxide, crystalline form, hydrate thereof, or a pharmaceutically acceptable salt thereof.

In some embodiments, the cancer cell overexpresses MycN and/or cMyc.

Another embodiment of the present disclosure is a method of modulating mTORC1/2 signaling activity in a cell. The method comprises contacting the cell with an effective amount of a compound having the structure of formula (I):

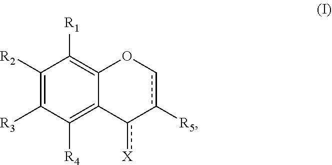

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO₂, SO₂N(R), S, SO, SO₂, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄alkyl, CF₃, and combinations thereof, or an N-oxide, crystalline form, hydrate thereof, or a pharmaceutically acceptable salt thereof.

As used herein, the terms “modulate”, “modulating”, “modulator” and grammatical variations thereof mean to change, such as decreasing or reducing mTORC1/2 signaling activity in a cell. In the present disclosure, “contacting” means bringing the compound and optionally one or more additional therapeutic agents into close proximity to the cells in need of such modulation. This may be accomplished using conventional techniques of drug delivery to the subject or in the in vitro situation by, e.g., providing the compound and optionally other therapeutic agents to a culture media in which the cells are located.

Another embodiment of the present disclosure is a method of modulating the activity of a Master Regulator for MycN in a subject having MycN-amplified neuroblastoma (MycN^{AMP} NBL). This method comprises administering to the subject a therapeutically effective amount of a compound having the structure of formula (I):

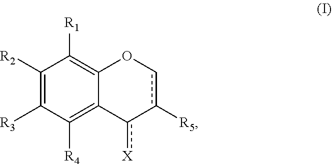

wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof, or an N-oxide, crystalline form, hydrate thereof, or a pharmaceutically acceptable salt thereof.

Another embodiment of the present disclosure is a method of modulating the activity of a Master Regulator for MycN in a subject having MycN-amplified neuroblastoma (MycN^{AMP} NBL). This method comprises administering to the subject a therapeutically effective amount of a compound selected from the group consisting of mycophenolate, NSC 80997, podofilox, cloxyquin, NSC 305798, NSC 255109, narasin, methylene blue, azure A, azure B, rapamycin, NSC 3905, and combinations thereof, or an N-oxide, crystalline form, hydrate thereof, or a pharmaceutically acceptable salt thereof.

In some embodiments, the modulation comprises reversing the NBL master regulatory activity for cMyc in the subject.

As used herein, “master regulators” or “MYC Master Regulators (MRs)” are transcriptional regulators, and their upstream signaling networks work coordinately to establish and maintain the aggressive phenotype of MYC-driven tumors. MRs are identified by analysis of gene regulatory and signaling networks.

Yet another embodiment of the present disclosure is a method of selectively treating or ameliorating effects of a cancer in a subject in need thereof. This method comprises the steps of: (a) obtaining a biological sample from the subject; (b) determining the expression level of MycN in the sample and comparing it with a predetermined reference; (c) identifying the subject as a MycN^{AMP} subtype if MycN in the sample is determined to be overexpressed in step (b); and (d) treating the MycN^{AMP} subtype subject with a therapeutically effective amount of a compound or a pharmaceutical composition disclosed herein.

Another embodiment of the present disclosure is a method of selectively treating or ameliorating effects of a cancer in a subject in need thereof. This method comprises the steps of: (a) obtaining a biological sample from the subject; (b) determining the expression level of cMyc in the sample and comparing it with a predetermined reference; (c) identifying the subject as a cMyc^{AMP} subtype if cMyc in the sample is determined to be overexpressed in step (b); and (d) treating the cMyc^{AMP} subtype subject with a therapeutically effective amount of a compound or a pharmaceutical composition disclosed herein.

Still another embodiment of the present disclosure is a method for identifying a compound that induces degradation of a cancer-related protein. This method comprises the steps of: (a) obtaining cancer cell lines that express the protein (AMP cell lines) and cancer cell lines that do not express the protein (NULL cell lines); (b) identifying compounds that are lethal to at least one of the cell lines; (c) identifying compounds that are selective for AMP cell lines from those identified in step (b) based on cell line subtype selectivity; (d) determining the expression level of the protein in AMP cell lines for each selective compound identified in step (c) by performing a high-throughput gene expression profiling; and (e) identifying a candidate compound that induces degradation of the cancer-related protein based on the result of step (d). In some embodiments, the cancer-related protein is MycN or cMyc. In some embodiments, the gene expression profiling in step (d) is performed by PLATE-Seq.

Another embodiment of the present disclosure is a compound having the structure of

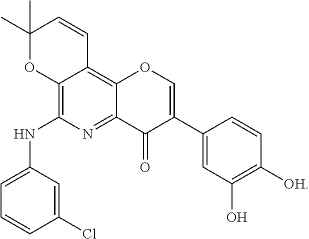 [SMILES: CC1(C)C=Cc2c(c(Nc3cccc(Cl)c3)nc3c(=O)c(-c4ccc(O)c(O)c4)coc23)O1]

In the present disclosure, the following definitions apply:

The term “aliphatic”, as used herein, refers to a group composed of carbon and hydrogen that do not contain aromatic rings. Accordingly, aliphatic groups include alkyl, alkenyl, alkynyl, and carbocyclyl groups. Additionally, unless otherwise indicated, the term “aliphatic” is intended to include both “unsubstituted aliphatics” and “substituted aliphatics”, the latter of which refers to aliphatic moieties having substituents replacing a hydrogen on one or more carbons of the aliphatic group. Such substituents can include, for example, a halogen, a deuterium, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic, or heteroaromatic moiety.

The term “alkyl” refers to the radical of saturated aliphatic groups that does not have a ring structure, including straight-chain alkyl groups, and branched-chain alkyl groups. In certain embodiments, a straight chain or branched chain alkyl has 6 or fewer carbon atoms in its backbone (e.g., C1-C6 for straight chains, C3-C6 for branched chains). Such substituents include all those contemplated for aliphatic groups, as discussed below, except where stability is prohibitive.

The term “alkenyl”, as used herein, refers to an aliphatic group containing at least one double bond and unless otherwise indicated, is intended to include both “unsubstituted alkenyls” and “substituted alkenyls”, the latter of which refers to alkenyl moieties having substituents replacing a hydrogen on one or more carbons of the alkenyl group. Such substituents include all those contemplated for aliphatic groups, as discussed below, except where stability is prohibitive. For example, substitution of alkenyl groups by one or more alkyl, carbocyclyl, aryl, heterocyclyl, or heteroaryl groups is contemplated.

Moreover, unless otherwise indicated, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Indeed, unless otherwise indicated, all groups recited herein are intended to include both substituted and unsubstituted options.

The term “C_{x-y}” when used in conjunction with a chemical moiety, such as, alkyl and cycloalkyl, is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The term “aryl” as used herein includes substituted or unsubstituted single-ring aromatic groups in which each atom of the ring is carbon. Preferably the ring is a 3- to 8-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “alkyl-aryl” refers to an alkyl group substituted with at least one aryl group.

The term “alkyl-heteroaryl” refers to an alkyl group substituted with at least one heteroaryl group.

The term “alkenyl-aryl” refers to an alkenyl group substituted with at least one aryl group.

The term “alkenyl-heteroaryl” refers to an alkenyl group substituted with at least one heteroaryl group.

The terms “carbocycle”, “carbocyclyl”, and “carbocyclic”, as used herein, refer to a non-aromatic saturated or unsaturated ring in which each atom of the ring is carbon. Preferably a carbocycle ring contains from 3 to 10 atoms, more preferably from 3 to 8 atoms, including 5 to 7 atoms, such as for example, 6 atoms. The term “carbocycle” also includes bicycles, tricycles and other multicyclic ring systems, including the adamantyl ring system.

The terms “halo” and “halogen” are used interchangeably herein and mean halogen and include chloro, fluoro, bromo, and iodo.

The term “heteroaryl” includes substituted or unsubstituted aromatic single ring structures, preferably 3- to 8-membered rings, more preferably 5- to 7-membered rings, even more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur; more preferably, nitrogen and oxygen.

The term “substituted” refers to moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As set forth previously, unless specifically stated as “unsubstituted,” references to chemical moieties herein are understood to include substituted variants. For example, reference to an “aryl” group or moiety implicitly includes both substituted and unsubstituted variants.

It is also understood that the disclosure of a compound herein encompasses all stereoisomers of that compound. As used herein, the term “stereoisomer” refers to a compound made up of the same atoms bonded by the same bonds but having different three-dimensional structures which are not interchangeable. The three-dimensional structures are called configurations. Stereoisomers include enantiomers and diastereomers.

The following examples are provided to further illustrate certain aspects of the present disclosure. These examples are illustrative only and are not intended to limit the scope of the disclosure in any way.

### EXAMPLES

### Example 1

### Materials and Methods

### Cell Culture, Chemical Screening and Cell Viability Assays

Neuroblastoma (SK-N-Be2; IMR-32; NLF; SK-N-AS) and lung cancer cell lines (NCI-H69; NCI-H526; NCI-H441-4) were grown in Advanced RPMI medium (Life Technologies) supplemented with 10% FBS (Gibco), 1% GlutaMAX (Gibco), and 1% Penicillin/Streptomycin. A549 cells (NSCLC) was grown in F-12K medium (Gibco) with 10% FBS (Gibco), and 1% Penicillin/Streptomycin. Cell cultures were incubated at 37° C. with 5% CO₂. For chemical screening, cells were trypsinized, counted and seeded into white opaque 384-well plates (Perkin Elmer) at a density of 1000 cells/well and incubated overnight. The following day, 384-well stock screening plates containing 10 mM compound dissolved in dimethyl sulfoxide (DMSO) were diluted to a concentration of 200 μM in daughter plates containing growth medium. From these plates, compounds were diluted 1/10 into the assay plates containing cells, resulting in a final assay concentration of 20 μM with DMSO at 0.2%. Cells were treated for 72 hrs, after which cell viability was determined using CellTiterGlo® luminescence assay, following the manufacturer's instructions (Promega). Three chemical libraries were screened for lethal activity: 727 compounds from the NIH Clinical Collection (National Institutes of Health), 2498 compounds from the NCI Diversity Set (National Cancer Institute) and 2400 compounds from the SPECTRUM Collection (MicroSource). Following the initial screen, compounds that were lethal to any of the four cell lines tested were rescreened across a series of concentrations ranging from 20 μM to 0.2 μM for 72 hrs, following which the cell viability was assayed using Cell Titer Glo. Cell viability data were analyzed and charted using PRISM v7.0 software (GraphPad). The dose-response curves were used to generate an inhibitory constant (IC₅₀) value, which was averaged across the two cell lines from each subtype (ie: SK-N-AS and NLF as MESN NBL subtype; IMR-32 and SK-N-Be2 as MycN^{AMP} subtype). The IC₅₀ values for each subtype were used to evaluate subtype selectivity of each compound, and identify compounds with enhanced potency in MycN^{AMP} NBL.

### PLATE-Seq

SK-N-Be2 cells were seeded in a 96 well plate at a density of 10 000 cells/well and incubated overnight. The following day, compounds were diluted from DMSO stock solutions to create a daughter plate with solutions of chemicals at 10× the final assay concentration, diluted in growth media. DMSO was added to each well to create equimolar DMSO across the plate at 0.1% final concentration. Duplicate plates were created for PLATE-Seq analysis by randomizing 90 lethal molecules across the plate, with the inclusion of six DMSO-only control wells. Following the addition of compounds to the final assay wells, the plates were incubated for 24 h. Following treatment, plates were rinsed twice with cold PBS, and 40 μL of Buffer SLC was added to the each well. Plates were frozen at −20° C. PLATE-Seq library prep, quality control analysis, and sequencing were performed at the JP Sulzberger genome center at Columbia University Medical Center (GUMC). PLATE-Seq data were analyzed by the virtual inference of protein activity by enriched regulon analysis (VIPER) and detecting mechanism of action by network dysregulation (DeMAND) algorithms.

Reads were mapped against the human reference genome version Grch38 using the STAR aligner, and used the variance stabilizing transformation from the DESeq2 package for R to normalize each plate. Next, we corrected for batch effect due to having compounds replicates on separate plates. We used the function combat from the sva package for R to compute the batch-corrected normalized gene expression, and fitted a linear model for each compound against the set of DMSO controls on each plate (6 wells per plate per DMSO-treated cells). We used the limma package for R to fit the linear model and to compute p-values and moderated t-statistics for each gene. For each compound, we used a vector of these statistics to generate a gene expression profile of z-scores representing the compound effect as differential between post- and pre-treatment.

Each compound gene expression profile was analyzed using the VIPER algorithm (Alvarez et al. 2016) with TARGET and NRC NBL interactomes, reverse-engineered as described in (Rajbhandari et al. 2018), resulting in two protein activity profiles for each compound that were integrated using Stouffer's z-score method. We ran the VIPER algorithm after having pruned each interactome by keeping for each protein regulon the top 100 targets with the highest likelihood, and excluding protein regulons with less than 30 targets, since fixing the number of targets makes more comparable the activity score of each protein within the same profile.

### VIKING Algorithm

The Virtual-Inference of Kinase INhibiton by druG (VIKING) algorithm consists in the following steps: 1) Inference of protein activity profiles of individual samples with VIPER, 2) Generation of high confidence PPI network with PrePPI, 3) Network perturbation analysis with DeMAND, 4) Filtering of top dysregulated kinases with negative NES as inferred by VIPER between post- and pre-treatment conditions.

We used ARACNe (Lachmann et al. 2016) to reverse-engineer a regulatory network based on recent RNA-Seq data of 157 tumors from NBL patients collected by the TARGET consortium. The resulting network consists in 2362 among TFs and co-TFs, and 3197 signaling molecules, yielding a total of 5,559 regulatory proteins, 21,664 targets and 1,889,970 interactions. This interactome was used to compute a sample-specific protein activity signature with the VIPER algorithm, for 6 isopomiferin-treated and 6 DMSO-treated samples collected at 24 hours. For this analysis, each regulon was pruned as described above.

We generated a PPI network from the PrePPI database (Zhang et al. 2012) by selecting for protein interactions having structural score above the median of the empirical distribution of scores present in the database. This list was further refined by filtering out proteins not known to have regulatory functions, keeping therefore Transcription Factors (TF), co-TF, and signaling proteins. These filters yield a PPI network of 131,258 high-confidence interactions. Next, the DeMAND algorithm was used to prioritize a list of 5,559 proteins based on the inferred dysregulation of a protein and all its predicted interaction partners between the set of treatment samples and the controls. The resulting list of MoA proteins was filtered accordingly several criteria, including a Bonferroni corrected p-value lesser than 0.01 for DeMAND scores, NES score lesser than −1.965 for VIPER scores, and filtering out proteins that are not kinases. We used annotation for 514 human kinases as described in Manning et al. 2002.

### RT-qPCR

Reverse transcription quantitative polymerase chain reaction (RT-qPCR) was used to quantify transcript abundance of genes of interest. Total RNA was isolated from cells using the Qiaquick RNeasy isolation kit following manufacturer's instruction (QIAGEN). RNA quality and abundance was measured using a nanodrop spectrophotometer. A total of 2 μg RNA was used as a template for reverse transcription reactions. cDNA synthesis was performed using TaqMan reverse transcriptase following the manufacturer's instructions, using both oligod(T) and random hexamers as primers for RT reactions. RNAse treatment of cDNA removed any residual RNA in cDNA samples. cDNA samples were diluted 10-fold into nuclease-free dH₂O, and used as the template for quantitative PCR reactions were performed in a Viia7 (BIORAD) thermocycler using SYBR green and gene-specific primers (Table 1). qPCR reactions were performed using the Viia7 Real-Time PCR system (Applied Biosystems), and relative transcript abundance evaluated using the deltaCT method, using the GAPDH housekeeping gene as normalization control.

TABLE 1
Primers used in qPCR experiments.

| Primer Name | Sequence |
|---|---|
| GAPDH FW | 5′ CTCCAAAATCAAGTGGGGCG 3′ |
| | (SEQ ID No. 1) |
| GAPDH RV | 5′ ATGACGAACATGGGGGCATC 3′ |
| | (SEQ ID No. 2) |
| MYCN FW | 5′ GCACAGACTGTAGCCATCCG 3′ |
| | (SEQ ID No. 3) |
| MYCN RV | 5′ TTTAATACCGGGGGTGCTTCC 3′ |
| | (SEQ ID No. 4) |
| cMYC FW | 5′ TCAAGAGGTGCCACGTCTCC 3′ |
| | (SEQ ID No. 5) |
| cMYC RV | 5′ TCTTGGCAGCAGGATAGTCCTT 3′ |
| | (SEQ ID No. 6) |
| PLK1 FW | 5′ TATATCCCTGCCCGTCTCCCC 3′ |
| | (SEQ ID No. 7) |
| PLK1 RV | 5′ CCTCACCTGTCTCTCGAACC 3′ |
| | (SEQ ID No. 8) |
| LIN28b FW | 5′ CCTTGAGTCAATACGGGT 3′ |
| | (SEQ ID No. 9) |
| LIN28b RV | 5′ GCTCTGACAGTAATGGCA 3′ |
| | (SEQ ID No. 10) |
| RCOR2 FW | 5′ TCTGGGATCCTGTCCCGTAG 3′ |
| | (SEQ ID No. 11) |
| RCOR2 RV | 5′ GGTAATTGGTTCCAACGCGG 3′ |
| | (SEQ ID No. 12) |
| BMI1 FW | 5′ TGGCTCGCATTCATTTTCTG 3′ |
| | (SEQ ID No. 13) |
| BMI1 RV | 5′ AGTAGTGGTCTGGTCTTGTG 3′ |
| | (SEQ ID No. 14) |
| CCL5 FW | 5′ GAGTATTTCTACACCAGTGGCAAG 3′ |
| | (SEQ ID No. 15) |
| CCL5 RV | 5′ TCCCGAACCCATTTCTTCTCT 3′ |
| | (SEQ ID No. 16) |
| CSNK2A1 FW | 5′ CTTCTCAGGGGAGGCAGGA 3′ |
| | (SEQ ID No. 17) |
| CSNK2A1 RV | 5′ CACACTTCCACAAGAGCCACT 3′ |
| | (SEQ ID No. 18) |
| CSNK2A2 FW | 5′ CACTTTTCCATAAGCAGAACAAGA 3′ |
| | (SEQ ID No. 19) |
| CSNK2A2 RV | 5′ TACATTCGGAAGTGAGGTTTGATA 3′ |
| | (SEQ ID No. 20) |

### Quantification of Cellular Accumulation of Compounds

Cellular accumulation of CX-4945 and pomiferin was quantified by liquid chromatography-mass spectrometry (LC-MS) following previously published protocols (Welsch et al. 2017; Colletti et al. 2008). In brief, SK-N-Be2 cells were seeded at 400 k cells/well in 6 w plates and incubated overnight. The following day, compounds were added to wells at indicated concentrations, with DMSO-only control added to non-treated wells. Following treatment for the indicated time periods, cells were trypsinized, rinsed twice with cold PBS to remove media, pelleted by centrifugation, and frozen at −20° C. To extract compounds, frozen pellets were resuspended in 150 μL of acetonitrile, sonicated for 2′, and centrifuged at 1400×g for 75′ at 4° C. Supernatant was collected and analyzed by LC-MS using a system comprised of a Thermo Scientific Dionex Ultimate 3000 and a Bruker amaZon SL equipped with an electrospray ionization source controlled by a Bruker Hystar 3.2. Compounds were separated by injecting 20 μL of supernatant onto an Agilent Eclipse Plus C18 column (2.1×50 mm, 3.5 μM) maintained at 20° C., with the flow rate set at 400 μL/min. Initial flow conditions were 60% solvent A (MilliQ H₂O, 0.1% acetic acid), and 40% solvent B (HPLC-grade MeOH, 0.1% acetic acid). Solvent B was raised to 60% over 0.25 min and to 70% by 6.75 min. Solvent B was raised to 95% by 7 min and lowered back to 40% by 8 minutes; total run time was 9 min (Bos et al. 2019).

### Biochemical Kinase Assays

Cell-free biochemical kinase assays were performed at Reaction Biology Corporation (Malvern, Pa.). In brief, kinase substrates were diluted into reaction buffer containing 20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO. Purified protein (CK2a1, CK2a2, or mTOR) was added to the substrate solution and gently mixed. Test compounds were diluted from 10 mM DMSO stock solutions into the reaction buffer using an Echo550 acoustic dispenser, followed by incubation at RT for 20 min. 33P-ATP (10 μCi/μL) was added to reaction mixture, following incubation for 2 h at RT. Reactions were then spotted onto P81 ion exchange paper and kinase activity was detected by filter binding method.

### In Vitro Metabolic Stability Assays

Mouse liver microsomes (Xenotech) were diluted to 0.5 mg/mL in a solution containing 100 mM PBS buffer at 7.4 pH, an NADPH regenerating system (Promega), and test compounds at 20 μM. The mixture was incubated at 37° C. under gentle rotation for the indicated time points. The reaction was quenched by aliquoting 15 μL of solution into 60 μL ice-cold acetonitrile containing an internal standard. Test compounds were quantified by LC-MS using a system comprised of a Thermo Scientific Dionex Ultimate 3000 and a Bruker amaZon SL equipped with an electrospray ionization source controlled by a Bruker Hystar 3.2. Compounds were separated by injecting 20 μL of sample onto an Agilent Eclipse Plus C18 column (2.1×50 mm, 3.5 μM) maintained at 20° C., with the flow rate set at 400 μL/min. Initial flow conditions were 60% solvent A (MilliQ H₂O, 0.1% acetic acid), and 40% solvent B (HPLC-grade MeOH, 0.1% acetic acid). Solvent B was raised to 60% over 0.25 min and to 70% by 6.75 min. Solvent B was raised to 95% by 7 min and lowered back to 40% by 8 minutes; total run time was 9 min.

Mouse plasma was diluted with 100 mM PBS buffer at 7.4 pH and at a 1:1 ratio, and warmed to 37° C. Test compounds were added to the plasma solution at 20 μM and incubated for the indicated time point. Reactions was quenched by aliquoting 15 μL of solution into 60 μL ice-cold acetonitrile containing an internal standard. Compounds were quantified by LC-MS, as detailed above.

### In Vivo Pharmacodynamic Studies

6-week old male mice were purchased from Charles River Laboratories, and housed in Columbia University's animal control barrier facilities, and mice were allowed one week to acclimate to their new environment. To establish tumor xenografts, 10×10⁶ SK-N-Be2 cells were suspended in a 200 μL volume of 50% matrigel slurry. The mixture was injected into the right flank and allowed to grow to ˜200 mm³, which was achieved after approximately 2 weeks. Mice were kept on standard chow diet (Purina), and their cage dressing changed twice weekly. Animals were investigated regularly for any sign of discomfort. Once the tumors had reached sufficient volume, a solution of isopomiferin and control treatments was prepared and stored at 4° C. until use. Isopomiferin is not soluble in aqueous buffers, so solubility is enhanced for in vivo studies by formulation with cyclodextrin-b. To create the stock solution of isopomiferin, 5 mg of isopomiferin (MicroSource) was dissolved into 50 μL dimethyl sulfoxide (DMSO). To this 450 μL of cyclodextrin solution was added dropwise. Cyclodextrin is dissolved as a 50% w/v solution in 40% EtOH. It is important to add the cyclodextrin slowly and with continual mixing to solubilize the compound. This solution was added to one volume of PBS and filter-sterilized. This solution was then diluted to achieve appropriate concentrations for a 200 μL volume administration at 10 mg/kg to each mouse. The solution was administered to animals via intraperitoneal injection (i.p.). The control mouse received a solvent-only treatment of PBS/cyclodextrin absent isopomiferin. After 24 hrs treatment, mice were euthanized by CO₂ inhalation, followed by cervical dislocation. Tumors were removed, dissected, and sampled for protein isolation. Protein isolation from tumor samples was performed by homogenizing frozen tumor samples using a tissuelyzer in presence of 300 μL ripa buffer, followed by centrifugation at 17,000×g for 15 min at 4° C.

### Western Blot and Protein Quantification

NBL and SCLC cells were seeded in 6-well plates at a density of 300 k cells/well, and incubated overnight. The following day, cells were treated with compounds by aspirating growth media, rinsing cells with sterile PBS, and adding fresh media containing either the compound dissolved in DMSO, or DMSO-only negative control. Following treatment, cells were trypsinized, and pelleted in 1.5 mL eppendorf tubes and frozen at −80° C. To isolate soluble proteins, cell pellets were resuspended in RIPA cell lysis buffer and incubated on ice for 10 min, after which the cells were briefly sonicated to disrupt membranes. Cell lysates were centrifuged at 17000×g for 10 min at 4° C. to remove cellular debris. Protein was denatured by boiling samples for 10 min in Laemmli buffer. Protein samples were run on 4-12% agarose gradient gels (Invitrogen), following by semi-dry transfer to nitrocellulose membranes using an iBlot system (Invitrogen). Membranes were blocked using Odyssey Blocking Buffer (LI-COR) for 1 hr at room temperature. Protein-specific primary antibodies were purchased from Cell Signaling Technologies (CST) and Santa Cruz Biotechnology (SCBT). Primary antibodies used in these studies include: MycN (CST; 1:500 dilution), Actin (SCBT; 1:2000 dil.), cMYC (CST; 1:500 dil.), pAKT (S473; CST; 1:500 dil.), AKT (CST; 1:2000), pP70 (T389; CST; 1:1000) P70 (CST; 1:1000). Primary antibodies were diluted in blocking buffer and incubated overnight at 4° C. Following incubation, membranes were washed 3×5 min in PBST and incubated with secondary antibodies at 1:5000 for 1 hr at RT. Following incubation with secondary antibody, membranes were washed 2×5 min with PBST, and 1×5 min in dH₂O. Dried membranes were visualized using Odyssey CLx imaging system (LI-COR).

### Protein Expression and Crystallization Studies

Recombinant HIS-tagged CK2a1 protein was expressed in BL21 Escherichia coli from a codon-optimized expression system driven by an IPTG-inducible promoter. Bacteria were selected from a single successful transformation on selectable agar plates, and cultured in 2-YT broth with 100 μg/mL ampicillin at 37° C. until the culture reached an OD600 of 0.8. The culture was then acclimated to 20° C. for one hour, following which IPTG was added at 0.5 mM and the culture was incubated overnight at 20° C. The following day, the culture was pelleted at 4000 rpm for 20 min at 4° C. The pellet was resuspended in buffer (50 mM Tris-Cl pH 8.0, 500 mM NaCl, and protease inhibitor tablets) and lysed by sonication. Bacterial lysate was centrifuged at 14000 rpm for 35 min at 4° C., and the supernatant was incubated with Ni sepharose 6 Fast Flow beads that were equilibrated in buffer (50 mM Tris-Cl pH 8.0, 500 mM NaCl). Affinity purification using was performed following manufacturer's instructions (GE Healthcare). Eluted protein was dialyzed overnight at 4° C. in buffer (50 mM Tris-Cl and 300 mM NaCl) to remove imidazole. The following day, dialyzed protein was concentrated by centrifugation through amicon ultracell 10 kDa cutoff filter columns (EMD Millipore), followed by FPLC protein purification. Protein abundance was quantified by Bradford protein assay (BioRAD) and purity evaluated by coomassie staining of SDS-PAGE gels and western blot. CK2 was crystallized by micro-batch under-oil method. 2 μL of protein solution (10 mg/ml) was mixed with a protein crystallization reagent containing 0.5 μl Silver bullet F2 and 0.5 μL 50 mM HEPES (pH 6.8) and 15% (w/v) PEG 3350. Large block crystals appeared after one week and grew to full-length after 2-3 weeks. They diffracted X-ray at the NE_CAT 24 ID_E beam line of Advanced Photon Source at 2 A resolution.

### RNASeq Analysis

To uncover changes in gene expression that underpin responses to pomiferin and isopomiferin, RNASeq analysis of SK-N-Be2 cells was performed following treatment with the two active prenylated isoflavonoids as well as a null analog that served as a negative control. SK-N-Be2 cells were treated with either 3 μM or 10 μM of isopomiferin or pomiferin and sampled after 3 h and 6 h. As dimethyl pomiferin does not revert the MYCN signature or suppress MYCN protein at 10 μM, expression analysis of cells treated with dimethyl pomiferin was performed to identify changes in gene expression induced by the scaffold. By comparing the active to the null analogs, downstream analyses can focus on changes in gene expression associated with the MYCN signature.

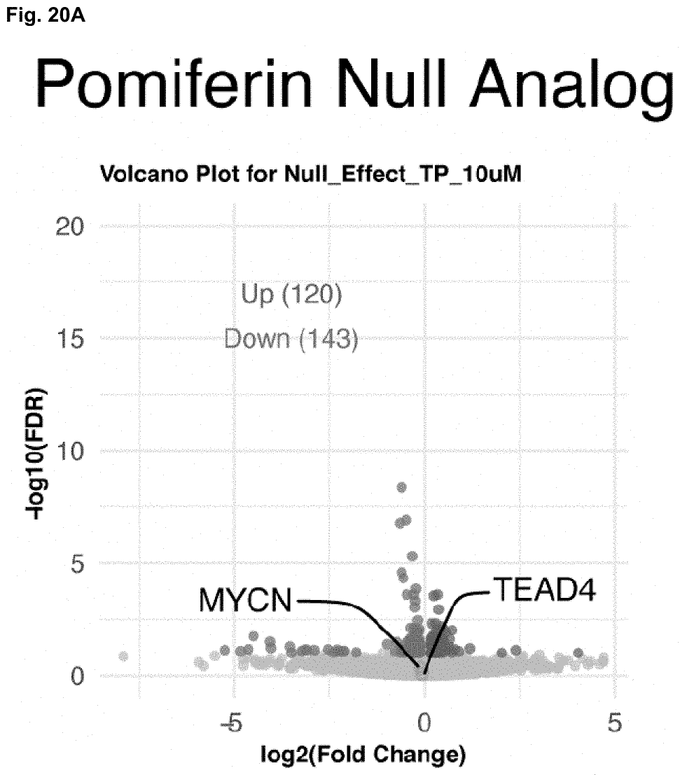

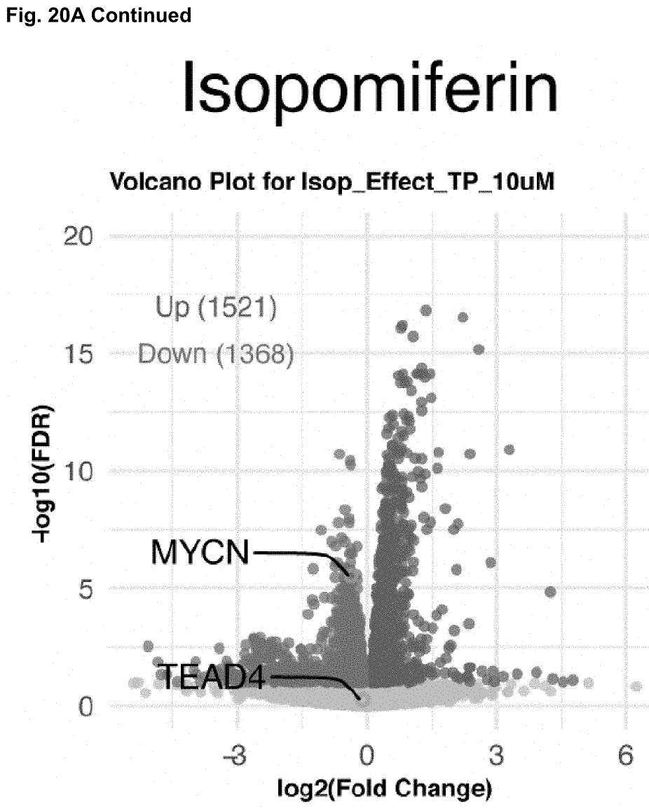

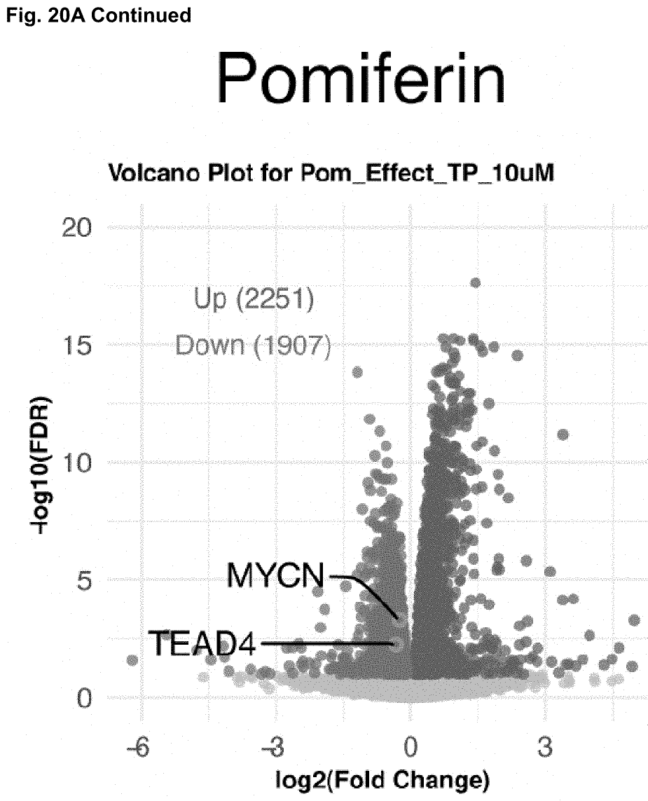

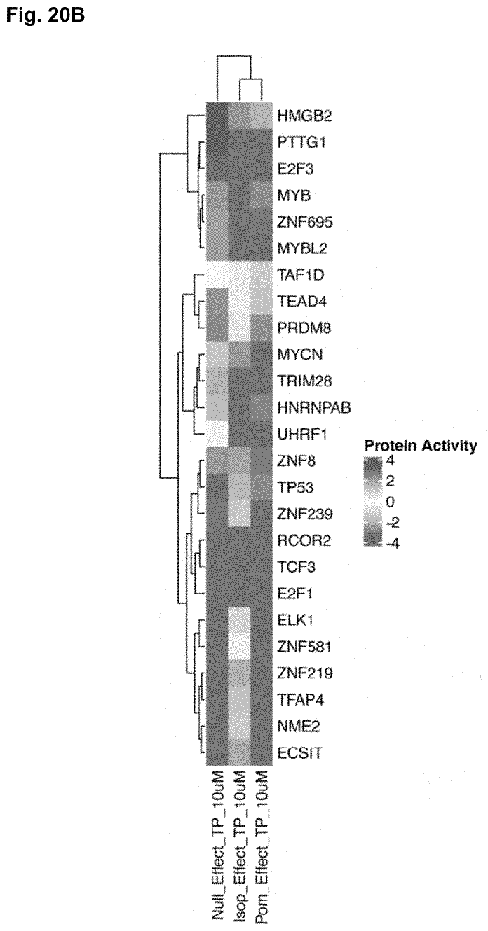

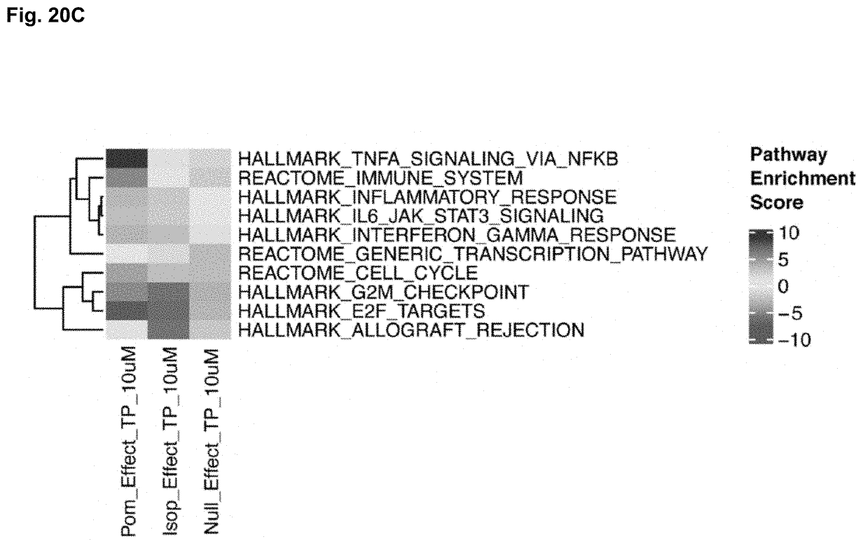

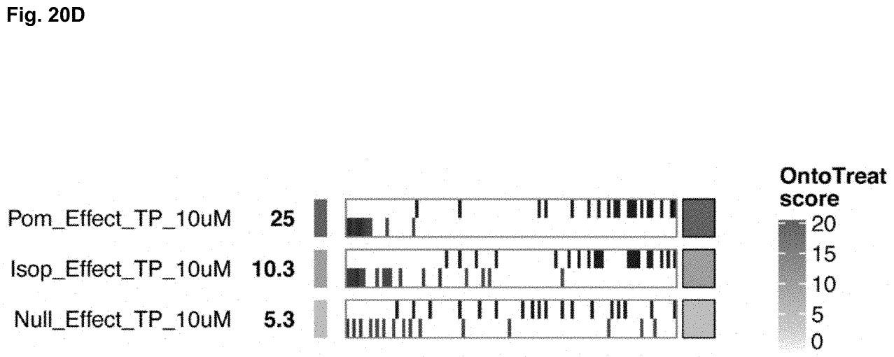

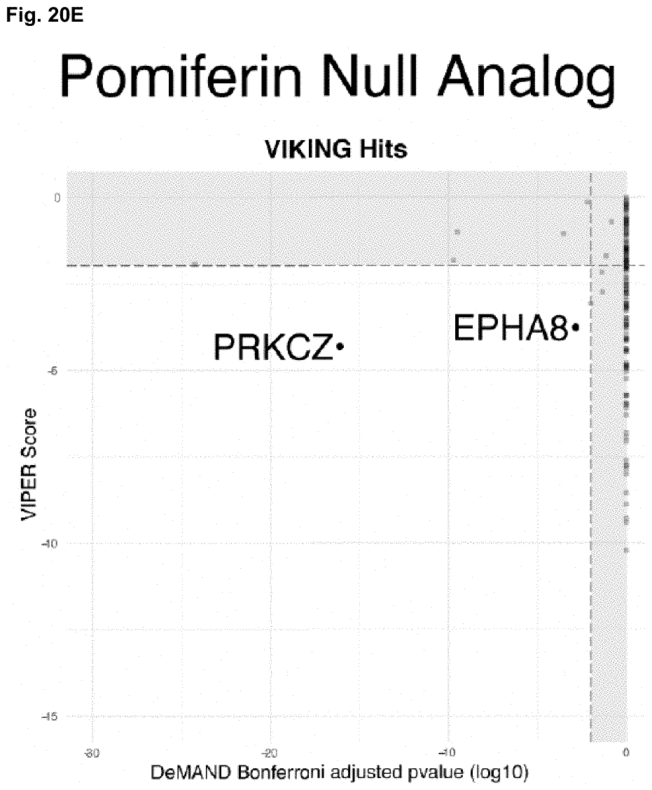

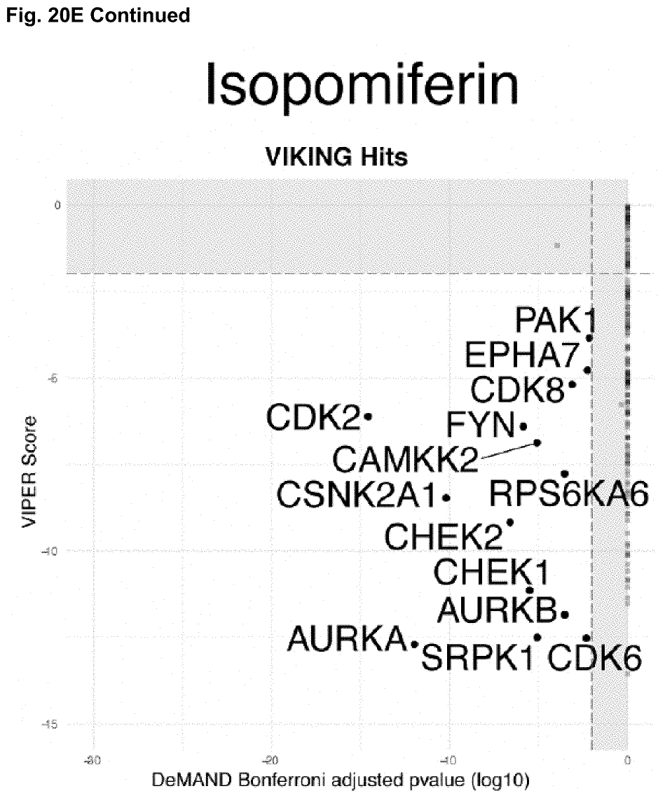

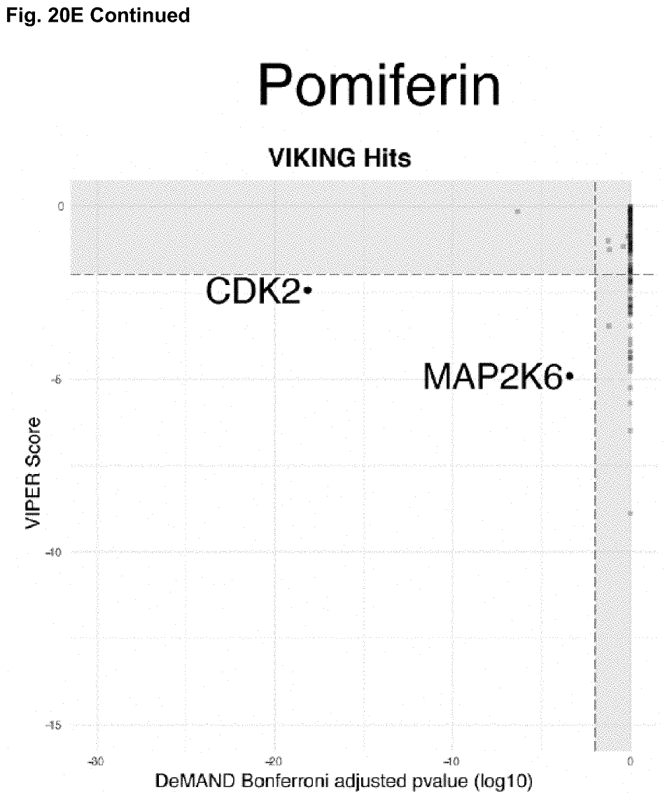

Transcriptome analysis revealed that both active analogs have profound impacts on gene expression, compared to dimethyl pomiferin (FIG. 20A). Consistent with previous qPCR analyses, pomiferin and isopomiferin suppressed MYCN transcript abundance, whereas the null analog did not. Importantly, the more potent analog, pomiferin, suppressed both MYCN and TEAD4 transcript, while isopomiferin did not affect TEAD4. As TEAD4 comprises a feedback loop with MYCN that drives the MR signature, this could be an important regulatory mechanism that underpins the potent activity of pomiferin. Dimethyl pomiferin did not affect either MYCN or TEAD4. A gene-by-gene analysis of transcripts that respond to pomiferin and isopomiferin is ongoing, with focus on transcripts that respond at early time points and which are not affected by the null analog.

VIPER analysis of expression profiles revealed that pomiferin had the strongest effect on the MYCN MR signature, whereas dimethyl pomiferin did not (FIG. 20D). When individual master regulators that comprise the signature were analyzed by VIPER, most MRs exhibited decreases in protein activity across all three compounds tested. Intriguingly, only six MRs were suppressed by pomiferin and isopomiferin, while being unaffected by the null analog dimethyl pomiferin (FIG. 20B). This suggests that these MRs could be more tightly linked to MYCN activity and relevant mechanistic drivers of the active compounds.

Gene set enrichment analysis revealed that the active analogs both suppressed transcripts associated with G2/M checkpoint and E2F pathways, consistent with previous results generated using PLATESeq (FIG. 20C). The null analog did not affect gene sets associated with G2/M or E2F pathways, consistent with the minimal effect this compound has on cell proliferation. Together, the data generated by RNASeq corroborated the earlier results with PLATSeq, which highlights the utility and robust results generated by this nascent technology. The advantage of RNAseq dataset is that we now have a list of responsive transcripts that can be mined to understand the mechanism of action of the prenylated isoflavonoids.

Extraction of Prenylated Isoflavonoids from Osage Orange

Osage oranges (Maclura pomifera) were collected, chopped up, oven dried at 80° C., and pulverized in a corn meal grinder. The resulting powder was placed in a Soxhlet extractor and extracted with hexane, followed by ether. The ether extracts were concentrated to give an orange crystalline solid containing approximately a 1:1 mixture of pomiferin and osajin, as determined by 1H NMR. Reversed phase HPLC separation (CH3CN/H2O/0.01% TFA, 60-90% CH3CN, 15-minute gradient) of 100 mg of the mixture gave two major and three minor peaks corresponding to pomiferin (44.6 mg yellow powder, RT=14.1 min), osajin (36.2 mg pale yellow powder, RT=17.1 min), AZ13-1 (RT=7.2 min), AZ13-2 (RT=9.9 min), and AZ13-3 (RT=12.3 min), respectively.

### AZVII-13-1 (5,7-dihydroxy-3-(4-hydroxyphenyl)-6-(3-methylbut-2-en-1-yl)-4H-chromen-4-one)

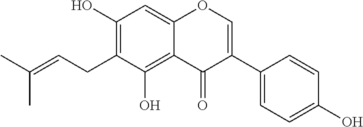 [SMILES: CC(C)=CCc1c(O)cc2occ(-c3ccc(O)cc3)c(=O)c2c1O]

(CD3OD) δ 8.03 (s, 1H), 7.37 (d, J=8.6 Hz, 2H), 6.85 (d, J=8.6 Hz, 2H), 6.38 (s, 1H), 5.21-5.27 (m, 1H), 1.78 (s, 3H), 1.66 (s, 3H). MS M+H=339.1

### AZVII-13-2 (Diprenylorobol; 3-(3,4-dihydroxyphenyl)-5,7-dihydroxy-6,8-bis(3-methylbut-2-en-1-yl)-4H-chromen-4-one)

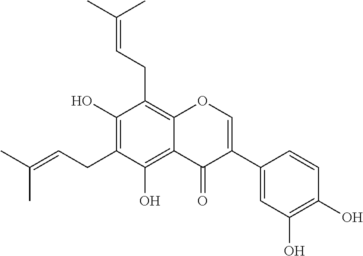 [SMILES: CC(C)=CCc1c(O)c(CC=C(C)C)c2occ(-c3ccc(O)c(O)c3)c(=O)c2c1O]

(CD3OD) δ 8.10 (s, 1H), 7.03 (d, J=1.9 Hz, 1H), 6.86 (dd, J=1.9, 8.2 Hz, 1H), 6.82 (d, J=8.2 Hz, 1H), 5.28-5.12 (m, 2H), 3.49 (d, J=7.0 Hz, 2H), 3.39 (d, J=7.1 Hz, 2H), 1.82 (s, 3H), 1.80 (s, 3H), 1.68 (s, 6H). MS M+H=422.2

### AZVII-13-3 (Diprenylgenistein; 5,7-dihydroxy-3-(4-hydroxyphenyl)-6,8-bis(3-methylbut-2-en-1-yl)-4H-chromen-4-one)

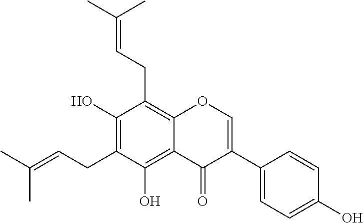 [SMILES: CC(C)=CCc1c(O)c(CC=C(C)C)c2occ(-c3ccc(O)cc3)c(=O)c2c1O]

(CD3OD) δ 8.12 (s, 1H), 7.38 (d, J=8.6 Hz, 2H), 6.85 (d, J=8.6 Hz, 2H), 5.24-5.15 (m, 2H), 3.49 (d, J=7.4 Hz, 2H), 3.39 (d, J=7.1 Hz, 2H), 1.82 (s, 3H), 1.80 (s, 3H), 1.68 (s, 6H). MS M+H=407.2

### Hydrogenation of Pomiferin and Isopomiferin

An approximately 1:1 pomiferin:osajin mixture (164 mg) in methanol (15 mL) was treated with 10% Pd/C (30 mg) and hydrogen gas (balloon pressure) at room temperature. After 18 h the mixture was filtered through celite and concentrated in vacuum to give a colorless solid. Separation by RP HPLC (CH3CN/H₂O/0.01% TFA, 60-90% CH₃CN, 15-minute gradient) gave two major and two minor peaks corresponding to AZVII-12P (RT=14.1 min), AZVII-120 (RT=16.9 min), AZVII-12-1 (RT=10.8 min), and AZVII-12-2 (RT=13.2 min), respectively.

### AZVII-12P (3-(3,4-dihydroxyphenyl)-5-hydroxy-6-isopentyl-8,8-dimethyl-2,3,9,10-tetrahydro-4H,8H-pyrano[2,3-f]chromen-4-one)

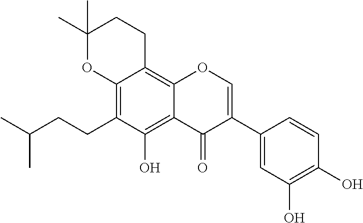 [SMILES: CC(C)CCc1c2c(c3occ(-c4ccc(O)c(O)c4)c(=O)c3c1O)CCC(C)(C)O2]

(CD3OD) δ 6.75 (d, J=8.2 Hz, 1H), 6.71 (d, J=2.1 Hz, 1H), 6.62 (dd, J=8.2, 2.1 Hz, 1H), 4.58 (dd, J=7.2, 11.3 Hz, 1H), 4.52 (dd, J=5.0, 11.3 Hz, 1H), 3.79 (dd, J=7.2, 5.0 Hz, 1H), 2.61 (t, J=6.9 Hz, 2H), 2.52 (t, J=6.9 Hz, 2H), 1.80 (t, J=6.9 Hz, 2H), 1.53 (sept, J=6.6 Hz, 1H), 1.36 (s, 3H), 1.35 (s, 3H), 0.94 (d, J=6.6 Hz, 6H) MS M+H=424.2

### AZVII-120 (5-hydroxy-3-(4-hydroxyphenyl)-6-isopentyl-8,8-dimethyl-2,3,9,10-tetrahydro-4H,8H-pyrano[2,3-f]chromen-4-one)

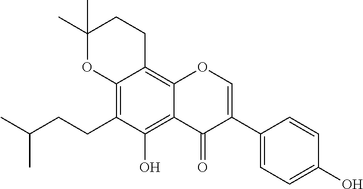 [SMILES: CC(C)CCc1c2c(c3occ(-c4ccc(O)cc4)c(=O)c3c1O)CCC(C)(C)O2]

(CD3OD) δ 7.10 (d, J=8.6 Hz, 2H), 6.76 (d, J=8.6 Hz, 2H), 4.58 (dd, J=5.3, 11.3 Hz, 1H), 4.53 (dd, J=7.9, 11.3 Hz, 1H), 3.89 (dd, J=5.3, 7.9 Hz, 1H), 2.61 (t, J=6.9 Hz, 2H), 2.52 (t, J=6.9 Hz, 2H), 1.80 (t, J=6.9 Hz, 2H), 1.52 (sept, J=6.6 Hz, 1H), 1.36 (s, 3H), 1.35 (s, 3H), 0.93 (d, J=6.6 Hz, 6H) MS M+H=408.2

### AZVII-12-1 (3-(3,4-dihydroxyphenyl)-5,7-dihydroxy-6,8-diisopentylchroman-4-one)

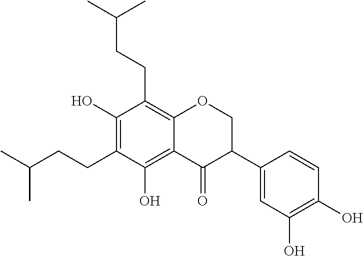 [SMILES: CC(C)CCc1c(O)c(CCC(C)C)c2c(c1O)C(=O)C(c1ccc(O)c(O)c1)CO2]

(CD3OD) δ 6.73 (d, J=8.2 Hz, 1H), 6.71 (d, J=2.2 Hz, 1H), 6.62 (dd, J=2.2, 8.2 Hz, 1H), 4.53 (dd, J=4.8, 11.3 Hz, 1H), 4.47 (dd, J=7.1, 11.3 Hz, 1H), 3.76 (dd, J=4.8, 7.1 Hz, 1H), 2.62-2.54 (m, 4H), 1.64-1.47 (m, 2H), 1.40-1.29 (m, 4H), 0.95 (d, J=5.3 Hz, 6H), 0.93 (d, J=5.3 Hz, 6H). MS M+H=428.2

### AZVII-12-2 (5,7-dihydroxy-3-(4-hydroxyphenyl)-6,8-diisopentylchroman-4-one)

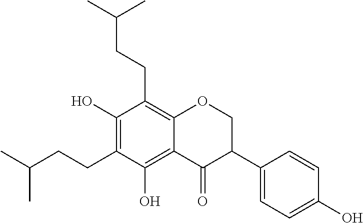 [SMILES: CC(C)CCc1c(O)c(CCC(C)C)c2c(c1O)C(=O)C(c1ccc(O)cc1)CO2]

(CD3OD) δ 7.11 (d, J=8.6 Hz, 2H), 6.75 (d, J=18.6 Hz, 2H), 4.54 (dd, J=5.0, 11.3 Hz, 1H), 4.48 (dd, J=7.5, 11.3 Hz, 1H), 3.84 (dd, J=5.0, 7.5 Hz, 1H), 2.53-2.63 (m, 4H), 1.65-1.48 (m, 2H), 1.42-1.24 (m, 4H), 0.95 (d, J=4.5 Hz, 6H), 0.93 (d, J=4.4 Hz, 6H). MS M+H=412.2

### Methylation of Pomiferin and Osajin Mixture

To an approximately 1:1 mixture of pomiferin:osajin (50 mg) in acetone (2 mL) was added K2CO3 (84 mg) and CH3I (15 μL). After 1 h an additional 30 μL of CH3I was added. After 48 h the reaction mixture was filtered, concentrated and purified by RP HPLC to give 5-hydroxy-3-(4-methoxyphenyl)-8,8-dimethyl-6-(3-methylbut-2-en-1-yl)-4H,8H-pyrano[2,3-f]chromen-4-one and 3-(3,4-dimethoxyphenyl)-5-hydroxy-8,8-dimethyl-6-(3-methylbut-2-en-1-yl)-4H,8H-pyrano[2,3-f]chromen-4-one.

### AZVII-89-11 3-(3,4-dimethoxyphenyl)-5-hydroxy-8,8-dimethyl-6-(3-methylbut-2-en-1-yl)-4H,8H-pyrano[2,3-f]chromen-4-one

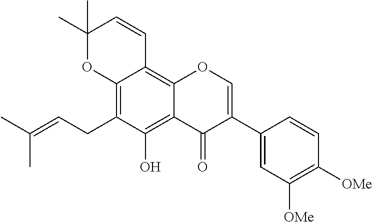 [SMILES: COc1ccc(-c2coc3c4c(c(CC=C(C)C)c(O)c3c2=O)OC(C)(C)C=C4)cc1OC]

(DMSO-d6) δ 13.40 (s, 1H), 8.49 (s, 1H), 7.17 (d, J=2.1 Hz, 1H), 7.14 (dd, J=8.3, 2.1 Hz, 2H), 7.03 (d, J=8.3 Hz, 1H), 6.69 (d, J=10.0 Hz, 1H), 5.80 (d, J=10.0 Hz, 1H), 5.22-5.08 (m, 1H), 3.79 (s, 3H), 3.78 (s, 3H), 3.25 (d, J=7.4 Hz, 2H), 1.75 (s, 3H), 1.63 (s, 3H), 1.44 (s, 6H). MS M+H=448.2

### AZVI-89-12 5-hydroxy-3-(4-methoxyphenyl)-8,8-dimethyl-6-(3-methylbut-2-en-1-yl)-4H,8H-pyrano[2,3-f]chromen-4-one

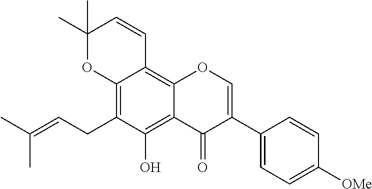 [SMILES: COc1ccc(-c2coc3c4c(c(CC=C(C)C)c(O)c3c2=O)OC(C)(C)C=C4)cc1]

(DMSO-d6) δ 13.38 (s, 1H), 8.46 (s, 1H), 7.51 (d, J=8.8 Hz, 2H), 7.01 (d, J=8.8 Hz, 2H), 6.69 (d, J=10.0 Hz, 1H), 5.80 (d, J=10.0 Hz, 1H), 5.31-4.89 (m, 1H), 3.79 (s, 3H), 3.27-3.22 (m, 2H), 1.75 (s, 3H), 1.63 (s, 3H), 1.44 (s, 6H). MS M+H=418.2

### Methylation of Pomiferin

To pomiferin (50 mg) in acetone (2 mL) was added K₂CO₃ (84 mg) and CH3I (30 μL). After 2 h an additional 30 μL of CH₃I was added. After 48 h the reaction mixture was filtered, concentrated and purified by RP HPLC to give (5-hydroxy-3-(3-hydroxy-4-methoxyphenyl)-8,8-dimethyl-6-(3-methylbut-2-en-1-yl)-4H,8H-pyrano[2,3-f]chromen-4-one).

### AZVII-4A (5-hydroxy-3-(3-hydroxy-4-methoxyphenyl)-8,8-dimethyl-6-(3-methylbut-2-en-1-yl)-4H,8H-pyrano[2,3-f]chromen-4-one)

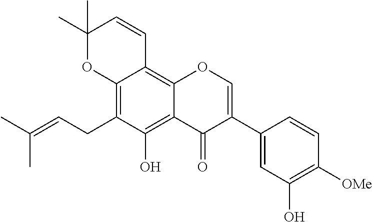 [SMILES: COc1ccc(-c2coc3c4c(c(CC=C(C)C)c(O)c3c2=O)OC(C)(C)C=C4)cc1O]

(CD3OD) δ 8.14 (s, 1H), 7.06 (bd s, 1H), 6.99 (bd s, 2H), 6.74 (d, J=9.8 Hz, 1H), 5.69 (d, J=9.8 Hz, 1H), 5.34-5.08 (m, 1H), 3.89 (s, 3H), 1.80 (s, 3H), 1.80 (s, 3H), 1.67 (s, 3H), 1.47 (s, 6H). MS M+H=434.2

### Triacetylation of Pomiferin

A mixture of pomiferin (100 mg) and NaOAc (600 mg) in acetic anhydride (7.5 mL) was heated in an oil bath at 150° C. for 3 h. After cooling to room temperature, the mixture was poured into water (20 mL) and stored at 4° C. overnight. Filtration gave 4-(5-acetoxy-8,8-dimethyl-6-(3-methylbut-2-en-1-yl)-4-oxo-4H,8H-pyrano[2,3-f]chromen-3-yl)-1,2-phenylene diacetate as a gray solid (96 mg).

### AZVII-44 4-(5-acetoxy-8,8-dimethyl-6-(3-methylbut-2-en-1-yl)-4-oxo-4H,8H-pyrano[2,3-f]chromen-3-yl)-1,2-phenylene diacetate

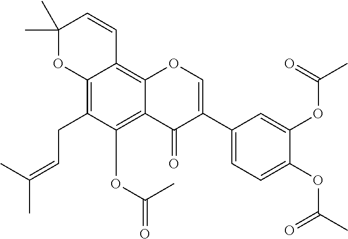 [SMILES: CC(=O)Oc1ccc(-c2coc3c4c(c(CC=C(C)C)c(OC(C)=O)c3c2=O)OC(C)(C)C=C4)cc1OC(C)=O]

(CDCl3) δ 7.85 (s, 1H), 7.41-7.32 (m, 2H), 7.22 (d, J=8.9 Hz, 1H), 6.76 (d, J=10.0 Hz, 1H), 5.69 (d, J=10.0 Hz, 1H), 5.24-4.87 (m, 1H), 2.43 (s, 3H), 2.29 (s, 6H), 1.78 (s, 3H), 1.67 (s, 3H), 1.49 (s, 6H). MS M+H=546.2

### Example 2

### Discovery of Isopomiferin and its Effect on MycN^{AMP} MR Regulatory Network

Isopomiferin was identified in a high-throughput chemical screen aimed at identifying lethal small molecules with enhanced activity in cell lines harboring MYCN amplifications (MycN^{AMP}). Two cell lines representing MycN^{AMP} neuroblastoma (SK-N-Be2 and IMR-32) and two neuroblastoma cell lines that do not express MycN (NLF and SK-N-AS) were seeded at 1000 cells/well in 384-well plates. Three chemical libraries, consisting of 5500 compounds were screened across the four NBL cell lines. The NCI Clinical Collection, the SPECTRUM Collection and the NIH Diversity Set were chosen because of their enrichment for compounds with bioactivity, the diversity in chemical structure, and the abundance of compounds with known mechanisms of action and safety profiles. Compounds in the Clinical Collection are notable due to their history of use in cancer research and pharmaceutical development. Having been tested in FDA clinical trials, these drugs often have known mechanisms of action and have been investigated for safety potential.

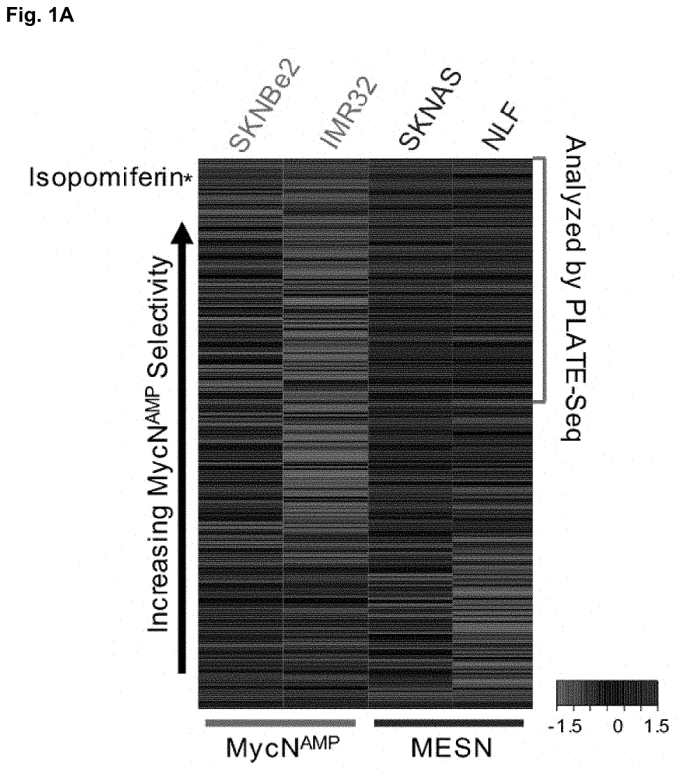

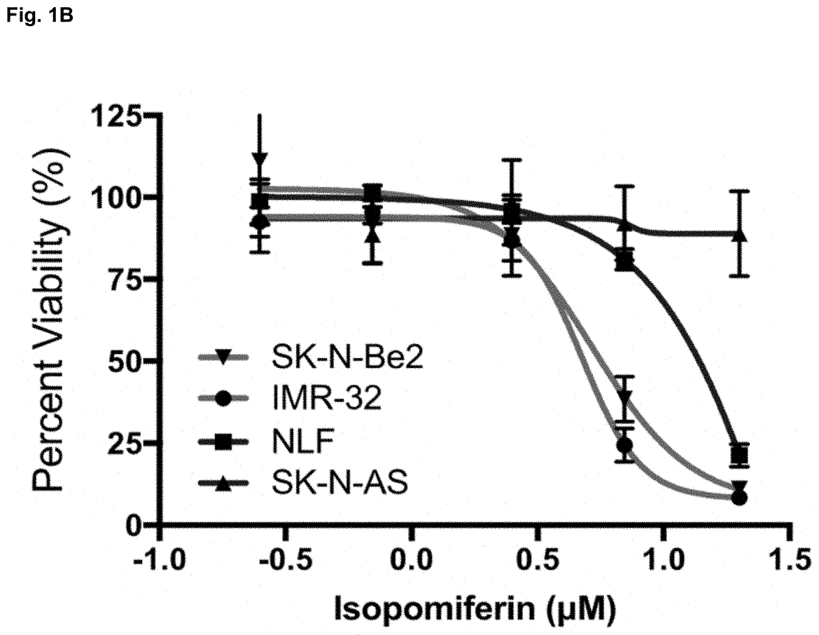

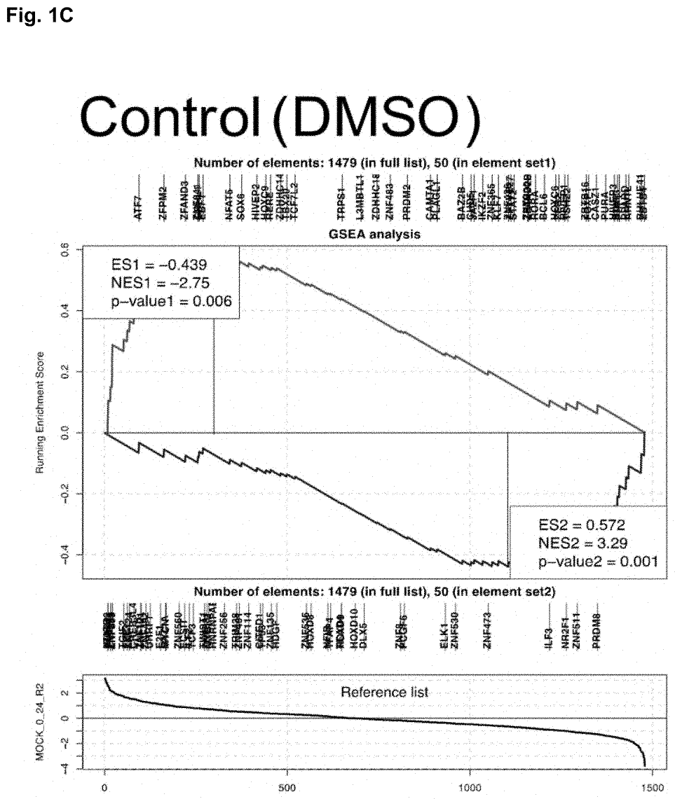

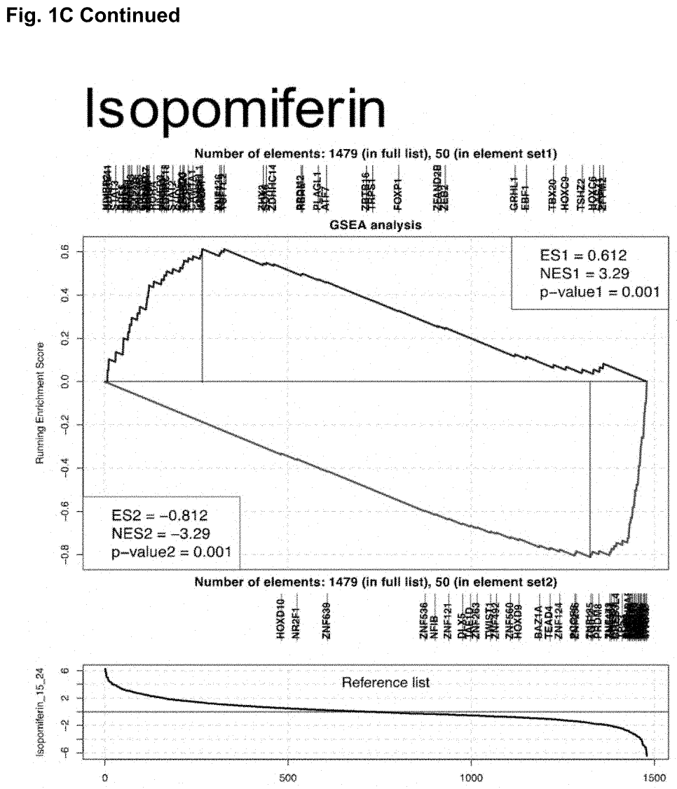

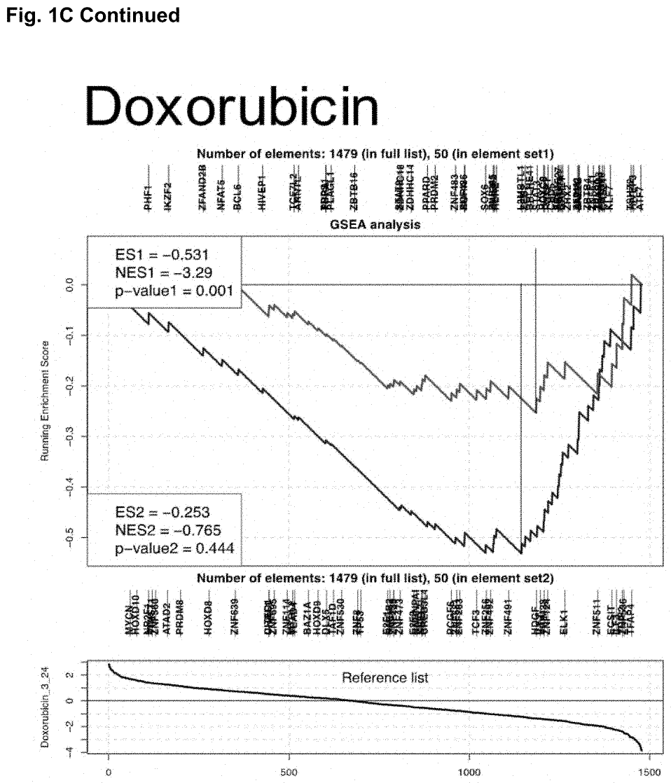

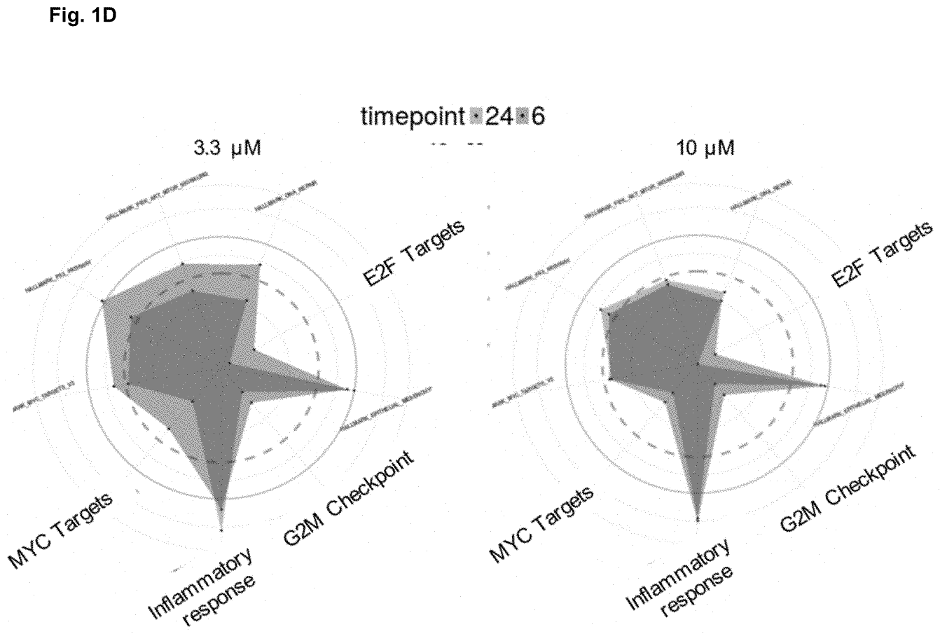

Compounds were screened at 20 μM for 72 hrs, to identify all compounds that were lethal to any of the four cell lines, which we defined as <10% viability relative to untreated control. Compounds that were lethal in at least one cell line were rescreened across a five-point dilution series ranging from 20 μM to ˜250 nM. Based on these dose-response curves, an IC₅₀ value was determined for each compound in each cell line. The average IC₅₀ from each subtype were calculated and a ratio was generated that ranked compounds based on subtype selectivity (FIG. 1A). Visualizing the IC₅₀ values for each compound across four NBL cell lines using a heatmap, it is apparent that the screening strategy successfully identified subtype-selective compounds for both the MycN^{AMP} and MESN subtypes. This screening method enabled us to identify subtype selective compounds that we could then retest using gene expression profiling (FIG. 1B).

Compounds that were selective for MycN^{AMP} cell lines were tested for their ability to suppress a regulatory module consisting of dysregulated transcription factors (aka Master Regulators). This regulatory signature has been proposed as a network that stabilizes and maintains MycN expression in MycN^{AMP} NBL. 90 compounds with increased potency in MycN^{AMP} lines were tested in a high-throughput gene expression profiling tool, called PLATE-Seq (Bush et al. 2017). PLATE-Seq works by performing RNA isolation and cDNA synthesis in wells, and appending a barcode to the transcripts in each well. The transcripts are pooled and sequenced, essentially creating 96 RNA-Seq experiments. Given that there is less starting material, PLATE-Seq generates fewer reads than traditional RNA-Seq (˜500 k-1 MM reads per well). As a result, PLATE-Seq is designed for use in conjunction with algorithm-based network analyses, such as VIPER and DeMAND (Woo et al. 2015; Alvarez et al. 2016), which can evaluate higher-order changes to the transcriptome landscape.

To evaluate the effect of MycN^{AMP}-selective compounds on the regulatory module, we treated SK-N-Be2 cells with the IC₂₀ of the compounds for 24 hrs, and then analyzed changes in expression profiles using the VIPER algorithm (Alvarez et al. 2016). This network based tool evaluates the change in expression of all targets of a given transcription factor and treats them as a single gene set. Performing gene set enrichment analysis of these “sets” can infer the changes to relative transcription factor activity following a specific treatment. Compounds were ranked based on their ability to revert the regulatory module in SK-N-Be2 cells. Of the hits that reverted the module, isopomiferin was the top compound of the 90 tested. To ensure that this effect was specific to the regulatory signature driving MycN^{AMP} cells, the effect of isopomiferin was compared to doxorubicin, a non-specific cytotoxic compound often used as a standard of care chemotherapeutic for NBL. While isopomiferin induced coordinated reversion of the signature, doxorubicin induced spurious effects (FIG. 1C).

We next performed gene set analysis using PLATE-Seq expression profiling by binning transcripts according to the Cancer Hallmarks gene sets found at the molecular signatures database. Curated by the Broad Institute, these fifty hallmark pathway sets represent well defined cancer processes or states associated with cancer malignancies. Notably, one set contains targets associated with MYC activity. Given that isopomiferin disrupted the MycN^{AMP} regulatory module, it was expected that this target would be suppressed by isopomiferin. We sought to understand how the compound is affecting cells by assessing whether any other pathways are affected by the compound. The GSEA analysis confirmed that isopomiferin treatment suppressed the MYC targets gene set, which was a validating control to demonstrate that our analysis is consistent across platforms (FIG. 1D). It was also observed that “E2F Targets” and “G2M Checkpoint” sets were suppressed as well, suggesting that these processes are inhibited by isopomiferin. E2F hands regulators act in a feedback loop with MycN and also regulate cell cycle progression (Strieder and Lutz 2003; Li et al. 2014), so this is an interesting finding that will be further investigated. MycN enables cancer cells to evade the immune system by suppressing antigens that can act as immune attractants necessary for detection and destruction of cancer cells (Brandetti et al. 2017; Topper et al. 2017). It is possible that enrichment of the “Inflammatory Response” gene set is a result of desuppression of these targets as a result of MycN inhibition.

### Example 3

### Isopomiferin Suppresses MycN Expression in NBL Cells and In Vivo

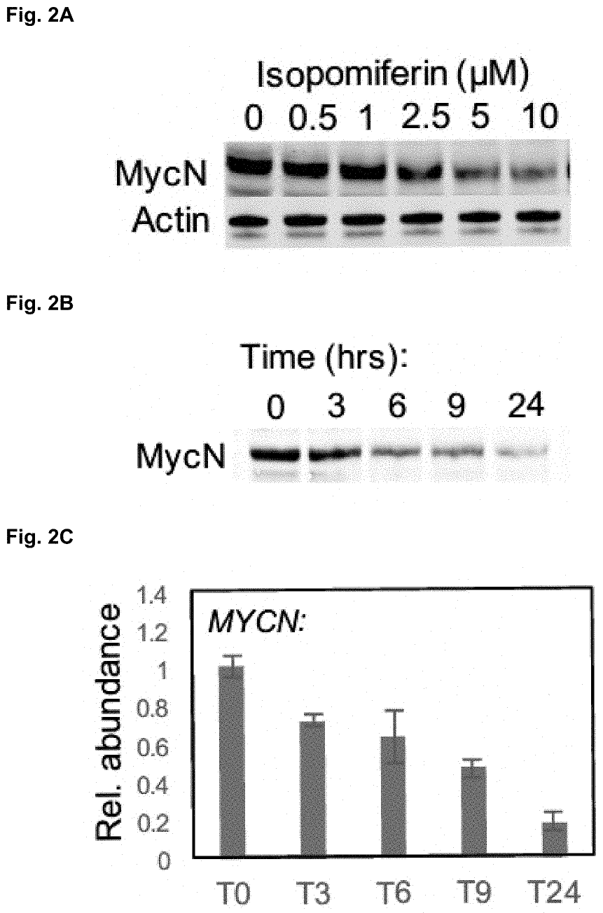

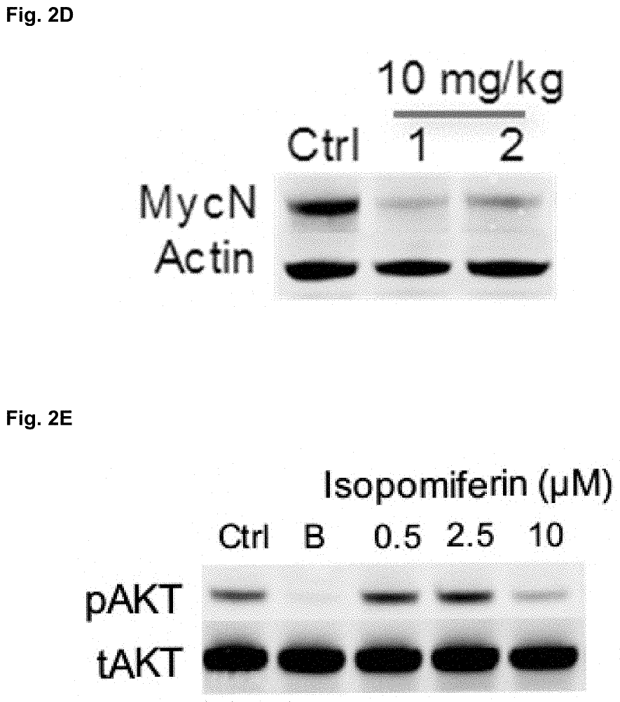

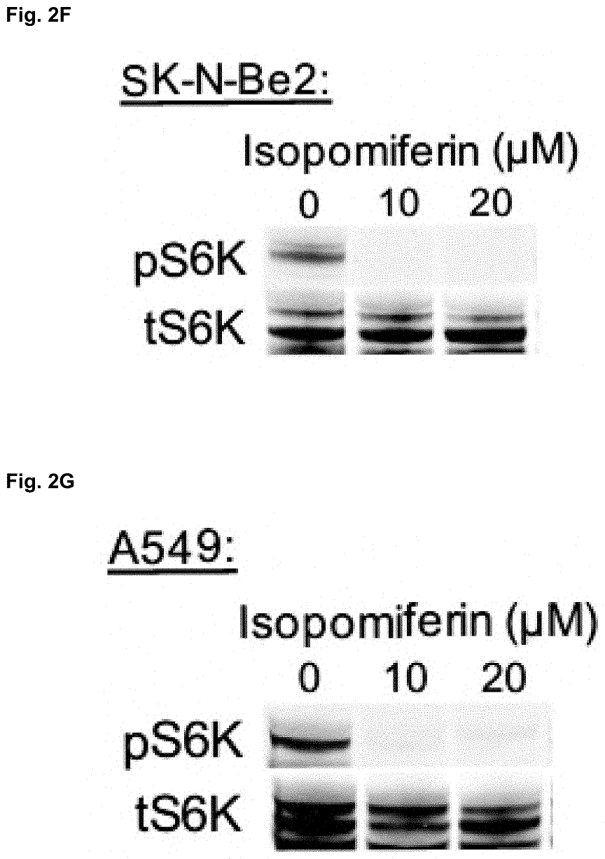

Given that isopomiferin disrupts a regulatory profile that centers on MycN activity, and that GSEA analysis found MYC targets were suppressed, we hypothesized that isopomiferin treatment would cause destabilization of MycN protein. SK-N-Be2 cells were treated with isopomiferin across a series of doses ranging from 0 μM to 10 μM for 24 hours. Protein was isolated and MycN abundance measured by western blot. Isopomiferin suppressed MycN abundance in a dose-dependent manner (FIG. 2A), while leaving Actin expression unaffected. As MycN protein regulates its own gene expression in a feed-forward loop (Huang and Weiss 2013), we tested whether isopomiferin affected MYCN gene expression. SK-N-Be2 cells were treated with 15 μM isopomiferin and sampled across time (0, 3, 6, 9, 24 hrs). When MYCN transcript was measured by RT-qPCR, it was found that transcript abundance decreased in a time-dependent manner that was consistent with protein suppression (FIG. 2B and FIG. 2C). Although it is presently uncertain whether isopomiferin inhibits MycN expression at the protein or the transcriptional level, it is apparent that the compound treatment ablates MycN protein abundance in NBL cell lines.

We tested whether isopomiferin was active in vivo, using subcutaneous mouse tumor xenografts. For these studies, tumor forming masses of SK-N-Be2 cells were injected into the right flanks of NCG mice. Once the tumors reached ˜200 mm³, a solution of isopomiferin was administered to the animals by intraperitoneal injection (i.p.). Two individual mice were administered isopomiferin at 10 mg/kg, while one individual was administered a solvent-only control. After 24 hrs, mice were euthanized and tumors excised for protein isolation and subsequent western blot analysis. One treatment with 10 mg/kg was sufficient to suppress MycN abundance in both treated mice (FIG. 2D). No toxicity issues were observed with the animals. This suggests that the compound is effective at suppressing MycN in animals, but further experimentation is needed to confirm that the compound suppresses tumor development in vivo. Given the association between MycN and tumor growth, it is likely that the ability to disrupt MycN is sufficient to cause tumor collapse.

### Example 4

### Isopomiferin Inhibits mTORC1/2 Signaling in NBL and Lung Cancer Cell Lines

Although the relevant molecular target of isopomiferin has not been reported, a related molecule pomiferin triacetate can inhibit mTOR activity (Bajer et al. 2014), a central regulator that is activated in many cancers (Guertin and Sabatini 2007; Populo et al. 2012). Importantly, pomiferin triacetate does not possess general kinase inhibition activity (Bajer et al. 2014), making it unique among mTOR inhibitors. We tested whether similar mechanisms could underpin isopomiferin activity in NBL and lung cancer cell lines. Depending on binding partners, mTOR can form two distinct protein complexes that act as a kinase that regulates downstream processes, such as cell proliferation and translation (Guertin and Sabatini 2007; Hay and Sonenberg 2004). An mTOR interaction with raptor comprises mTOC1, whereas binding with rictor forms mTORC2 (Guertin and Sabatini 2007; Hay and Sonenberg 2004). Each complex has its own distinct set of downstream targets and regulators. mTORC1 regulates translation through pS6K phosphorylation, and mTORC2 phosphoactivates AKT, to drive cell proliferation (Hay and Sonenberg 2004). By evaluating the effect of isopomiferin activity on these downstream targets, we were able to test the hypothesis that isopomiferin acts through mTOR in cells.

We first tested whether isopomiferin disrupted mTORC1 signaling in NBL by treating SK-N-Be2 cells to isopomiferin and blotting for pAKT abundance. Treatment with epithelial growth factor (EGF) induces pAKT1 through mTOR signaling. We tested whether isopomiferin had the ability to disrupt mTOR signaling by pre-treating cells with isopomiferin for 2 hrs, following which cells were treated with 10 ng/ml EGF for 30 minutes to induce pAKT1. As a positive control, the highly potent dual PI3K/mTOR inhibitor NVP-BEZ 235 was tested at 100 nM to demonstrate that we can detect inhibition of the mTOR signaling pathway using this experimental design. Treatment with 10 μM isopomiferin blocked pAKT accumulation in presence of EGF treatment, confirming that mTORC1 signaling activity is suppressed by isopomiferin (FIG. 2E). Total AKT abundance was used as a loading control to confirm that isopomiferin was simply not suppressing expression of AKT abundance.

We then tested whether mTORC2 complex was disrupted by isopomiferin treatment. mTORC2 phosphoactivates the ribosomal protein pS6K to enable translation. We tested whether phosphorylation of S6K was inhibited by isopomiferin by treating SK-N-Be2 cells to 10 μM and 20 μM isopomiferin for 24 hours and blotting for pS6K, using total S6K protein as a loading control. Treatment with isopomiferin completely inhibited phosphorylation of S6K protein after 24 hours at both concentrations tested (FIG. 2F), supporting the hypothesis that mTORC2 signaling is disrupted by isopomiferin in NBL. We then tested whether this was consistent across cell lines by performing a similar experiment in an NSCLC cell line, A549. Similar to NBL, isopomiferin suppressed pS6K in A549 cells (FIG. 2G). Together, these data suggest that isopomiferin blocks signaling activity of mTORC1/2, and that this effect is not specific to NBL.

### Example 5

### Isopomiferin is Selective for MycN-Driven Lung Cancers and Suppresses MycN

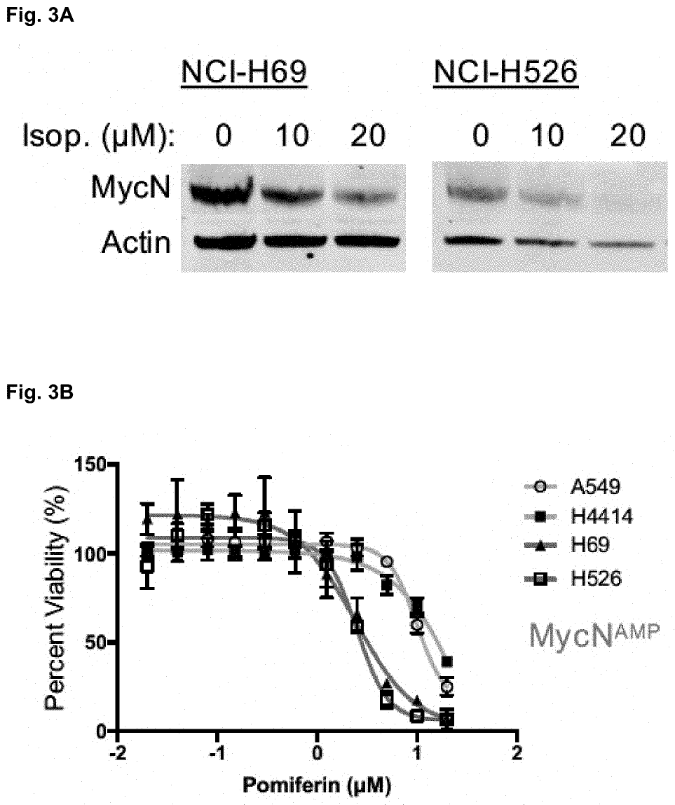

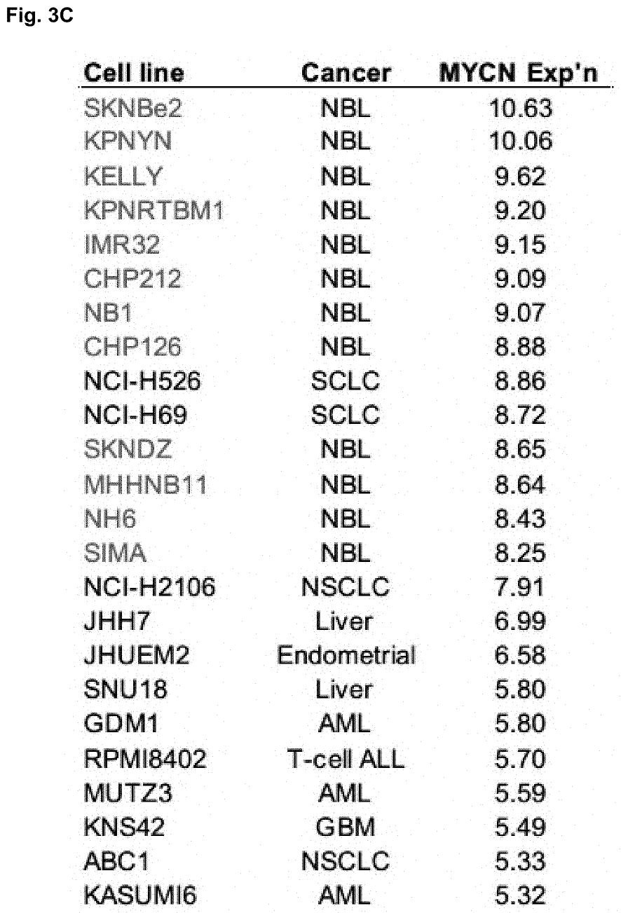

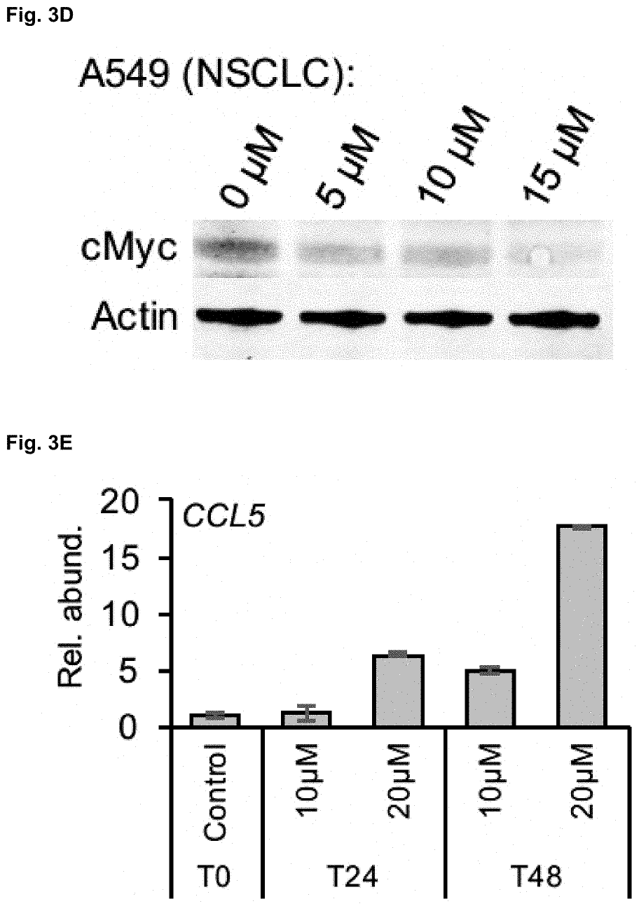

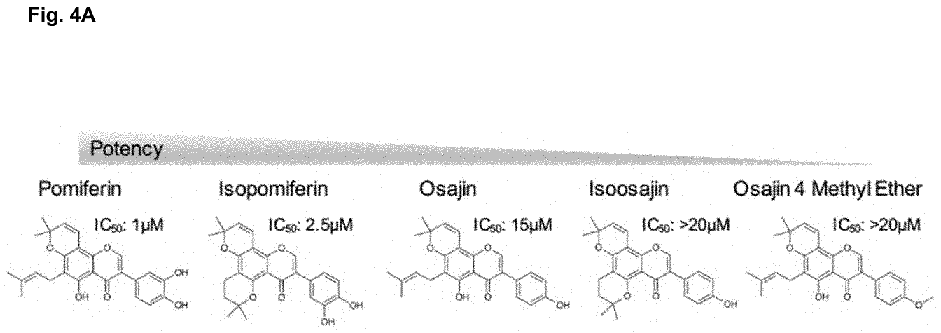

Having confirmed that isopomiferin suppresses MycN in NBL, we wanted to test whether the compound has broader application to other MycN-driven cancers. In addition to Wilms' tumor, a pediatric tumor with a MycN^{AMP} subtype (Williams et al. 2015), there are small patient subpopulations with dysregulated MycN expression in a variety of adult cancers (Beltran 2014; Lee et al. 2016; Liu et al. 2016; Yue et al. 2017). To identify cell models that would enable us to test whether isopomiferin has activity in these tumors, expression profiles from the cancer cell line encyclopedia (CCLE) were ranked based on MycN transcript abundance. This enabled us to identify cell models from NSCLC, SCLC, AML, and liver cancer that could be used to test whether isopomiferin suppresses MycN in additional MycN-driven cancers (FIG. 3C). We acquired the small cell lung cancer (SCLC) cell lines NCI-H69 and NCI-H526, and will test cell models from the other cancer types in the near future.

Two SCLC cell lines were acquired and tested for sensitivity to isopomiferin. NCI-H69 and NCI-H526 cells were treated with isopomiferin at 10 μM to 20 μM for 24 hrs, following which proteins were isolated for western blot analysis. Using MycN-specific primary antibody, it was found that isopomiferin suppresses MycN in SCLC in a dose-dependent manner (FIG. 3A). We then tested whether isopomiferin is selective for MycN-expressing cell lines. We used the CCLE expression profiles to identify and acquire two lung cancer cell lines that do not express MycN (NCI-H4414 and A549), and compared the four lines for sensitivity to isopomiferin. Cell lines were treated with a series of isopomiferin ranging from 20 μM to 0.2 μM for 48 hrs. Cell viability was then assayed using cell titer glo. Similar to trends observed in NBL, the MycN-expressing lines were more sensitive to isopomiferin treatment (FIG. 3B). We believe this phenotype can now form the basis of using MycN expression as a biomarker for isopomiferin sensitivity in tumors, which will be developed further and tested in the future.

### Example 6

### Isopomiferin Suppresses cMyc Activity in NSCLC

There are some shared upstream regulatory pathways that govern both MycN and cMyc expression, including mTOR signaling cascade (Guertin and Sabatini 2007; Hay and Sonenberg 2004). We hypothesized that isopomiferin could disrupt the expression of cMyc as well. We identified a cMyc-expressing lung cancer line, A549, and treated cells with 5, 10, or 15 μM isopomiferin for 24 hrs (FIG. 3D). Isopomiferin suppressed cMyc protein abundance in A549 in a dose dependent manner, suggesting that isopomiferin has utility as a cMyc suppressing compound that acts at the protein level to suppress cMyc abundance.

In lung cancer, cMyc overexpression suppresses CCL5, an antigen that enables the immune system to detect cancer cells (Topper et al. 2017). By suppressing CCL5, cMyc enables tumors to evade immune surveillance (Topper et al. 2017). We hypothesized that isopomiferin's ability to suppress cMyc in lung cancer cells may restore CCL5 expression. To test this hypothesis, A549 cells were treated with isopomiferin at 10 and 20 μM, and cells were sampled at 24 hrs and 48 hrs. CCL5 transcript was quantified by qPCR, which revealed that isopomiferin increased CCL5 expression by 15-fold at the 48 hr time-point (FIG. 3E). Given that CCL5 is an important factor for regulating immune detection of cancer cells, it is possible that isopomiferin can be developed as a co-immunotherapy that enhances immune system surveillance of tumor cells.

### Example 7

### Analogs Reveal that Small Structural Changes Impact Scaffold Efficacy

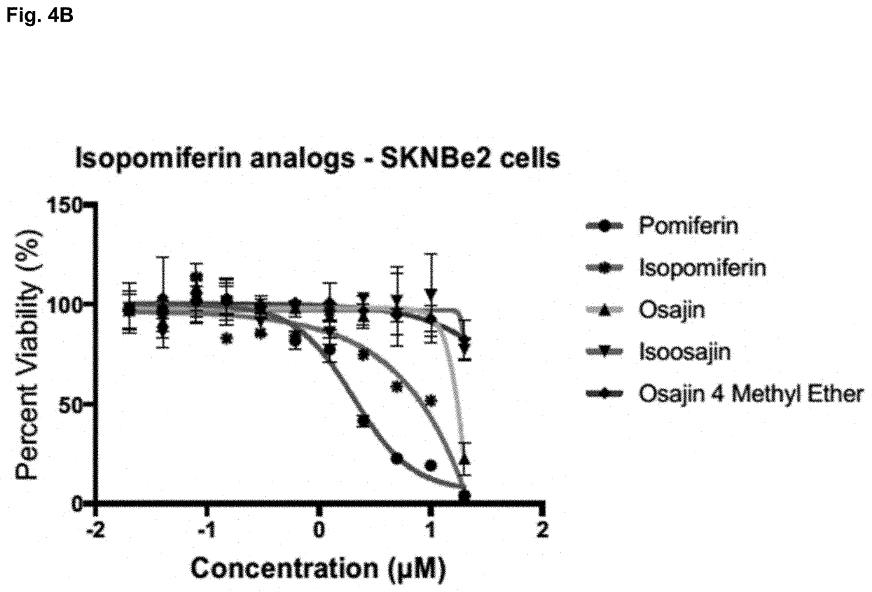

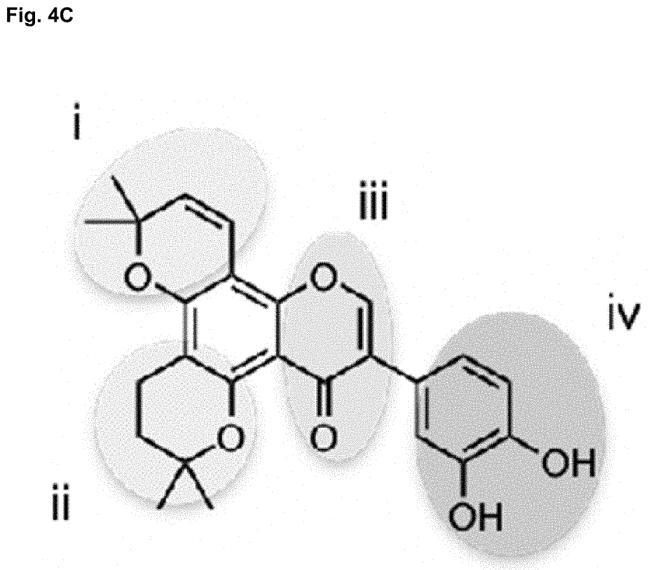

Commercially available structural analogs of osajin and pomferin were tested in SK-N-Be2 cells. Isopomiferin, isoosajin, and osajin 4-methyl ether had different IC₅₀ values in the SK-N-Be2 cells than pomiferin and osajin, showing that activity was dependent on modifications of the structure and supporting the synthesis of additional analogs to further explore SAR (FIGS. 4A-4C). In this set of 5 compounds, conversion of the hydroxy groups on the phenyl ring to methyl ethers, and cyclization of the isoprenyl moieties onto the hydroxyl group, influences the IC₅₀ in SK-N-Be2 cells.

Pomiferin and osajin were isolated from the fruit of the osage orange (Maclura pomifera) according to a literature procedure (Walter, E. D. et. al. J. Am. Chem. Soc. 1938, 60, 574-577). The fruits were cut into small pieces and dried at 80° C., then ground into a powder. The powder was placed in a soxhlet extractor and treated with hexane, followed by ether. The ether extract was concentrated to give ˜5% yield, by dry weight, of a yellow powder consisting of equal amounts of osajin and pomiferin. This material was dissolved in acetone and treated with K₂CO₃ and CH₃I. RP HPLC gave osajin methyl ether, pomiferin mono-methyl ether and pomiferin dimethyl ether. Dissolution of the osajin and pomiferin mixture in CH₂Cl₂ followed by treatment with mCPBA gave the corresponding epoxides on the trisubstituted olefins after purification by RP HPLC (Scheme 1). The osajin diepoxide and pomiferin diepoxide can be synthesized in a similar manner (Scheme 2).

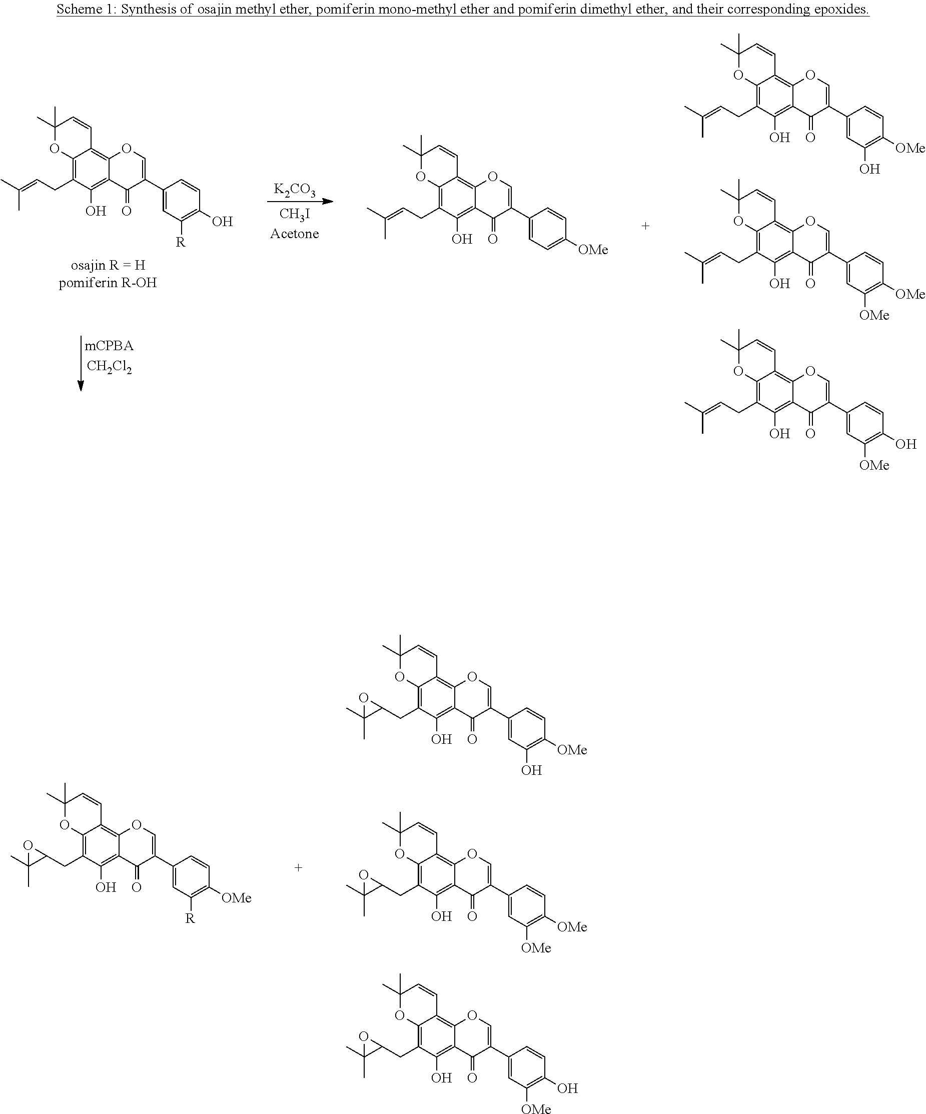

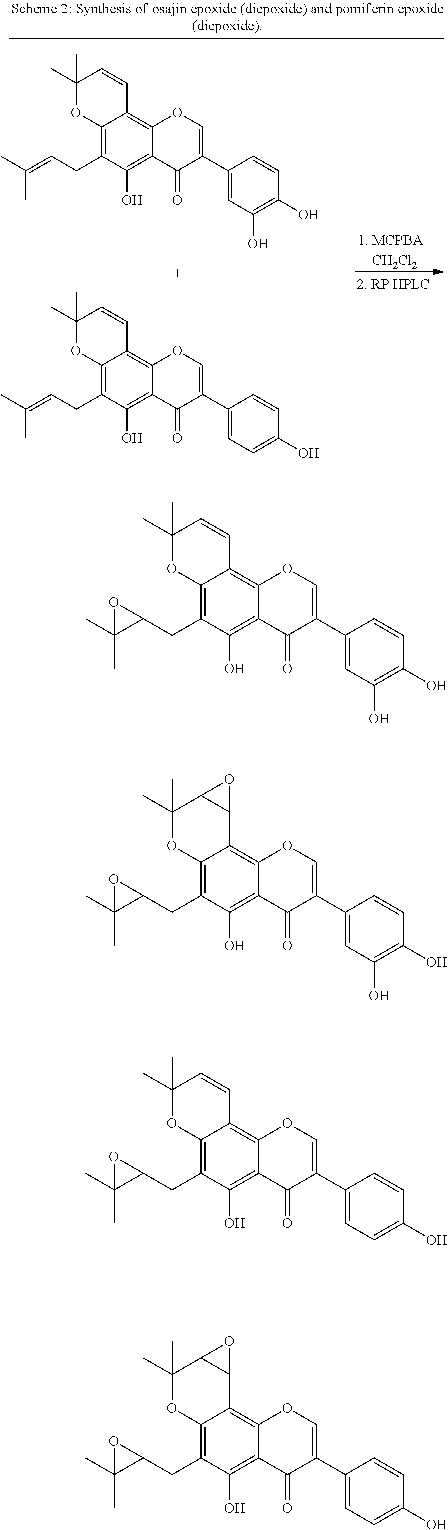

Further analogues will be made by semi-synthesis from multi-gram quantities of pomiferin and osajin isolated from osage orange fruits. Analogs will also be made using synthesis routes starting with commercially available building blocks. For example, hydrogenated analogs can be synthesized by adding hydrogens to the structures (Scheme 3). Water-solubilizing groups such as amines could be incorporated by cleavage of the tri-substituted double bond in the isoprene moiety of pomiferin or osajin using MCPBA and periodic acid. The resulting aldehyde would give the corresponding amine by reductive amination (Scheme 4). The phenol and catechol rings of osajin and pomiferin, respectively, could be replaced by bioisosteres. Suzuki coupling chemistry with commercially available or readily synthesized boronic acids would give pomeferin and osajin analogs with bioisostere replacements of the phenolic groups (Scheme 5). Introduction of nitrogen into the phenol or catechol ring will also be explored. Introduction of water solubilizing groups onto these phenol bioisosteres will also be done.

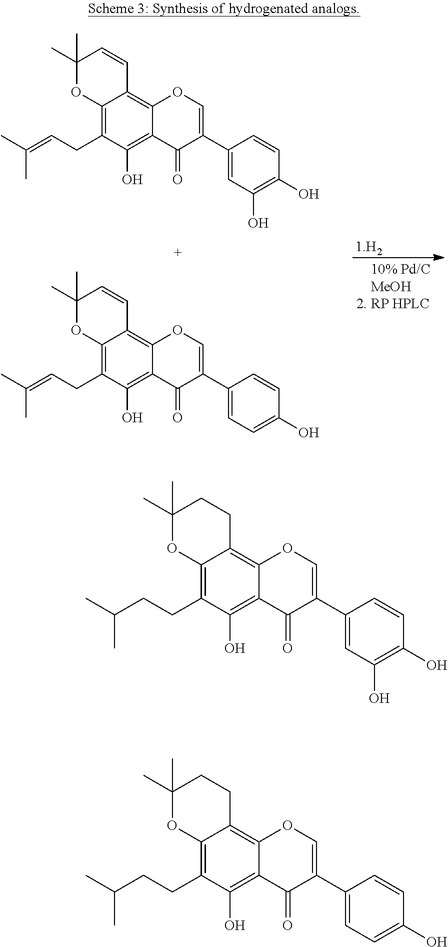 [SMILES: CC(C)=CCc1c2c(c3occ(-c4ccc(O)c(O)c4)c(=O)c3c1O)C=CC(C)(C)O2 ; "1.H2" ; CC(C)=CCc1c2c(c3occ(-c4ccc(O)cc4)c(=O)c3c1O)C=CC(C)(C)O2 ; CC(C)CCc1c2c(c3occ(-c4ccc(O)c(O)c4)c(=O)c3c1O)CCC(C)(C)O2 ; CC(C)CCc1c2c(c3occ(-c4ccc(O)cc4)c(=O)c3c1O)CCC(C)(C)O2]

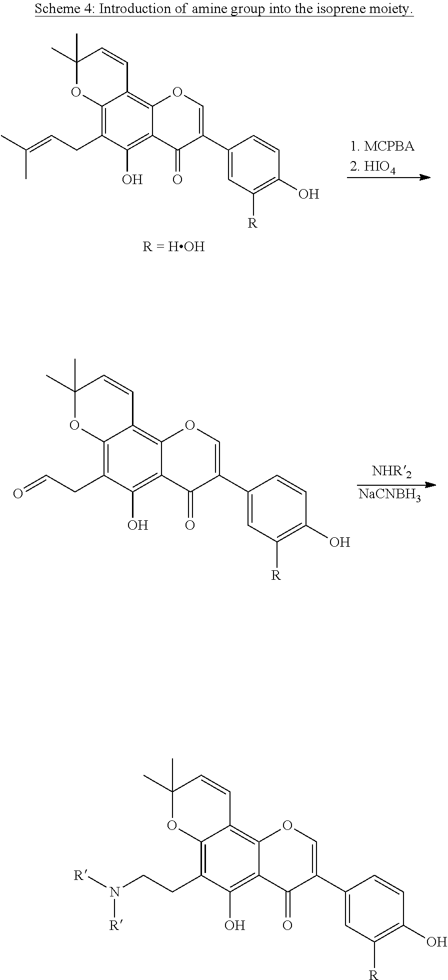

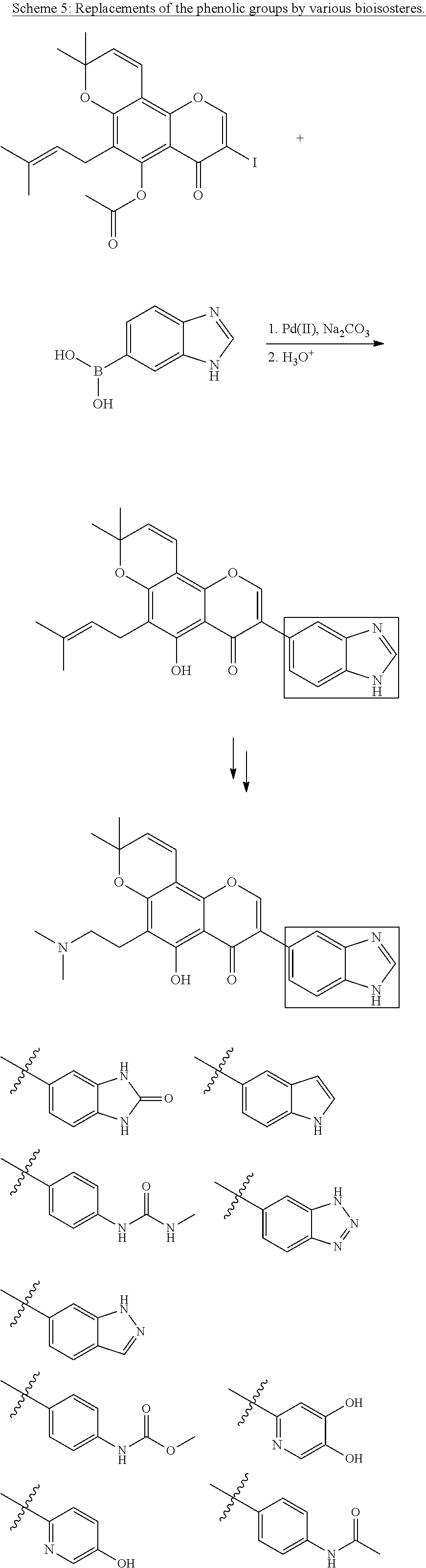

### Example 8

### Discussion

We believe that the target patient population that can benefit from a novel therapeutic extends beyond the MycN^{AMP} NBL patient group. Isopomiferin suppresses MycN in lung cell lines, suggesting the novel therapeutic could benefit all patients suffering from MycN^{AMP} tumors, independent of cancer type. This is significant, as there are subpopulations of MycN^{AMP} tumors from a number of cancers. For example, 12% of Wilms' tumor (WT), neuroendocrine prostate cancers (NEPC), ˜2-3% of non-small cell lung cancer (NSCLC), ˜2-3% of liver cancers are MycN^{AMP}, representing a substantial patient population that could benefit from an isopomiferin-based therapeutic. This is particularly important because MycN-driven tumors are noted for their aggressive phenotype and high mortality rate (Huang and Weiss 2013), and MycN-amplification occurs in later stages of adult cancers to exacerbate tumor development at a time when patients have often exhausted other options (Rickman et al. 2018). Furthermore, some MycN^{AMP} tumor types are understudied, due to a deficiency in cell models (specifically, WT and NEPC), and as a result they are limited in therapeutic options (Williams et al. 2015; Lee et al. 2016). Demonstrating benefits in MycN^{AMP} NBL and SCLC can expedite clinical testing to these underserved patient populations, especially if a basket trial approach can be pursued.

Using PLATE-Seq as an expression profile screening tool for network disruption is a unique approach to drug discovery that offers a competitive advantage over recent attempts to target MycN^{AMP} tumors. Our work advances PLATE-Seq methodology by crafting it as a tool that expedites drug development by prioritizing novel structural analogs based on their ability to revert a regulatory signature, and by identifying chemical substructures that induce off-target effects, particularly those associated with toxicity (Waring et al. 2001; Waring et al. 2001). PLATE-Seq lowers the cost of traditional expression profiling substantially (Bush et al. 2017), so it is feasible to screen a wider range of structures than traditional RNA-Seq. Introducing PLATE-Seq early in the development program can identify problematic structures that will be avoided in future iterations of the molecule, saving time and resources.

Selectivity for MycN^{AMP} cells will be used to develop MycN as a predictive biomarker of isopomiferin sensitivity. This biomarker will enable clinicians to identify responsive individuals, stratify patients at clinical trials, and draw meaningful comparisons between groups. This will enhance the probability of success in future clinical trials, and ensure that only responsive patients receive therapy. A basket trial design could be explored in future, in which MycN^{AMP} patients from across cancer types are binned based on specific tumor drivers. This trial design may be particularly important for rare tumors, such as WT and NEPC, and may be the quickest way to get these cancers tested for response to the novel therapeutic.

Encouraged by our preliminary data, a few key preclinical experiments will advance the lead compound toward clinical testing for a class of tumors for which there are limited therapeutic options. Isopomiferin suppresses MycN in xenografts, and we will now test efficacy using clinically-relevant orthotopic models that mimic disease pathophysiology. We will assess the stability of isopomiferin and optimized analogs, and will evaluate the compounds in drug safety panels to ensure their development potential. There is evidence that isopomiferin is safe for use in patients, overcoming one of the major hurdles in early drug development (Bowes et al. 2012). Isopomiferin is a prenylated isoflavonoid isolated from the wild citrus species M. pomifera (Darji et al. 2013); related isoflavonoids from soy are used as antioxidants in health beverages, and are generally regarded as safe (GRAS) by the US FDA (www.fda.gov). Two studies compared the anti-proliferative effect of pomiferin in cancer cell lines and non-transformed cells and found pomiferin >10-fold more potent in transformed cells (Darji et al. 2013; Son et al. 2007), suggesting that the scaffold is likely non-toxic to healthy tissues. Furthermore, adult tissues do not express MycN, which is normally confined to developing neural tissues. Inhibiting MycN should therefore not be problematic for healthy tissues. This may be an important feature that could benefit sensitive pediatric patients.

### Example 9

### Identification of Other Compounds that Revert the MR Module

To evaluate the effects of lethal molecules on MycN^{AMP} regulatory network, 90 selective compounds were analyzed by transcriptome analysis using PLATE-Seq methodology, followed by analysis with VIPER algorithm. This network based analysis evaluates the change in expression of all cognate targets of a given transcription factor and treats them as a single gene set, inferring the relative activity of a given regulator protein. The VIPER plot ranks TF regulons on the basis of activity, from highest to lowest along the x-axis, highlighting the rank of the activated MRs (red) and the suppressed MRs (blue) along the top and bottom of the GSEA chart. The running normalized enrichment score (NES) indicates the relative enrichment of the genes included in the regulons, such that the higher the NES score, the more enriched the MR regulons are.

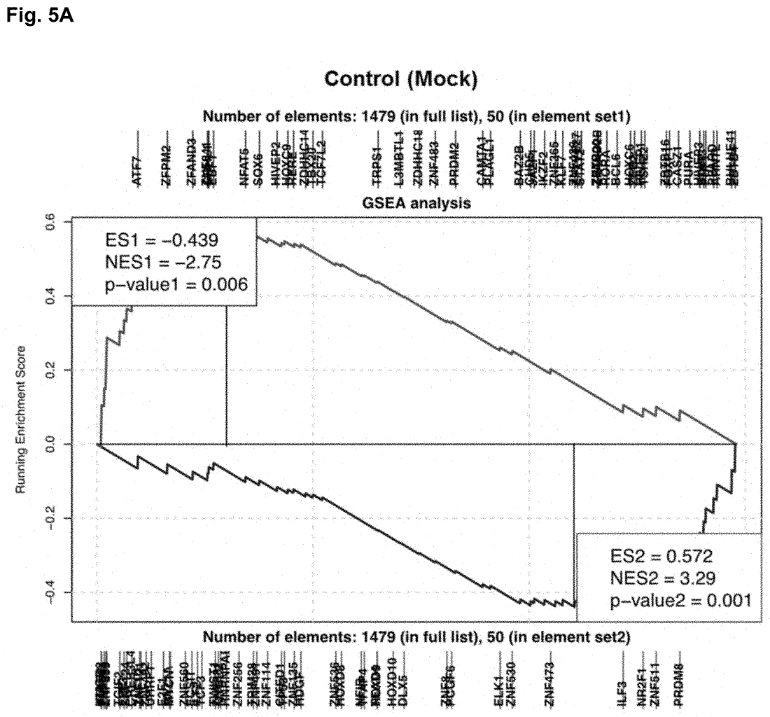

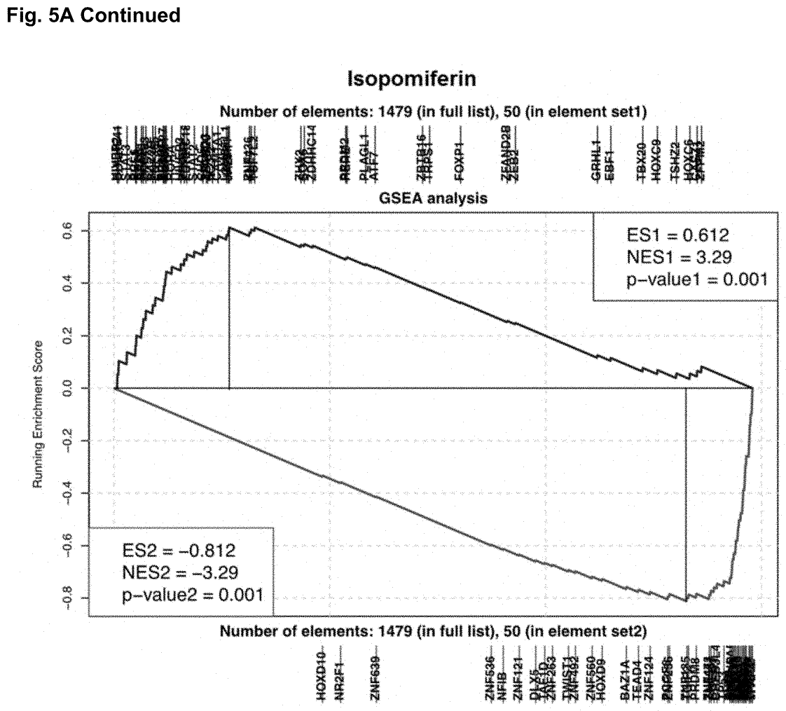

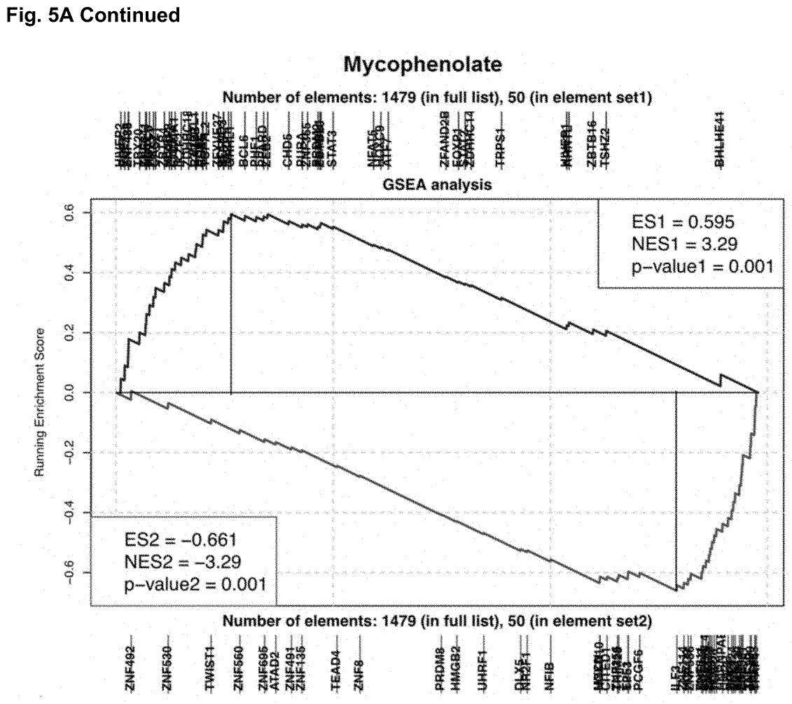

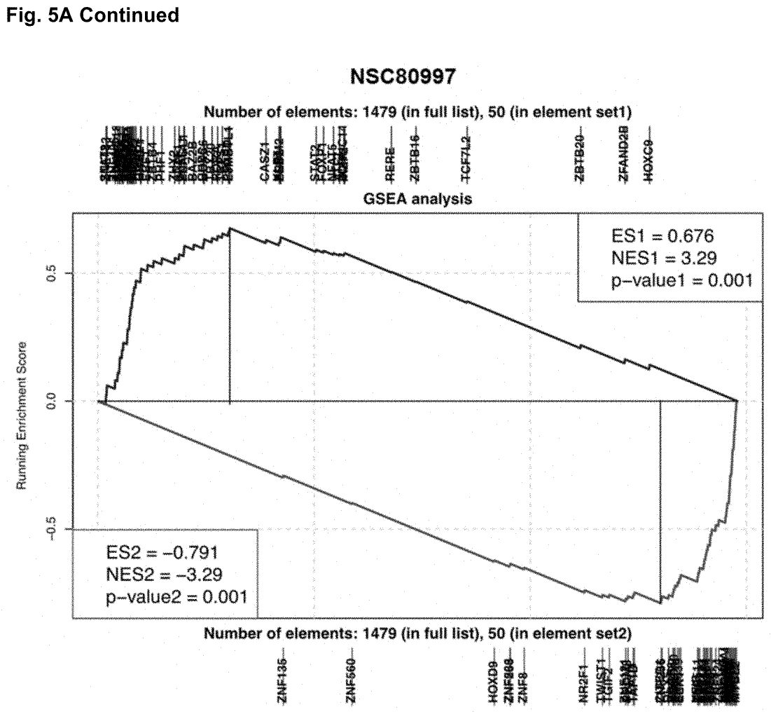

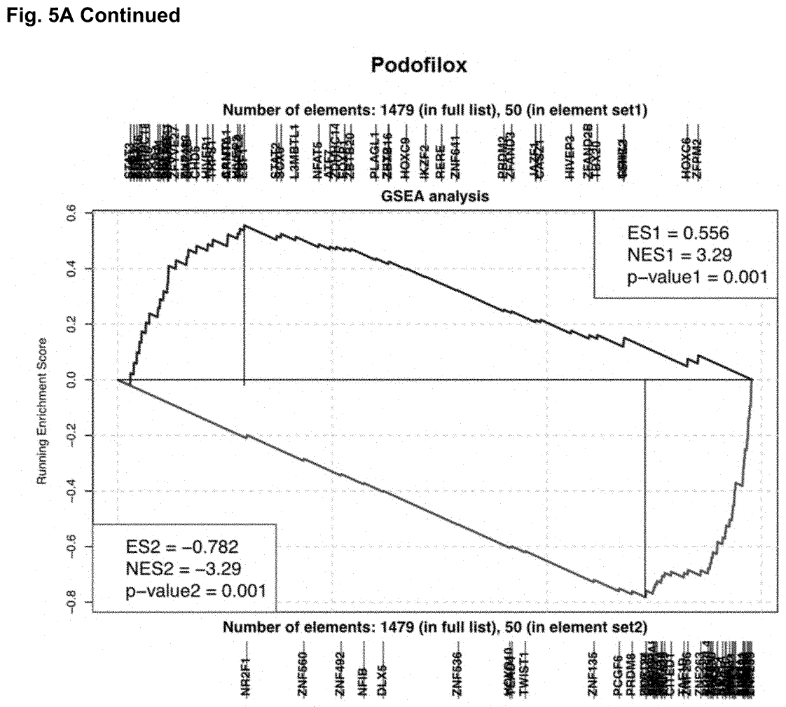

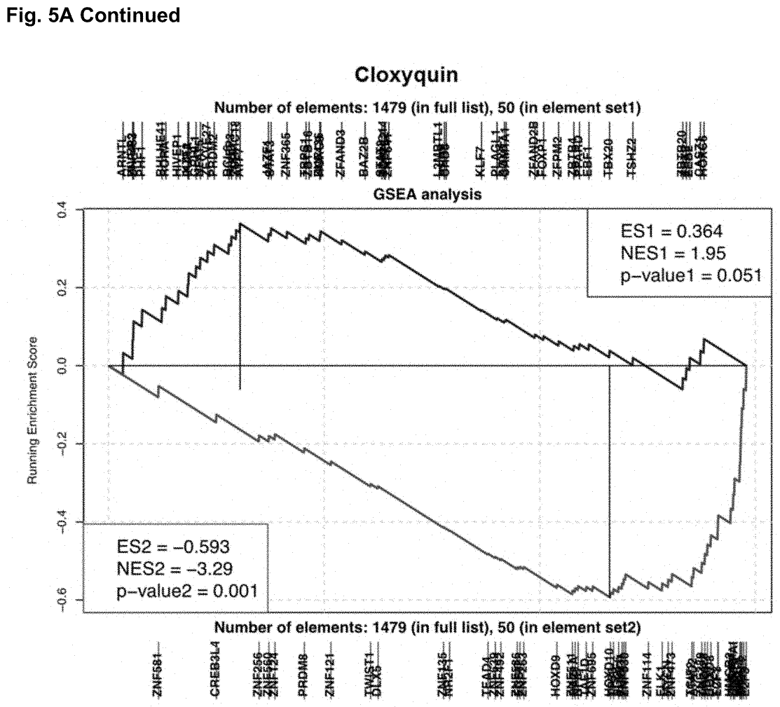

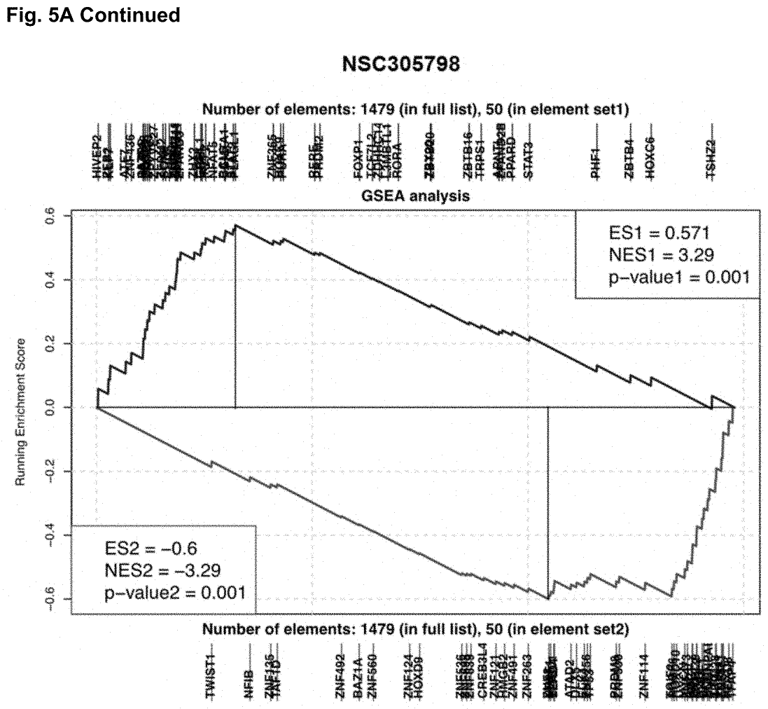

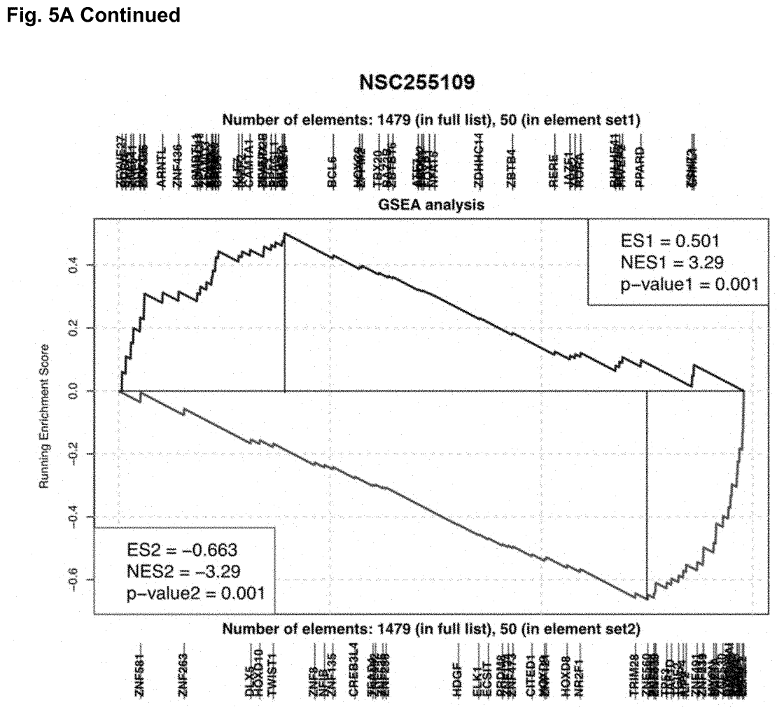

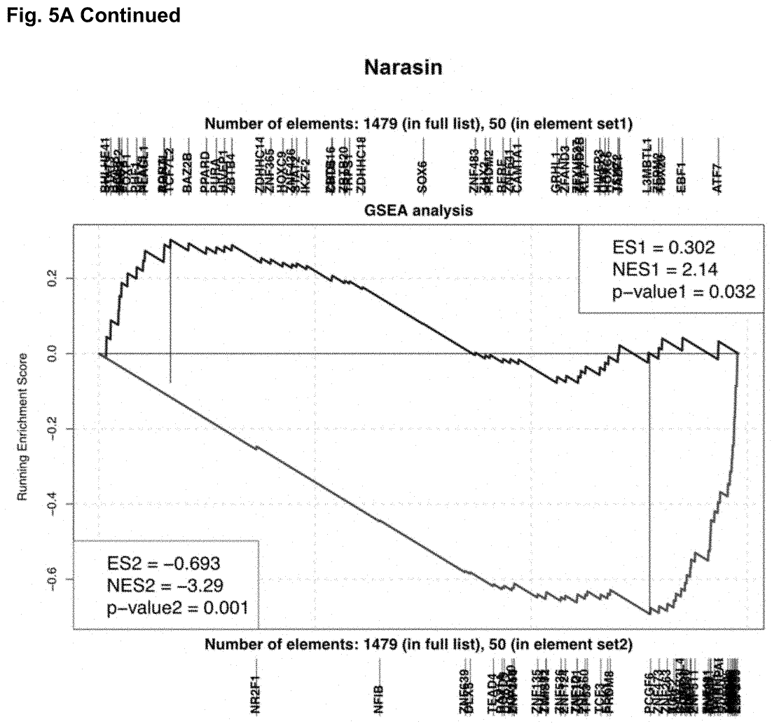

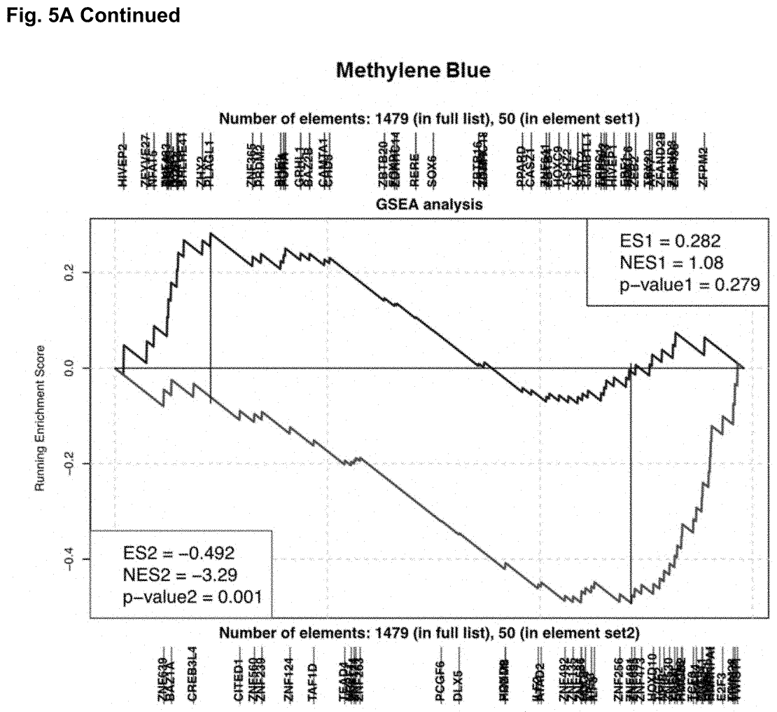

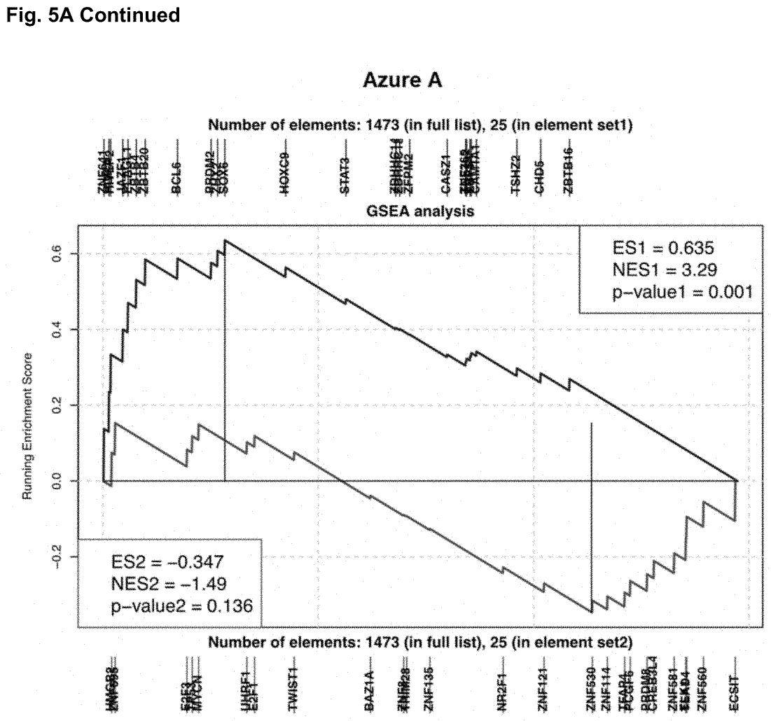

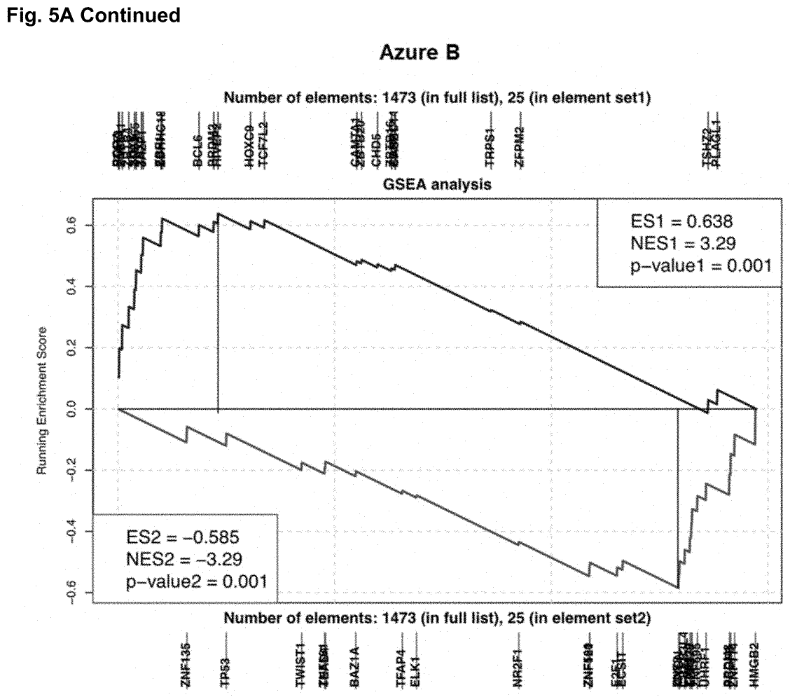

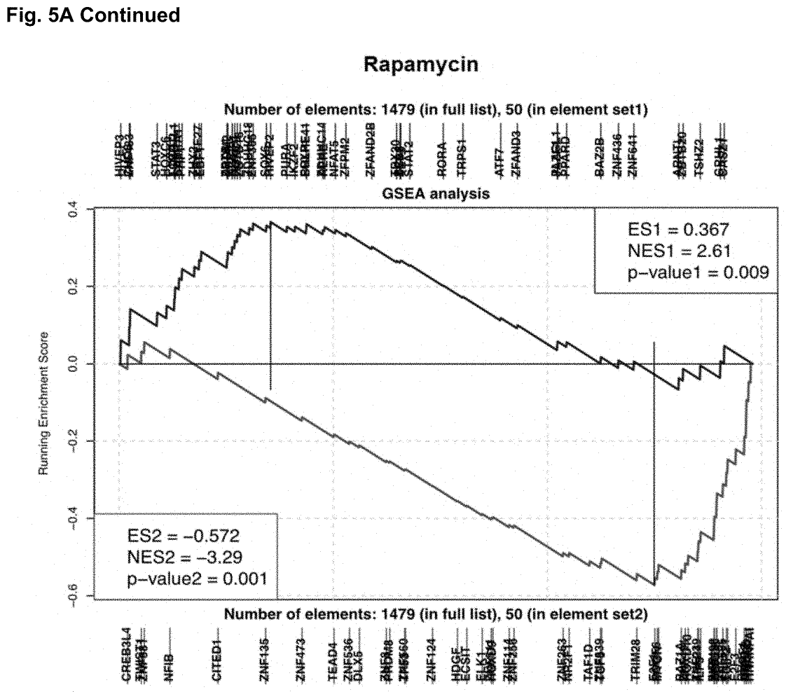

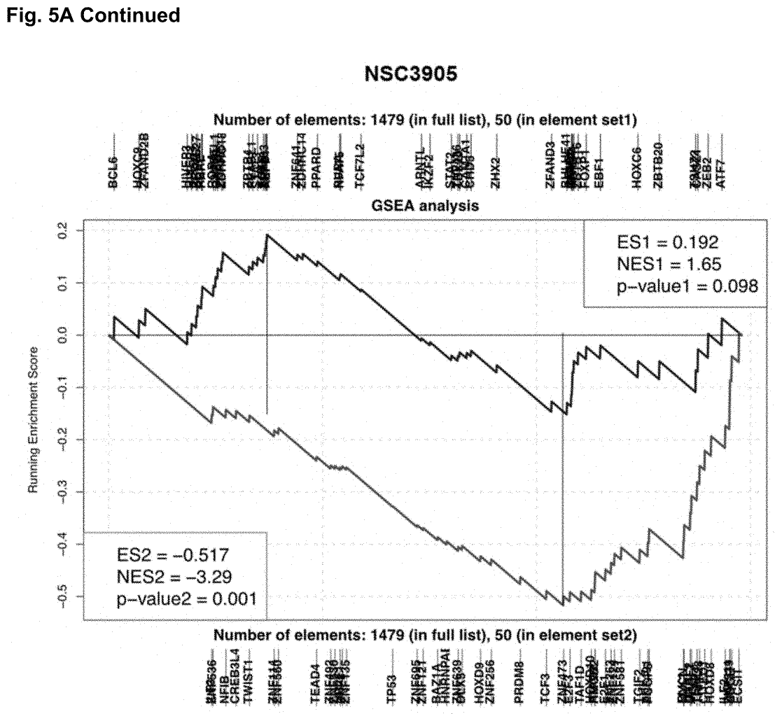

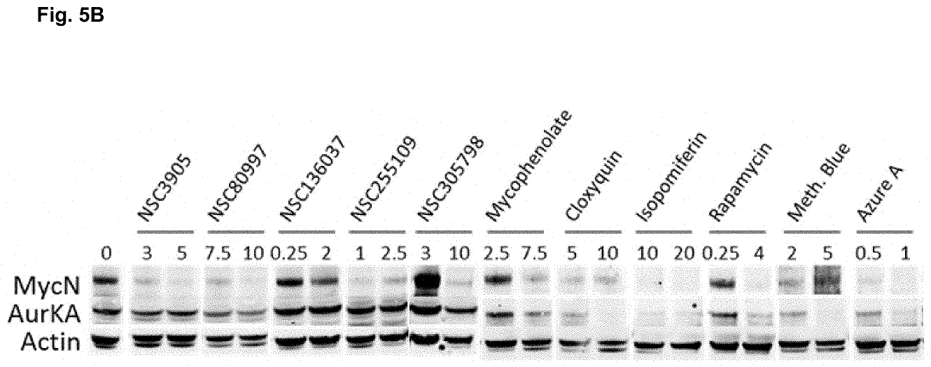

To evaluate the effect of subtype selective compounds on the regulatory module, SK-N-Be2 cells were treated with the IC₂₀ concentration of each compound for 24 hrs, which had been determined empirically previously. We hypothesized that subtype-selective compounds disrupt the MR regulatory module driving the MycN^{AMP} NBL. We identified 50 activated and 50 repressed master regulators in MycN^{AMP} NBL, which revealed many key regulator proteins previously found to support MycN expression, including MycN itself. Compounds were ranked based on their normalized enrichment score, an indication for the effect of the compound on the MR module. This analysis revealed 13 compounds that revert the regulatory module driving MycN^{AMP} NBL (Table 2 and FIG. 5A).

TABLE 2
Other compounds that revert the MR signature
and their known bioactivity.

| Compound | IC₂₀ (μM) | Known Bioactivity |
|---|---|---|
| Podofilox | 1 | Microtubule destabilization by |
| | | binding at colchicine binding site |
| | | (Lu et al) |
| Mycophenolic | 2.5 | Inhibits de novo guanosine |
| acid | | biosynthesis through reversible |
| | | inhibition of IMPDH |
| Isopomiferin | 10 | |
| Narasin | 1 | Analog of salinomycin, selectively |
| | | lethal to CSCs (Gupta 2009). |
| | | Inhibits Wnt signaling in CLL |
| Methylene Blue | 2 | |
| Azure A | 1 | |
| Cloxyquin | 5 | Activator of TRESK Ka+ channel |
| | | (Wright et al), autophagy inducer |
| | | (Zhang 2016) |
| NSC80997 | 7.5 | Analog of Glucocorticoid Receptor |
| | | agonist Cortivazol |
| NSC305798 | 5 | |
| NSC255109 | 2.5 | Analog of Geldanamycin and 17 |
| | | DMAG; HSP90 inhibitor and |
| | | c-Myc suppressor |
| Azure B | | |
| NSC3905 | 3 | |

After having identified thirteen compounds that revert the MR module, we wanted to confirm that the repression of the regulatory network ameliorates MycN expression. By targeting the supporting network, we believe that these compounds have a higher chance of successfully suppressing MycN abundance in vivo. Eleven reverting compounds were tested at their IC₂₀ and IC₅₀ concentrations. All 11 compounds suppressed MycN abundance after 24 hours, though a few of the treatments required the IC₅₀ to induce an effect (FIG. 5B).

### Example 10

### Methylene Blue and Azure a Suppress AurKA Expression and Destabilize MycN

Aurora kinase A (AurKA) dimerizes with MycN in NBL cells, blocking a phosphorylation site and subsequently protecting MycN from proteolytic degradation (Gustafson et al., 2014; Otto et al., 2009). Disrupting the interaction between Aurora A and MycN causes protein destabilization and cell death in MycN-driven neuroblastoma in preclinical cellular models and in-vivo (Carol et al., 2011; Faisal et al., 2011; Gustafson et al., 2014; Maris et al., 2010; Otto et al., 2009). As a result, AurKA inhibitors have been proposed as effective therapies for MycN driven Neuroblastoma.

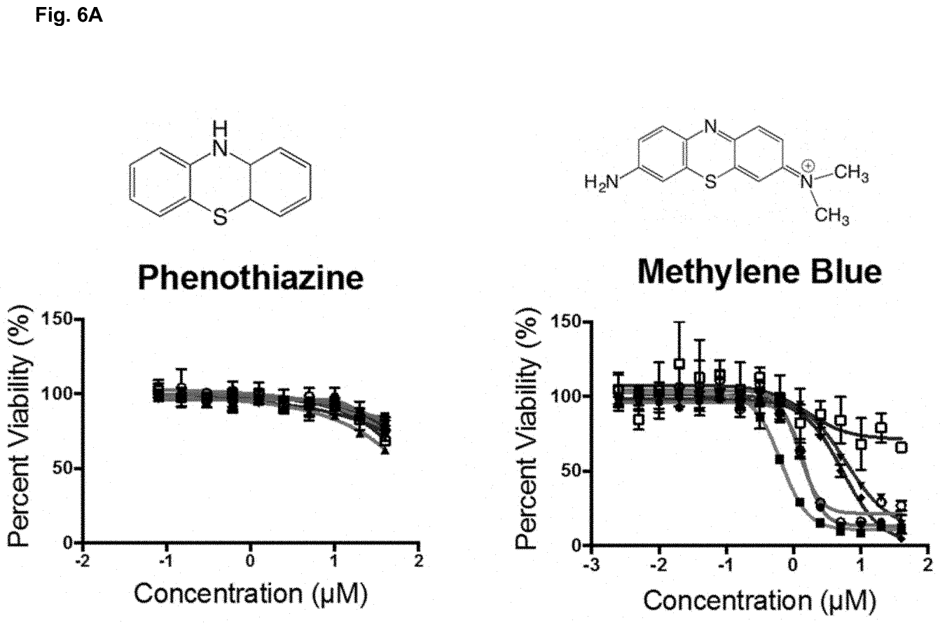

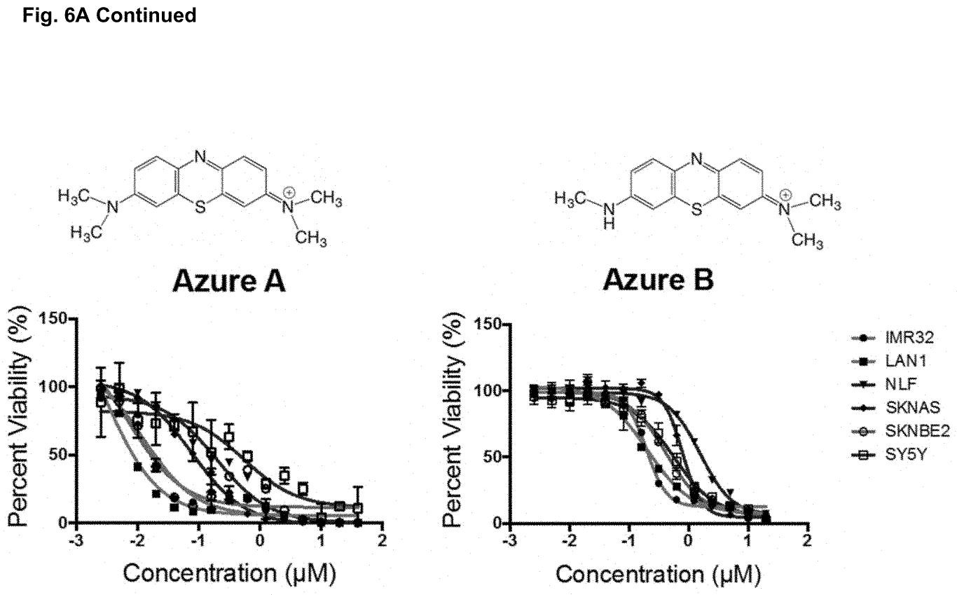

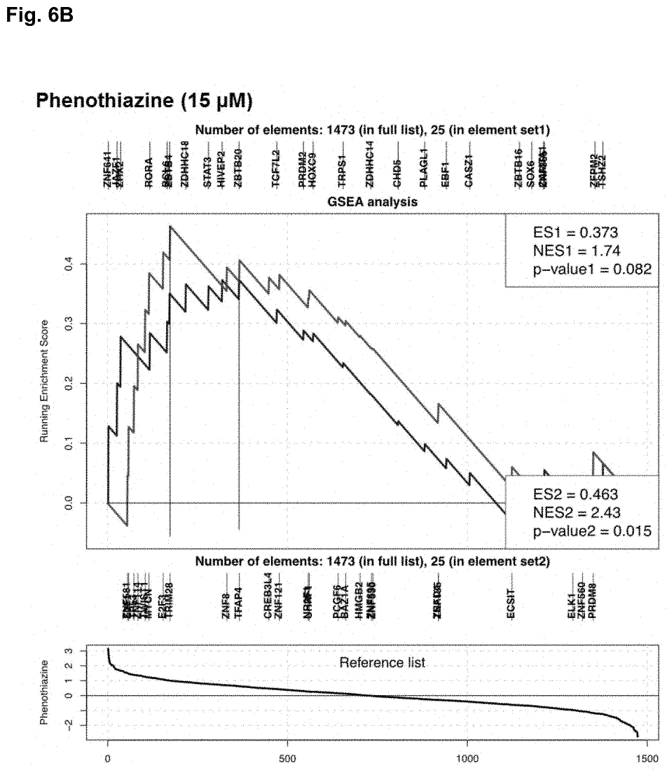

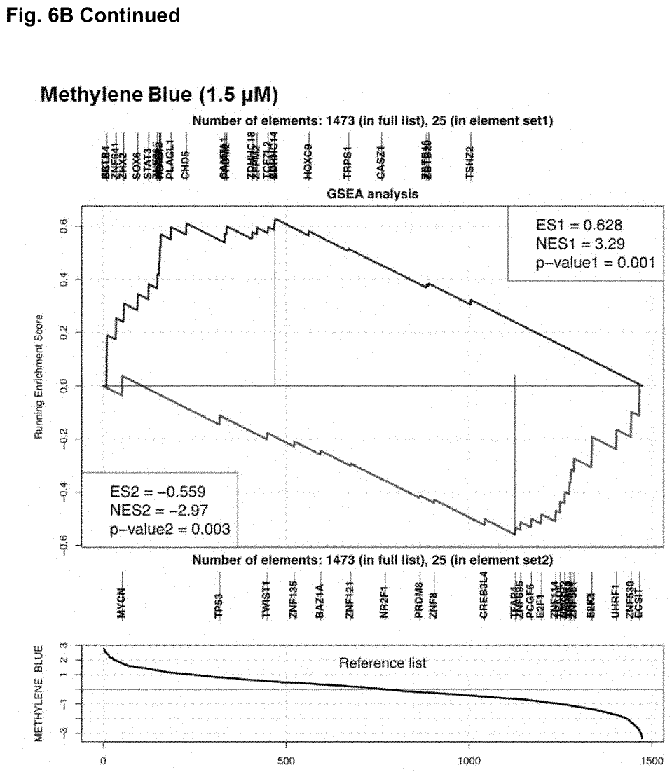

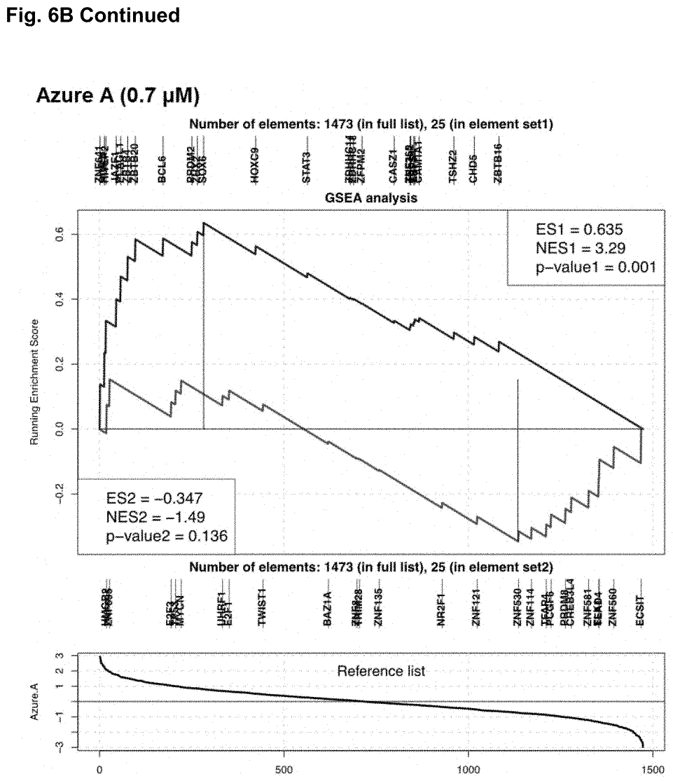

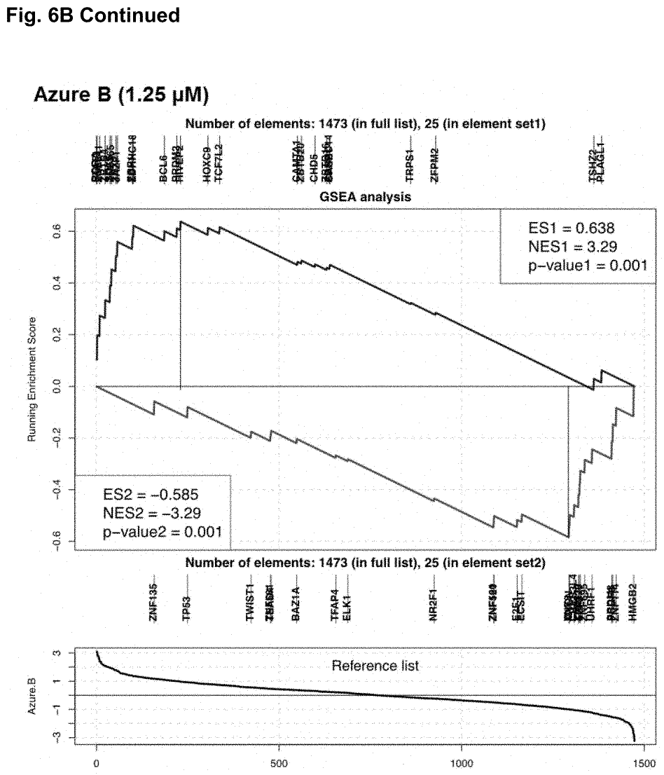

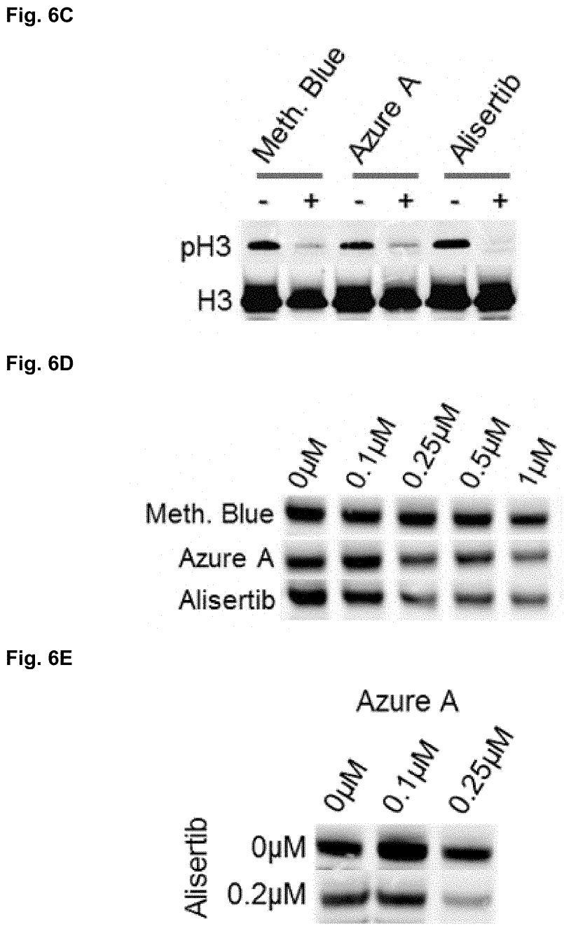

We hypothesized that AurKA inhibition may be a mechanism through which some of the compounds that disrupt the regulatory module suppress MycN. We evaluated the ability of the MR-reverting compounds to suppress Aurora Kinase A expression at the IC₂₀ and IC₅₀ of each compound. Five of the 11 compounds tested were able to inhibit AurKA abundance at the IC₅₀, including Methylene blue and its analog Azure A (FIG. 6A). Azure A was able to completely suppress AurKA at 1 μM, making it more potent than methylene blue. MB and Azure A vary in their structure at the methylation state of the amine. Though Azure A is more potent than MB, it is notable that the core structure, phenothiazine, is inactive in NBL (FIG. 6B). Based on these findings, it appears that modification of the core structure influences drug potency, and offers insight into how the drug may be modified through medicinal chemistry to improve the drug characteristics.

Since Azure A was the most potent drug tested at AurKA suppression, we investigated the ability of MB and Azure A to inhibit MycN across a range of doses and in direct comparison with the AurKA inhibitor alisertib, in order to assess the relative potency of these two drugs against the clinical candidate. We also compared the potency of these drugs against a recently developed AurKA inhibitor that acts by disrupting protein confirmation rather than simply blocking the protein:protein interaction site as alisertib does. All four AurKA suppressors blocked Histone 3 phosphorylation at 1 μM, indicating that all compound achieved AurKA inhibition at this dose (FIG. 6C). When SK-N-Be2 cells were treated to the four AurKA inhibiting compounds, Azure A suppressed MycN abundance at a dose similar to the clinical candidate alisertib, whereas the Azure A prodrug MB was less effective (FIG. 6D). Of the four compounds tested, the conformation disrupting compound (CD532) was more potent at MycN suppression than MLN8237, consistent with earlier reports (Gustafson et al., 2014). Together, these findings suggest that the Azure A compound, which has been designated as safe for administration to children, could be as equally potent at MycN suppression as MLN8237, a drug recently tested in Phase II trials for pediatric neuroblastoma.

As alisertib inhibits AurKA activity without affecting its protein expression, we hypothesized that the ability of Azure A to suppress AurKA abundance might represent an opportunity to develop combination treatments that synergize to suppress MycN. By combining low-dose treatments of Azure A and alisertib, we found that co-treatment of alisertib and either methylene blue or azure A was more effective than either compound individually (FIG. 6E)

### Example 11

### Chemical Screen Identifies MYCNA-Selective Inhibitors

Genetic inhibition of MycN expression induces apoptosis in MYCNA cells, so we hypothesized that a phenotype-based screen for compounds with enhanced potency in MYCNA lines would enrich for MycN-suppressing compounds. Expression profiles from 39 NBL cell lines were evaluated to identify cell models that recapitulate the regulatory networks observed in MYCNA primary tumors. Using the VIPER algorithm, which measures transcription factor activity based on regulon enrichment (Alvarez et al. 2016), the activity profile of 25 Master Regulators from NBL primary tumors was assessed in cell lines, which revealed SK-N-Be2 and IMR-32 as most closely resembling the MR profile observed in primary tumors (Rajbhandari et al. 2018). Two cell lines representing the Mesenchymal NBL subtype (MES) were included to contrast the MYCNA subtype. These cell models were selected based on expression of a mesenchymal gene expression signature that defines the subtype (Rajbhandari et al. 2018; Phillips et al. 2006). SK-N-AS and NLF were chosen to model MES NBL, because of high expression of the MES signature.

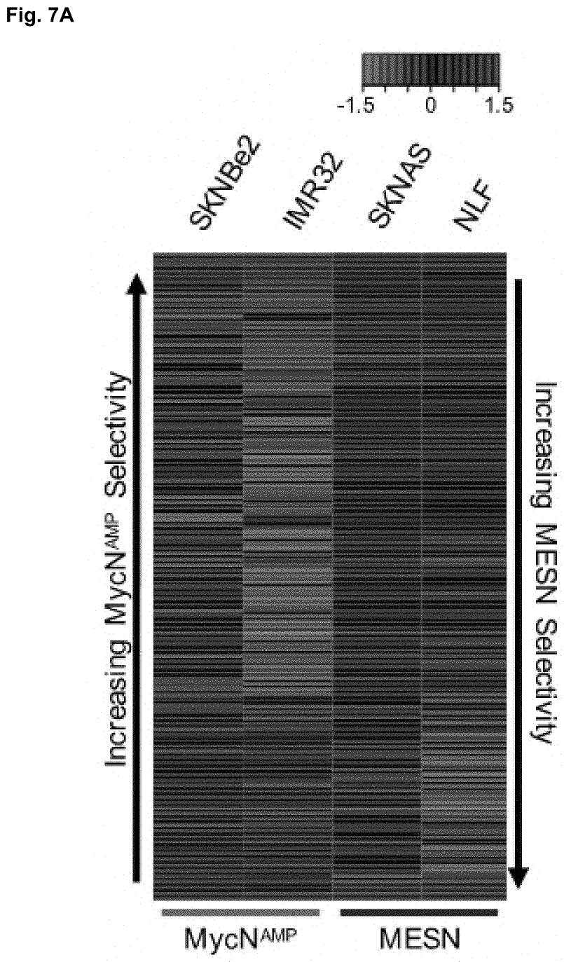

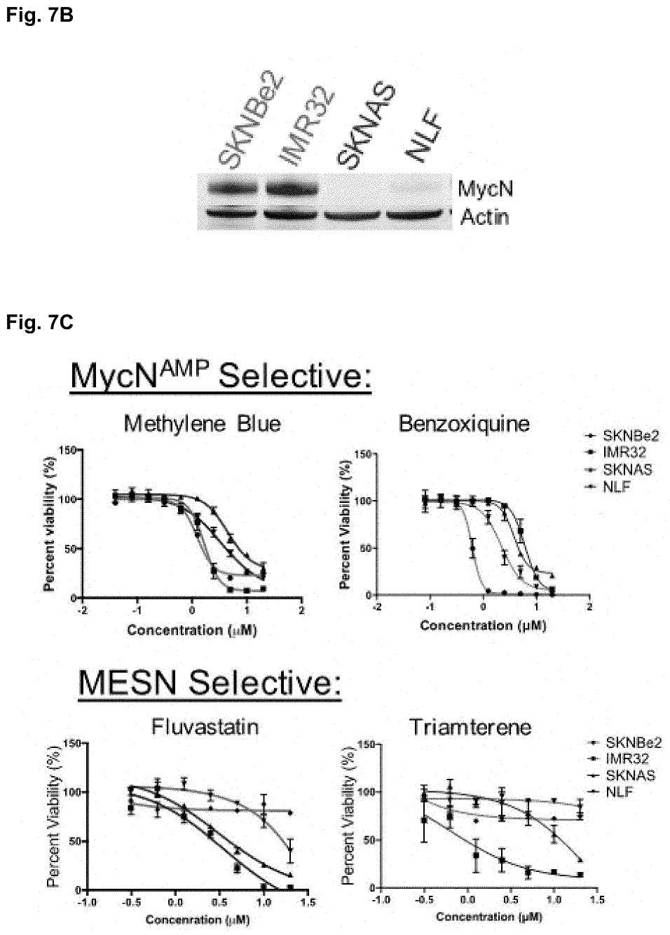

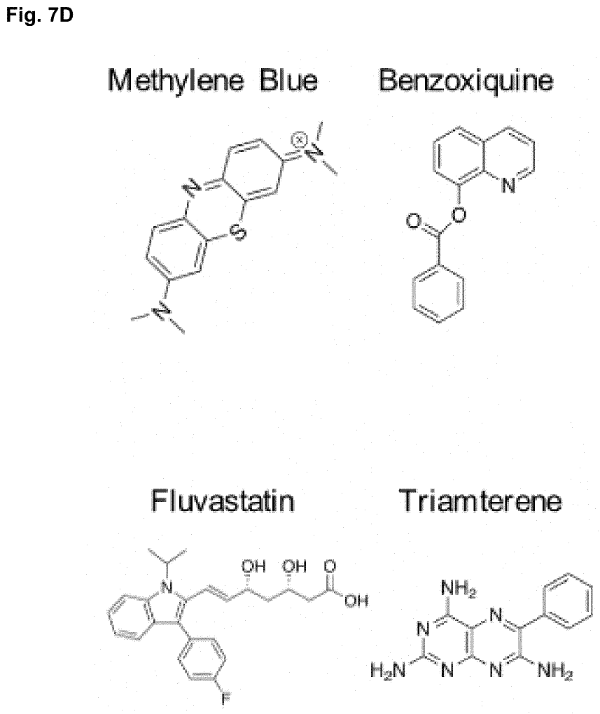

To identify subtype-selective inhibitors, we systematically screened ˜5500 compounds from three chemical libraries to identify molecules with greater potency in MYCNA lines. The NCI Clinical Collection, the SPECTRUM Collection and the NIH Diversity Set were chosen because of their enrichment for bioactive molecules, diversity in chemical structure, and for inclusion of compounds with known mechanisms of action. Compounds were initially tested at a single concentration and time-point (20 μM for 72 h), which identified compounds that were lethal to any one of the four cell lines (<10% viability). All lethal compounds were rescreened across a five-point dilution series ranging from 20 μM to ˜250 nM, which enabled calculation of an IC₅₀ value for each compound in each of the four cell lines. By ranking compounds based on average IC₅₀ values for the two subtypes, compounds that were more potent in either MYCNA or MES cell lines were identified (FIGS. 7A-7D).

### Example 12

### Activity Inhibition of Master Regulator Proteins Prioritizes MYCNA-Selective Compounds

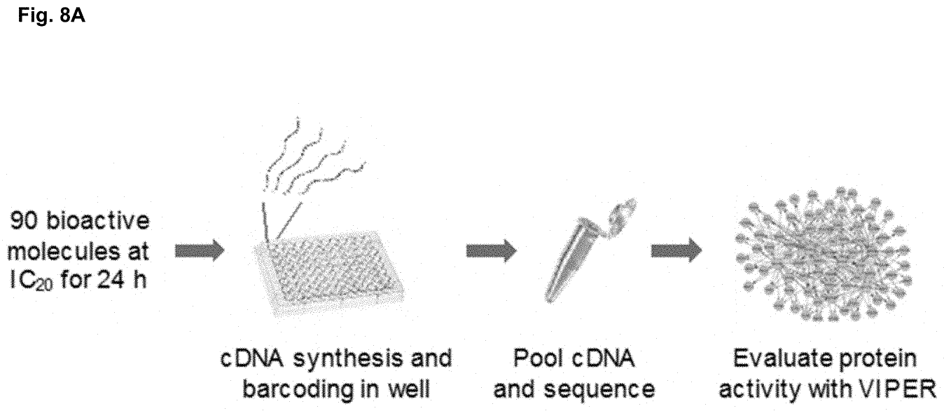

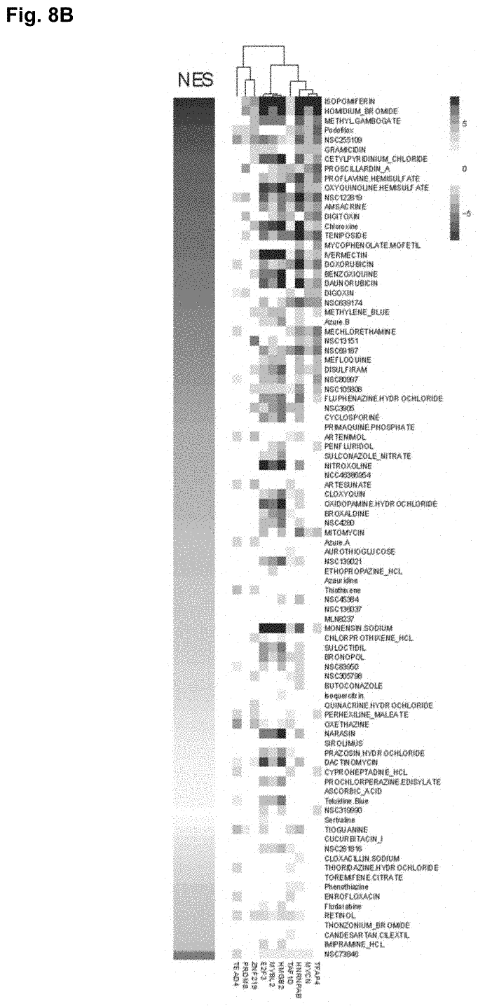

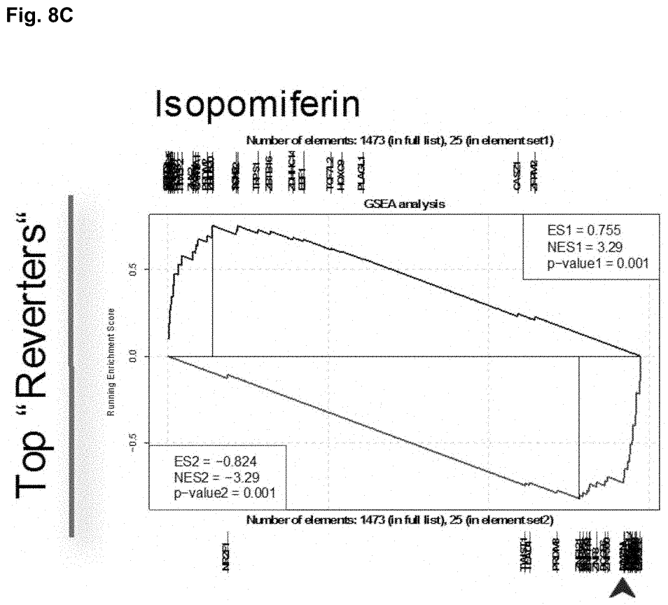

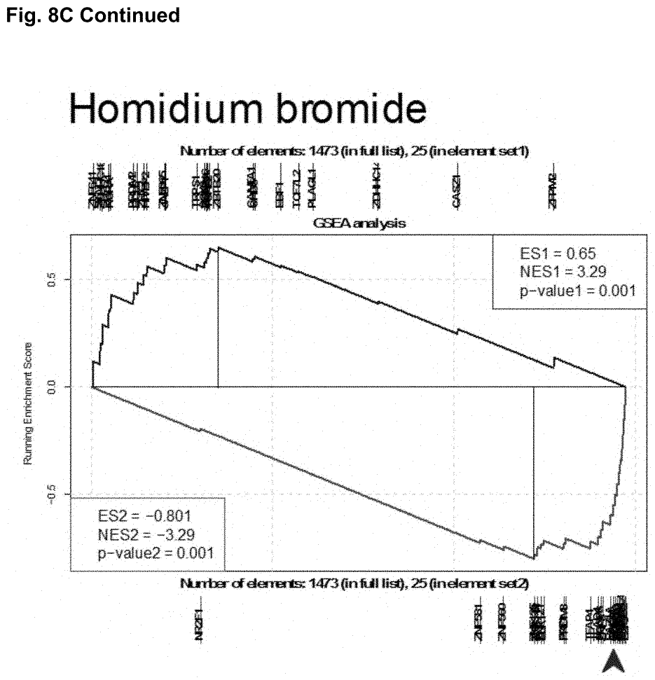

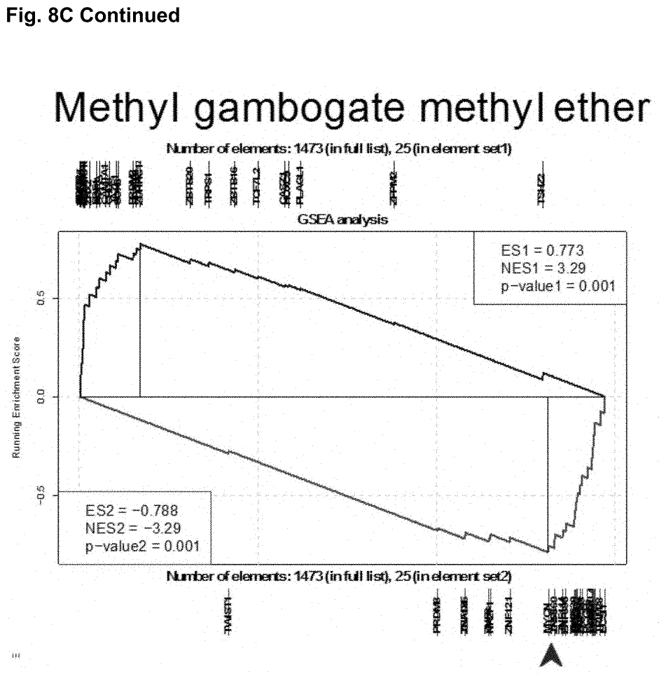

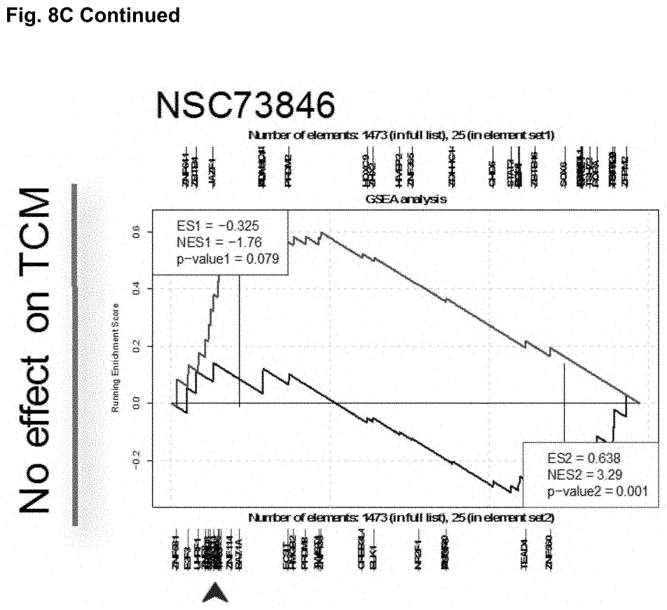

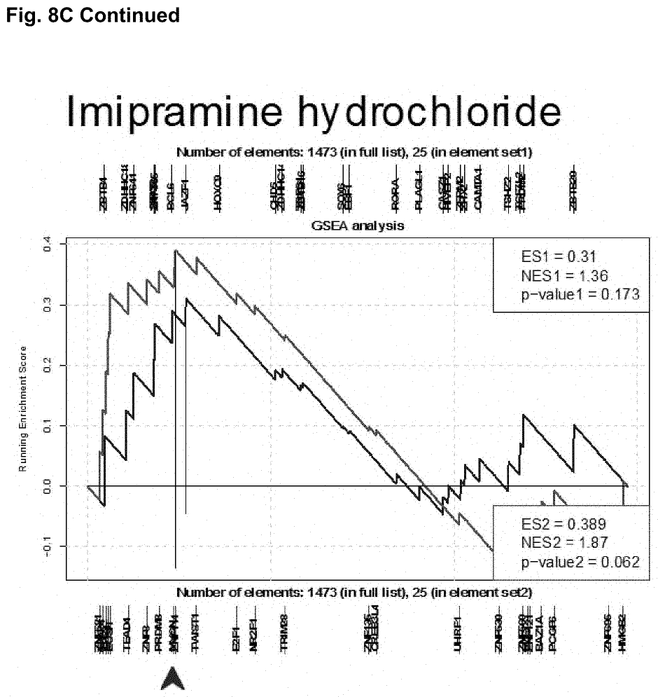

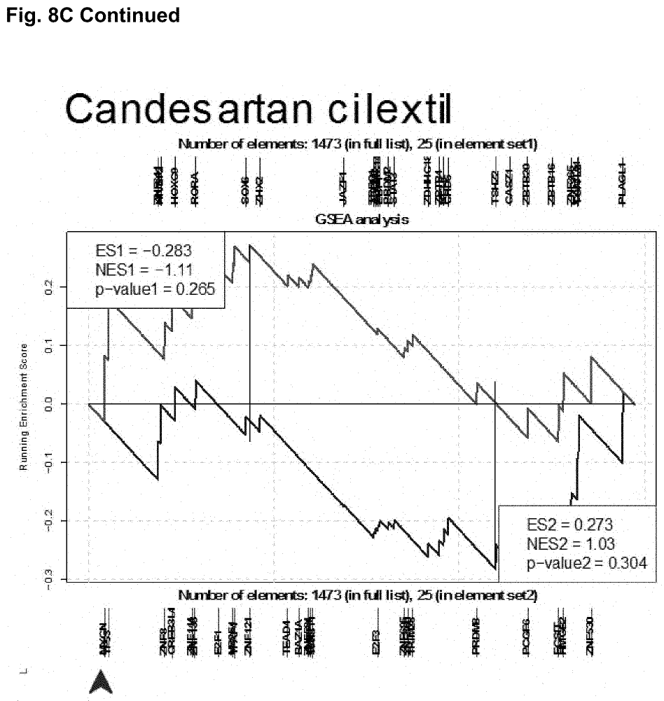

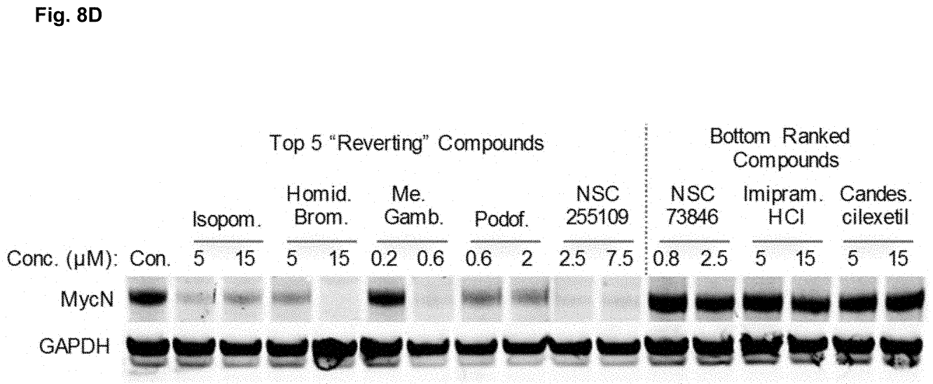

After ranking compounds based on subtype-selectivity, the top 90 MYCNA-selective compounds were evaluated using a high-throughput expression profiling tool, called PLATE-Seq (FIG. 8A) (Bush et al. 2017), to identify compounds that collapse the MYCNA subtype tumor checkpoint module. SK-N-Be2 MYCNA cells were treated to the top 90 lethal compounds at their IC20 concentration, which was determined empirically.

We developed a software pipeline for the analysis of perturbational PLATE-Seq expression profiles. A set of 50 candidate MRs from the MYCNA signature was selected to look for activity reversion in drug signatures, enabling us to prioritize compounds based on the ability to collapse the MYCNA TCM, the core set of ten transcriptional regulators that drive aggressiveness of the MYCNA tumor subtype (Rajbhandari et al. 2018). The effect of each compound on transcription factor activity profiles was inferred using VIPER analysis of PLATE-Seq expression profiles. Gene set enrichment analysis was used to compute Normalized Enrichment Scores (NES) on VIPER-inferred drug signatures as readout of their efficacy to experimentally reverse the TCM. Highly negative NES indicates strong activity reversion, driven by MR activity suppression by the compound. FIG. 8B shows the ranked list of 90 compounds ranked by their inferred efficacy using this approach. Of all 90 compounds tested using this methodology, isopomiferin had the strongest effect on the TCM, suppressing the activity of all ten proteins that comprise the MYCNA TCM.

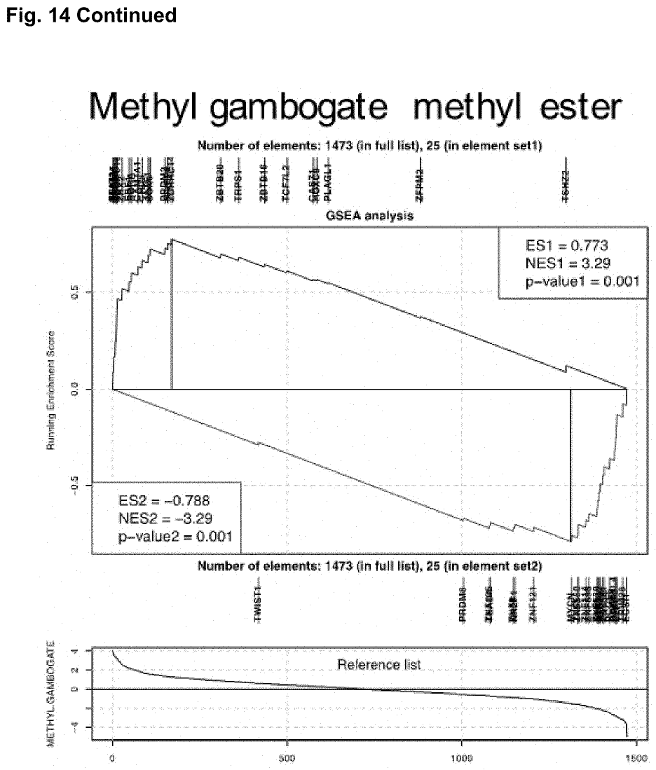

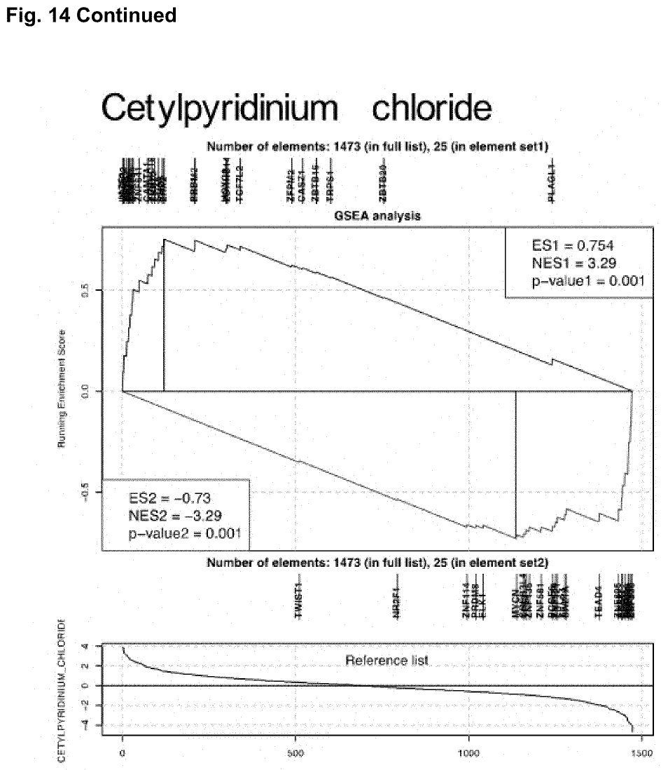

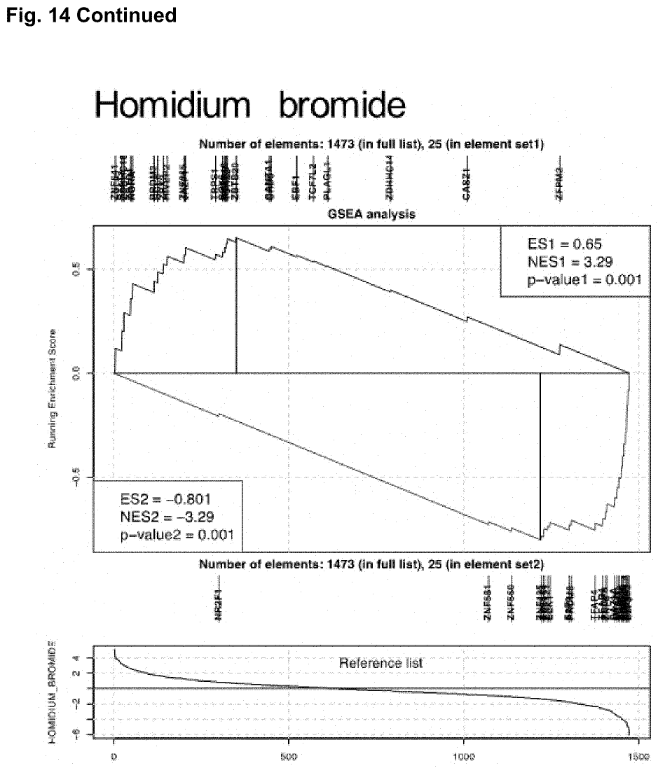

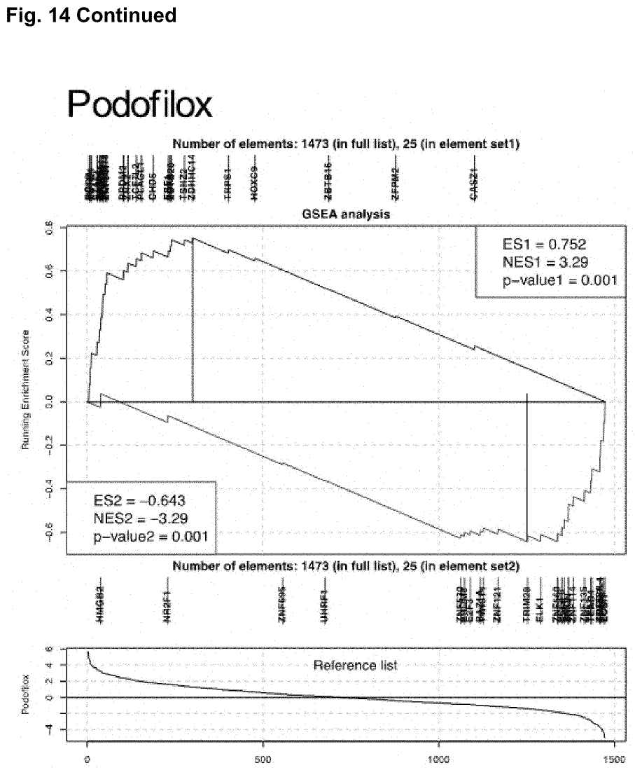

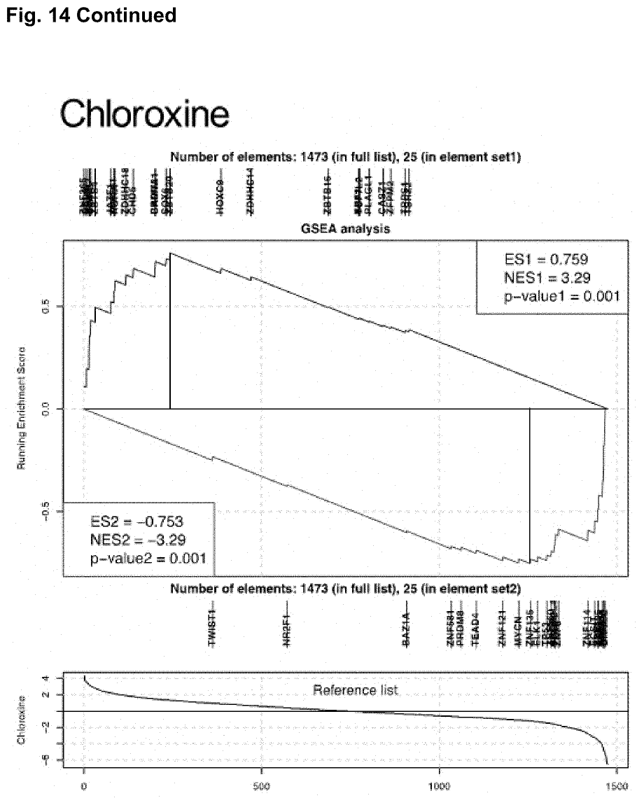

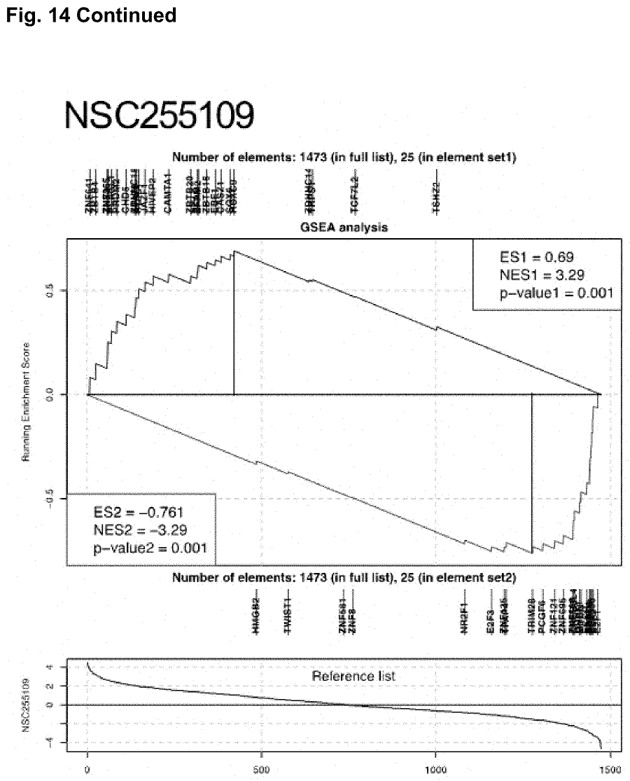

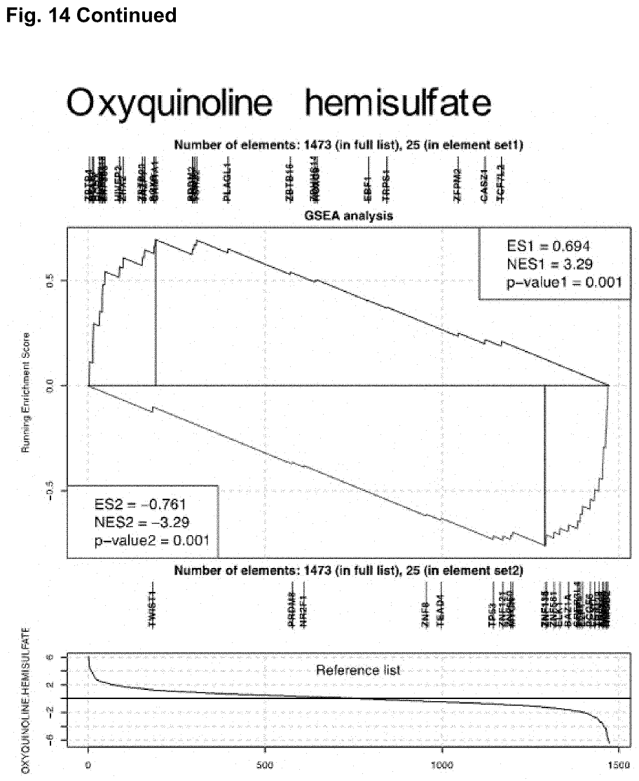

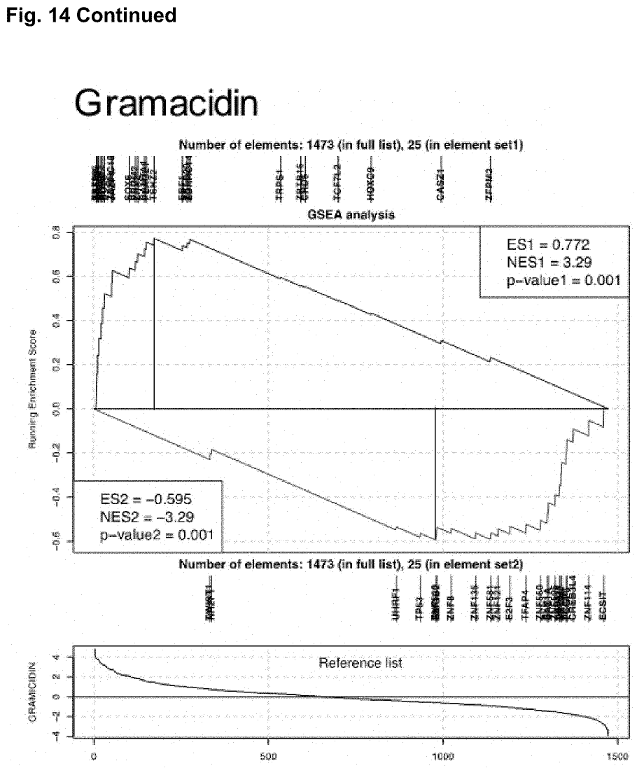

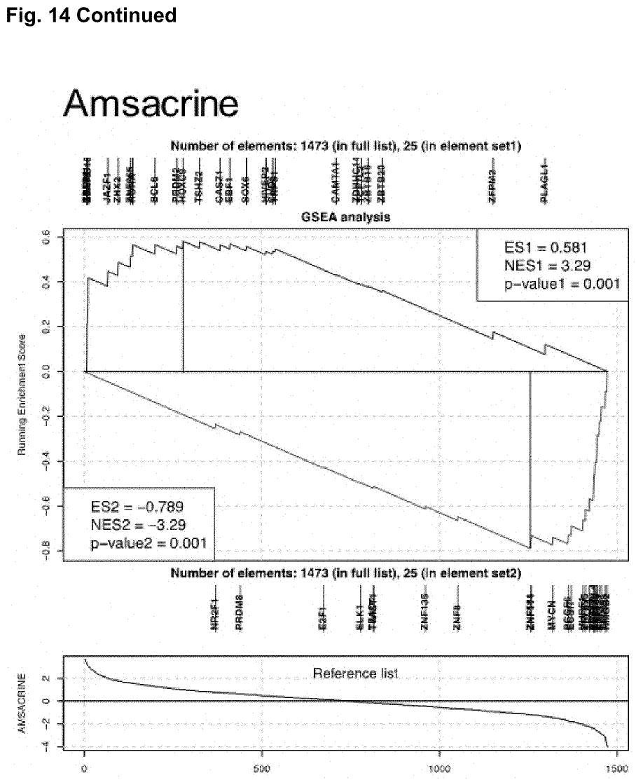

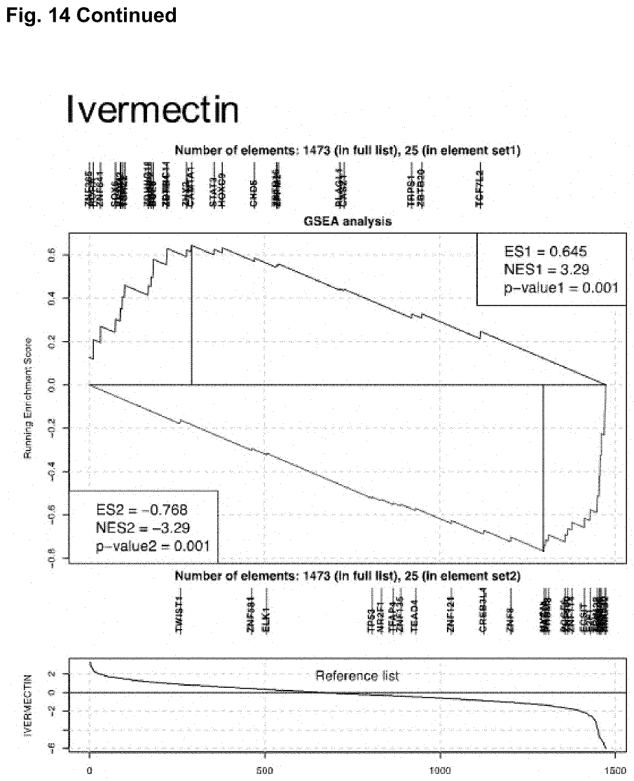

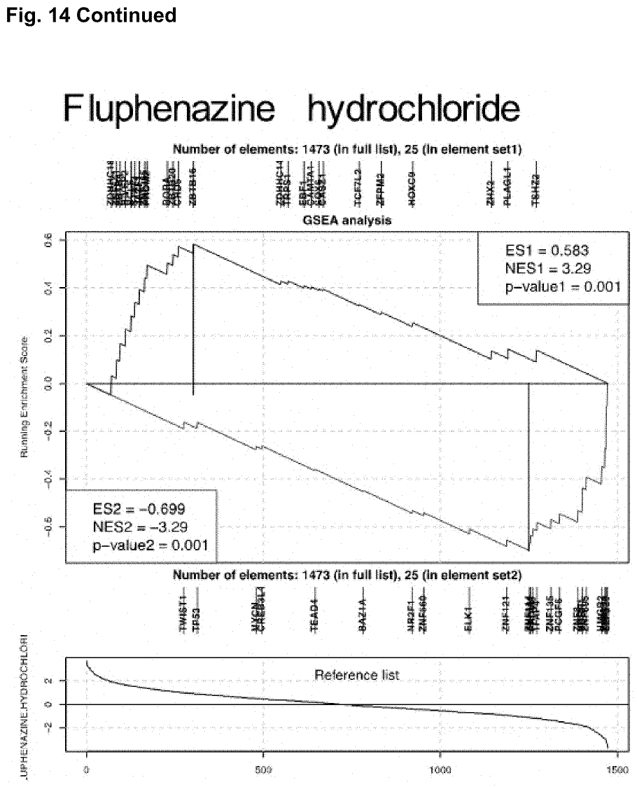

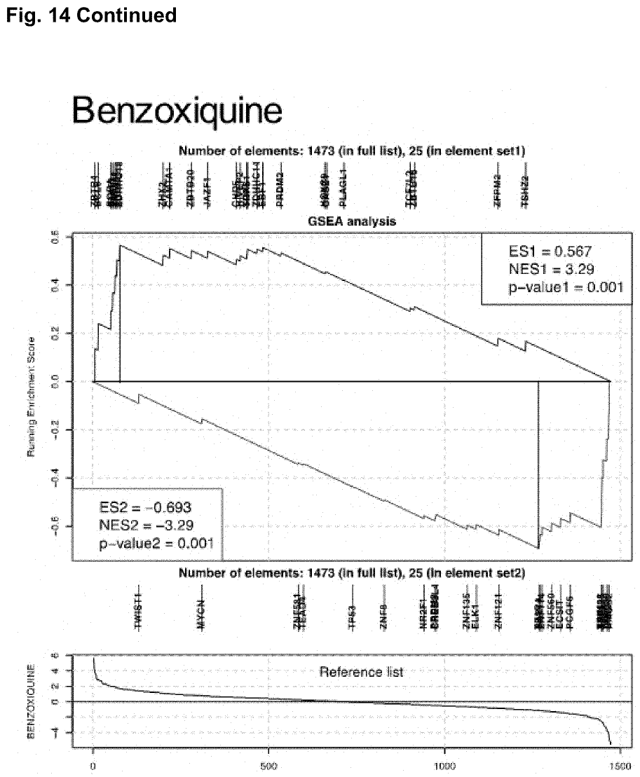

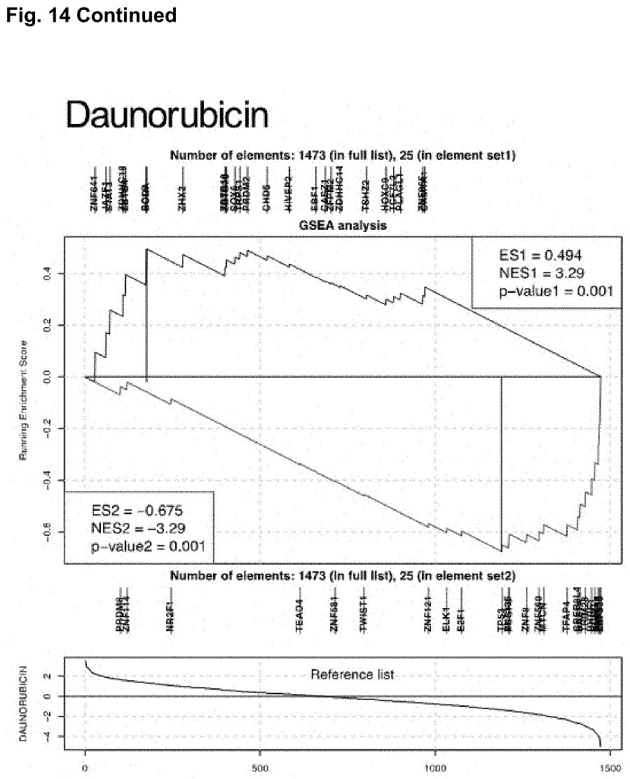

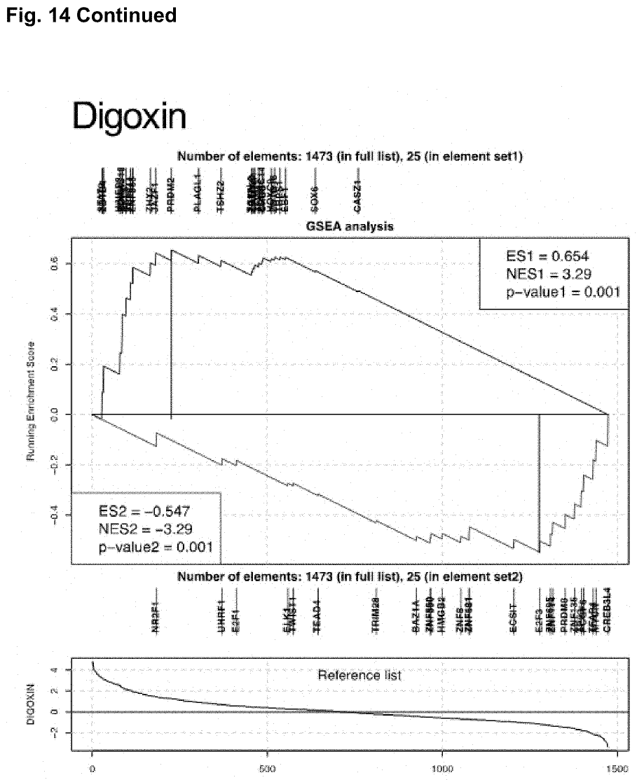

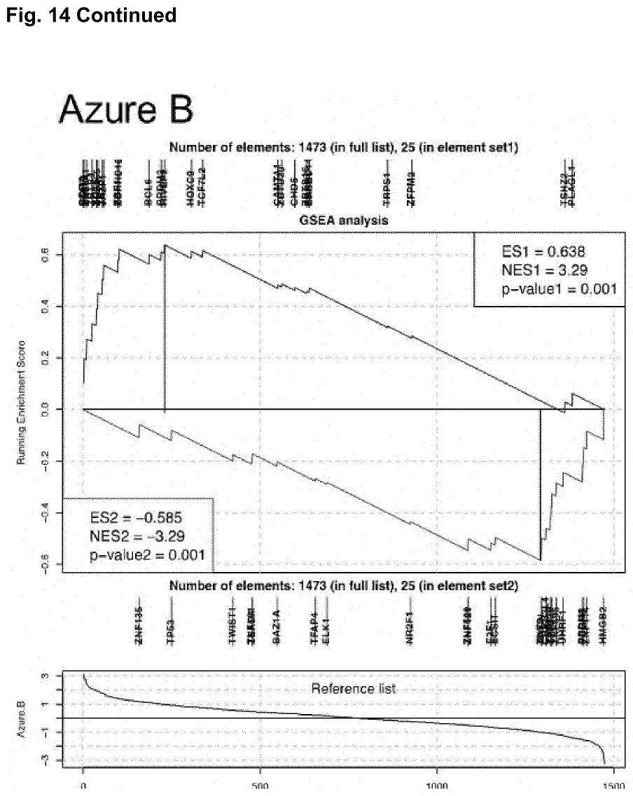

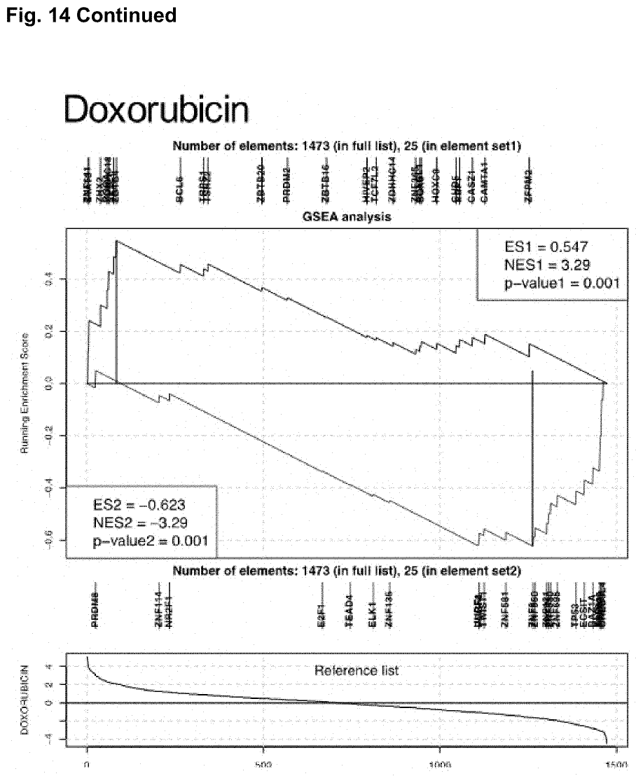

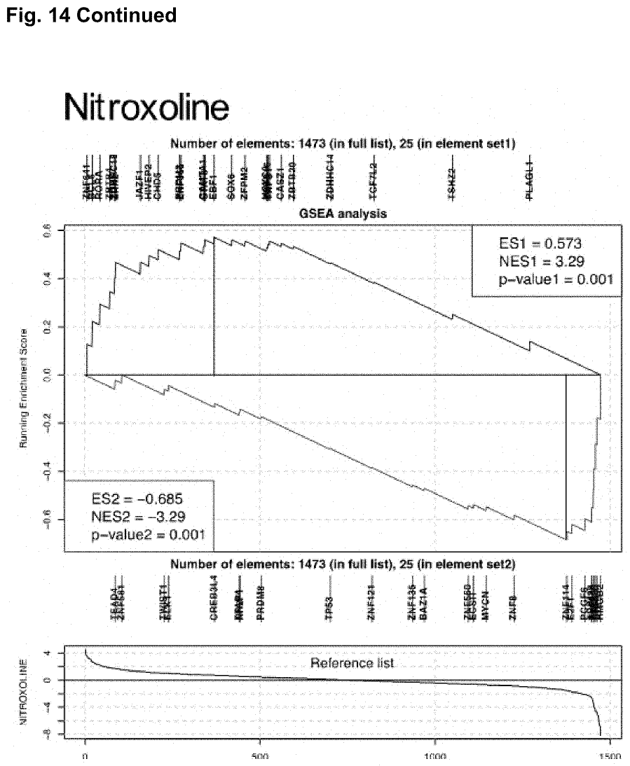

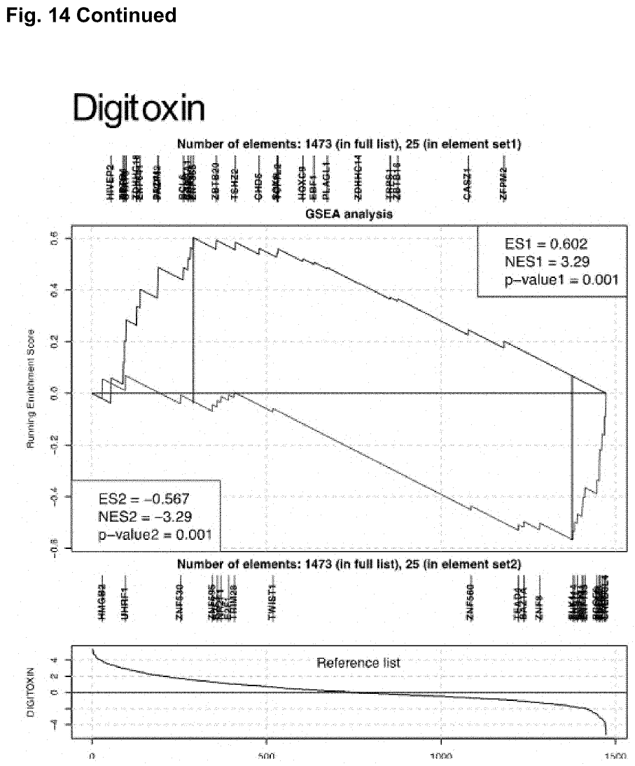

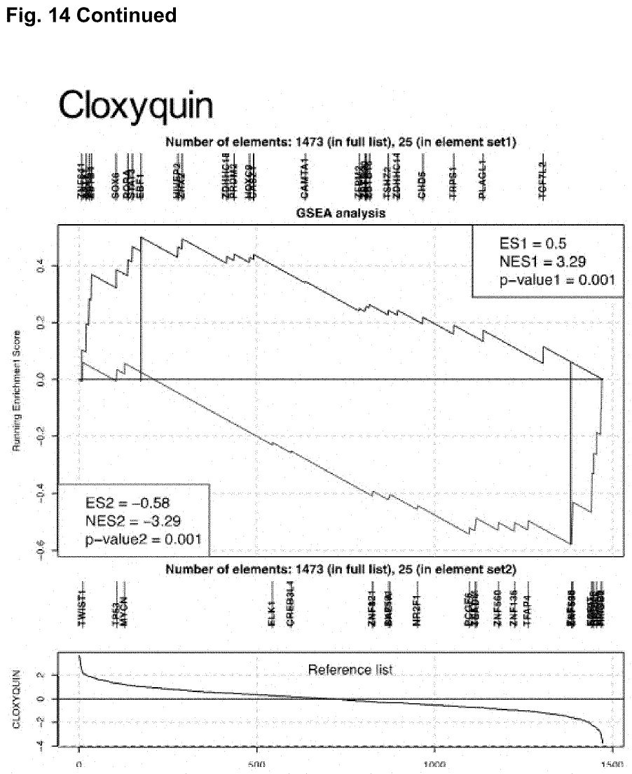

Compounds that collapse the TCM revert the gene 50 candidate MR expression profile highlighted by VIPER plots (FIG. 8C; FIG. 14). The VIPER algorithm ranks transcription factors based on the expression of their regulon and plots their activity along the x-axis of a GSEA plot (Alvarez et al. 2016). This enables the visualization of compound activity on the MR transcription factors aberrantly activated in MYCNA NBL (red) and the MRs suppressed specifically in the MYCNA subtype (blue). Treatment with compounds that collapse the TCM suppressed the activated MRs and restored activity to the MRs typically suppressed in MYCNA NBL (FIG. 2C). Importantly, the TCM-collapsing compounds suppressed the activity of MycN (arrowhead), while compounds that did not revert the signature had negligible effect on MycN activity.

The TCM module acts coordinately to establish and maintain the MYCNA tumor subtype. These core ten protein members were previously validated as interconnected drivers that center on a MycN-TEAD4 regulatory interaction; genetic inhibition of these drivers resulted in MycN suppression and induction of cell death (Rajbhandari et al. 2018). It was hypothesized that compounds that disrupt the TCM would drive MycN suppression in cell line models of MYCNA NBL. The top five TCM-collapsing compounds (isopomiferin, homidium bromide, methyl gambogate methyl ether, podofillotoxin, and NSC255109) were compared to the bottom-ranked compounds, which did not affect the MYCNA TCM (FIG. 8D). As the potency of each molecule differed between each molecule, each compound was tested at its IC₂₀ concentration as well as a three-fold dilution. Consistent with the VIPER analyses that predicted changes to MycN activity, the top-ranked TCM collapsing compounds all suppressed MycN protein abundance, while the bottom ranked compounds had no effect on MycN expression. Although isopomiferin was not the most potent inhibitor of cell viability, it had the strongest effect on the TCM, and suppressed MycN at approximately 5 μM after 24 hours. Little is known about the mechanism of action through which this compound affects cells, so it was chosen for further investigation.

### Example 13

### Isopomiferin Collapses the MYCNA Tumor Checkpoint Module

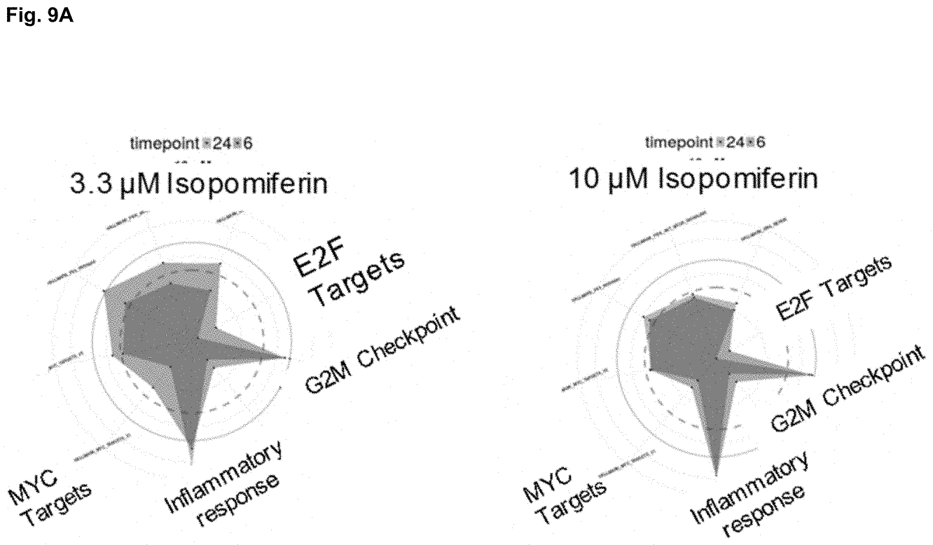

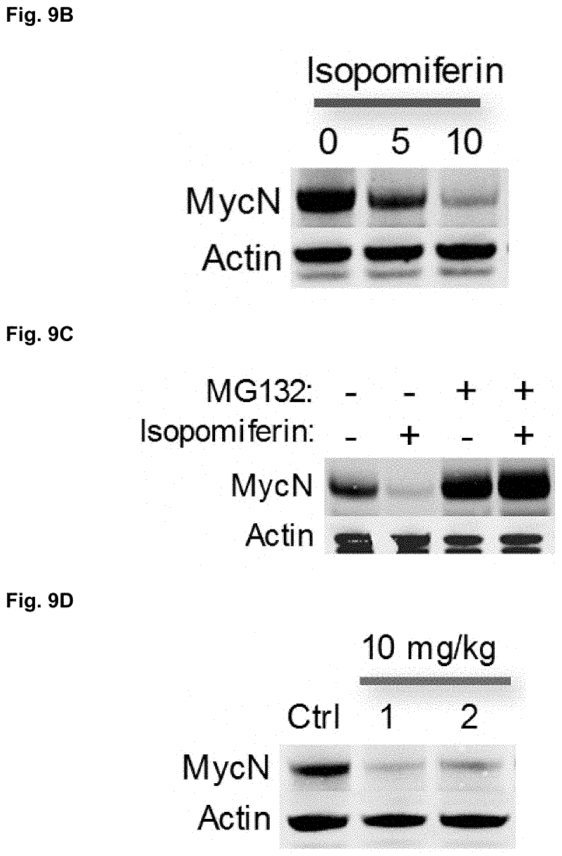

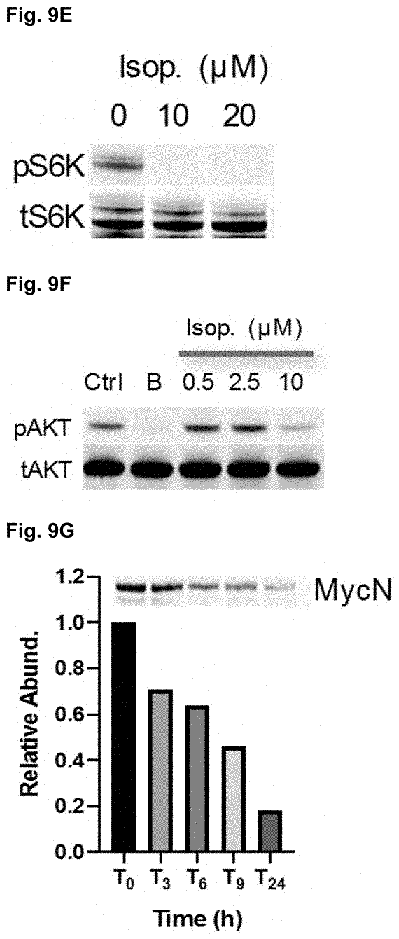

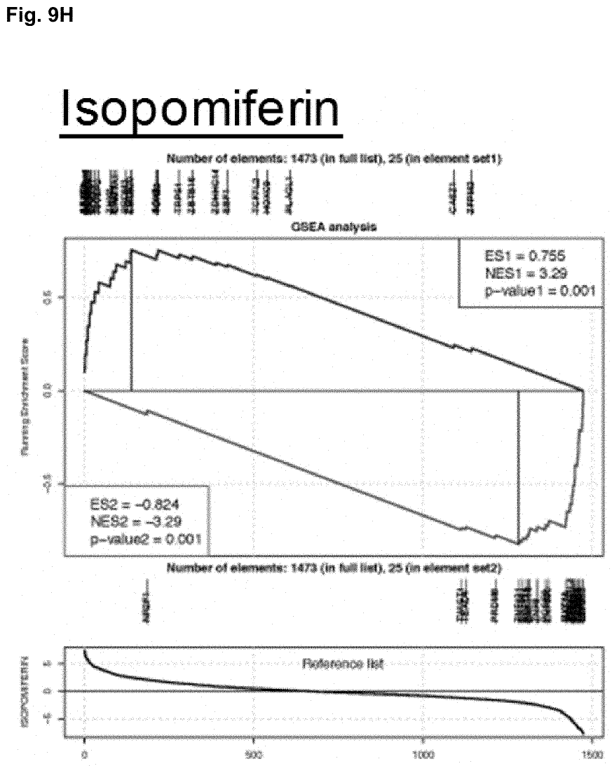

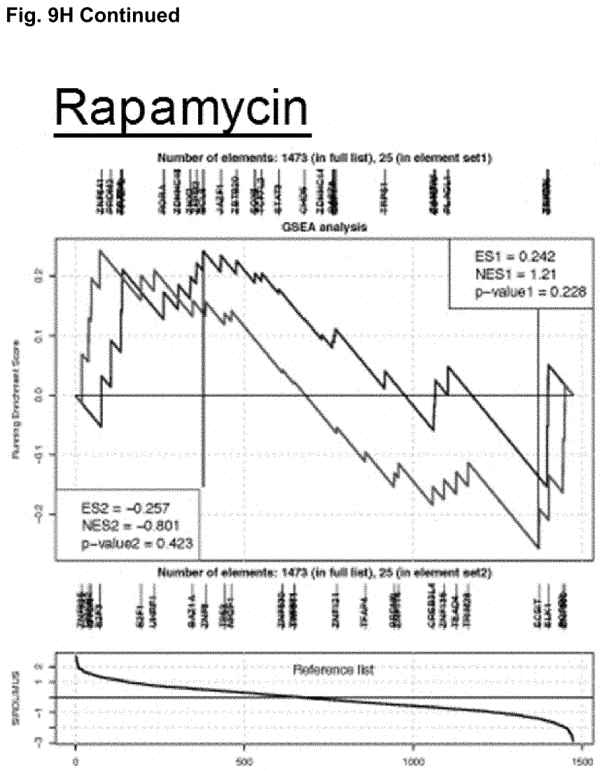

We sought to investigate biological pathways affected by isopomiferin by performing pathway enrichment analysis on a set of 50 hallmarks of cancer. These gene sets are managed and curated by the Broad Institute, and comprise sets of genes involved in cancer-specific bioprocesses. For this analysis, PLATE-Seq data were generated from SK-N-Be2 cells treated with isopomiferin at 3.3 μM and 10 μM for 6 h and 24 h. Of the fifty Hallmarks of Cancer Genesets, nine sets with highest variability from the group mean were displayed on a radar plot to visualize enrichment. Gee set data points that lie inside of the green dash line, or outside the solid red line indicate statistically-significant differences in enrichment. Consistent with the effect of isopomiferin on MycN expression and subsequent cell death, gene sets associated with MYC activity (“MYC Targets”), and cell-cycle regulation (“E2F Targets”, and “G2M Checkpoint”) were suppressed by isopomiferin treatment in a time- and dose-dependent manner (FIG. 9A). Surprisingly, isopomiferin enriched for transcripts associated with “Inflammatory Response” and “Epithelial-to-Mesenchymal Transition”, suggesting profound changes to the cellular state that could be resultant of collapse of the TCM and relieving the suppressive effects of MycN on these pathways.

MycN is regulated at the protein level by the relative rates of de novo synthesis and degradation. As such, mechanisms that perturb either production or protein turnover will affect MycN stability. Isopomiferin suppresses MycN abundance in SK-N-Be2 cells in a dose dependent manner, and this appears dependent on activity of the proteasome (FIGS. 9B and 9C). Inclusion of the protease inhibitor MG132 disrupted the ability of isopomiferin to suppress MycN, suggesting that the active proteasome is essential for isopomiferin activity.

To evaluate in vivo pharmacodynamic effects, isopomiferin was tested for the ability to suppress MycN in mouse tumor xenografts. To evaluate the pharmacodynamic effect of isopomiferin in tumor xenografts, a tumor forming mass of SK-N-Be2 cells was injected into the flank of male NCG mice and allowed to grow to ˜100 mm³ in size. Mice were administered 10 mg/kg of isopomiferin by intraperitoneal injection. After 24 h, tumors were sampled and MycN abundance was evaluated by western blot. Treatment with isopomiferin decreased MycN abundance relative to solvent-only control mice (FIG. 9D). Based on its favorable physicochemical properties, as well as its ability to suppress MycN in cells and xenografts, we decided to elucidate the mechanisms through which isopomiferin collapses the TCM in MYCNA cells.

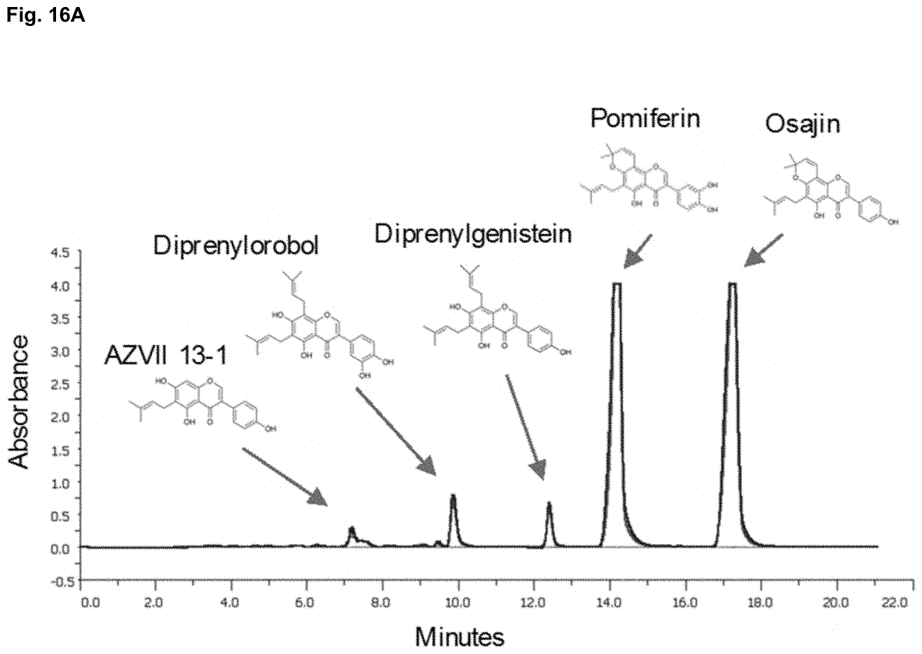

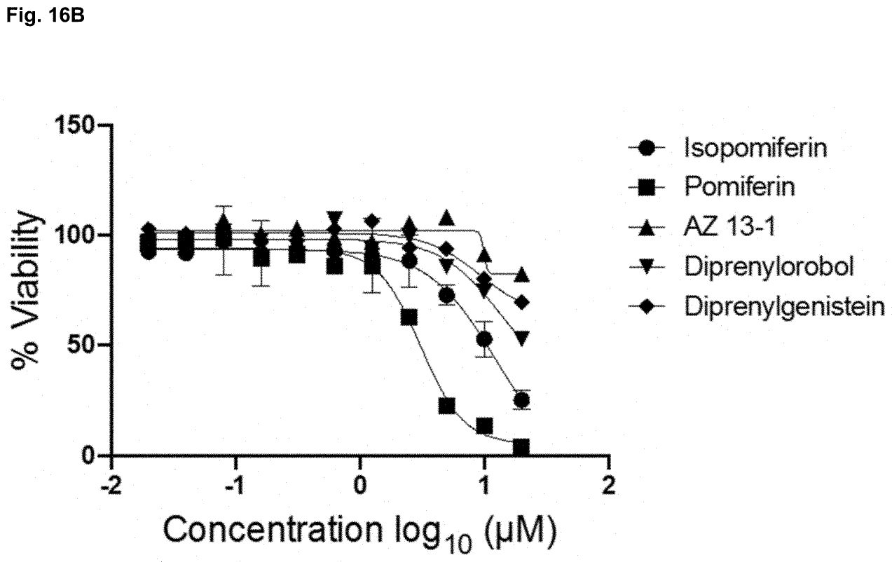

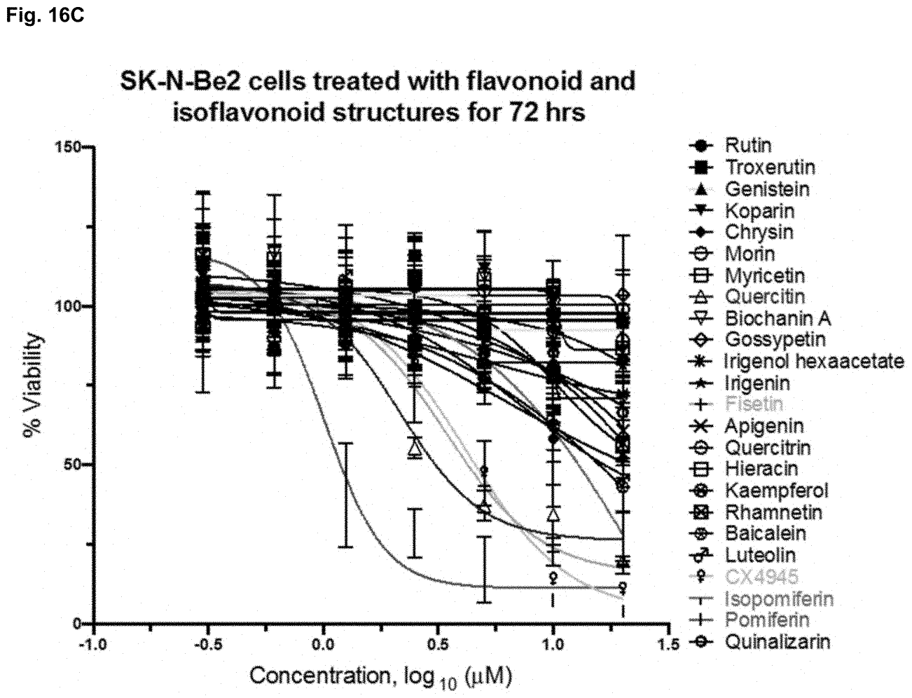

Mechanistic studies using isopomiferin-related structures have suggested potential mechanisms through which prenylated isoflavonoids inhibit cell proliferation. One study tested the effect of pomiferin triacetate on mTOR activity, and found the compound acted through mTOR kinase inhibition (Bajer et al. 2014). mTOR forms kinase subunit of two separate complexes (mTORC1/2) that regulate cell proliferation in response to intrinsic and environmental cues (Kim et al. 2017; Populo et al. 2012; Ballou and Lin, 2008). Phosphorylation of P70^{S6K} is a marker for mTORC1 activity, whereas phosphorylation of AKT (Ser473) serves as a signaling output for mTORC2 (Sarbassov et al. 2005). Isopomiferin inhibited phosphorylation of P70^{S6K}, as well as the IGF1-induced phosphoactivation of AKT (Ser473), suggesting that signaling through both mTORC1/2 was disrupted (FIGS. 9E and 9F). However, isopomiferin did not directly inhibit mTOR kinase activity in cell-free biochemical assays (FIG. 16B), and treatment with the mTOR inhibitor rapamycin did not revert the MR signature (FIG. 9H). Together, these findings suggest that isopomiferin may act through mTORC1/2, but is not a direct mTOR inhibitor.

### Example 14

### Semi-Synthesis of Pomiferin and Osajin Analogs

Pomiferin and osajin were isolated from Maclura pomifera by Soxhlet extraction of the dried fruit with hexane and ether to give a 1:1 mixture of the two. RP HPLC separated pomiferin and osajin and small amounts of structurally related isoflavones. Treatment of the mixture of pomiferin and osajin with iodomethane and potassium carbonate in acetone gave the di-O-methyl and mono-O-methyl derivatives, respectively (Scheme 6). By treatment of pomiferin with iodomethane and potassium carbonate in acetone the O-methyl analog could be isolated (Scheme 7). Catalytic hydrogenation of the pomiferin/osajin mixture gave the respective hexahydro derivatives (Scheme 8) after purification by RP HPLC as well as minor amounts of reduced structurally related isoflavones. More than one amine groups may be introduced to the structure (Scheme 9). The de novo synthesis of pramiverin derivatives is shown in Scheme 10. Interesting moieties often enhance water solubility could be introduced into side chain of pomiferin (Scheme 11). Pomiferin-amino acid conjugate could potentially improve water solubility, stability, and cell permeability compared with parent drug Pomiferin (Scheme 12). Reagents and conditions for the conjugate synthesis comprise: (a) (4-NO2-PhO)2CO, DIPEA, THF/DMF, 0° C. to room temperature; (b) pomiferin, DIPEA, DMF, 0° C. to room temperature; (c) TFA, DCM, 0° C. to room temperature. Also, the presence of a sugar moiety usually increases the solubility in water solutions (Scheme 13).

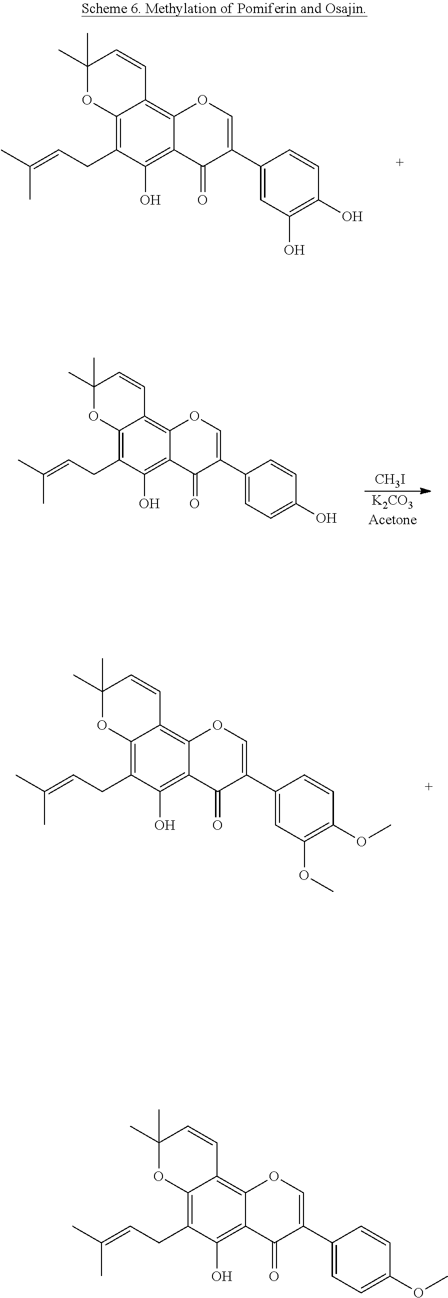

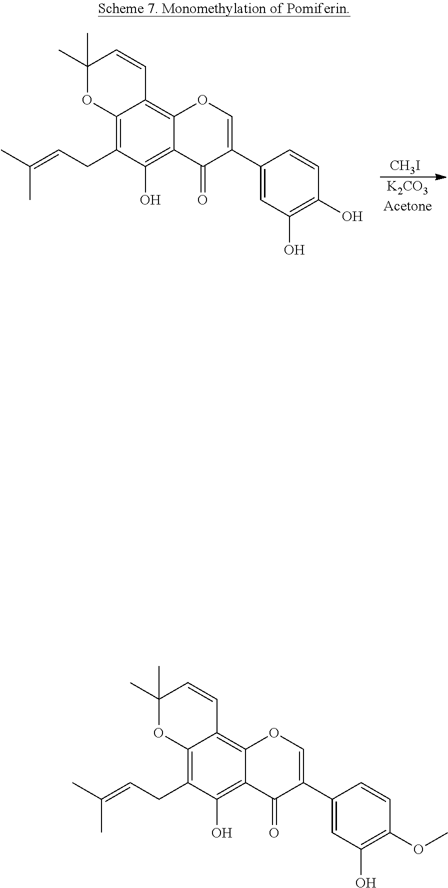 [SMILES: CC(C)=CCc1c2c(c3occ(-c4ccc(O)c(O)c4)c(=O)c3c1O)C=CC(C)(C)O2 ; "CH3I" ; "K2CO3Acetone" ; COc1ccc(-c2coc3c4c(c(CC=C(C)C)c(O)c3c2=O)OC(C)(C)C=C4)cc1O]

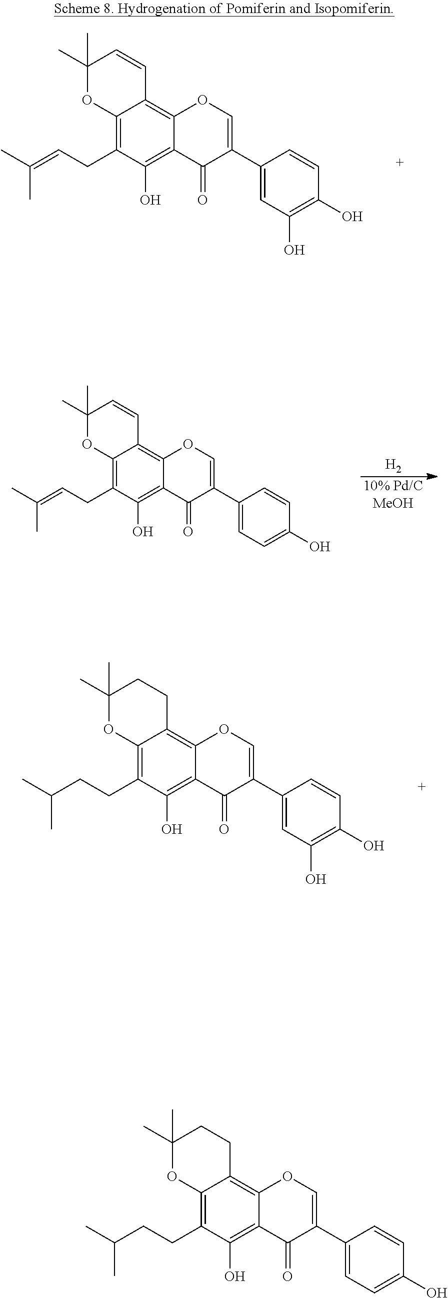 [SMILES: CC(C)=CCc1c2c(c3occ(-c4ccc(O)c(O)c4)c(=O)c3c1O)C=CC(C)(C)O2 ; CC(C)=CCc1c2c(c3occ(-c4ccc(O)cc4)c(=O)c3c1O)C=CC(C)(C)O2 ; "H2" ; "10% Pd/CMeOH" ; CC(C)CCc1c2c(c3occ(-c4ccc(O)c(O)c4)c(=O)c3c1O)CCC(C)(C)O2 ; CC(C)CCc1c2c(c3occ(-c4ccc(O)cc4)c(=O)c3c1O)CCC(C)(C)O2]

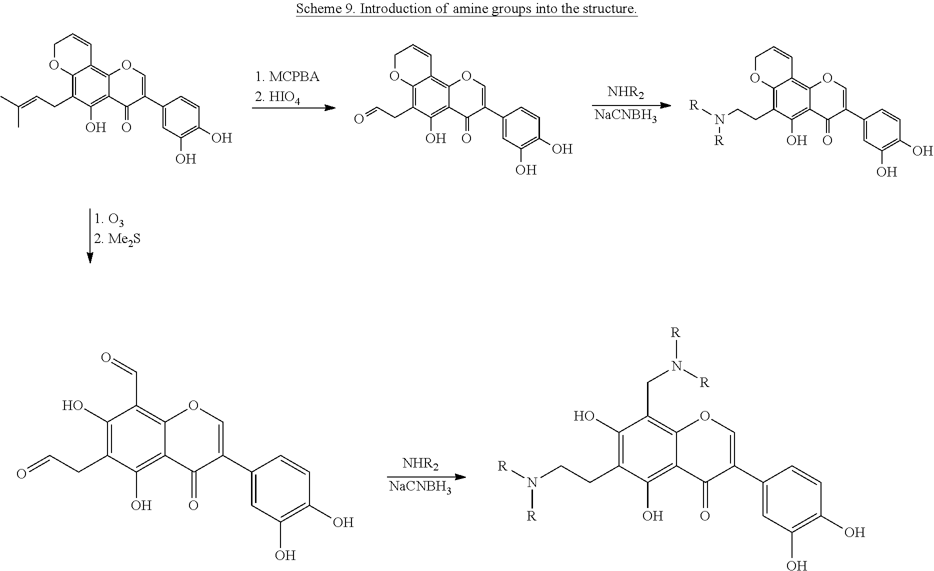

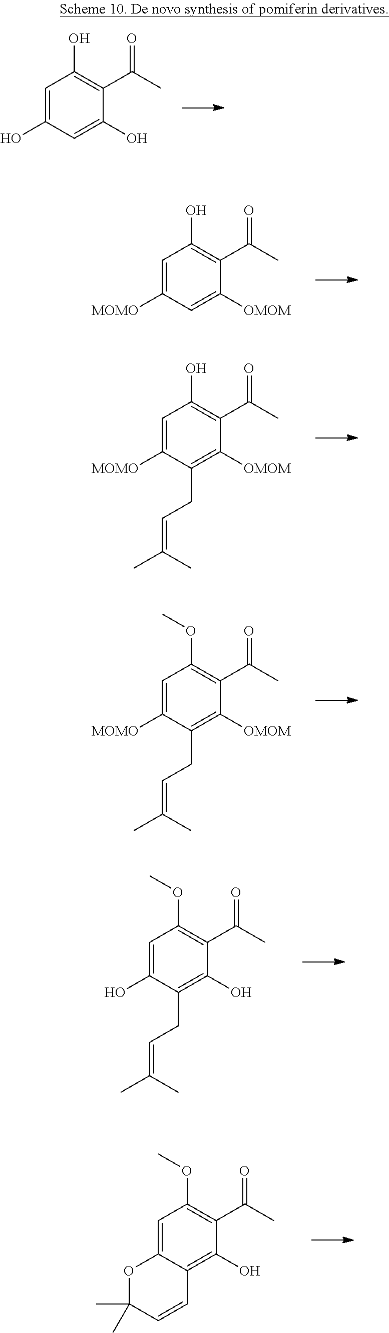

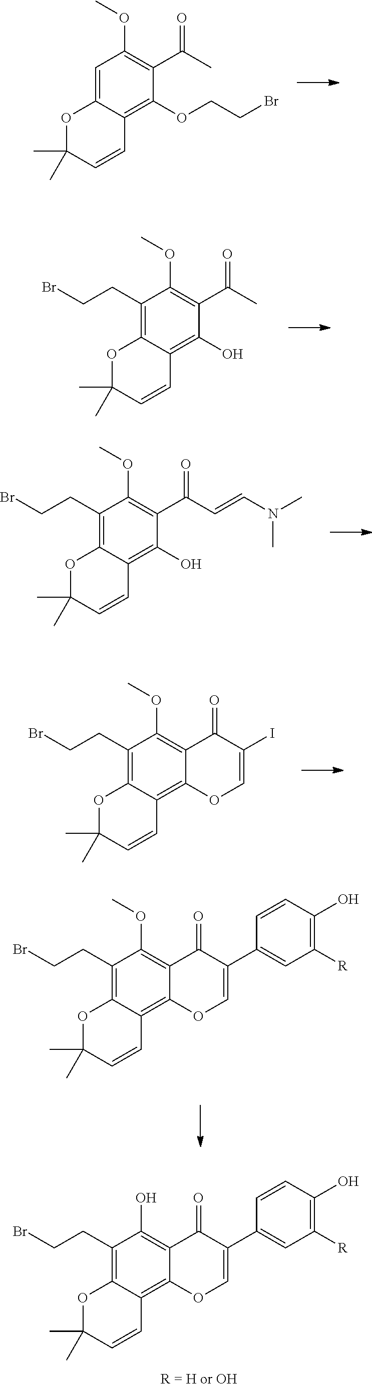

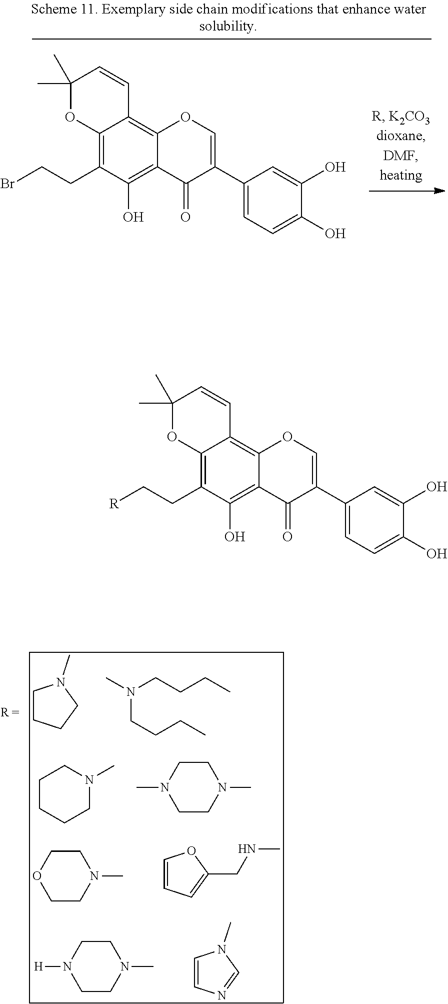

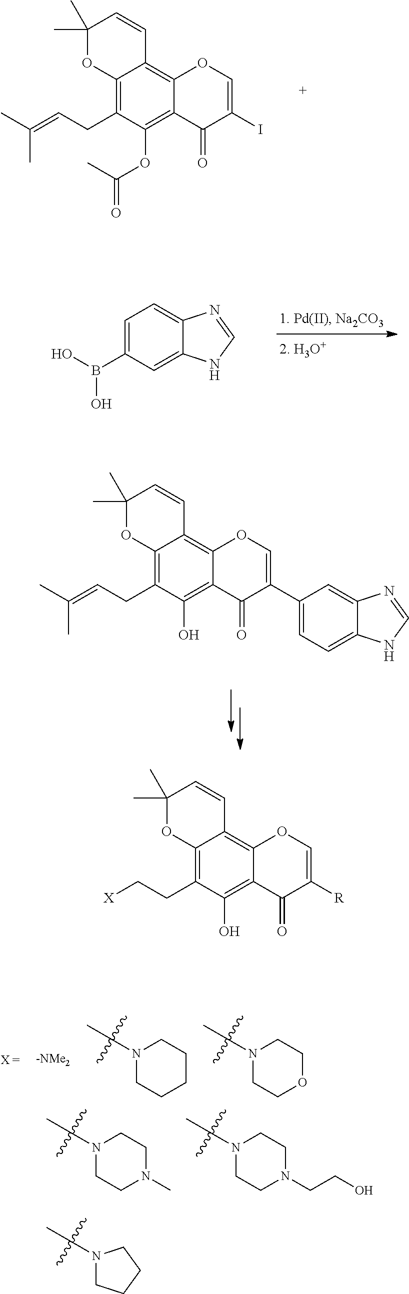

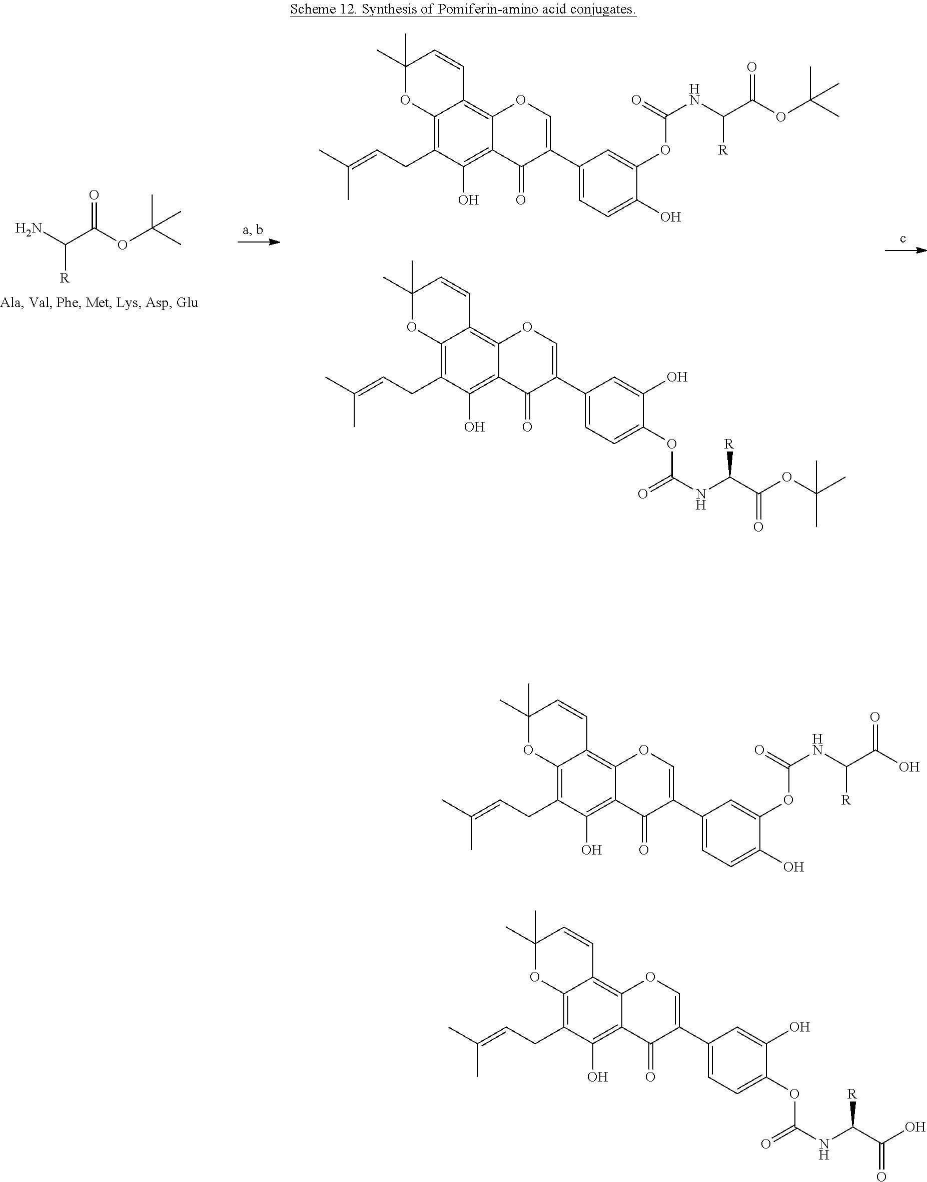

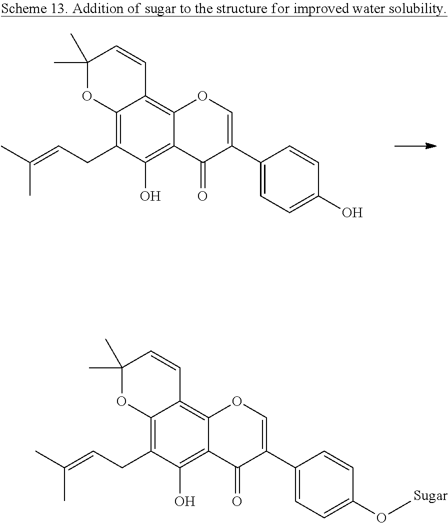 [SMILES: CC(C)=CCc1c2c(c3occ(-c4ccc(O)cc4)c(=O)c3c1O)C=CC(C)(C)O2 ; COc1ccc(-c2coc3c4c(c(CC=C(C)C)c(O)c3c2=O)OC(C)(C)C=C4)cc1]

### Example 15

### Structural Analogs Reveal Potent Analog and Structure-Activity Relationship Data

Isopomiferin is a prenylated isoflavonoid isolated from the wild citrus species Maclura pomifera (Darji et al. 2013; Wolfrom et al. 1946), with chemical properties that make it an appealing scaffold for further development. The compound has low molecular weight, and is compliant with Lipinski's “Rule-of-Five” for optimal drug properties in humans (Lipinski et al. 1997). Furthermore, the scaffold is amenable to modification by semi-synthesis and total synthesis to produce analogs for optimization of its properties.

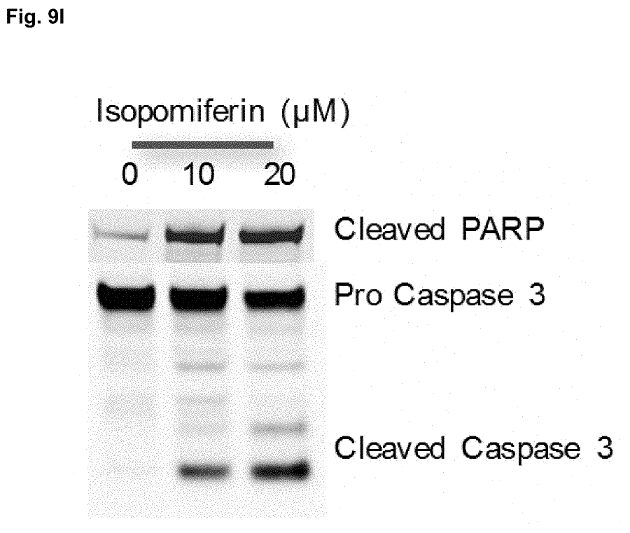

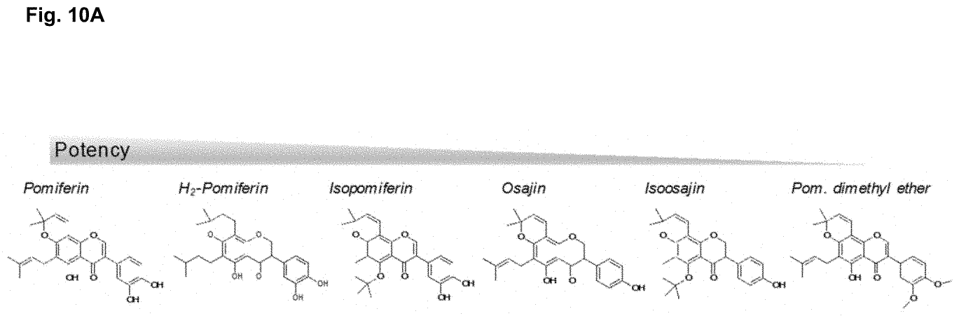

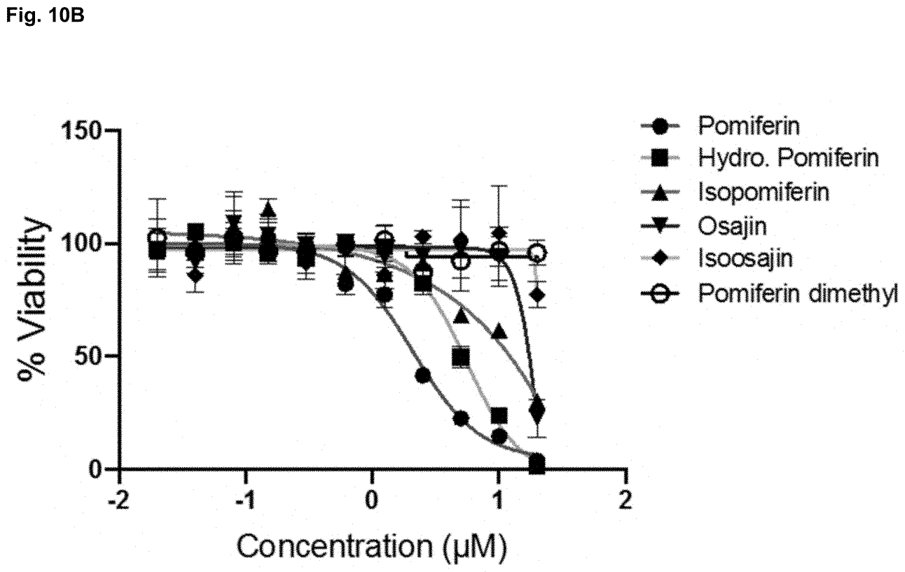

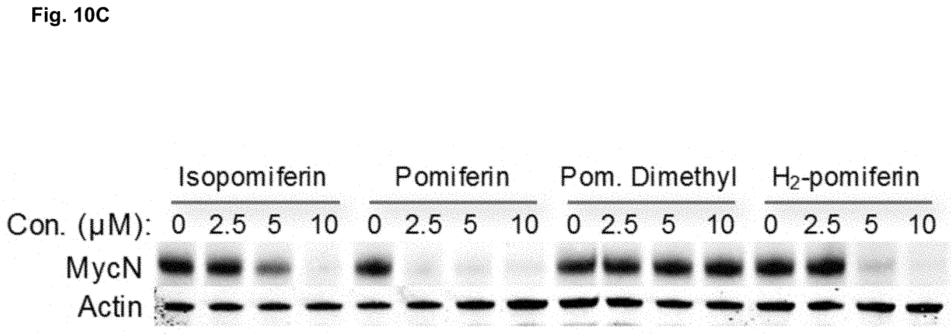

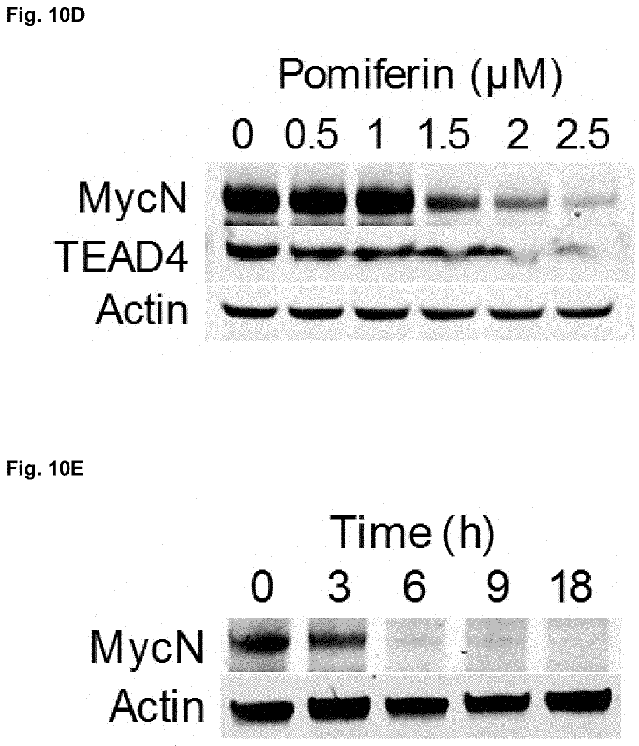

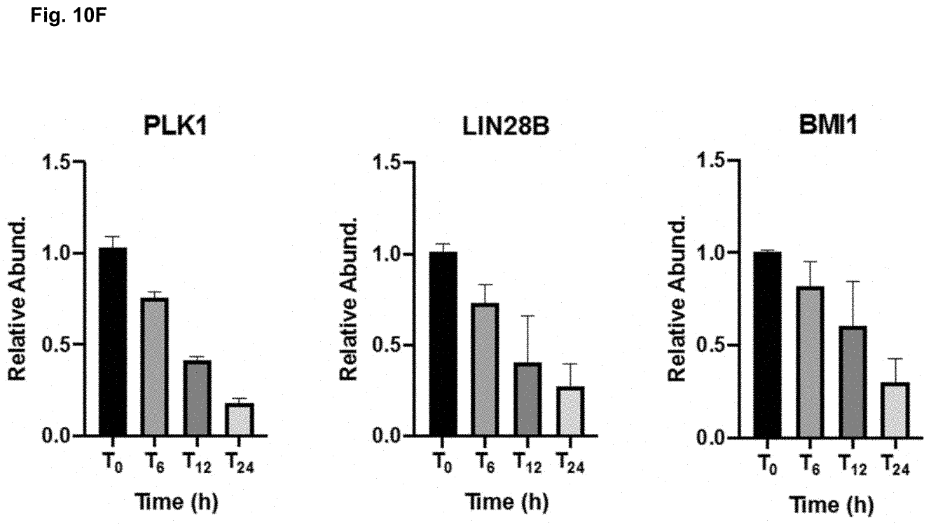

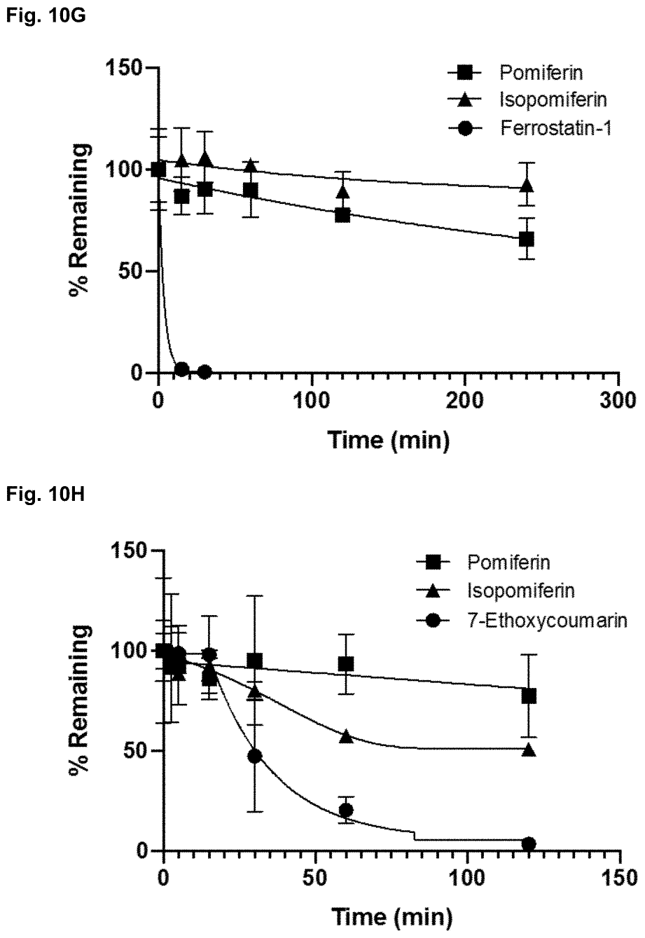

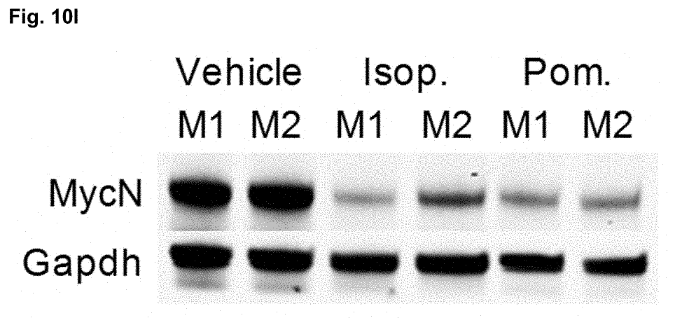

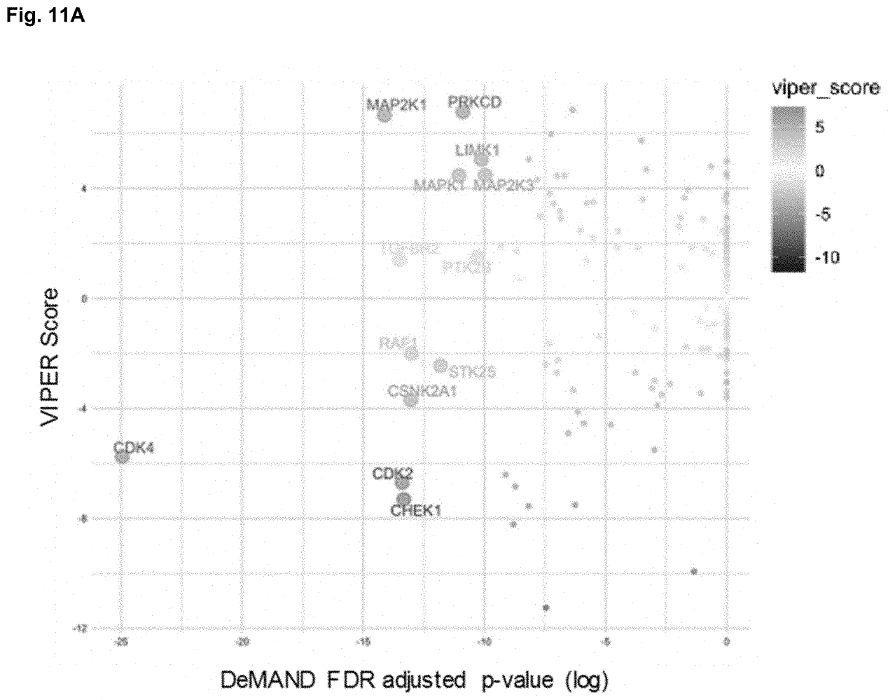

In an effort to identify a potent analog of isopomiferin, we screened a small collection of structurally-related analogs and tested them in SK-N-Be2 cells. These compounds were collected from commercial vendors, isolated from natural sources, or are novel products created through semi-synthesis based on the pomiferin natural scaffold as starting material. Subtle structural modifications resulted in profound changes in compounds ability to suppress MycN or induce cell death (FIGS. 10A-10C; FIGS. 16A-16C). Notably, conversion of hydroxy functional groups on the phenyl ring to methoxy groups removed lethal activity of the molecule at the concentrations tested. Molecular modeling studies of pomiferin docked in CK2 show that these hydroxyl groups make key hydrogen bonding interactions with amino acid residues in the protein that would be disrupted by their methylation (See below). Comparing the activity of six closely related structural analogs revealed that pomiferin is the most potent compound of the group. A broader panel of various flavonoid and isoflavonoid structures confirmed that pomiferin stood out as a uniquely potent inhibitor of SK-N-Be2 cell viability (FIGS. 16A-16C). These structure-activity-relationship data provide key insights into the activity of the molecule and will inform continued medicinal chemistry efforts aimed at the development of potent MycN suppressing compounds.

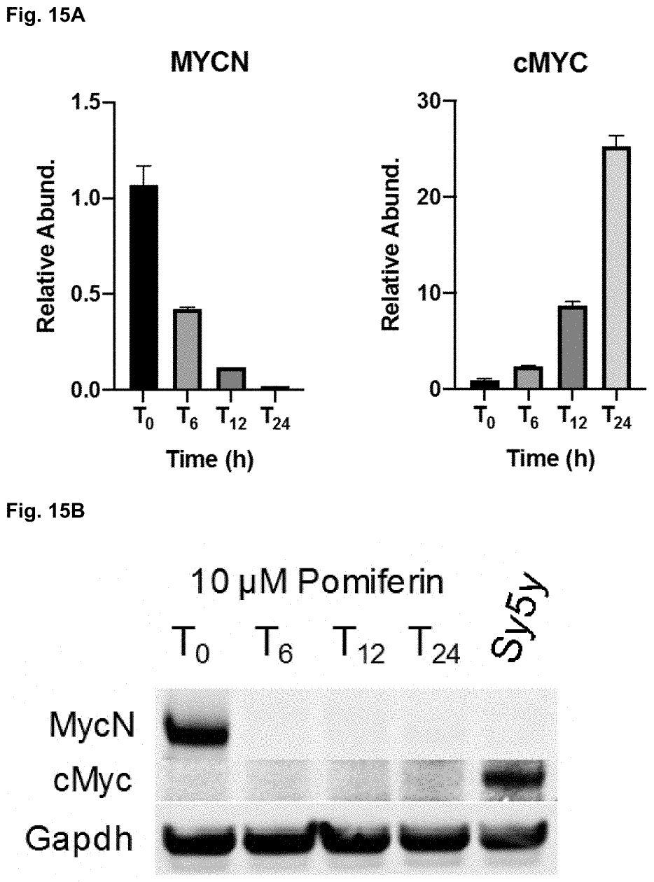

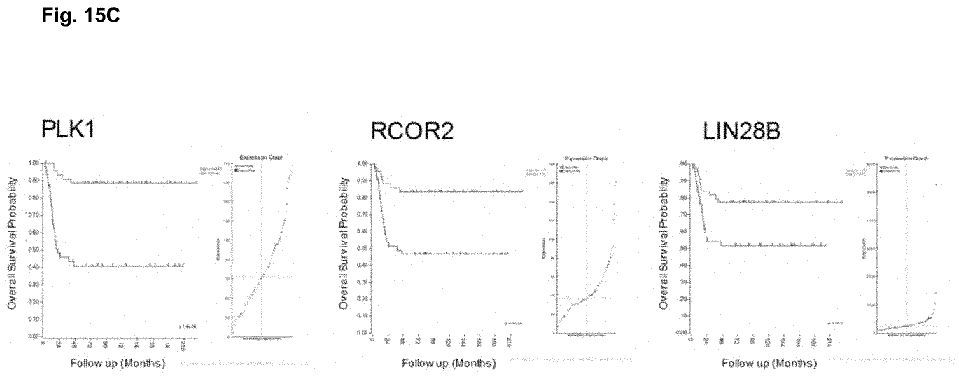

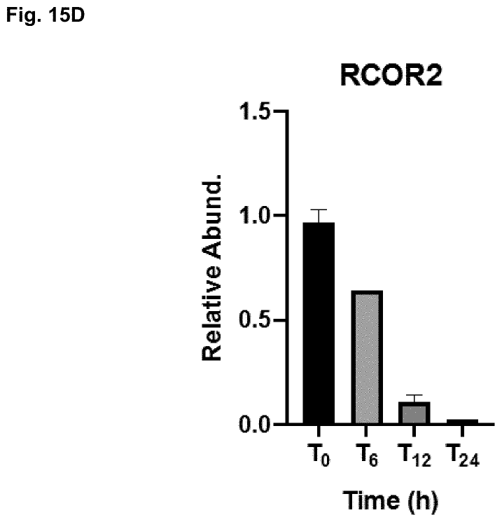

To confirm whether the lethal action of isopomiferin analogs was related to MycN suppression, the effect of four closely-related structures was tested in cell-based assays. SK-N-Be2 cells were treated with isopomiferin, the more potent structure pomiferin, a hydrogenated version of pomiferin with improved solubility, or the inactive pomiferin dimethyl ether. Consistent with their effects on cell viability, pomiferin was the most potent compound and depleted both MycN and TEAD4 at concentrations as low as 1.5 μM and with 6 h of treatment (FIGS. 10C-10E). Isopomiferin and hydrogenated pomiferin had similar effects on MycN abundance, although hydrogenated pomiferin was slightly more active at 5 μM. Consistent with its inactivity at 20 μM, pomiferin dimethyl ether had no effect on MycN accumulation at the concentrations tested. To test whether pomiferin was sufficient to disrupt MycN-regulated gene activation, we confirmed that pomiferin inhibited the transcript accumulation of three downstream targets of MycN associated with poor patient outcome in NBL, including PLK1, LIN28B, and BMI1 (FIG. 10F; FIGS. 15A-15D). By 24 h, all three transcripts tested showed marked decrease in accumulation, demonstrating that pomiferin disrupts oncogenic signaling driven by the MycN regulatory axis. Given that pomiferin was the most effective analog tested, it was chosen exclusively for use in future experimentation.

In vitro metabolic stability assays were performed to evaluate the potential activity of isopomiferin and pomiferin. Both compounds were incubated alongside ferrostatin-1 in mouse plasma for 4 h, and compounds were quantified by LC-MS. Isopomiferin was completely stable in mouse plasma, while approximately 75% pomiferin remained following incubation (FIG. 10G). Similarly, both pomiferin and isopomiferin were reasonably stable to metabolic activation by mouse liver microsomes following 2 h incubation. Approximately ˜80% pomiferin and ˜50% isopomiferin remained, suggesting that the prenylated isoflavonoids are metabolically stable (FIG. 10H). The in vivo pharmacodynamic effects of the compounds was then compared by treating tumor-bearing mice to daily i.p. treatments of isopomiferin and pomiferin at 20 mg/kg for 72 h (FIG. 10I). Both compounds suppressed MycN expression in tumor xenografts, supporting good pharmacodynamic activity of the scaffold in vivo.

### Example 16

### Casein Kinase 2a is a Direct Functional Target of Pomiferin

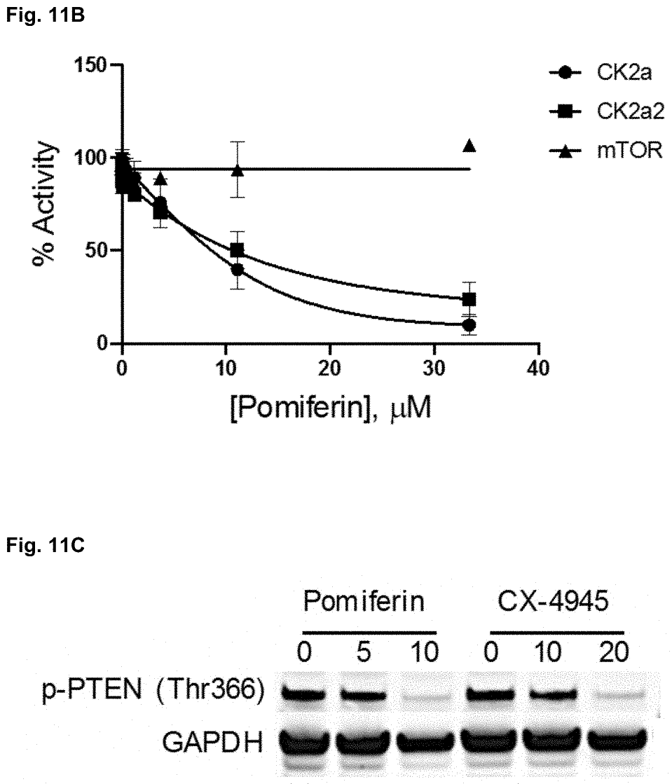

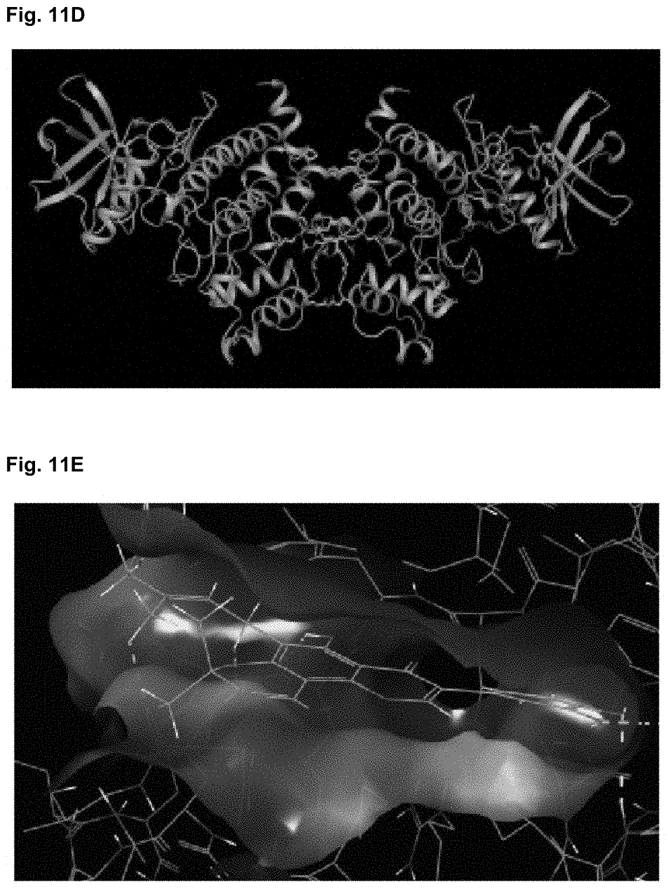

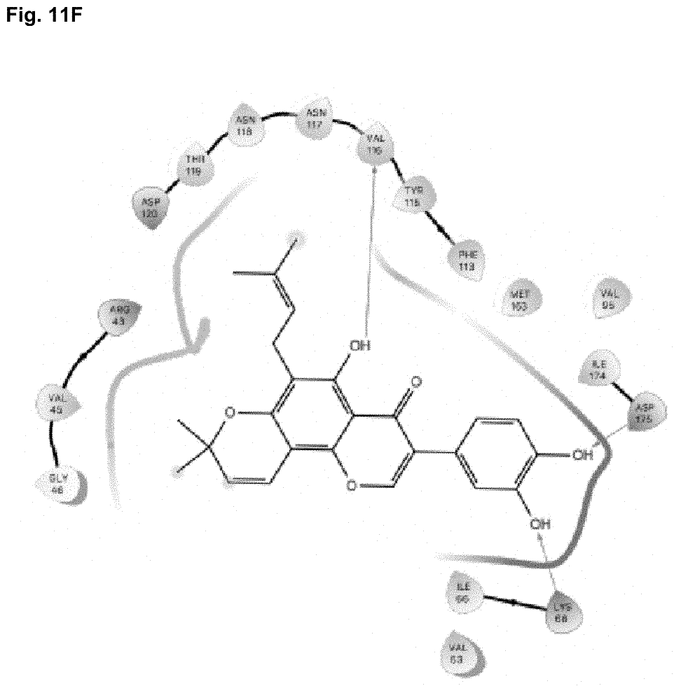

To uncover the target of isopomiferin and functional analogs, we devised a novel algorithm to enable the virtual-inference of kinase inhibition by drugs (VIKING). This analysis prioritizes kinase targets based on dysregulation of their predicted protein activity based on PLATE-Seq expression profiles. VIKING makes use of a transcriptional regulatory network to infer the protein activity profile of more than 6,000 regulators based on differential gene expression profiles through the VIPER algorithm. These protein activity profiles are then used as input to Determination of Mechanism of Action by Network Dysregulation (DeMAND), which evaluates network dysregulation to pinpoint cellular mechanisms of action [19]. VIKING uses a protein-protein interaction (PPI) network as input of DeMAND. Specifically, the PrePPI database is queried to select for interactions between signaling proteins (SIG), such as kinases, and transcriptional regulators. The DeMAND output list of putative cellular MoAs is then cross referenced with VIPER scores of differential activity in response to chemical treatment. Negative NES scores represent the loss of protein activity by possible chemical inhibition. This list is filtered for human kinases to prioritize them as targets of the compound. As a result, VIKING outputs a list of potential cellular mechanisms of action that includes kinases and effector proteins as potential targets of isopomiferin. This analysis highlighted a number if putative targets of isopomiferin (FIG. 11A), which were evaluated as potential direct targets of the compound.

One of the predictions of VIKING was CK2a1, the active subunit of the pleiotropic casein kinase 2, which regulates MYC proteins through direct and indirect mechanisms (Wang et al. 2009; Bousset et al. 1993; Luscher et al. 1989), and phosphorylates a wide number of protein targets that drive cell proliferation and support pro-survival mechanisms (Turowec et al. 2010; Litchfield, 2003). Casein kinase functions as a heterotetrameric complex, with two active kinase subunits (CK2a and CK2a′), and two additional beta subunits providing spatiotemporal regulation (Trembley et al. 2009; Litchfield, 2003; Litchfield and Luscher, 1993). We tested whether CK2 was a direct target on in cell-free biochemical kinase assays. Pomiferin disrupted kinase activity of both alpha subunits in cell-free biochemical kinase assays, inhibiting both isoforms equally in a concentration-dependent manner (FIG. 11B). Supporting the hypothesis that CK2 may be a direct target of the prenylated isoflavonoids.

Molecular modeling was used to validate the direct interaction between pomiferin and CK2a, and to uncover the molecular interactions that enable inhibition of kinase activity. The keto and hydroxyl groups of the isoflavone core are in hydrogen bonding proximity to the hinge region Val116 and make key interactions with it. The hydroxyl groups of the phenyl ring interact with Lys68 and Asp175, consistent with the decrease in activity of the O-methyl analogs (Schemes 1 and 2). The isoprene portions of the molecule are solvent exposed, consistent with the retention of activity on reduction of their double bonds. In addition, reduction of the double bond in the isoflavone ring, while slightly changing the shape of the molecule, allows it to maintain the key binding interactions (Scheme 3).

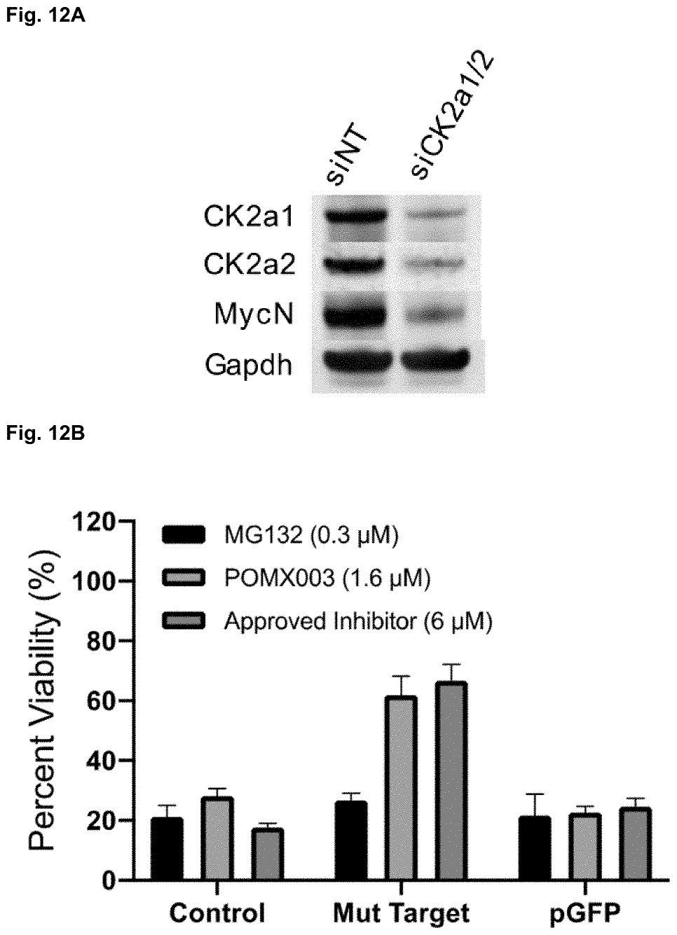

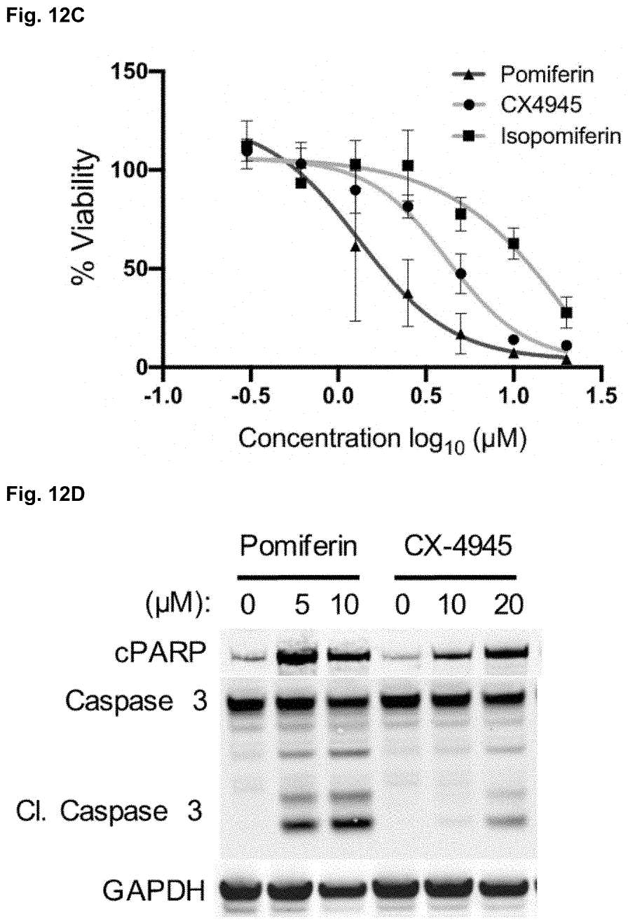

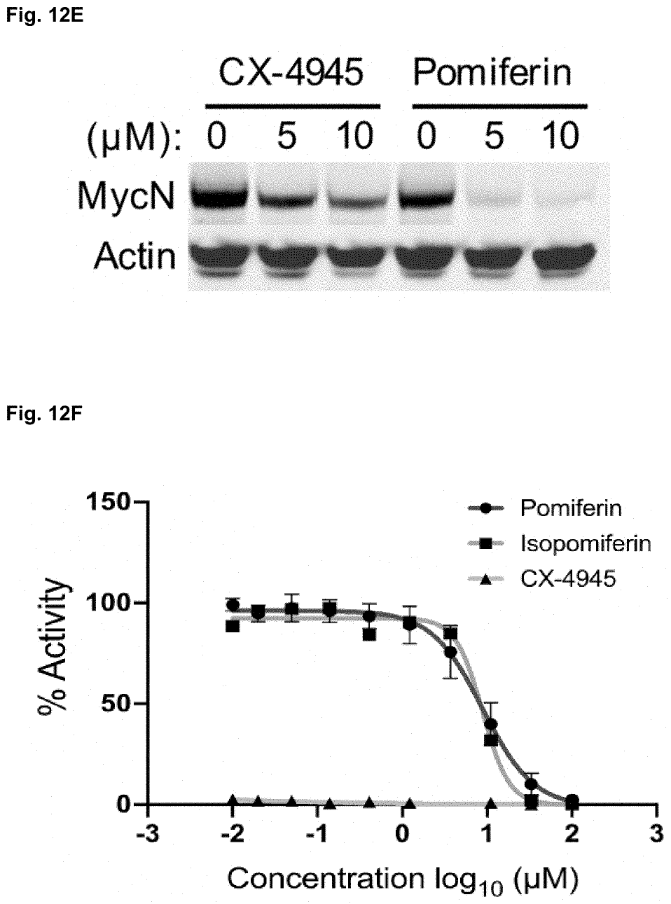

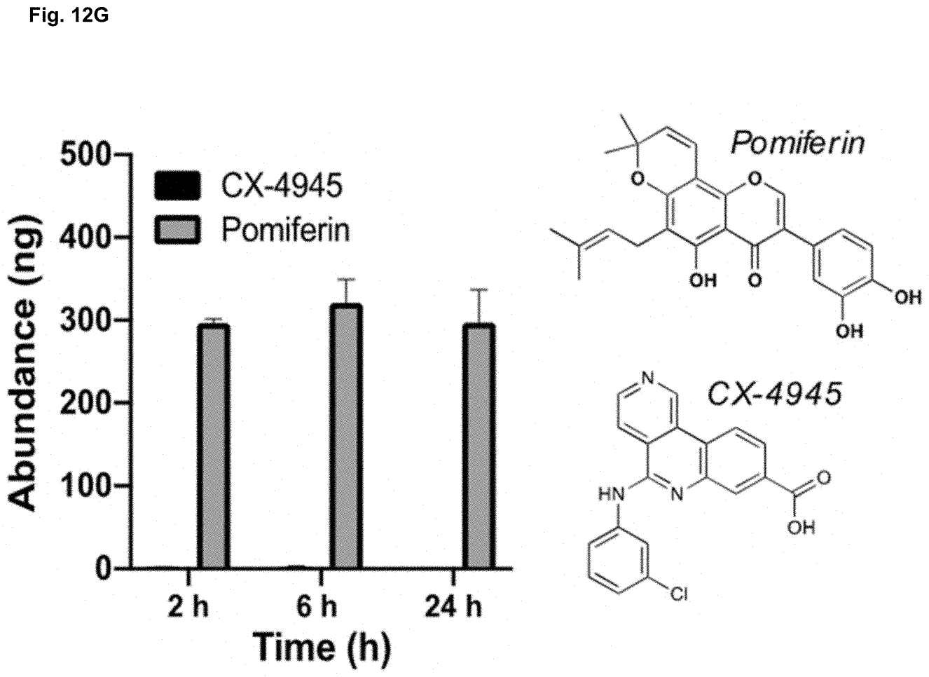

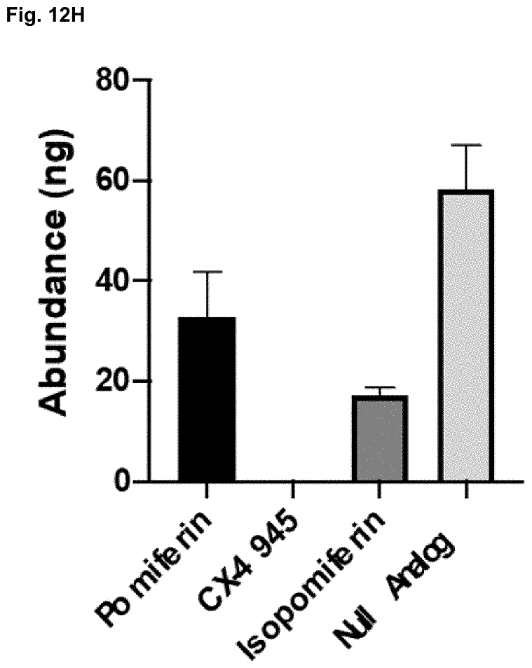

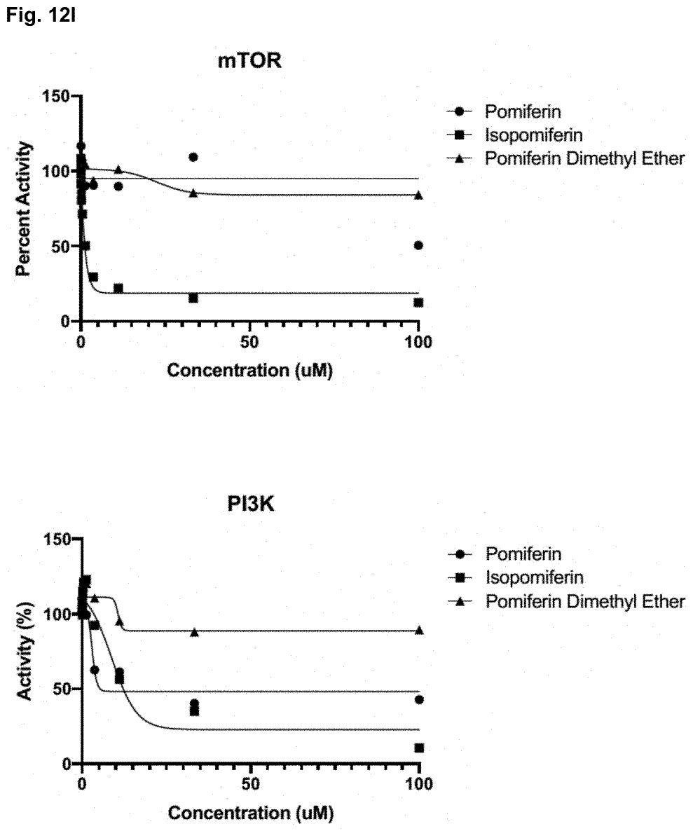

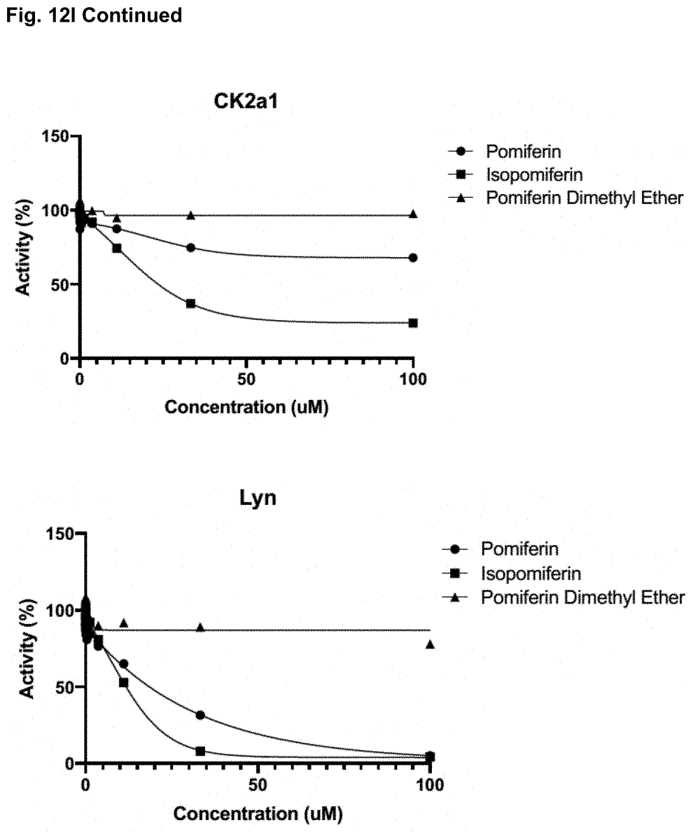

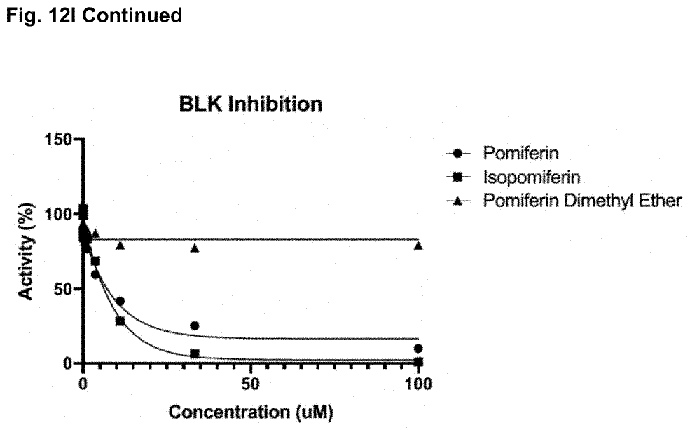

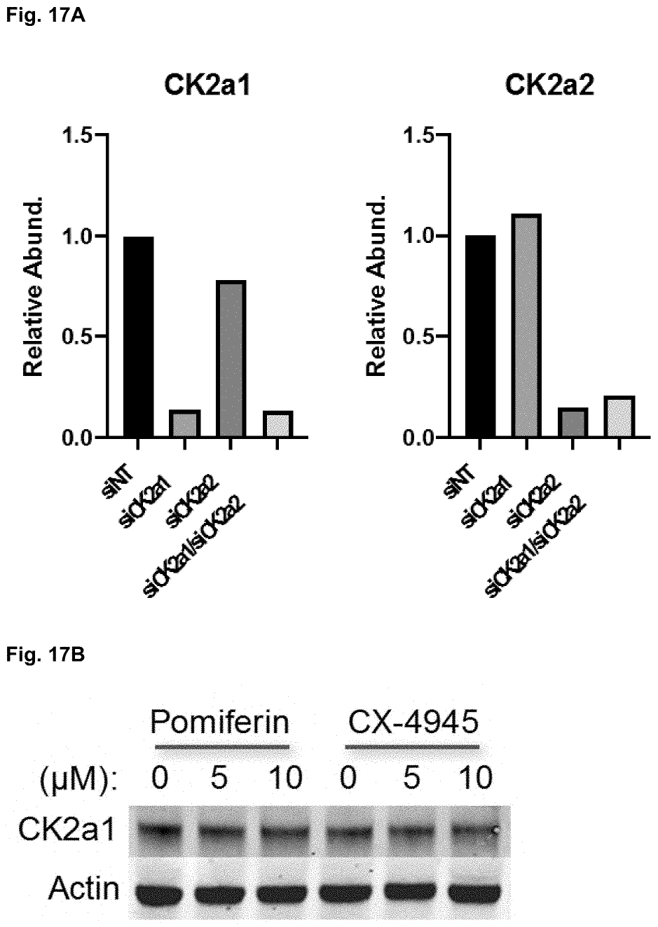

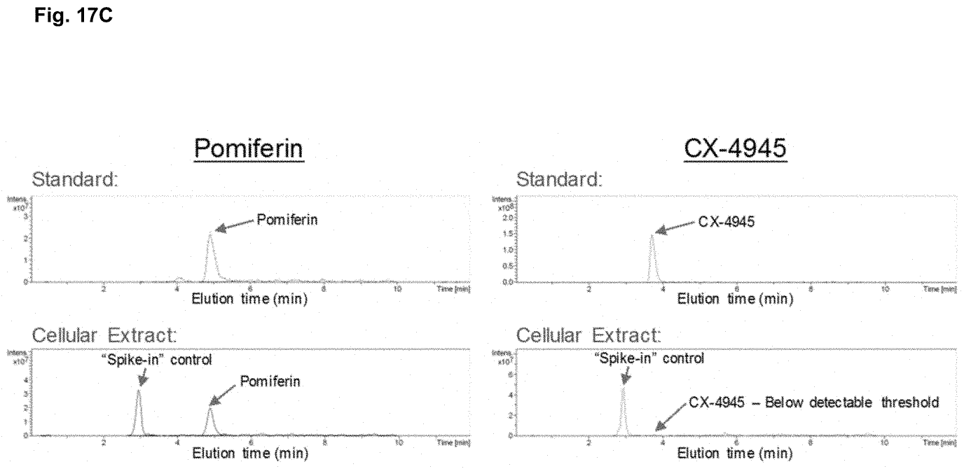

The functional relationship between CK2 and MycN was assessed by knocking down both CK2a1 and CK2a2 subunits and evaluating the effect on MycN abundance. Gene specific siRNAs targeting each alpha subunit were pooled and transfected into SK-N-Be2 cells. Western blot and qPCR confirmed sufficient knockdown of both isoforms (FIG. 12A; FIG. 17A). Similar to pomiferin treatment, knocking down CK2a1/2 suppresses MycN protein (FIG. 12A), suggesting that the combined activity of the two subunits help stabilize MycN protein.

Chemical inhibitors of CK2 are available to probe the role of CK2 on MycN stability, and to validate this as a mechanism of pomiferin activity. One such compound, CX-4945 (silmitasertib), is a potent CK2 inhibitor designed using in silico docking based methods, and has shown activity in a variety of cancer models (Prins et al. 2013; Ferrer-Font et al. 2017; Siddiqui-Jain et al. 2010; Drygin et al. 2009). The compound received orphan designation status by the FDA, and is currently in clinical testing for cholangiocarcinoma, and other solid tumors (Ghon et al. 2015) (www.clinicaltrials.gov). We used this inhibitor to validate CK2 as a relevant target in MYCNA NBL, and to evaluate how prenylated isoflavonoids compare to CK2 inhibitors currently in development (Siddiqui-Jain et al. 2010; Anderes et al. 2009).

We validated CK2a as a functional target of pomiferin by expressing a mutant isoform of CK2a1 harboring amino acid substitutions at residues essential for interaction with CX-4945. Despite considerable effort, we were only able to achieve minimal plasmid transfection efficiency, which is an inherent challenge of NBL cell models. Despite this, cells expressing the mutant CK2 isoform exhibited modest resistance to both pomiferin and CX-4945, while cells transfected with a GFP-expressing plasmid maintained sensitivity. To assess the specificity of this response, the proteasome inhibitor MG132 was tested alongside the two putative CK2 inhibitors, and no resistance was conferred by the plasmid. Together, these findings support the hypothesis that CK2a is a direct mechanistic target of pomiferin in MYCNA NBL.

The ability to induce cell death was compared between CX-4945, pomiferin and isopomiferin in cell based viability assays, hypothesizing that CX-4945 would be a potent inhibitor of SK-N-Be2 cells. Dose-response curves of SK-N-Be2 cells treated with the three compounds reveled pomiferin was the most potent inhibitor of viability, followed by CX-4945, and then isopomiferin (FIG. 12C). Consistent with this pomiferin was a more potent inducer of apoptosis than CX-4945, as evidenced by PARP and caspase3 cleavage (FIG. 12D). To assess how these differences in potency related to MycN-suppression, MycN abundance quantified by western blot following treatment for 24 h. Similar to the cell-based assays, pomiferin was a more potent MycN suppressor than CX-4945 (FIG. 12E). Finding that the selective CK2 inhibitor CX-4945 suppresses MycN abundance supports the hypothesis that CK2 is a relevant mechanism for targeting MYCNA NBL, and is consistent with the proposed mechanism of pomiferin activity in cells.

In contrast with the observations in cell-based assays, CX-4945 is an incredibly potent inhibitor of CK2a kinase activity in cell-free biochemical assays (FIG. 12F). The Ki of CX-4945 for CK2a has been reported in the single-digit nanomolar range (Siddiqui-Jain et al. 2010), and in our assays CX-4945 completely inhibited kinase activity of CK2a at all concentrations tested. In contrast, both isopomiferin and pomiferin had similar IC₅₀ concentrations of ˜7 μM, which was far less potent than CX-4945. This finding challenges the model that CK2a is the target of pomiferin in that if CK2 were the single target mechanism, then pomiferin should exhibit potent inhibition of CK2 in cell free assays relative to CX-4945 to be consistent with viability and MycN suppression.

To assess whether changes in cellular accumulation could underpin the differential activity between cell-based and cell-free assays, we assessed the cellular accumulation of each compound by LC-MS following incubation with CX-4945 or pomiferin. We initially confirmed the ability to detect and quantify pomiferin and CX-4945 by running standard samples of compound added directly to acetonitrile and injected into the LC-MS, which indicated that both compounds ionized readily and were detectable on the instrument (FIGS. 17A-17C). SK-N-Be2 cells were then treated with 20 μM of CX-4945 or pomiferin and sampled across time. Pomiferin readily accumulated in cells, as ˜300 ng of compound was detected in the sample (FIG. 12G). Contrary to this, only 2.2 ng of CX-4945 was detected by 6 h, indicating that the CX-4945 compound suffers from poor cell permeability. Consistent with this hypothesis, CX-4945 structure contains a carboxylic acid functional group, which is likely charged at physiological pH (FIG. 12G). Charged molecules are generally impeded from passing the phospholipid bilayer, and generally have poor cell permeability. This suggests that the discrepancy between kinase assays and cell viability tests related to cell permeability, although the possible contribution by secondary targets cannot be ruled out and it is likely that a second mechanism contributes to the potent MycN suppression of pomiferin.

### Example 17

Pomiferin Suppresses MYC Proteins Across Cancer Models

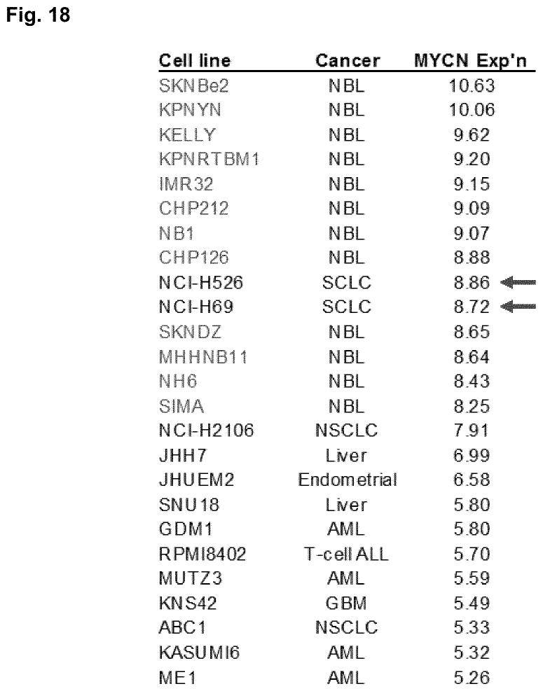

MYCN-amplification is commonly associated with the aggressive NBL subtype, but MycN dysregulation is associated with a small subset of many aggressive tumors (Rickman et al. 2018), including Wilms' tumor (Williams et al. 2015), neuroendocrine prostate cancer (Lee et al. 2016), and lung cancers (Liu et al. 2016; Funa et al. 1987; Wong et al. 1986; Nau et al. 1986). Although Wilms' tumor and neuroendocrine prostate cancer are deficient in cell models, there are many lung cancer cell lines available to evaluate the potential of pomiferin to suppress drivers of these cancers. Expression profiles from the cancer cell line encyclopedia (CCLE) were ranked by MYCN transcript abundance to identify cell lines with dysregulated MYCN expression across a variety of cancers. Ranking over 1000 cell lines based on MYCN transcript abundance, revealed that 12 of the top 14 cell lines were NBL, while the other 2 cell lines were derived from small cell lung cancer (SCLC; FIG. 18). Approximately 20% of SCLC tumors are associated with amplification of MYC family proto-oncogenes (Bragelmann et al. 2017), and finding that these cell lines among the top ranked reflects this observation.

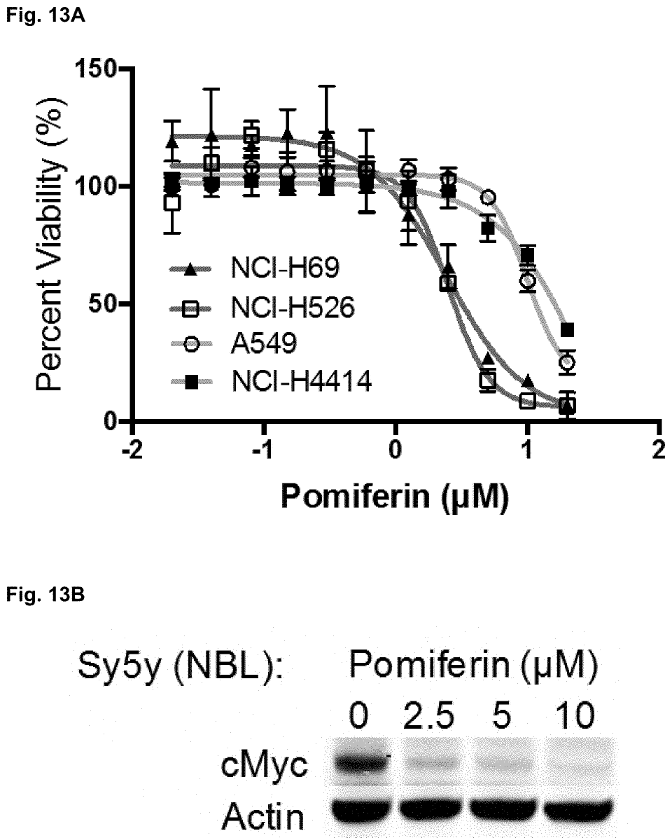

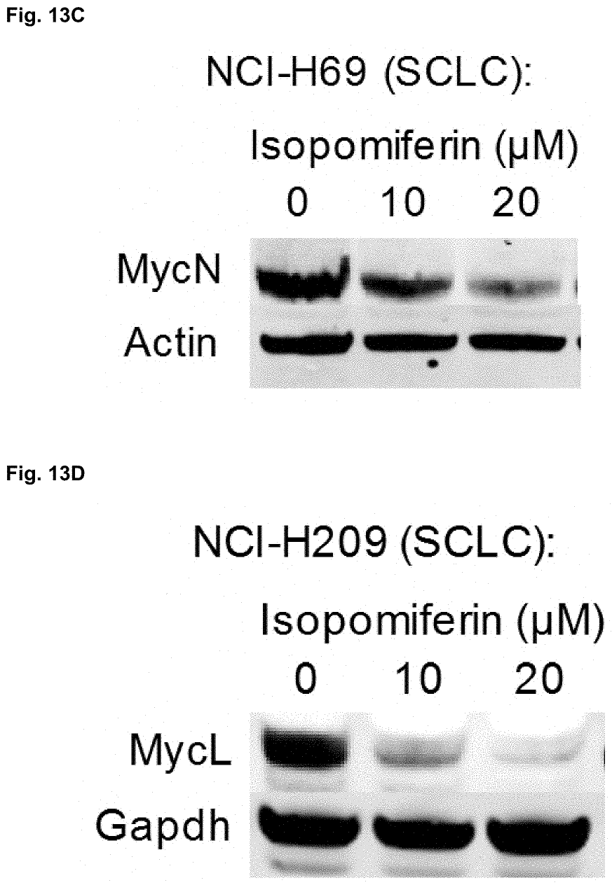

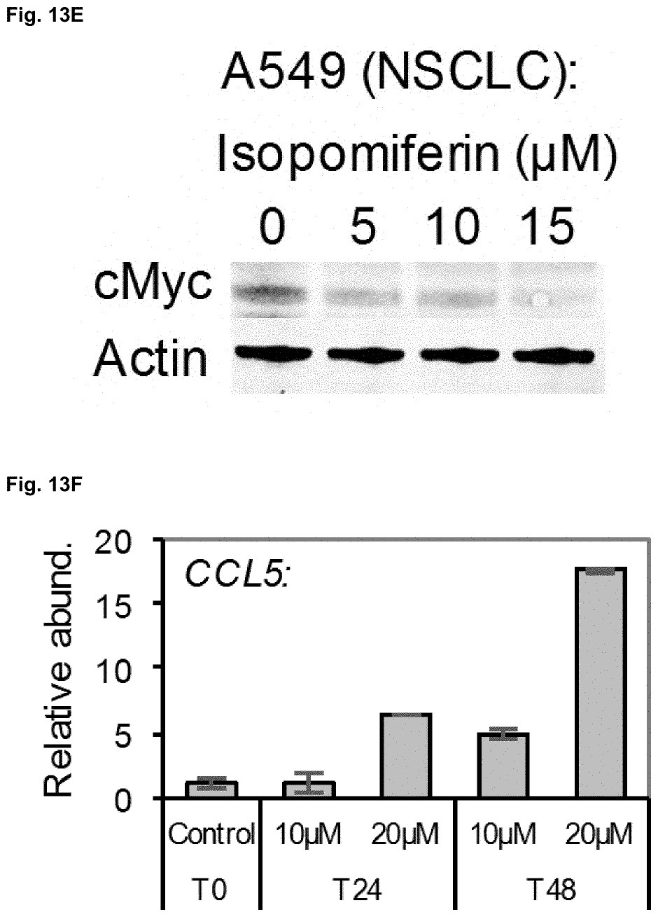

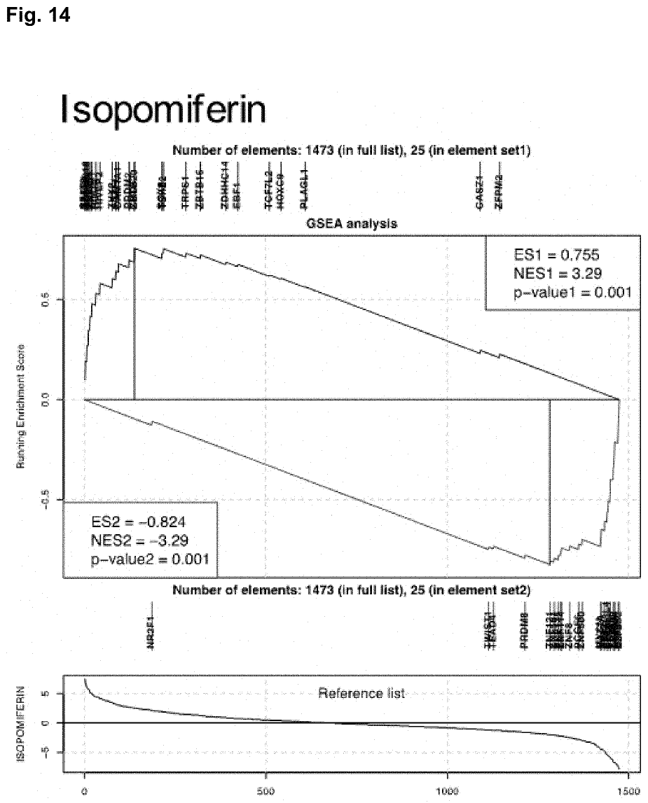

Pomiferin sensitivity was evaluated in two MycN-driven SCLC cell lines (NCI-H69 and NCI-H526) and two cMyc-driven lung cancer models (A549 and NCI-H4414). These four cell lines were treated to pomiferin across a series of doses, which demonstrated MycN-driven cell lines were more sensitive to the compound (FIG. 13A). To test whether this was associated with MycN depletion, NCI-H69 cells were treated with isopomiferin for 24 h, which resulted in concentration-dependent inhibition of MycN protein abundance (FIG. 13C).

As CK2 phosphorylates cMyc and regulates activity and protein abundance (Luscher et al. 1989; Bousset et al. 1994; Channavajhala and Seldin, 2002), it was hypothesized that pomiferin could deplete cMyc and MycL in MYC-driven cell lines. We tested whether pomiferin could suppress cMyc in Sy5y, a cMyc-driven NBL cell line (Zimmerman et al. 2018). Similar to observations in MYCNA cells, pomiferin suppressed cMyc in Sy5y (FIG. 13B). We then tested whether isopomiferin could similarly suppress cMyc or MycL in SCLC lung cancer cell lines by treating NCI-H69 and NCI-H209 with isopomiferin for 24 h. Isopomiferin suppressed MYC protein in both SCLC lines (FIGS. 13C and 13D), confirming that MYC suppression not limited to NBL. cMyc expression may limit tumor response to immunotherapies, as cMyc suppresses key factors enabling immune surveillance, such as CCL5, a secreted T-Cell chemoattractant (Topper et al. 2017). Isopomiferin treatment restored CCL5 expression in NSCLC A549 cells, inducing a 15-fold induction of transcript abundance by 48 h (FIG. 13F). This induction of immune components reflects previous GSEA analyses of Cancer Hallmark pathways (FIG. 9A), which revealed enrichment of the “Inflammatory Response” gene set that includes members of the CCL family (Topper et al. 2017).

### Example 18

### Pomiferin Suppresses Tumor Growth In Vivo

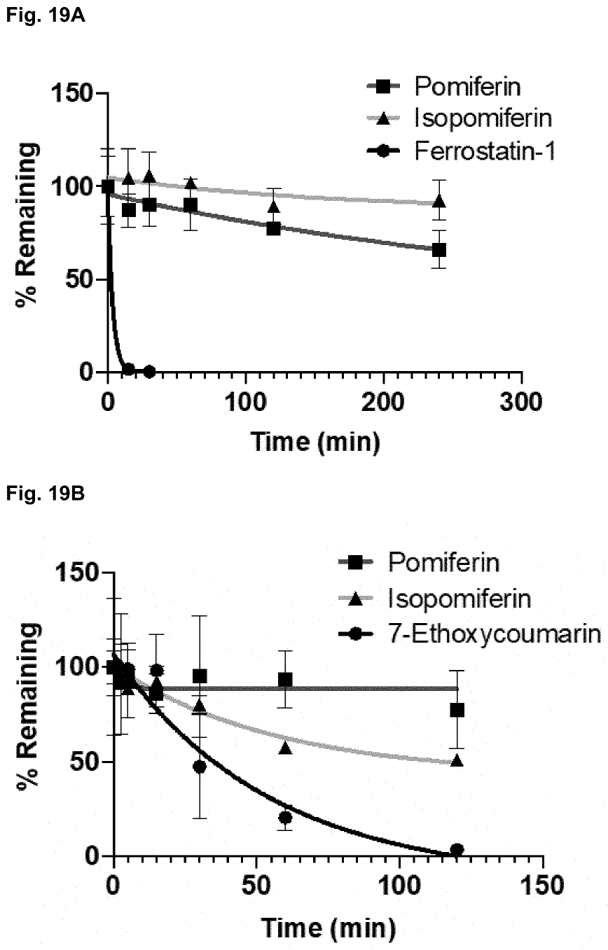

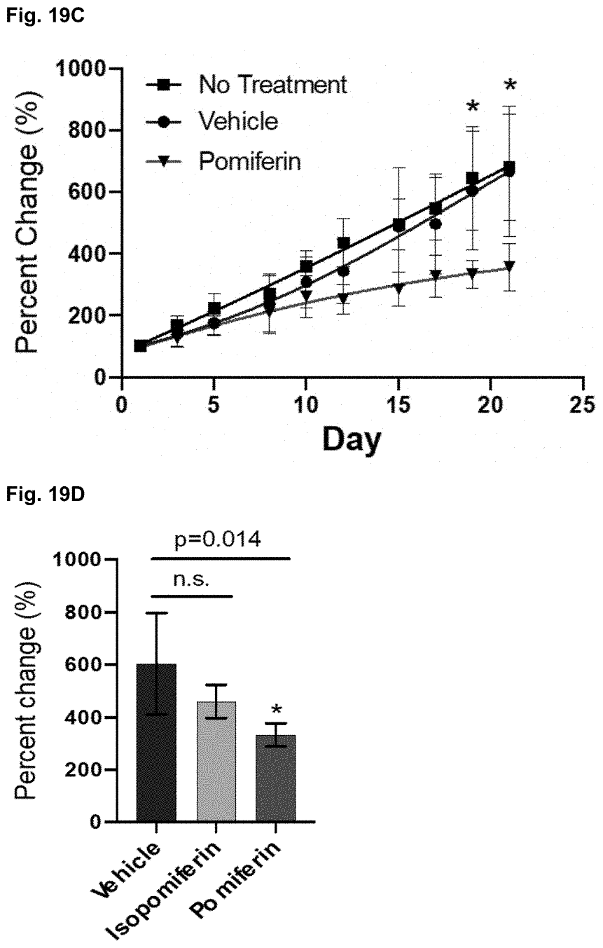

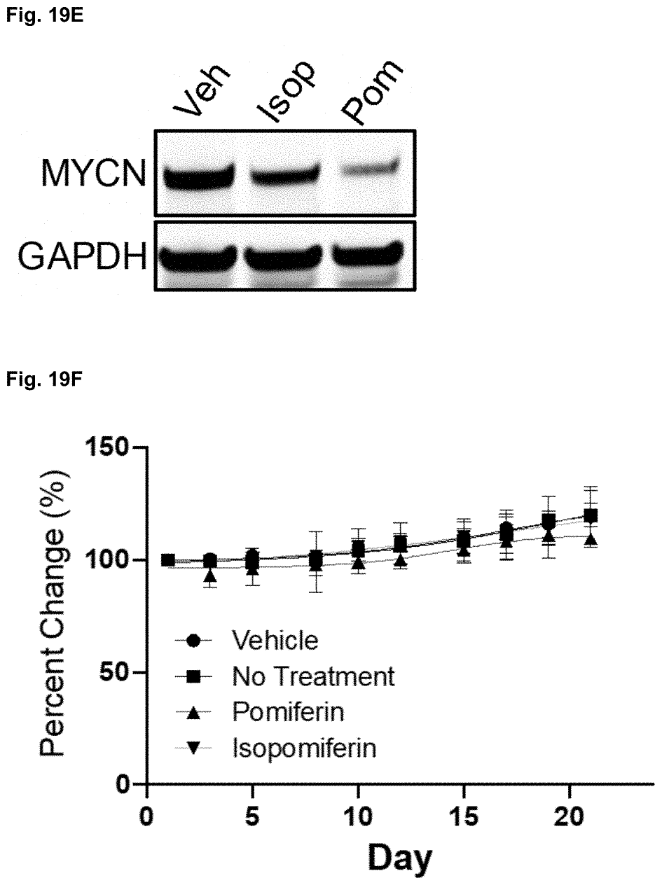

In vitro metabolic stability assays were performed to assess suitability for in vivo studies. Both compounds were incubated alongside a positive control compound (ferrostatin-1) in mouse plasma for 4 h, and quantified by liquid chromatography-mass spectrometry (LC-MS). Isopomiferin was completely stable in mouse plasma, while approximately 75% pomiferin remained following incubation (FIG. 19A). Similarly, both pomiferin and isopomiferin were reasonably stable to metabolic activation by mouse liver microsomes. Approximately ˜80% pomiferin and ˜50% isopomiferin remained after 2 hours incubation, while the positive control compounds 7-ethoxycoumarin was metabolized across the duration of the assay, suggesting that the prenylated isoflavonoids are metabolically stable (FIG. 19B).

The ability of the compounds to inhibit growth of MYCNA tumor xenografts was evaluated by treating tumor bearing mice with daily i.p. injections of 20 mg/kg isopomiferin or pomiferin. Mice that received daily treatments of pomiferin exhibited reduced rates of tumor proliferation that were significant from the vehicle-only control arm by day 19 of the study (FIGS. 19C and 19D). Mice treated with isopomiferin trended toward having smaller tumors than control arms, but these differences were not significant (FIG. 19D). MYCN abundance was evaluated mid-experiment, following 14 days of treatment. Pomiferin caused a substantial decrease in MYCN protein in tumors, relative to both vehicle-only and isopomiferin treated animals (FIG. 19E). Importantly, the mean body weight between each experimental arm was not significant, suggesting that treatment with prenylated isoflavonoids are tolerated in mice at 20 mg/kg (FIG. 19F).

### Example 19

### Discussion

The current standard of care for MYCNA NBL is particularly grueling for pediatric patients, and can have long-lasting implications for growth and development (Cohen et al. 2014; Laverdiere et al. 2005; Laverdiere et al. 2009). Children that receive high-dose radiotherapy and chemotherapy experience reduced growth rates throughput adolescence and higher incidence of hypothyroidism, ovarian failure, hearing loss and dental issues as adults (Cohen et al. 2014). A novel targeted therapy that disrupts core regulatory drivers of MYCNA NBL could improve clinical outcomes for patients and reduce the long-term health effects caused by current treatment modalities. Two studies compared the anti-proliferative effect of pomiferin in cancer cell lines and non-transformed cells and found pomiferin >10-fold more potent in transformed cells (Darji et al. 2013; Son et al. 2007), suggesting that the scaffold is likely non-toxic to healthy tissues. Furthermore, adult tissues do not express MycN, which is normally confined to developing neural tissues. Inhibiting MycN should therefore not be problematic for healthy tissues, and could benefit sensitive pediatric patients.

A targeted therapy that disrupts the key regulatory drivers of MYCNA NBL is a significant improvement to current approaches because it disrupts the feedback mechanisms that drive drug resistance. Recent clinical failure of aurora kinase A inhibitor alisertib highlights the complex regulatory nature and aggressive phenotype of these recalcitrant tumors. Alisertib suppresses MycN by disrupting a physical interaction between MycN and AurKA (Gustafson et al. 2014; Otto et al. 2009), yet failed to induce a significant change in tumor growth in NBL patients (www.clinicaltrials.gov). We observed that alisertib does not suppress the regulatory signature in the same way as isopomiferin, which we believe will be essential to sustain tumor inhibition in patients.

The target patient population that can benefit from a novel MycN-suppressing therapeutic extends beyond the MYCNA NBL patient group. Pomiferin suppresses MycN in lung cell lines, suggesting the novel therapeutic could benefit all patients suffering from all MycN-driven tumors, independent of cancer type. This is significant, as there are subpopulations of MYCNA tumors from a number of aggressive cancers. For example, 12% of Wilms' tumor (WT), neuroendocrine prostate cancers (NEPC), ˜2-3% of non-small cell lung cancer (NSCLC), ˜2-3% of liver cancers are MYCNA, representing a substantial patient population that could benefit from an isopomiferin-based therapeutic. This is particularly important because MycN-driven tumors are noted for their aggressive phenotype and high mortality rate (Huang and Weiss, 2013). MycN-amplification occurs in later stages of adult cancers to exacerbate tumor development at a time when patients have often exhausted other options (Rickman et al. 2018).

Selectivity for MYCNA cells could be used to develop MycN as a predictive biomarker of isopomiferin sensitivity. This biomarker will enable clinicians to identify responsive individuals, stratify patients at clinical trials, and draw meaningful comparisons between groups. This will enhance the probability of success in a clinical setting, and ensure that only responsive patients receive therapy. A basket trial design could be explored in future, in which MYCNA patients from across cancer types are binned based on specific tumor drivers. This may be particularly important for rare tumors, such as WT and NEPC, and may be the quickest way to get these cancers tested for response to a novel therapeutic.

Using PLATE-Seq as an expression profile screening tool for network disruption is a unique approach to drug discovery that offers a competitive advantage over recent attempts to target MycN-driven tumors. PLATE-Seq lowers the cost of traditional expression profiling substantially (Bush et al. 2017), so it is feasible to screen a wider range of structures than traditional RNA-Seq. Introducing PLATE-Seq early in the development program can identify problematic structures that will be avoided in future iterations of the molecule, saving time and resources. Screening across tumor subtypes in combination with high-throughput network analysis is a unique approach to rapidly identifying targeted agents with high therapeutic index. Having successfully identified a scaffold for recalcitrant MycN-driven tumors that can be optimized into a novel therapeutic compound, future researchers can apply this methodology to their cancer of interest. As informatic approaches became more adept at identifying and characterizing molecular tumor subtypes, new target patient populations will be identified that can form the basis of future discovery.

### DOCUMENTS CITED

1. Alvarez, M. J., et al., Functional characterization of somatic mutations in cancer using network-based inference of protein activity. Nat Genet, 2016. 48(8): p. 838-47.
2. Anderes, K., et al., Discovery and characterization of CX-4945 a selective orally bioavailable small molecule inhibitor of protein kinase CK2: Phase 1 initiated. Cancer Research, 2009. 69.
3. Bajer, M. M., et al., Characterization of pomiferin triacetate as a novel mTOR and translation inhibitor. Biochemical Pharmacology, 2014. 88(3): p. 313-321.
4. Ballou, L. M. and R. Z. Lin, Rapamycin and mTOR kinase inhibitors. J Chem Biol, 2008. 1(1-4): p. 27-36.
5. Beltran, H., The N-myc Oncogene: Maximizing its Targets, Regulation, and Therapeutic Potential. Molecular Cancer Research, 2014. 12(6): p. 815-822.
6. Bos, P. H., et al., Development of MAP4 Kinase Inhibitors as Motor Neuron-Protecting Agents. Cell Chem Biol, 2019.
7. Bousset, K., et al., Identification of Casein Kinase-Ii Phosphorylation Sites in Max—Effects on DNA-Binding Kinetics of Max Homodimer and Myc/Max Heterodimers. Oncogene, 1993. 8(12): p. 3211-3220.
8. Bousset, K., et al., Regulation of Transcription Factors C-Myc, Max, and C-Myb by Casein Kinase-Ii. Cellular & Molecular Biology Research, 1994. 40(5-6): p. 501-511.
9. Bowes, J., et al., Reducing safety-related drug attrition: the use of in vitro pharmacological profiling. Nat Rev Drug Discov, 2012. 11(12): p. 909-22.
10. Bragelmann, J., et al., Family matters: How MYC family oncogenes impact small cell lung cancer. Cell Cycle, 2017. 16(16): p. 1489-1498.
11. Brandetti, E., et al., MYCN is an immunosuppressive oncogene dampening the expression of ligands for NK-cell-activating receptors in human high-risk neuroblastoma. Oncoimmunology, 2017. 6(6): p. e1316439.
12. Brodeur, G. M. and R. Bagatell, Mechanisms of neuroblastoma regression. Nat Rev Clin Oncol, 2014. 11(12): p. 704-13.
13. Brodeur, G. M., et al., Amplification of N-myc in untreated human neuroblastomas correlates with advanced disease stage. Science, 1984. 224(4653): p. 1121-4.
14. Bush, E. C., et al., PLATE-Seq for genome-wide regulatory network analysis of high-throughput screens. Nat Commun, 2017. 8(1): p. 105.
15. Califano, A. and M. J. Alvarez, The recurrent architecture of tumour initiation, progression and drug sensitivity. Nat Rev Cancer, 2017. 17(2): p. 116-130.
16. Channavajhala, P. and D. C. Seldin, Functional interaction of protein kinase CK2 and c-Myc in lymphomagenesis. Oncogene, 2002. 21(34): p. 5280-5288.
17. Cheung, N. K. and M. A. Dyer, Neuroblastoma: developmental biology, cancer genomics and immunotherapy. Nat Rev Cancer, 2013. 13(6): p. 397-411.
18. Chon, H. J., et al., The casein kinase 2 inhibitor, CX-4945, as an anti-cancer drug in treatment of human hematological malignancies. Front Pharmacol, 2015. 6: p. 70.
19. Cohen, L. E., et al., Late effects in children treated with intensive multimodal therapy for high-risk neuroblastoma: high incidence of endocrine and growth problems. Bone Marrow Transplant, 2014. 49(4): p. 502-8.
20. Colletti, L. M., et al., Methods to measure the intracellular concentration of unlabeled compounds within cultured cells using liquid chromatography/tandem mass spectrometry. Analytical Biochemistry, 2008. 383(2): p. 186-193.
21. Darji, K., et al., HPLC Determination of Isoflavone Levels in Osage Orange from the Midwest and Southern United States. Journal of Agricultural and Food Chemistry, 2013. 61(28): p. 6806-6811.
22. Drygin, D., et al., CX-4945, a novel small molecule inhibitor of CK2 protein kinase, reduces hyperactivated Akt signaling and synergizes with Akt inhibitors in breast cancer cells. Molecular Cancer Therapeutics, 2009. 8(12).
23. Ferrer-Font, L., et al., Targeting Protein Kinase CK2: Evaluating CX-4945 Potential for GL261 Glioblastoma Therapy in Immunocompetent Mice. Pharmaceuticals (Basel), 2017. 10(1).
24. Funa, K., et al., Increased Expression of N-Myc in Human Small-Cell Lung-Cancer Biopsies Predicts Lack of Response to Chemotherapy and Poor Prognosis. American Journal of Clinical Pathology, 1987. 88(2): p. 216-220.
25. Guertin, D. A. and D. M. Sabatini, Defining the role of mTOR in cancer. Cancer Cell, 2007. 12(1): p. 9-22.
26. Gustafson, W. C., et al., Drugging MYCN through an allosteric transition in Aurora kinase A. Cancer Cell, 2014. 26(3): p. 414-27.
27. Hay, N. and N. Sonenberg, Upstream and downstream of mTOR. Genes Dev, 2004. 18(16): p. 1926-45.
28. Huang, M. and W. A. Weiss, Neuroblastoma and MYCN. Cold Spring Harb Perspect Med, 2013. 3(10): p. a014415.
29. Huang, R., et al., MYCN and MYC regulate tumor proliferation and tumorigenesis directly through BMI1 in human neuroblastomas. Faseb Journal, 2011. 25(12): p. 4138-4149.
30. Irwin, M. S. and J. R. Park, Neuroblastoma: paradigm for precision medicine. Pediatr Clin North Am, 2015. 62(1): p. 225-56.
31. Kim, L. C., R. S. Cook, and J. Chen, mTORC1 and mTORC2 in cancer and the tumor microenvironment. Oncogene, 2017. 36(16): p. 2191-2201.
32. Koppen, A., et al., Dickkopf-1 is down-regulated by MYCN and inhibits neuroblastoma cell proliferation. Cancer Lett, 2007. 256(2): p. 218-28.
33. Lachmann, A., et al., ARACNe-AP: gene network reverse engineering through adaptive partitioning inference of mutual information. Bioinformatics, 2016. 32(14): p. 2233-5.
34. Laverdiere, C., et al., Long-term complications in survivors of advanced stage neuroblastoma. Pediatr Blood Cancer, 2005. 45(3): p. 324-32.
35. Laverdiere, C., et al., Long-term outcomes in survivors of neuroblastoma: a report from the Childhood Cancer Survivor Study. J Natl Cancer Inst, 2009. 101(16): p. 1131-40.
36. Lee, J. K., et al., N-Myc Drives Neuroendocrine Prostate Cancer Initiated from Human Prostate Epithelial Cells. Cancer Cell, 2016. 29(4): p. 536-547.
37. Li, Y. G., et al., microRNA202 suppresses MYCN expression under the control of E2F1 in the neuroblastoma cell line LAN5. Mol Med Rep, 2014. 9(2): p. 541-6.
38. Lipinski, C. A., et al., Experimental and computational approaches to estimate solubility and permeability in drug discovery and development settings. Advanced Drug Delivery Reviews, 1997. 23(1-3): p. 3-25.
39. Litchfield, D. W. and B. Luscher, Casein Kinase-Ii in Signal-Transduction and Cell-Cycle Regulation. Molecular and Cellular Biochemistry, 1993. 128: p. 187-199.
40. Litchfield, D. W., Protein kinase CK2: structure, regulation and role in cellular decisions of life and death. Biochem J, 2003. 369(Pt 1): p. 1-15.
41. Liu, K., et al., Overexpression of MYCN promotes proliferation of non-small cell lung cancer. Tumor Biology, 2016. 37(9): p. 12855-12866.
42. Louis, C. U. and J. M. Shohet, Neuroblastoma: molecular pathogenesis and therapy. Annu Rev Med, 2015. 66: p. 49-63.
43. Luscher, B., et al., Myc Oncoproteins Are Phosphorylated by Casein Kinase-Ii. Embo Journal, 1989. 8(4): p. 1111-1119.
44. Ma, L., et al., miR-9, a MYC/MYCN-activated microRNA, regulates E-cadherin and cancer metastasis. Nature Cell Biology, 2010. 12(3): p. 247-U52.
45. Manning, G., et al., The protein kinase complement of the human genome. Science, 2002. 298(5600): p. 1912-34.
46. Marshall, G. M., et al., The prenatal origins of cancer. Nat Rev Cancer, 2014. 14(4): p. 277-89.
47. Nau, M. M., et al., Human Small-Cell Lung Cancers Show Amplification and Expression of the N-Myc Gene. Proceedings of the National Academy of Sciences of the United States of America, 1986. 83(4): p. 1092-1096.
48. Ora, I. and A. Eggert, Progress in treatment and risk stratification of neuroblastoma: impact on future clinical and basic research. Semin Cancer Biol, 2011. 21(4): p. 217-28.
49. Otto, T., et al., Stabilization of N-Myc is a critical function of Aurora A in human neuroblastoma. Cancer Cell, 2009. 15(1): p. 67-78.
50. Phillips, H. S., et al., Molecular subclasses of high-grade glioma predict prognosis, delineate a pattern of disease progression, and resemble stages in neurogenesis. Cancer Cell, 2006. 9(3): p. 157-173.
51. Populo, H., J. M. Lopes, and P. Soares, The mTOR Signalling Pathway in Human Cancer. International Journal of Molecular Sciences, 2012. 13(2): p. 1886-1918.
52. Prins, R. C., et al., CX-4945, a selective inhibitor of casein kinase-2 (CK2), exhibits anti-tumor activity in hematologic malignancies including enhanced activity in chronic lymphocytic leukemia when combined with fludarabine and inhibitors of the B-cell receptor pathway. Leukemia, 2013. 27(10): p. 2094-2096.
53. Rajbhandari, P., et al., Cross-cohort analysis identifies a TEAD4<-->MYCN positive-feedback loop as the core regulatory element of high-risk neuroblastoma. Cancer Discov, 2018.
54. Richards, M. W., et al., Structural basis of N-Myc binding by Aurora-A and its destabilization by kinase inhibitors. Proc Natl Acad Sci USA, 2016. 113(48): p. 13726-13731.
55. Rickman, D. S., J. H. Schulte, and M. Eilers, The Expanding World of N-MYC-Driven Tumors. Cancer Discov, 2018. 8(2): p. 150-163.
56. Sarbassov, D. D., et al., Phosphorylation and regulation of Akt/PKB by the rictor-mTOR complex. Science, 2005. 307(5712): p. 1098-1101.
57. Seeger, R. C., et al., Association of multiple copies of the N-myc oncogene with rapid progression of neuroblastomas. N Engl J Med, 1985. 313(18): p. 1111-6.
58. Shimada, H., et al., International neuroblastoma pathology classification for prognostic evaluation of patients with peripheral neuroblastic tumors—A report from the Children's Cancer Group. Cancer, 2001. 92(9): p. 2451-2461.
59. Siddiqui-Jain, A., et al., CX-4945, an Orally Bioavailable Selective Inhibitor of Protein Kinase CK2, Inhibits Prosurvival and Angiogenic Signaling and Exhibits Antitumor Efficacy. Cancer Research, 2010. 70(24): p. 10288-10298.
60. Son, I. H., et al., Pomiferin, histone deacetylase inhibitor isolated from the fruits of Maclura pomifera. Bioorganic & Medicinal Chemistry Letters, 2007. 17(17): p. 4753-4755.
61. Strieder, V. and W. Lutz, E2F proteins regulate MYCN expression in neuroblastomas. Journal of Biological Chemistry, 2003. 278(5): p. 2983-2989.
62. Topper, M. J., et al., Epigenetic Therapy Ties MYC Depletion to Reversing Immune Evasion and Treating Lung Cancer. Cell, 2017. 171(6): p. 1284-1300 e21.
63. Trembley, J. H., et al., CK2: A key player in cancer biology. Cellular and Molecular Life Sciences, 2009. 66(11-12): p. 1858-1867.
64. Turowec, J. P., et al., Protein kinase CK2 is a constitutively active enzyme that promotes cell survival: strategies to identify CK2 substrates and manipulate its activity in mammalian cells. Methods Enzymol, 2010. 484: p. 471-93.
65. Wang, K., et al., Genome-wide identification of post-translational modulators of transcription factor activity in human B cells. Nat Biotechnol, 2009. 27(9): p. 829-39.
66. Waring, J. F., et al., Clustering of hepatotoxins based on mechanism of toxicity using gene expression profiles. Toxicol Appl Pharmacol, 2001. 175(1): p. 28-42.
67. Waring, J. F., et al., Microarray analysis of hepatotoxins in vitro reveals a correlation between gene expression profiles and mechanisms of toxicity. Toxicol Lett, 2001. 120(1-3): p. 359-68.
68. Welsch, M. E., et al., Multivalent Small-Molecule Pan-RAS Inhibitors. Cell, 2017. 168(5): p. 878-+.
69. Williams, R. D., et al., Multiple mechanisms of MYCN dysregulation in Wilms tumour. Oncotarget, 2015. 6(9): p. 7232-43.
70. Wolfrom, M. L., et al., Osage Orange Pigments .11. Complete Structures of Osajin and Pomiferin. Journal of the American Chemical Society, 1946. 68(3): p. 406-418.
71. Wong, A. J., et al., Gene Amplification of C-Myc and N-Myc in Small Cell-Carcinoma of the Lung. Science, 1986. 233(4762): p. 461-464.
72. Woo, J. H., et al., Elucidating Compound Mechanism of Action by Network Perturbation Analysis. Cell, 2015. 162(2): p. 441-451.
73. Yue, Z. X., et al., MYCN amplification predicts poor prognosis based on interphase fluorescence in situ hybridization analysis of bone marrow cells in bone marrow metastases of neuroblastoma. Cancer Cell Int, 2017. 17: p. 43.
74. Zhang, Q. C., et al., Structure-based prediction of protein-protein interactions on a genome-wide scale. Nature, 2012. 490(7421): p. 556-60.
75. Zimmerman, M. W., et al., MYC Drives a Subset of High-Risk Pediatric Neuroblastomas and Is Activated through Mechanisms Including Enhancer Hijacking and Focal Enhancer Amplification. Cancer Discov, 2018. 8(3): p. 320-335.

All patents, patent applications, and publications cited above are incorporated herein by reference in their entirety as if recited in full herein.

The disclosure being thus described, it will be obvious that the same may be varied in many ways. Such variations are not to be regarded as a departure from the spirit and scope of the disclosure and all such modifications are intended to be included within the scope of the following claims.

What is claimed is:

1. A compound having the formula (I): 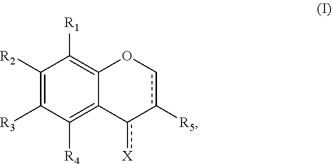
wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof,
or an N-oxide, crystalline form, hydrate thereof, or a pharmaceutically acceptable salt thereof,
with the proviso that the compound is not

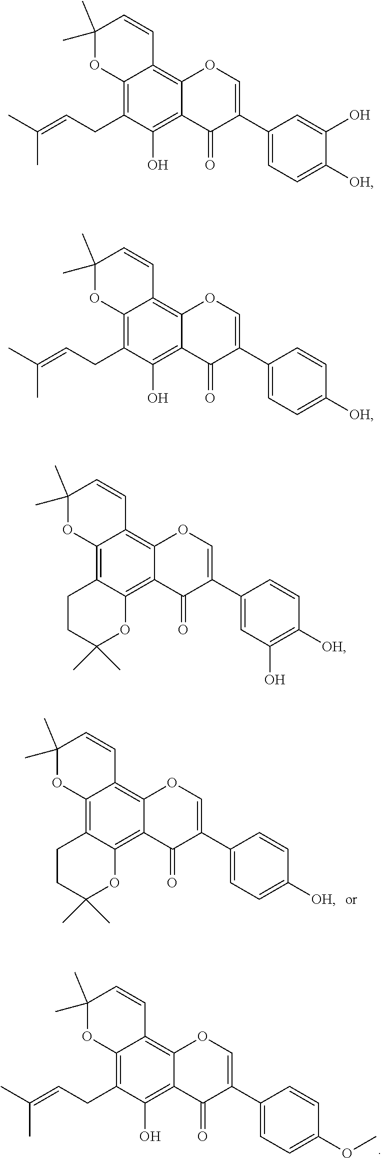 [SMILES: CC(C)=CCc1c2c(c3occ(-c4ccc(O)c(O)c4)c(=O)c3c1O)C=CC(C)(C)O2 ; CC(C)=CCc1c2c(c3occ(-c4ccc(O)cc4)c(=O)c3c1O)C=CC(C)(C)O2 ; CC1(C)C=Cc2c(c3c(c4c(=O)c(-c5ccc(O)c(O)c5)coc24)OC(C)(C)CC3)O1 ; CC1(C)C=Cc2c(c3c(c4c(=O)c(-c5ccc(O)cc5)coc24)OC(C)(C)CC3)O1 ; COc1ccc(-c2coc3c4c(c(CC=C(C)C)c(O)c3c2=O)OC(C)(C)C=C4)cc1]

2. The compound according to claim 1, which is selected from the group consisting of: 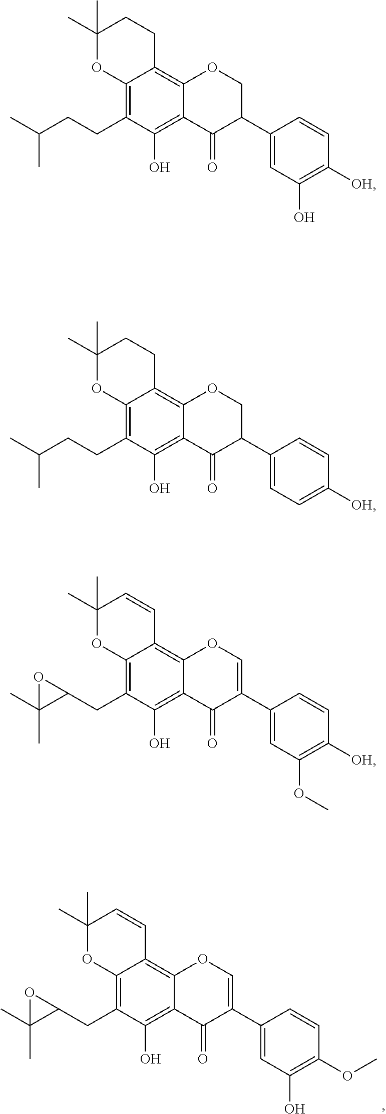 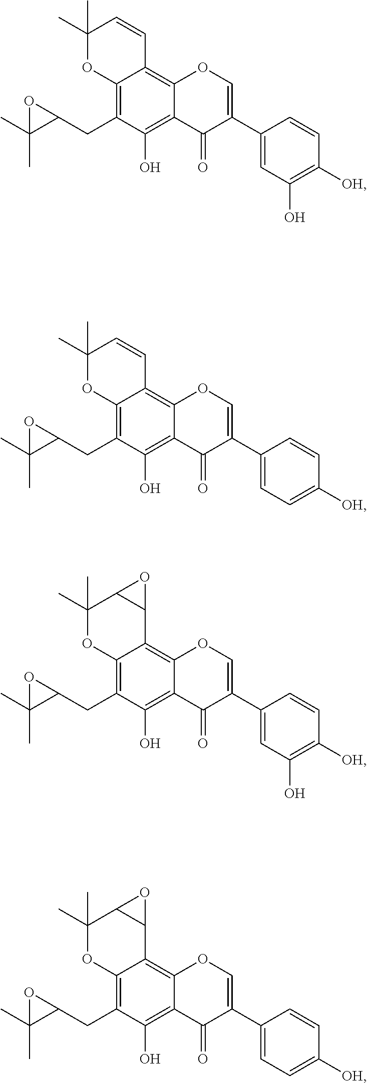 [SMILES: CC1(C)C=Cc2c(c(CC3OC3(C)C)c(O)c3c(=O)c(-c4ccc(O)c(O)c4)coc23)O1 ; CC1(C)C=Cc2c(c(CC3OC3(C)C)c(O)c3c(=O)c(-c4ccc(O)cc4)coc23)O1 ; CC1(C)OC1Cc1c2c(c3occ(-c4ccc(O)c(O)c4)c(=O)c3c1O)C1OC1C(C)(C)O2 ; CC1(C)OC1Cc1c2c(c3occ(-c4ccc(O)cc4)c(=O)c3c1O)C1OC1C(C)(C)O2] 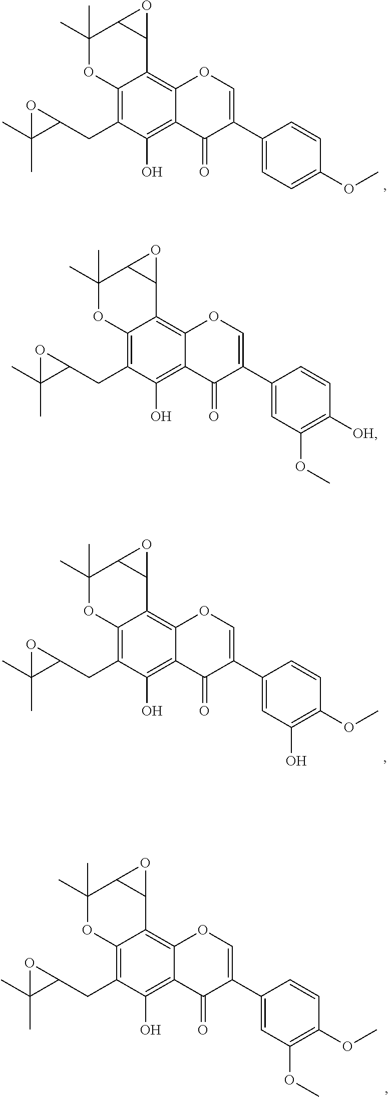 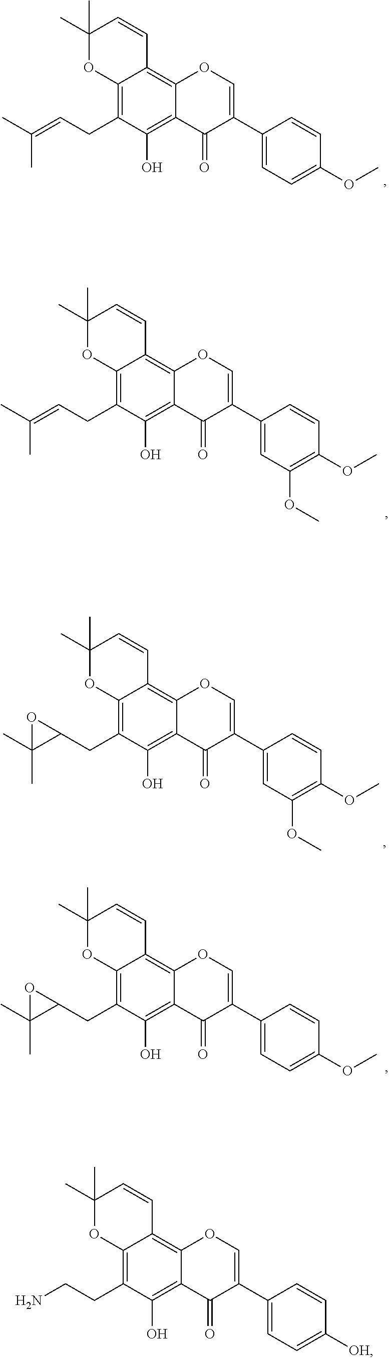 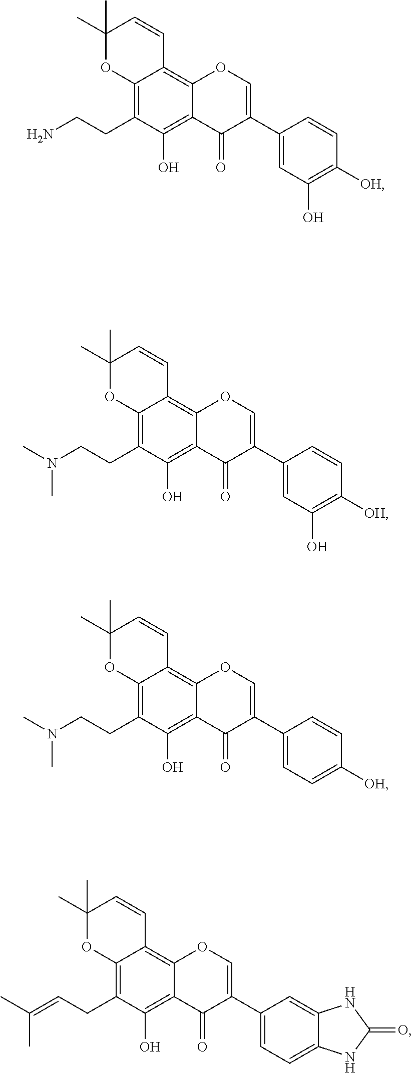 [SMILES: CC1(C)C=Cc2c(c(CCN)c(O)c3c(=O)c(-c4ccc(O)c(O)c4)coc23)O1 ; CN(C)CCc1c2c(c3occ(-c4ccc(O)c(O)c4)c(=O)c3c1O)C=CC(C)(C)O2 ; CN(C)CCc1c2c(c3occ(-c4ccc(O)cc4)c(=O)c3c1O)C=CC(C)(C)O2 ; CC(C)=CCc1c2c(c3occ(-c4ccc5[nH]c(=O)[nH]c5c4)c(=O)c3c1O)C=CC(C)(C)O2] 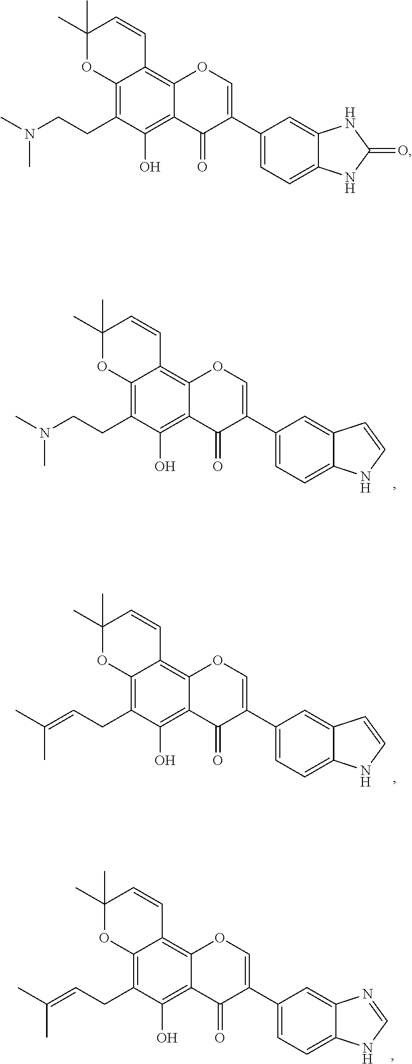 [SMILES: CN(C)CCc1c2c(c3occ(-c4ccc5[nH]c(=O)[nH]c5c4)c(=O)c3c1O)C=CC(C)(C)O2 ; CN(C)CCc1c2c(c3occ(-c4ccc5[nH]ccc5c4)c(=O)c3c1O)C=CC(C)(C)O2 ; CC(C)=CCc1c2c(c3occ(-c4ccc5[nH]ccc5c4)c(=O)c3c1O)C=CC(C)(C)O2 ; CC(C)=CCc1c2c(c3occ(-c4ccc5[nH]cnc5c4)c(=O)c3c1O)C=CC(C)(C)O2] 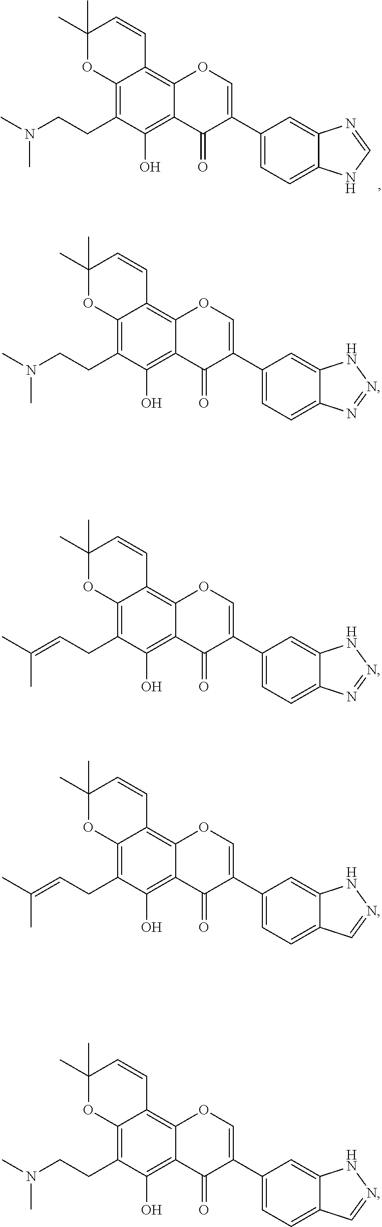 [SMILES: CN(C)CCc1c2c(c3occ(-c4ccc5[nH]cnc5c4)c(=O)c3c1O)C=CC(C)(C)O2 ; CN(C)CCc1c2c(c3occ(-c4ccc5nn[nH]c5c4)c(=O)c3c1O)C=CC(C)(C)O2 ; CC(C)=CCc1c2c(c3occ(-c4ccc5nn[nH]c5c4)c(=O)c3c1O)C=CC(C)(C)O2 ; CC(C)=CCc1c2c(c3occ(-c4ccc5cn[nH]c5c4)c(=O)c3c1O)C=CC(C)(C)O2 ; CN(C)CCc1c2c(c3occ(-c4ccc5cn[nH]c5c4)c(=O)c3c1O)C=CC(C)(C)O2] 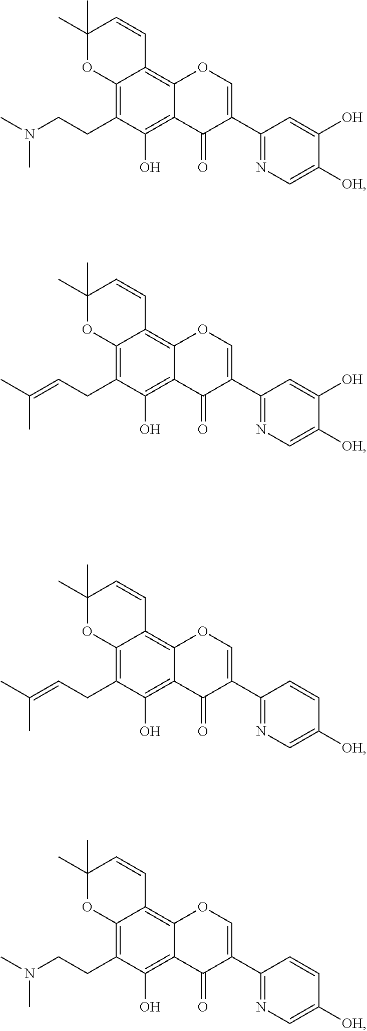 [SMILES: CN(C)CCc1c2c(c3occ(-c4cc(O)c(O)cn4)c(=O)c3c1O)C=CC(C)(C)O2 ; CC(C)=CCc1c2c(c3occ(-c4cc(O)c(O)cn4)c(=O)c3c1O)C=CC(C)(C)O2 ; CC(C)=CCc1c2c(c3occ(-c4ccc(O)cn4)c(=O)c3c1O)C=CC(C)(C)O2 ; CN(C)CCc1c2c(c3occ(-c4ccc(O)cn4)c(=O)c3c1O)C=CC(C)(C)O2] 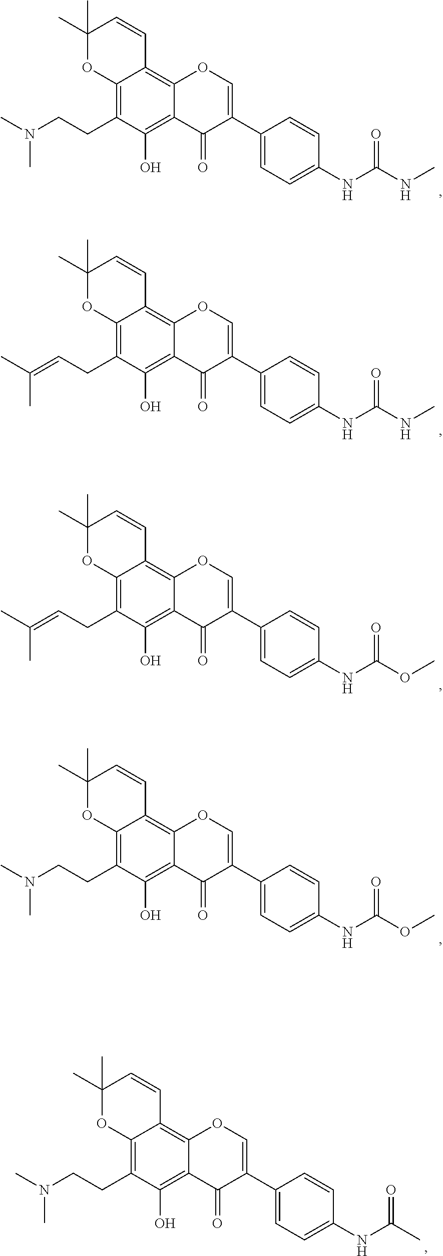 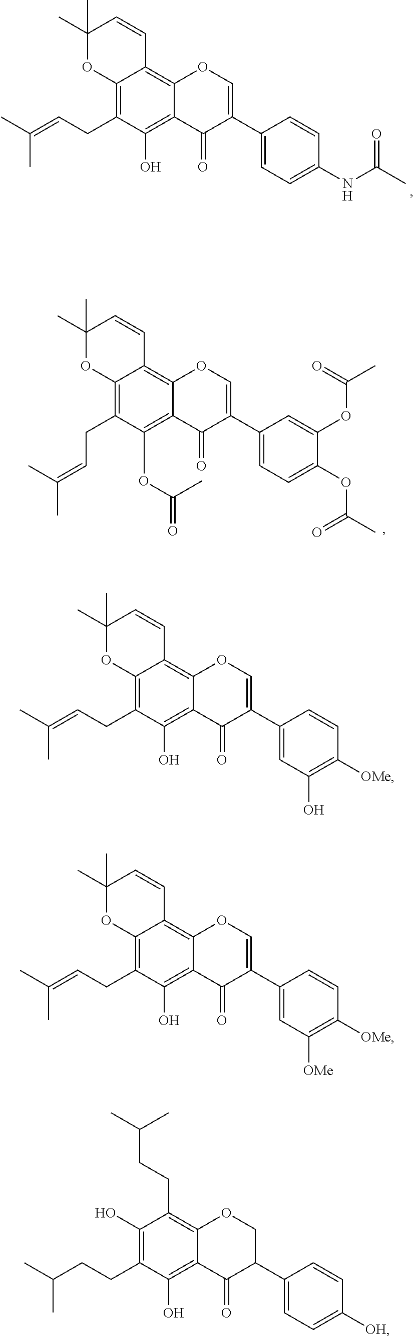 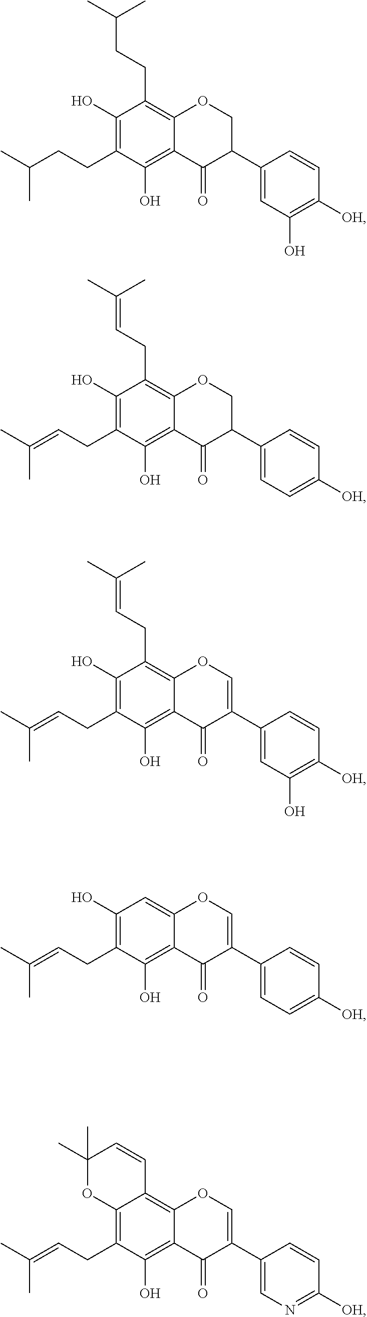 [SMILES: CC(C)CCc1c(O)c(CCC(C)C)c2c(c1O)C(=O)C(c1ccc(O)c(O)c1)CO2 ; CC(C)=CCc1c(O)c(CC=C(C)C)c2c(c1O)C(=O)C(c1ccc(O)cc1)CO2 ; CC(C)=CCc1c(O)c(CC=C(C)C)c2occ(-c3ccc(O)c(O)c3)c(=O)c2c1O ; CC(C)=CCc1c(O)cc2occ(-c3ccc(O)cc3)c(=O)c2c1O ; CC(C)=CCc1c2c(c3occ(-c4ccc(O)nc4)c(=O)c3c1O)C=CC(C)(C)O2] 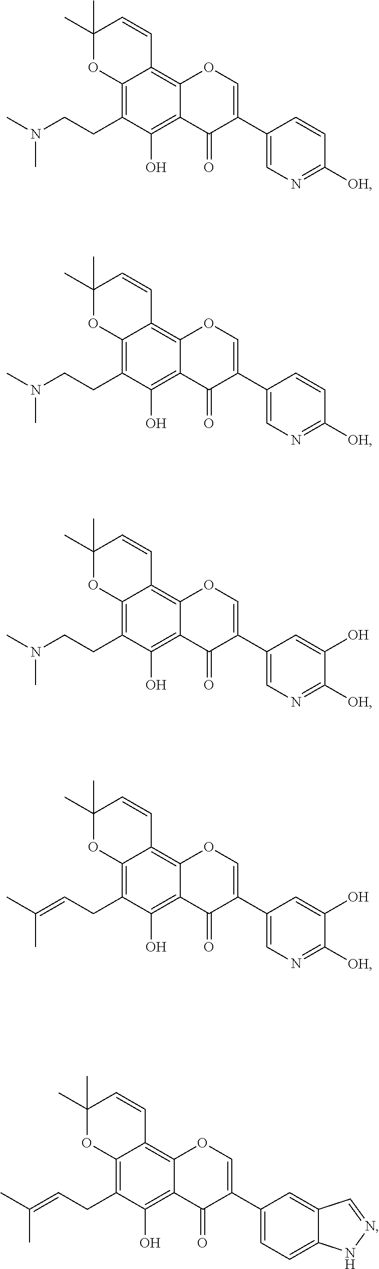 [SMILES: CN(C)CCc1c2c(c3occ(-c4ccc(O)nc4)c(=O)c3c1O)C=CC(C)(C)O2 ; CN(C)CCc1c2c(c3occ(-c4ccc(O)nc4)c(=O)c3c1O)C=CC(C)(C)O2 ; CN(C)CCc1c2c(c3occ(-c4cnc(O)c(O)c4)c(=O)c3c1O)C=CC(C)(C)O2 ; CC(C)=CCc1c2c(c3occ(-c4cnc(O)c(O)c4)c(=O)c3c1O)C=CC(C)(C)O2 ; CC(C)=CCc1c2c(c3occ(-c4ccc5[nH]ncc5c4)c(=O)c3c1O)C=CC(C)(C)O2] 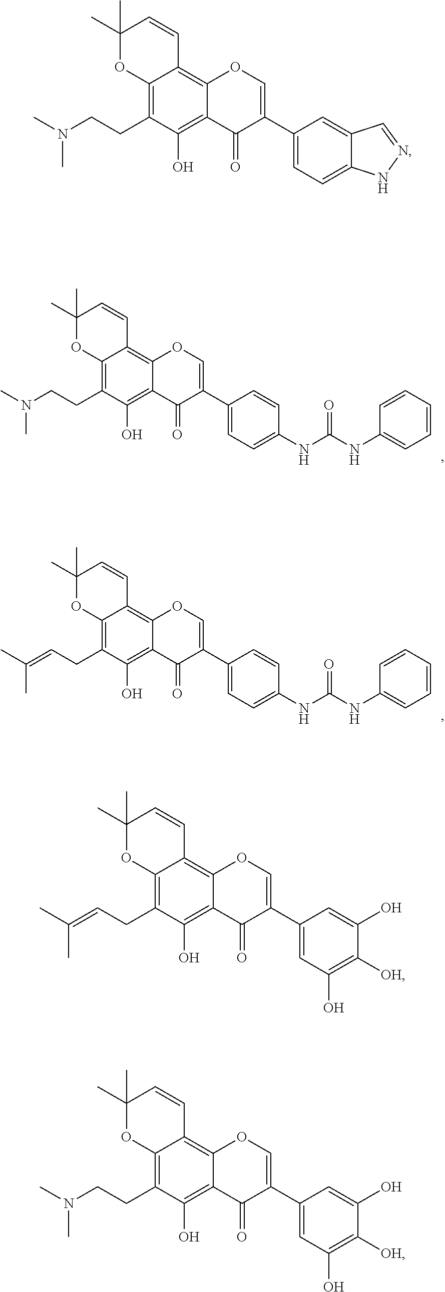 [SMILES: CN(C)CCc1c2c(c3occ(-c4ccc5[nH]ncc5c4)c(=O)c3c1O)C=CC(C)(C)O2 ; CN(C)CCc1c2c(c3occ(-c4ccc(NC(=O)Nc5ccccc5)cc4)c(=O)c3c1O)C=CC(C)(C)O2 ; CC(C)=CCc1c2c(c3occ(-c4ccc(NC(=O)Nc5ccccc5)cc4)c(=O)c3c1O)C=CC(C)(C)O2 ; CC(C)=CCc1c2c(c3occ(-c4cc(O)c(O)c(O)c4)c(=O)c3c1O)C=CC(C)(C)O2 ; CN(C)CCc1c2c(c3occ(-c4cc(O)c(O)c(O)c4)c(=O)c3c1O)C=CC(C)(C)O2] 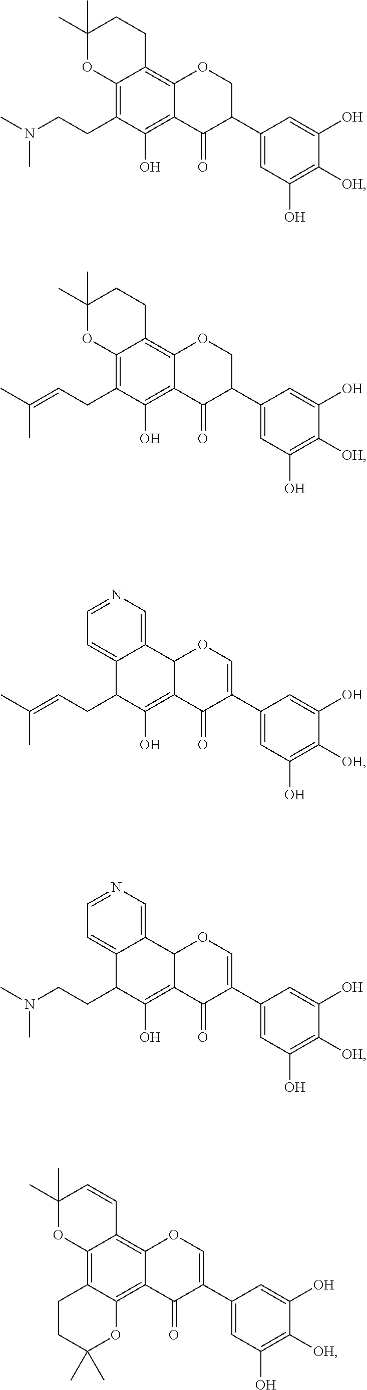 [SMILES: CN(C)CCc1c(O)c2c(c3c1OC(C)(C)CC3)OCC(c1cc(O)c(O)c(O)c1)C2=O ; CC(C)=CCc1c(O)c2c(c3c1OC(C)(C)CC3)OCC(c1cc(O)c(O)c(O)c1)C2=O ; CC(C)=CCC1C(O)=C2C(=O)C(c3cc(O)c(O)c(O)c3)=COC2c2cnccc21 ; CN(C)CCC1C(O)=C2C(=O)C(c3cc(O)c(O)c(O)c3)=COC2c2cnccc21 ; CC1(C)C=Cc2c(c3c(c4c(=O)c(-c5cc(O)c(O)c(O)c5)coc24)OC(C)(C)CC3)O1]
and combinations thereof,
or an N-oxide, crystalline form, hydrate thereof, or a pharmaceutically acceptable salt thereof.

3. A pharmaceutical composition comprising a pharmaceutically acceptable carrier or diluent and a compound according to formula (I): 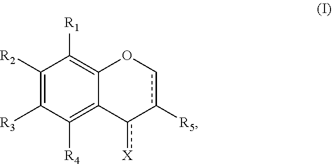
wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄alkyl, CF₃, and combinations thereof,
or an N-oxide, crystalline form, hydrate thereof, or a pharmaceutically acceptable salt thereof,
with the proviso that the compound is not

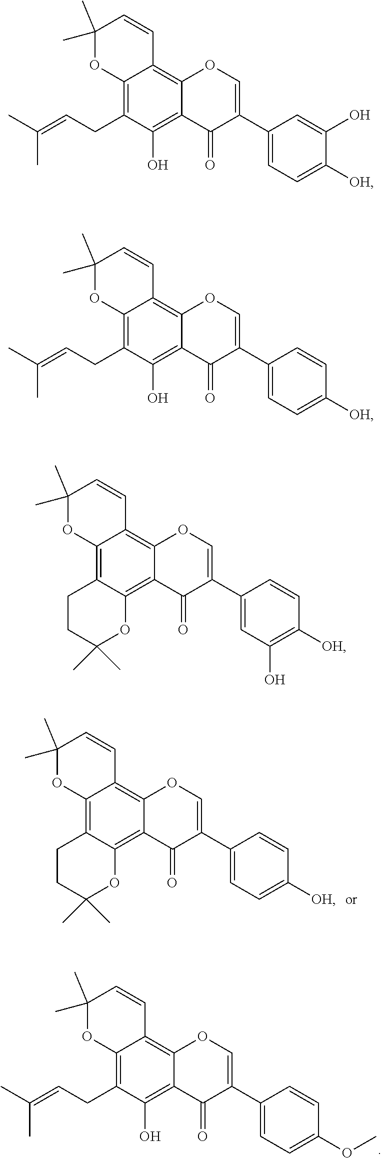 [SMILES: CC(C)=CCc1c2c(c3occ(-c4ccc(O)c(O)c4)c(=O)c3c1O)C=CC(C)(C)O2 ; CC(C)=CCc1c2c(c3occ(-c4ccc(O)cc4)c(=O)c3c1O)C=CC(C)(C)O2 ; CC1(C)C=Cc2c(c3c(c4c(=O)c(-c5ccc(O)c(O)c5)coc24)OC(C)(C)CC3)O1 ; CC1(C)C=Cc2c(c3c(c4c(=O)c(-c5ccc(O)cc5)coc24)OC(C)(C)CC3)O1 ; COc1ccc(-c2coc3c4c(c(CC=C(C)C)c(O)c3c2=O)OC(C)(C)C=C4)cc1]

4. A pharmaceutical composition comprising a pharmaceutically acceptable carrier or diluent and a compound having a structure that is selected from the group consisting of: 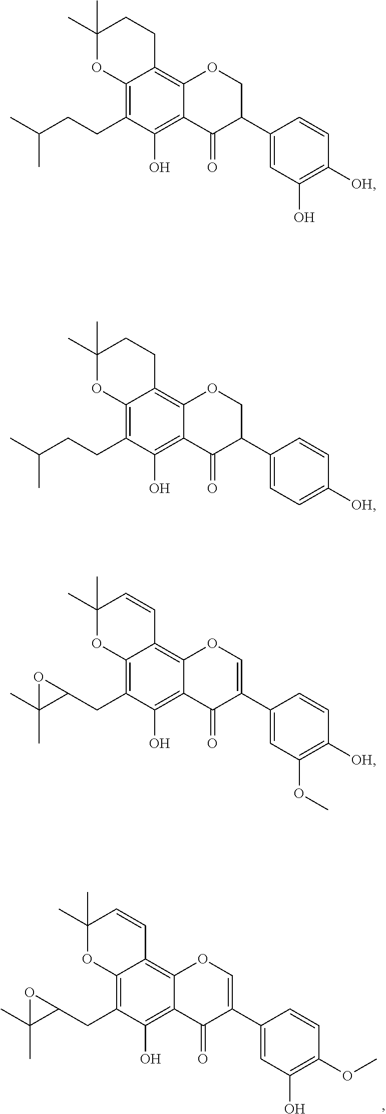 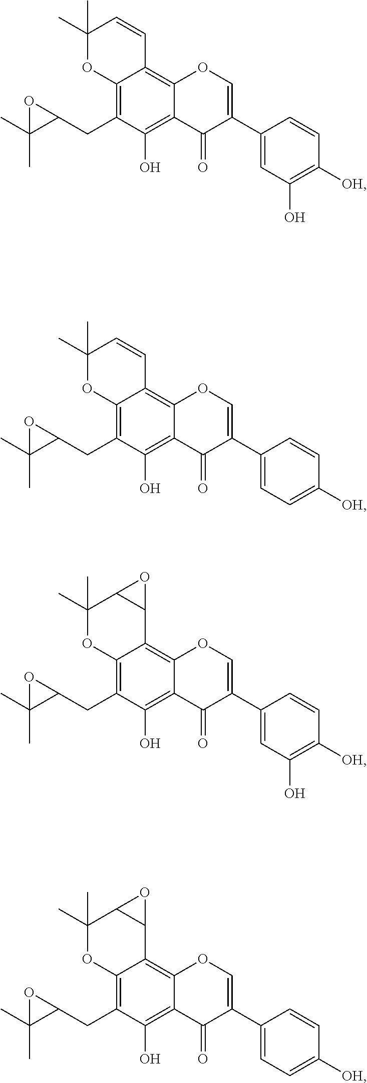 [SMILES: CC1(C)C=Cc2c(c(CC3OC3(C)C)c(O)c3c(=O)c(-c4ccc(O)c(O)c4)coc23)O1 ; CC1(C)C=Cc2c(c(CC3OC3(C)C)c(O)c3c(=O)c(-c4ccc(O)cc4)coc23)O1 ; CC1(C)OC1Cc1c2c(c3occ(-c4ccc(O)c(O)c4)c(=O)c3c1O)C1OC1C(C)(C)O2 ; CC1(C)OC1Cc1c2c(c3occ(-c4ccc(O)cc4)c(=O)c3c1O)C1OC1C(C)(C)O2] 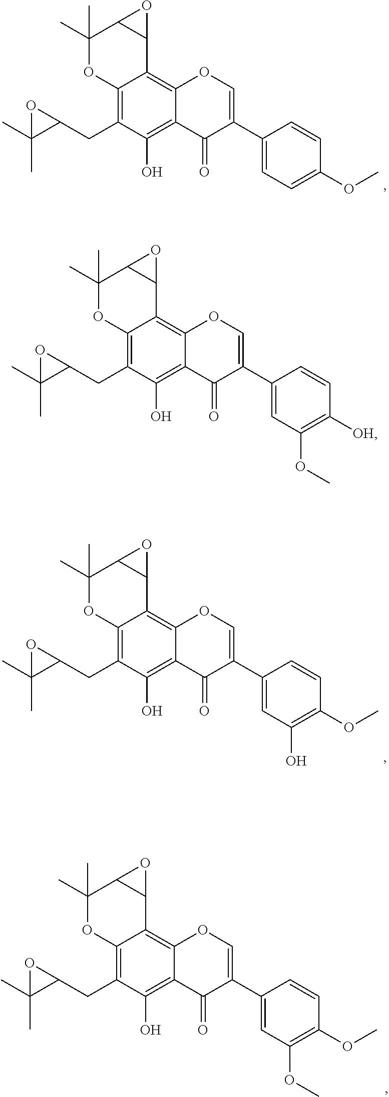 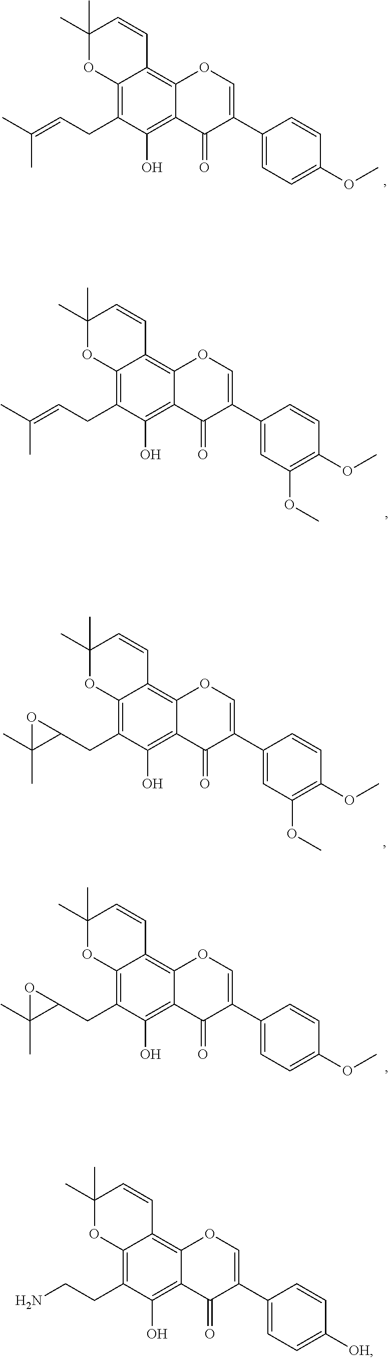 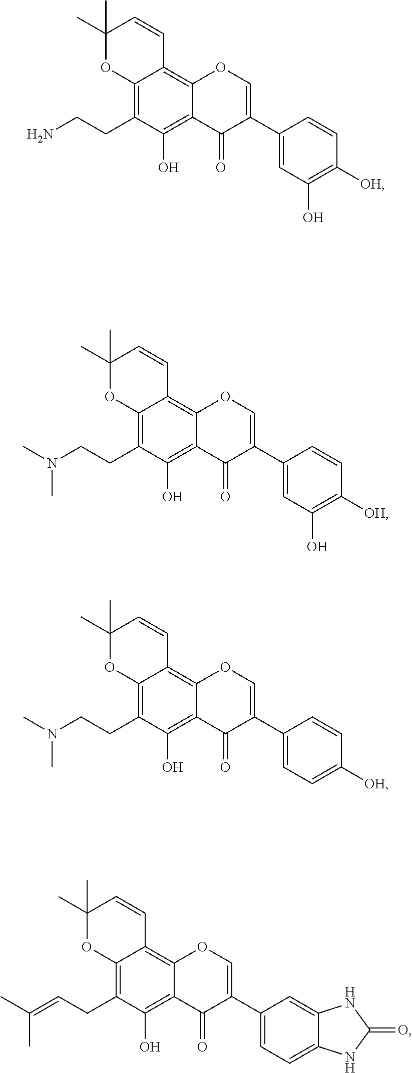 [SMILES: CC1(C)C=Cc2c(c(CCN)c(O)c3c(=O)c(-c4ccc(O)c(O)c4)coc23)O1 ; CN(C)CCc1c2c(c3occ(-c4ccc(O)c(O)c4)c(=O)c3c1O)C=CC(C)(C)O2 ; CN(C)CCc1c2c(c3occ(-c4ccc(O)cc4)c(=O)c3c1O)C=CC(C)(C)O2 ; CC(C)=CCc1c2c(c3occ(-c4ccc5[nH]c(=O)[nH]c5c4)c(=O)c3c1O)C=CC(C)(C)O2] 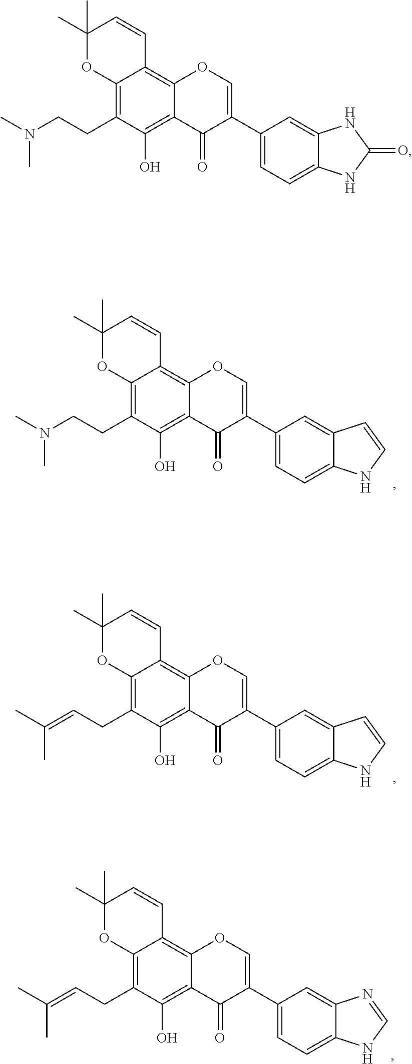 [SMILES: CN(C)CCc1c2c(c3occ(-c4ccc5[nH]c(=O)[nH]c5c4)c(=O)c3c1O)C=CC(C)(C)O2 ; CN(C)CCc1c2c(c3occ(-c4ccc5[nH]ccc5c4)c(=O)c3c1O)C=CC(C)(C)O2 ; CC(C)=CCc1c2c(c3occ(-c4ccc5[nH]ccc5c4)c(=O)c3c1O)C=CC(C)(C)O2 ; CC(C)=CCc1c2c(c3occ(-c4ccc5[nH]cnc5c4)c(=O)c3c1O)C=CC(C)(C)O2] 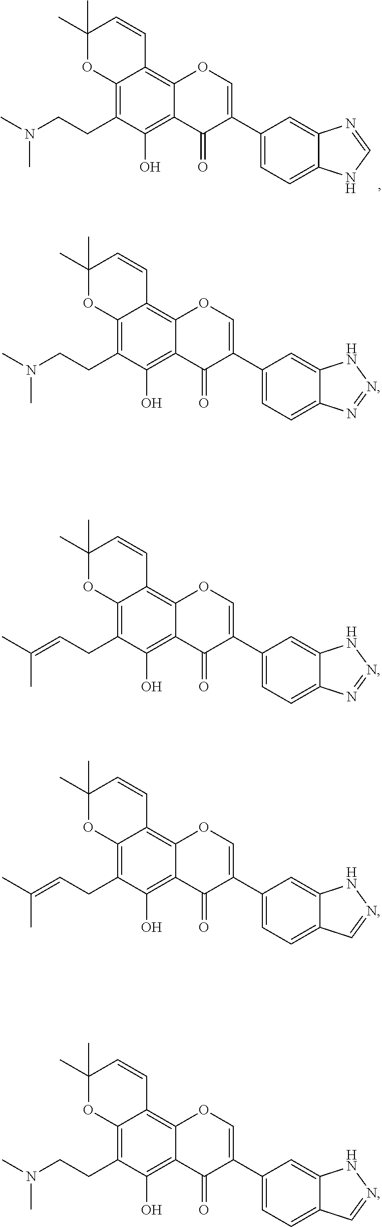 [SMILES: CN(C)CCc1c2c(c3occ(-c4ccc5[nH]cnc5c4)c(=O)c3c1O)C=CC(C)(C)O2 ; CN(C)CCc1c2c(c3occ(-c4ccc5nn[nH]c5c4)c(=O)c3c1O)C=CC(C)(C)O2 ; CC(C)=CCc1c2c(c3occ(-c4ccc5nn[nH]c5c4)c(=O)c3c1O)C=CC(C)(C)O2 ; CC(C)=CCc1c2c(c3occ(-c4ccc5cn[nH]c5c4)c(=O)c3c1O)C=CC(C)(C)O2 ; CN(C)CCc1c2c(c3occ(-c4ccc5cn[nH]c5c4)c(=O)c3c1O)C=CC(C)(C)O2] 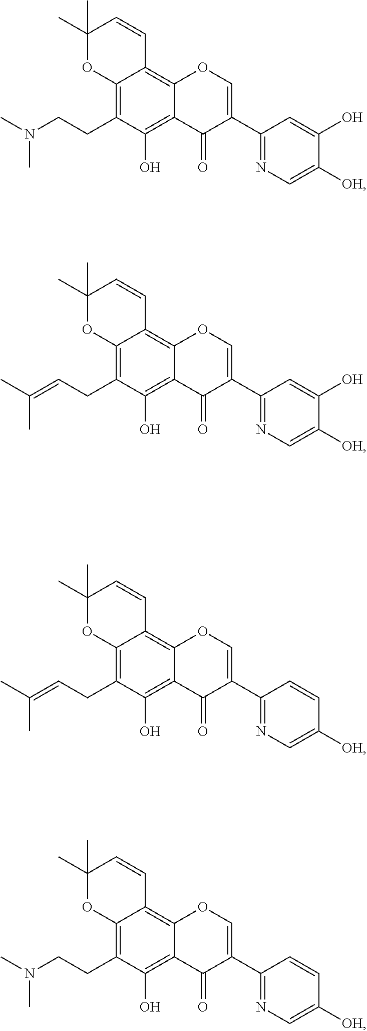 [SMILES: CN(C)CCc1c2c(c3occ(-c4cc(O)c(O)cn4)c(=O)c3c1O)C=CC(C)(C)O2 ; CC(C)=CCc1c2c(c3occ(-c4cc(O)c(O)cn4)c(=O)c3c1O)C=CC(C)(C)O2 ; CC(C)=CCc1c2c(c3occ(-c4ccc(O)cn4)c(=O)c3c1O)C=CC(C)(C)O2 ; CN(C)CCc1c2c(c3occ(-c4ccc(O)cn4)c(=O)c3c1O)C=CC(C)(C)O2] 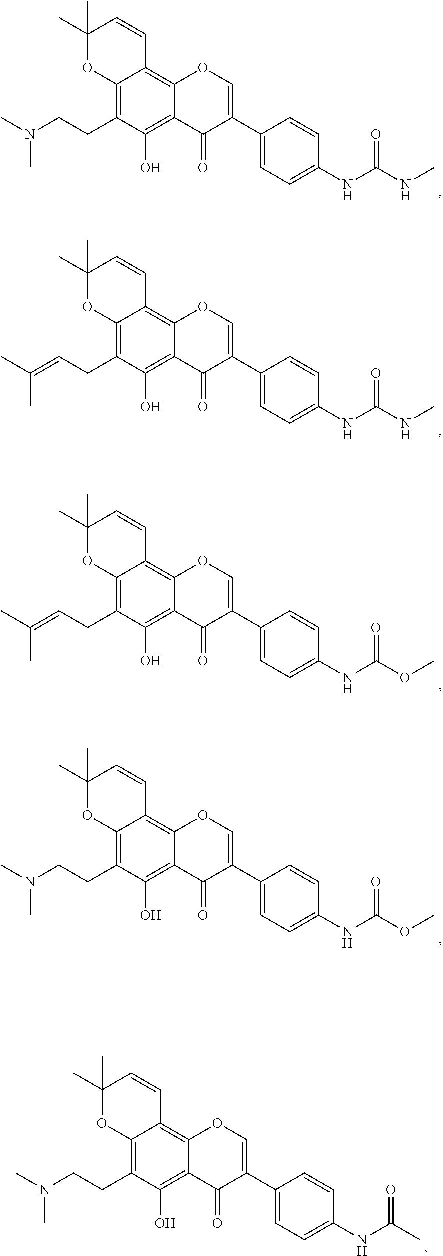 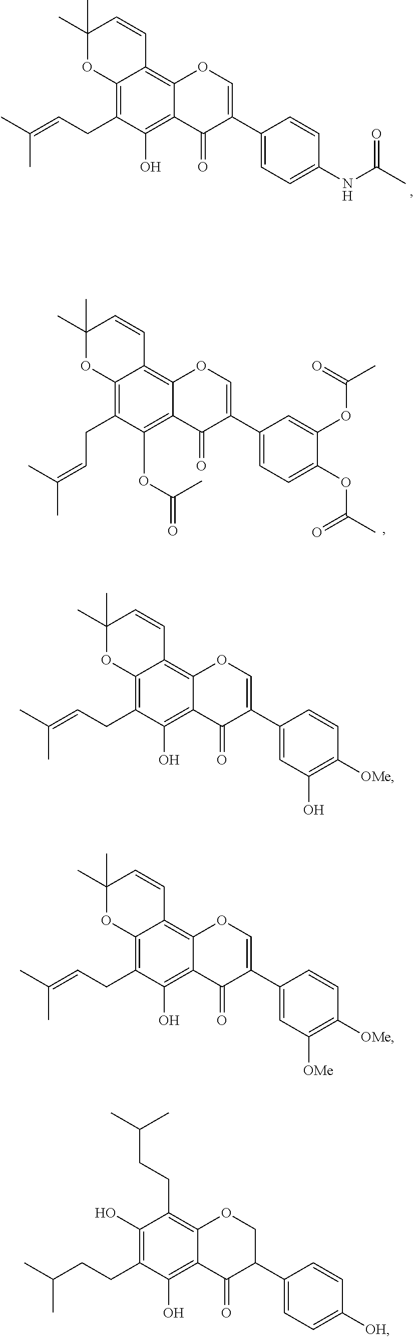 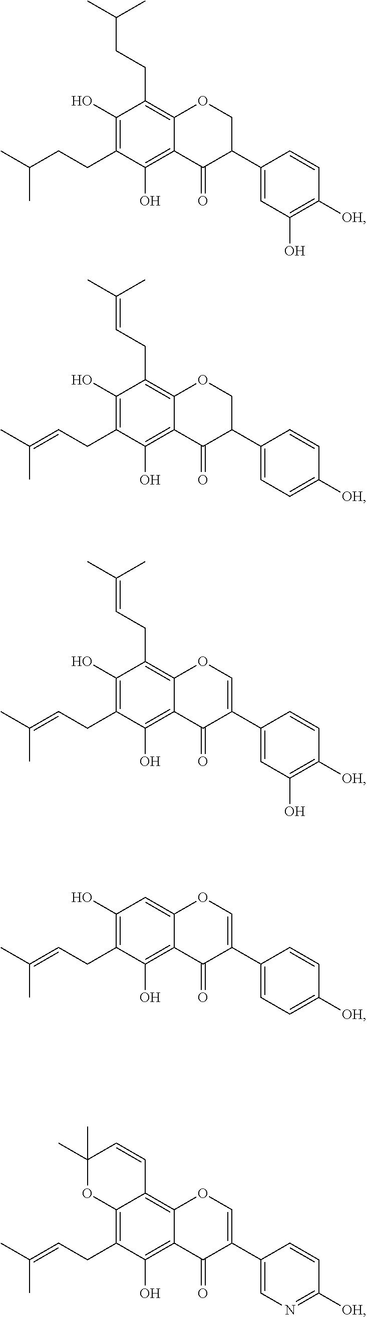 [SMILES: CC(C)CCc1c(O)c(CCC(C)C)c2c(c1O)C(=O)C(c1ccc(O)c(O)c1)CO2 ; CC(C)=CCc1c(O)c(CC=C(C)C)c2c(c1O)C(=O)C(c1ccc(O)cc1)CO2 ; CC(C)=CCc1c(O)c(CC=C(C)C)c2occ(-c3ccc(O)c(O)c3)c(=O)c2c1O ; CC(C)=CCc1c(O)cc2occ(-c3ccc(O)cc3)c(=O)c2c1O ; CC(C)=CCc1c2c(c3occ(-c4ccc(O)nc4)c(=O)c3c1O)C=CC(C)(C)O2] 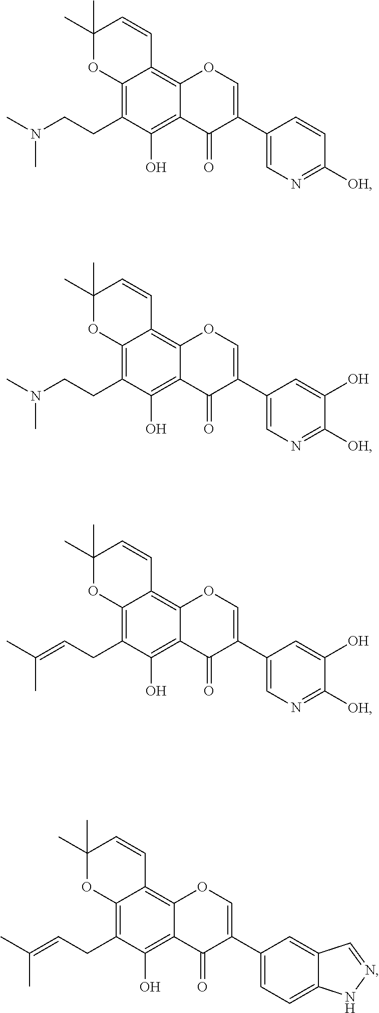 [SMILES: CN(C)CCc1c2c(c3occ(-c4ccc(O)nc4)c(=O)c3c1O)C=CC(C)(C)O2 ; CN(C)CCc1c2c(c3occ(-c4cnc(O)c(O)c4)c(=O)c3c1O)C=CC(C)(C)O2 ; CC(C)=CCc1c2c(c3occ(-c4cnc(O)c(O)c4)c(=O)c3c1O)C=CC(C)(C)O2 ; CC(C)=CCc1c2c(c3occ(-c4ccc5[nH]ncc5c4)c(=O)c3c1O)C=CC(C)(C)O2] 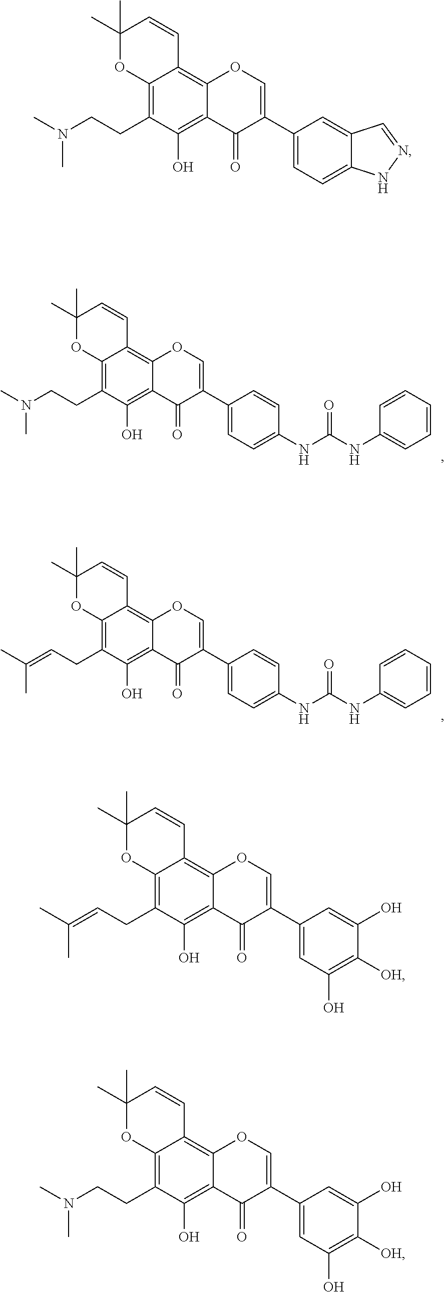 [SMILES: CN(C)CCc1c2c(c3occ(-c4ccc5[nH]ncc5c4)c(=O)c3c1O)C=CC(C)(C)O2 ; CN(C)CCc1c2c(c3occ(-c4ccc(NC(=O)Nc5ccccc5)cc4)c(=O)c3c1O)C=CC(C)(C)O2 ; CC(C)=CCc1c2c(c3occ(-c4ccc(NC(=O)Nc5ccccc5)cc4)c(=O)c3c1O)C=CC(C)(C)O2 ; CC(C)=CCc1c2c(c3occ(-c4cc(O)c(O)c(O)c4)c(=O)c3c1O)C=CC(C)(C)O2 ; CN(C)CCc1c2c(c3occ(-c4cc(O)c(O)c(O)c4)c(=O)c3c1O)C=CC(C)(C)O2] 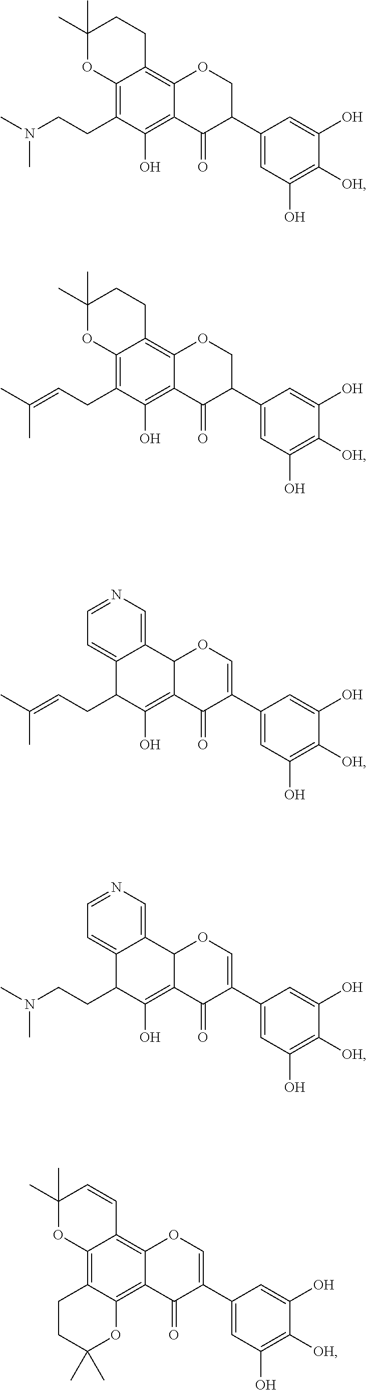 [SMILES: CN(C)CCc1c(O)c2c(c3c1OC(C)(C)CC3)OCC(c1cc(O)c(O)c(O)c1)C2=O ; CC(C)=CCc1c(O)c2c(c3c1OC(C)(C)CC3)OCC(c1cc(O)c(O)c(O)c1)C2=O ; CC(C)=CCC1C(O)=C2C(=O)C(c3cc(O)c(O)c(O)c3)=COC2c2cnccc21 ; CN(C)CCC1C(O)=C2C(=O)C(c3cc(O)c(O)c(O)c3)=COC2c2cnccc21 ; CC1(C)C=Cc2c(c3c(c4c(=O)c(-c5cc(O)c(O)c(O)c5)coc24)OC(C)(C)CC3)O1]
and combinations thereof,
or an N-oxide, crystalline form, hydrate thereof, or a pharmaceutically acceptable salt thereof.

5. A kit comprising a compound according to claim 1 together with instructions for the use of the compound.

6. A kit comprising a pharmaceutical composition according to claim 3 together with instructions for the use of the pharmaceutical composition

7. A method for treating or ameliorating the effects of a cancer in a subject, comprising administering to the subject a therapeutically effective amount of a compound having the structure of formula (I): 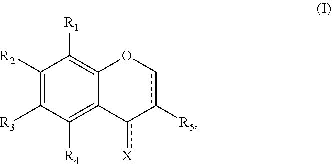
wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄alkyl, CF₃, and combinations thereof, or an N-oxide, crystalline form, hydrate thereof, or a pharmaceutically acceptable salt thereof.

8. The method of claim 7, wherein the compound is selected from the group consisting of: 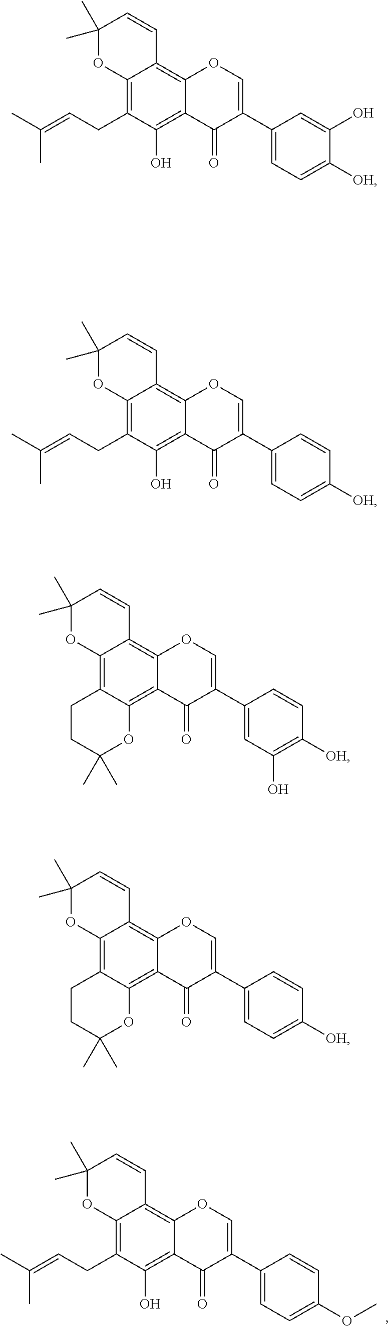 [SMILES: CC(C)=CCc1c2c(c3occ(-c4ccc(O)c(O)c4)c(=O)c3c1O)C=CC(C)(C)O2 ; CC(C)=CCc1c2c(c3occ(-c4ccc(O)cc4)c(=O)c3c1O)C=CC(C)(C)O2 ; CC1(C)C=Cc2c(c3c(c4c(=O)c(-c5ccc(O)c(O)c5)coc24)OC(C)(C)CC3)O1 ; CC1(C)C=Cc2c(c3c(c4c(=O)c(-c5ccc(O)cc5)coc24)OC(C)(C)CC3)O1 ; COc1ccc(-c2coc3c4c(c(CC=C(C)C)c(O)c3c2=O)OC(C)(C)C=C4)cc1] 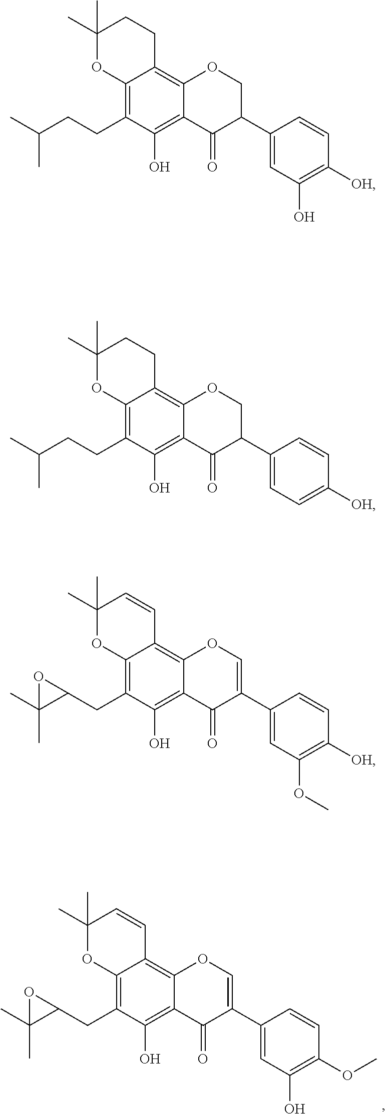 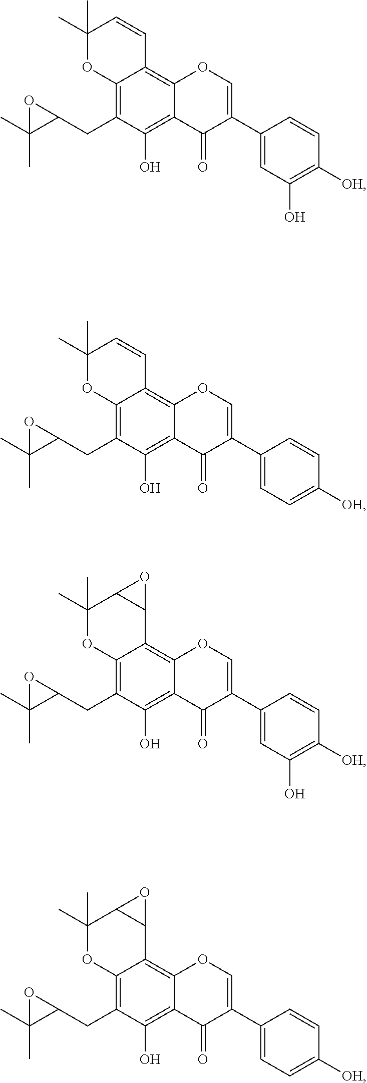 [SMILES: CC1(C)C=Cc2c(c(CC3OC3(C)C)c(O)c3c(=O)c(-c4ccc(O)c(O)c4)coc23)O1 ; CC1(C)C=Cc2c(c(CC3OC3(C)C)c(O)c3c(=O)c(-c4ccc(O)cc4)coc23)O1 ; CC1(C)OC1Cc1c2c(c3occ(-c4ccc(O)c(O)c4)c(=O)c3c1O)C1OC1C(C)(C)O2 ; CC1(C)OC1Cc1c2c(c3occ(-c4ccc(O)cc4)c(=O)c3c1O)C1OC1C(C)(C)O2] 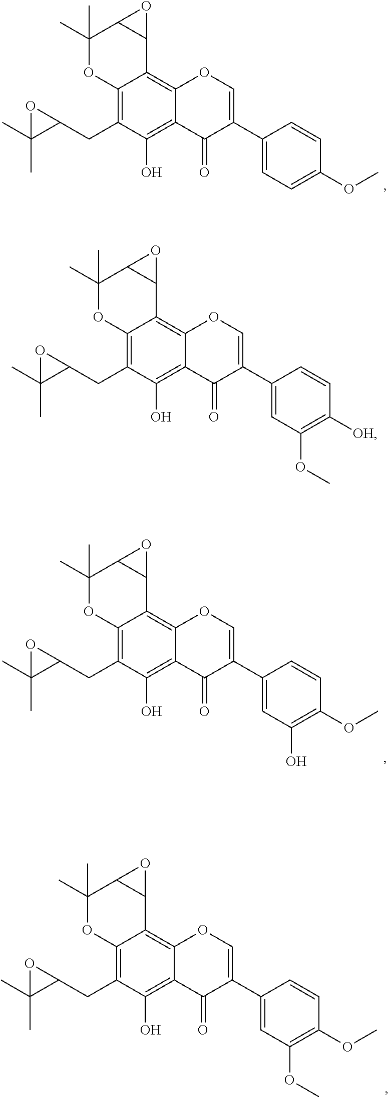 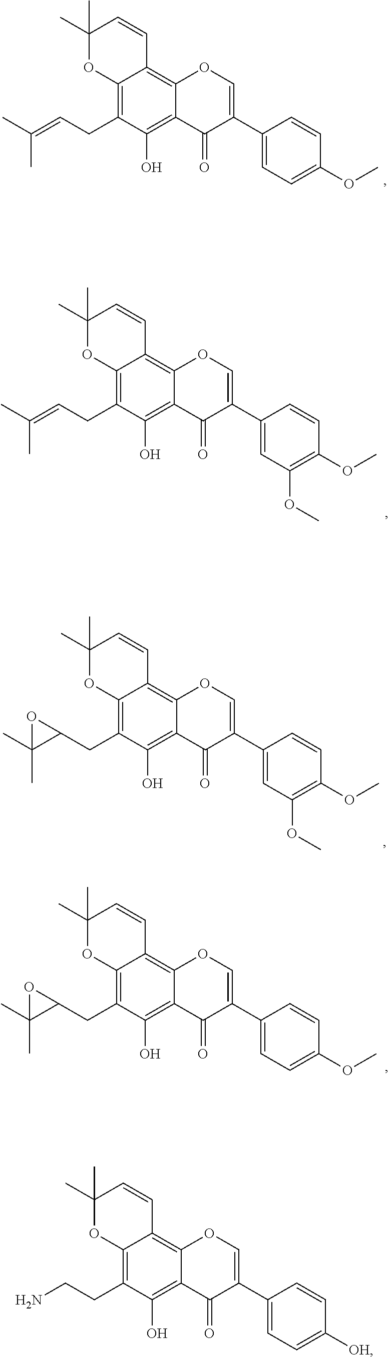 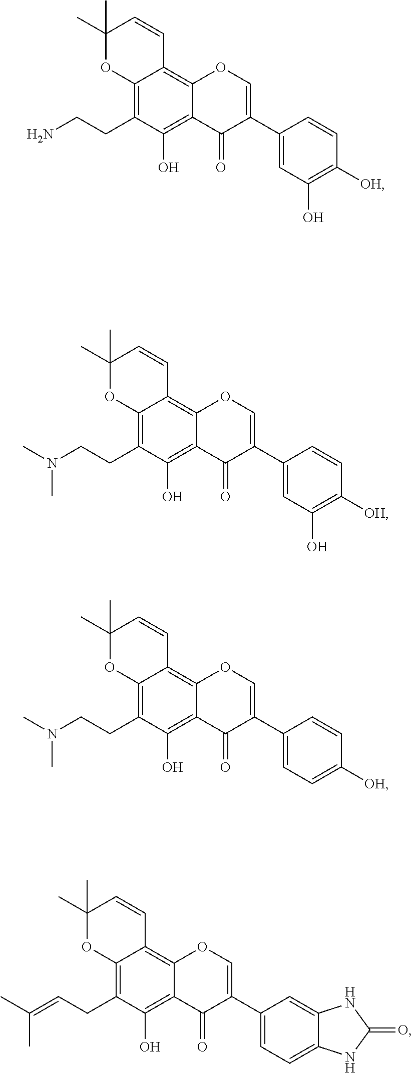 [SMILES: CC1(C)C=Cc2c(c(CCN)c(O)c3c(=O)c(-c4ccc(O)c(O)c4)coc23)O1 ; CN(C)CCc1c2c(c3occ(-c4ccc(O)c(O)c4)c(=O)c3c1O)C=CC(C)(C)O2 ; CN(C)CCc1c2c(c3occ(-c4ccc(O)cc4)c(=O)c3c1O)C=CC(C)(C)O2 ; CC(C)=CCc1c2c(c3occ(-c4ccc5[nH]c(=O)[nH]c5c4)c(=O)c3c1O)C=CC(C)(C)O2] 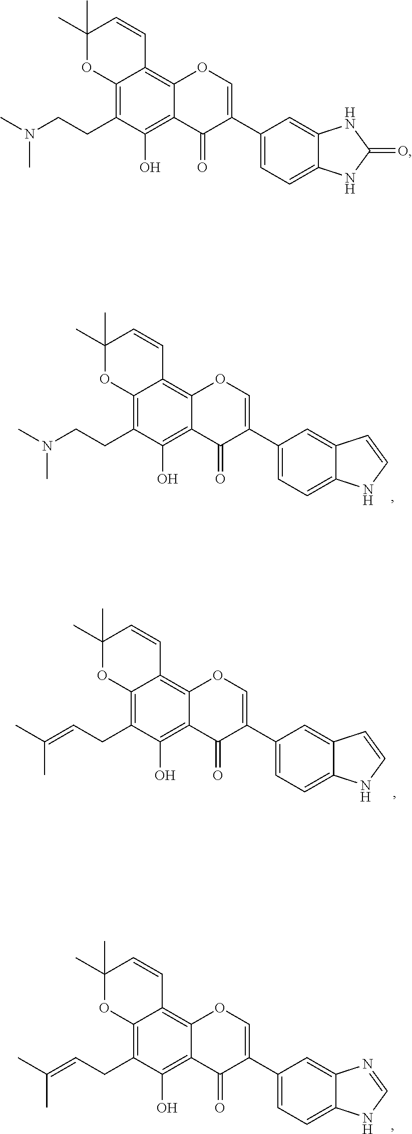 [SMILES: CN(C)CCc1c2c(c3occ(-c4ccc5[nH]c(=O)[nH]c5c4)c(=O)c3c1O)C=CC(C)(C)O2 ; CN(C)CCc1c2c(c3occ(-c4ccc5[nH]ccc5c4)c(=O)c3c1O)C=CC(C)(C)O2 ; CC(C)=CCc1c2c(c3occ(-c4ccc5[nH]ccc5c4)c(=O)c3c1O)C=CC(C)(C)O2 ; CC(C)=CCc1c2c(c3occ(-c4ccc5[nH]cnc5c4)c(=O)c3c1O)C=CC(C)(C)O2] 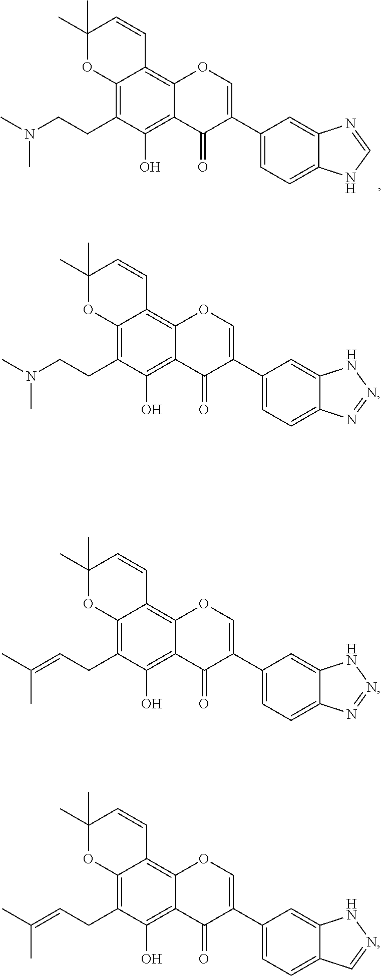 [SMILES: CN(C)CCc1c2c(c3occ(-c4ccc5[nH]cnc5c4)c(=O)c3c1O)C=CC(C)(C)O2 ; CN(C)CCc1c2c(c3occ(-c4ccc5nn[nH]c5c4)c(=O)c3c1O)C=CC(C)(C)O2 ; CC(C)=CCc1c2c(c3occ(-c4ccc5nn[nH]c5c4)c(=O)c3c1O)C=CC(C)(C)O2 ; CC(C)=CCc1c2c(c3occ(-c4ccc5cn[nH]c5c4)c(=O)c3c1O)C=CC(C)(C)O2] 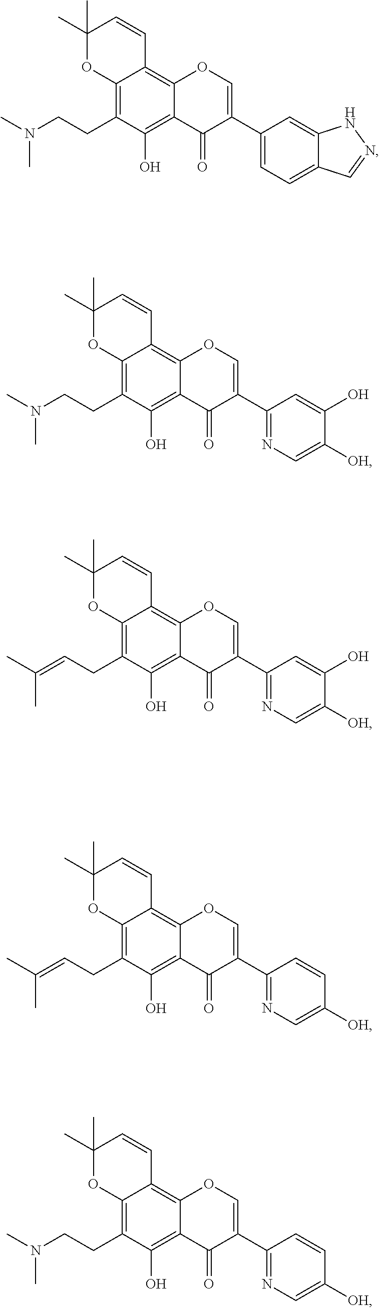 [SMILES: CN(C)CCc1c2c(c3occ(-c4ccc5cn[nH]c5c4)c(=O)c3c1O)C=CC(C)(C)O2 ; CN(C)CCc1c2c(c3occ(-c4cc(O)c(O)cn4)c(=O)c3c1O)C=CC(C)(C)O2 ; CC(C)=CCc1c2c(c3occ(-c4cc(O)c(O)cn4)c(=O)c3c1O)C=CC(C)(C)O2 ; CC(C)=CCc1c2c(c3occ(-c4ccc(O)cn4)c(=O)c3c1O)C=CC(C)(C)O2 ; CN(C)CCc1c2c(c3occ(-c4ccc(O)cn4)c(=O)c3c1O)C=CC(C)(C)O2] 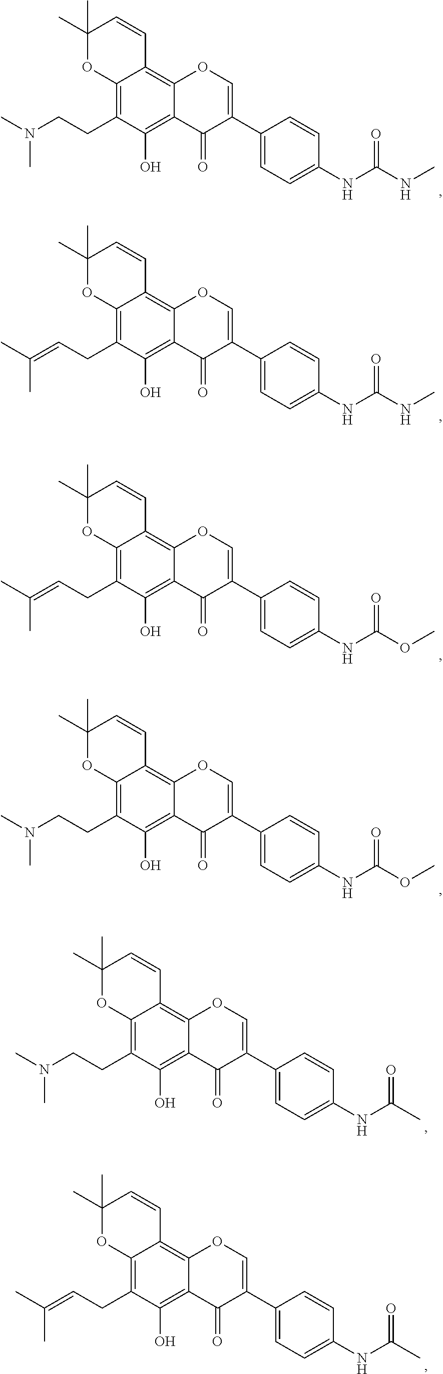 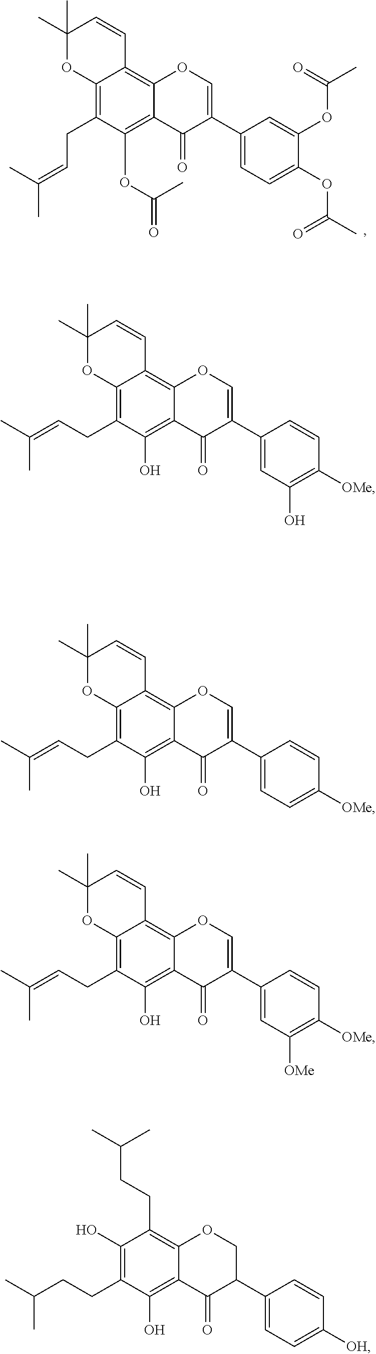 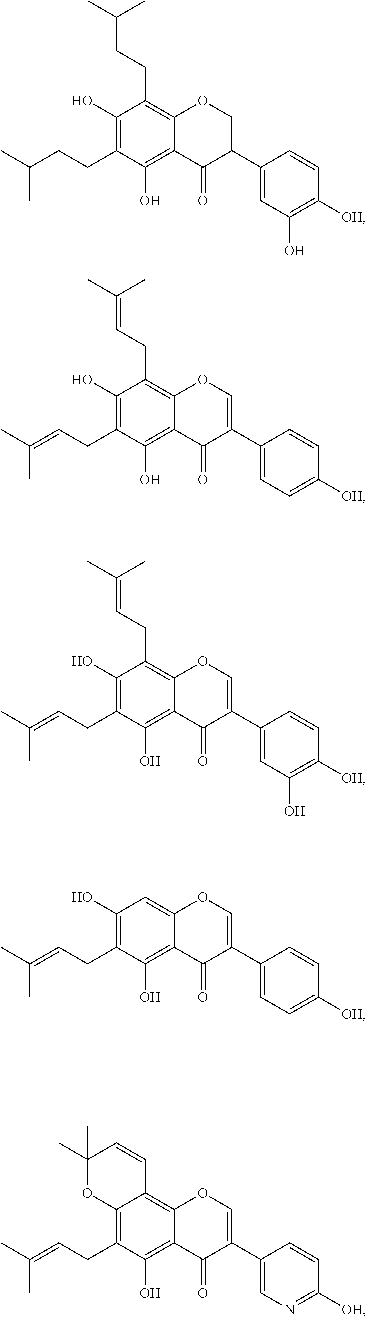 [SMILES: CC(C)CCc1c(O)c(CCC(C)C)c2c(c1O)C(=O)C(c1ccc(O)c(O)c1)CO2 ; CC(C)=CCc1c(O)c(CC=C(C)C)c2occ(-c3ccc(O)cc3)c(=O)c2c1O ; CC(C)=CCc1c(O)c(CC=C(C)C)c2occ(-c3ccc(O)c(O)c3)c(=O)c2c1O ; CC(C)=CCc1c(O)cc2occ(-c3ccc(O)cc3)c(=O)c2c1O ; CC(C)=CCc1c2c(c3occ(-c4ccc(O)nc4)c(=O)c3c1O)C=CC(C)(C)O2] 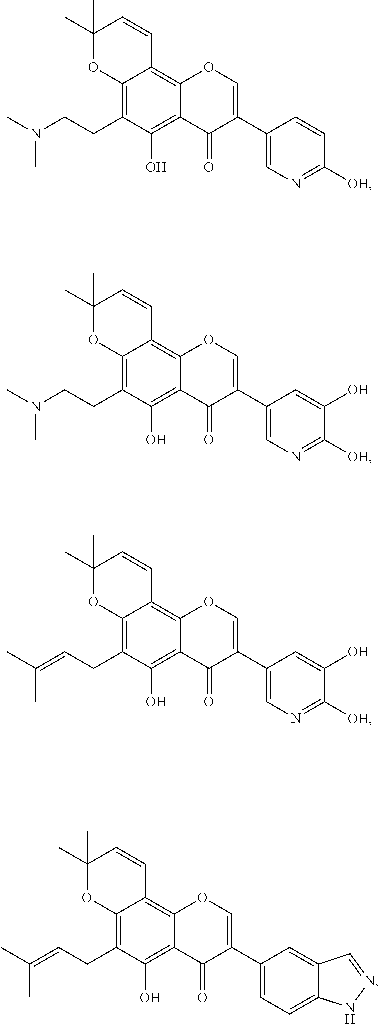 [SMILES: CN(C)CCc1c2c(c3occ(-c4ccc(O)nc4)c(=O)c3c1O)C=CC(C)(C)O2 ; CN(C)CCc1c2c(c3occ(-c4cnc(O)c(O)c4)c(=O)c3c1O)C=CC(C)(C)O2 ; CC(C)=CCc1c2c(c3occ(-c4cnc(O)c(O)c4)c(=O)c3c1O)C=CC(C)(C)O2 ; CC(C)=CCc1c2c(c3occ(-c4ccc5[nH]ncc5c4)c(=O)c3c1O)C=CC(C)(C)O2] 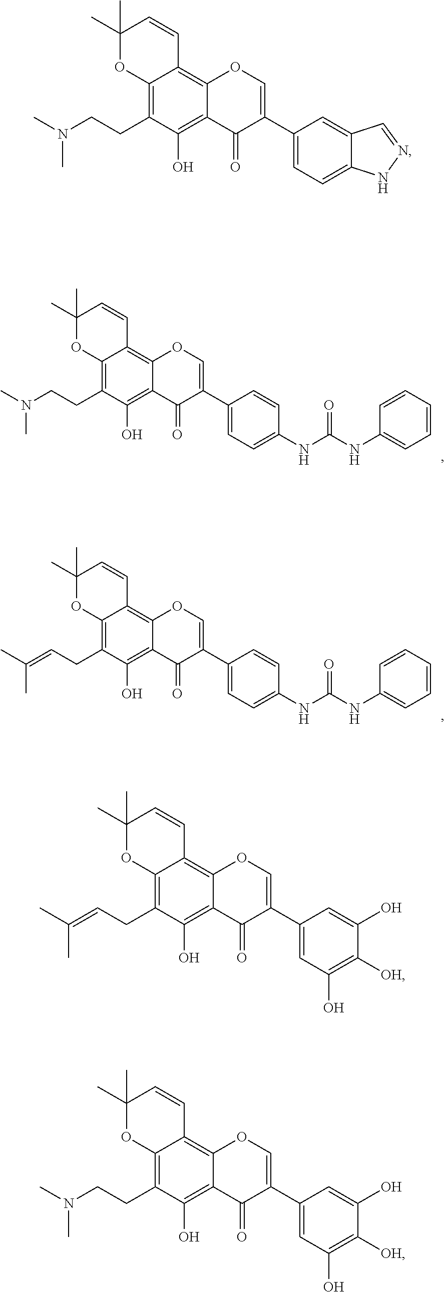 [SMILES: CN(C)CCc1c2c(c3occ(-c4ccc5[nH]ncc5c4)c(=O)c3c1O)C=CC(C)(C)O2 ; CN(C)CCc1c2c(c3occ(-c4ccc(NC(=O)Nc5ccccc5)cc4)c(=O)c3c1O)C=CC(C)(C)O2 ; CC(C)=CCc1c2c(c3occ(-c4ccc(NC(=O)Nc5ccccc5)cc4)c(=O)c3c1O)C=CC(C)(C)O2 ; CC(C)=CCc1c2c(c3occ(-c4cc(O)c(O)c(O)c4)c(=O)c3c1O)C=CC(C)(C)O2 ; CN(C)CCc1c2c(c3occ(-c4cc(O)c(O)c(O)c4)c(=O)c3c1O)C=CC(C)(C)O2] 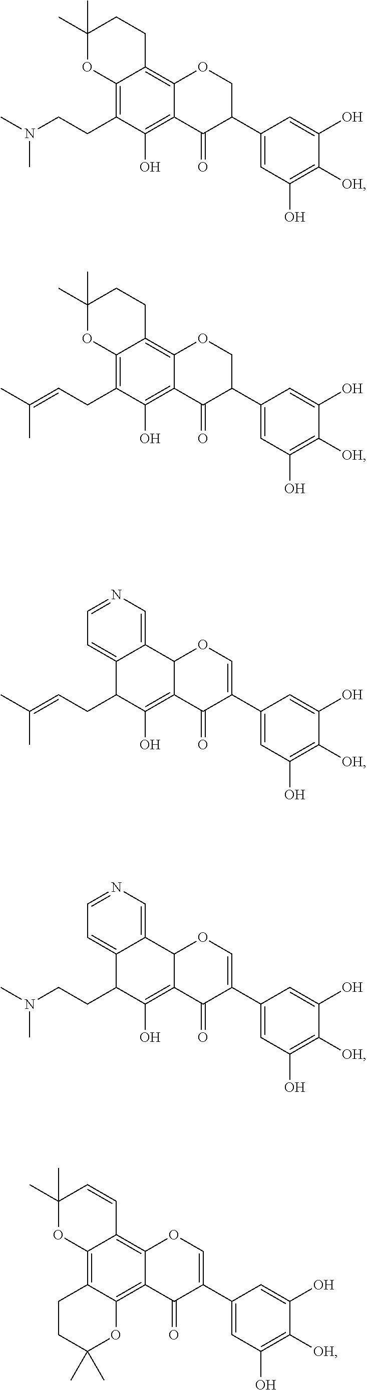 [SMILES: CN(C)CCc1c(O)c2c(c3c1OC(C)(C)CC3)OCC(c1cc(O)c(O)c(O)c1)C2=O ; CC(C)=CCc1c(O)c2c(c3c1OC(C)(C)CC3)OCC(c1cc(O)c(O)c(O)c1)C2=O ; CC(C)=CCC1C(O)=C2C(=O)C(c3cc(O)c(O)c(O)c3)=COC2c2cnccc21 ; CN(C)CCC1C(O)=C2C(=O)C(c3cc(O)c(O)c(O)c3)=COC2c2cnccc21 ; CC1(C)C=Cc2c(c3c(c4c(=O)c(-c5cc(O)c(O)c(O)c5)coc24)OC(C)(C)CC3)O1]
and combinations thereof,
or an N-oxide, crystalline form, hydrate thereof, or a pharmaceutically acceptable salt thereof.

9. The method of claim 7, wherein the compound is selected from the group consisting of: 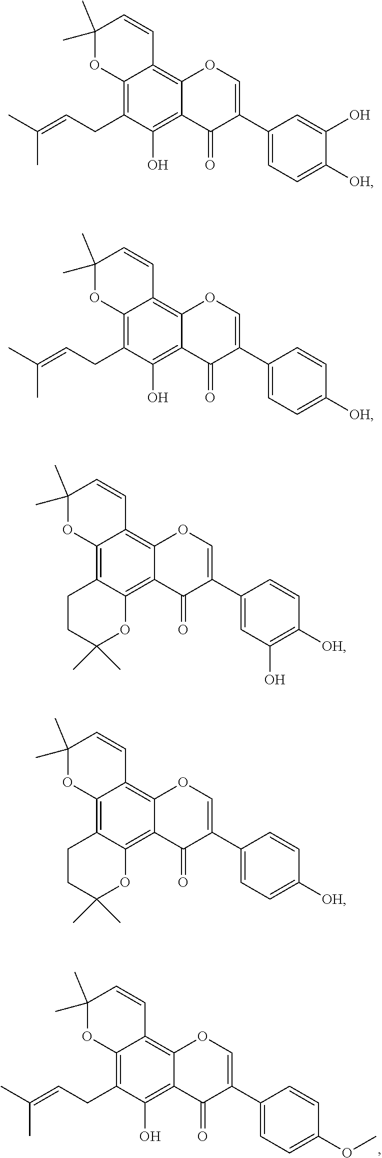 [SMILES: CC(C)=CCc1c2c(c3occ(-c4ccc(O)c(O)c4)c(=O)c3c1O)C=CC(C)(C)O2 ; CC(C)=CCc1c2c(c3occ(-c4ccc(O)cc4)c(=O)c3c1O)C=CC(C)(C)O2 ; CC1(C)C=Cc2c(c3c(c4c(=O)c(-c5ccc(O)c(O)c5)coc24)OC(C)(C)CC3)O1 ; CC1(C)C=Cc2c(c3c(c4c(=O)c(-c5ccc(O)cc5)coc24)OC(C)(C)CC3)O1 ; COc1ccc(-c2coc3c4c(c(CC=C(C)C)c(O)c3c2=O)OC(C)(C)C=C4)cc1]
and combinations thereof,
or an N-oxide, crystalline form, hydrate thereof, or a pharmaceutically acceptable salt thereof.

10. The method of claim 7, wherein the compound is: 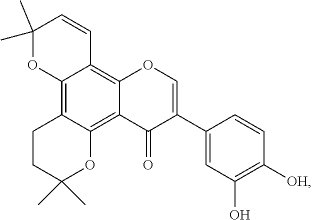 [SMILES: CC1(C)C=Cc2c(c3c(c4c(=O)c(-c5ccc(O)c(O)c5)coc24)OC(C)(C)CC3)O1]
or an N-oxide, crystalline form, hydrate thereof, or a pharmaceutically acceptable salt thereof.

11. The method of claim 7, wherein the cancer is selected from the group consisting of glioma, thyroid cancer, lung cancer, liver cancer, pancreatic cancer, head and neck cancer, stomach cancer, colorectal cancer, urothelial cancer, renal cancer, prostate cancer, testis cancer, breast cancer, cervical cancer, ovarian cancer, endometrial cancer, melanoma, lymphoma, acute myeloid leukemia (AML), neuroblastoma, medulloblastoma, retinoblastoma, astrocytoma, glioblastoma multiforme, castration-resistant prostate cancer (CRPC), neuroendocrine prostate cancer (NEPC), hematologic malignancies, rhabdomyosarcoma, Wilms tumors, non-small cell lung cancer (NSCLC) and small cell lung cancer (SCLC).

12. The method of claim 7, wherein the cancer is driven by MycN and/or cMyc.

13. The method of claim 12, wherein the cancer is MycN-amplified neuroblastoma (MycN^{AMP} NBL).

14. The method of claim 7, further comprising co-administering to the subject a chemotherapy drug selected from the group consisting of cisplatin, temozolomide, doxorubicin, cyclophosphamide, methotrexate, 5-fluorouracil, vinorelbine, docetaxel, bleomycin, vinblastine, dacarbazine, mustine, vincristine, procarbazine, prednisolone, etoposide, epirubicin, capecitabine, methotrexate, folinic acid, oxaliplatin, and combinations thereof.

15. The method of claim 7, further comprising co-administering radiotherapy to the subject.

16. The method of claim 7, wherein the subject is a mammal.

17. The method of claim 16, wherein the mammal is selected from the group consisting of humans, veterinary animals, and agricultural animals.

18. The method of claim 7, wherein the subject is a human.

19. The method of claim 7, wherein the subject is a pediatric patient.

20. A method for selectively killing a cancer cell, comprising contacting the cancer cell with an effective amount of a compound having the structure of formula (I): 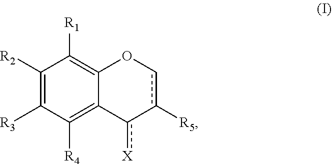
wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄alkyl, CF₃, and combinations thereof,
or an N-oxide, crystalline form, hydrate thereof, or a pharmaceutically acceptable salt thereof.

21. The method of claim 20, wherein the cancer cell overexpresses MycN and/or cMyc.

22. A method of modulating mTORC1/2 signaling activity in a cell, comprising contacting the cell with an effective amount of a compound having the structure of formula (I): 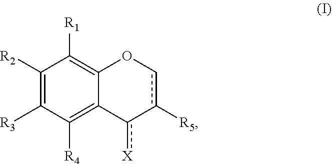
wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄ alkyl, CF₃, and combinations thereof, or an N-oxide, crystalline form, hydrate thereof, or a pharmaceutically acceptable salt thereof.

23. A method of modulating the activity of a Master Regulator for MycN in a subject having MycN-amplified neuroblastoma (MycN^{AMP} NBL), comprising administering to the subject a therapeutically effective amount of a compound having the structure of formula (I): 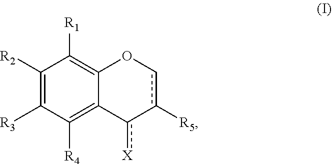
wherein:
a dashed line indicates the presence of an optional double bond;
X is selected from the group consisting of no atom, H, and O;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₀carbocycle that may be optionally substituted with an atom or a group selected from the group consisting of O, N, halo, C₁₋₄alkyl, CF₃, and combinations thereof;
R₃ and R₄ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₃ and R₄ may together form a C₃₋₁₀carbocycle that may be optionally substituted with an atom or a group selected from the group consisting of O, N, halo, C₁₋₄alkyl, CF₃, and combinations thereof; and
R₅ is selected from the group consisting of NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, aryl, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of —OH, halo, C₁₋₄alkyl, CF₃, and combinations thereof, or an N-oxide, crystalline form, hydrate thereof, or a pharmaceutically acceptable salt thereof.

24. The method of claim 23, wherein the modulation comprises reversing the NBL master regulatory activity for cMyc in the subject.

25. A method of selectively treating or ameliorating effects of a cancer in a subject in need thereof, comprising the steps of:
(a) obtaining a biological sample from the subject;
(b) determining the expression level of MycN in the sample and comparing it with a predetermined reference;
(c) identifying the subject as a MycN^{AMP} subtype if MycN in the sample is determined to be overexpressed in step (b); and
(d) treating the MycN^{AMP} subtype subject with a therapeutically effective amount of a compound according to any one of claims 1-2 or a pharmaceutical composition according to any one of claims 3-4.

26. A method of selectively treating or ameliorating effects of a cancer in a subject in need thereof, comprising the steps of:
(a) obtaining a biological sample from the subject;
(b) determining the expression level of cMyc in the sample and comparing it with a predetermined reference;
(c) identifying the subject as a cMyc^{AMP} subtype if cMyc in the sample is determined to be overexpressed in step (b); and
(d) treating the cMyc^{AMP} subtype subject with a therapeutically effective amount of a compound according to any one of claims 1-2 or a pharmaceutical composition according to any one of claims 3-4.

27. A method for identifying a compound that induces degradation of a cancer-related protein, comprising the steps of:
(a) obtaining cancer cell lines that express the protein (AMP cell lines) and cancer cell lines that do not express the protein (NULL cell lines);
(b) identifying compounds that are lethal to at least one of the cell lines;
(c) identifying compounds that are selective for AMP cell lines from those identified in step (b) based on cell line subtype selectivity;
(d) determining the expression level of the protein in AMP cell lines for each selective compound identified in step (c) by performing a high-throughput gene expression profiling; and
(e) identifying a candidate compound that induces degradation of the cancer-related protein based on the result of step (d).

28. The method of claim 27, wherein the cancer-related protein is MycN or cMyc.

29. The method of claim 27, wherein the gene expression profiling in step (d) is performed by PLATE-Seq.

30. A method for treating or ameliorating the effects of a cancer in a subject, comprising administering to the subject a therapeutically effective amount of a compound selected from the group consisting of mycophenolate, NSC 80997, podofilox, cloxyquin, NSC 305798, NSC 255109, narasin, methylene blue, azure A, azure B, rapamycin, NSC 3905, and combinations thereof, or an N-oxide, crystalline form, hydrate thereof, or a pharmaceutically acceptable salt thereof.

31. The method of claim 30, wherein the cancer is selected from the group consisting of glioma, thyroid cancer, lung cancer, liver cancer, pancreatic cancer, head and neck cancer, stomach cancer, colorectal cancer, urothelial cancer, renal cancer, prostate cancer, testis cancer, breast cancer, cervical cancer, ovarian cancer, endometrial cancer, melanoma, lymphoma, acute myeloid leukemia (AML), neuroblastoma, medulloblastoma, retinoblastoma, astrocytoma, glioblastoma multiforme, castration-resistant prostate cancer (CRPC), neuroendocrine prostate cancer (NEPC), hematologic malignancies, rhabdomyosarcoma, Wilms tumors, non-small cell lung cancer (NSCLC) and small cell lung cancer (SCLC).

32. The method of claim 30, wherein the cancer is driven by MycN and/or cMyc.

33. The method of claim 32, wherein the cancer is MycN-amplified neuroblastoma (MycN^{AMP} NBL).

34. The method of claim 30, further comprising co-administering to the subject a chemotherapy drug selected from the group consisting of cisplatin, temozolomide, doxorubicin, cyclophosphamide, methotrexate, 5-fluorouracil, vinorelbine, docetaxel, bleomycin, vinblastine, dacarbazine, mustine, vincristine, procarbazine, prednisolone, etoposide, epirubicin, capecitabine, methotrexate, folinic acid, oxaliplatin, and combinations thereof.

35. The method of claim 30, further comprising co-administering radiotherapy to the subject.

36. The method of claim 30, further comprising co-administering to the subject an effective amount of an aurora A kinase inhibitor.

37. The method of claim 36, wherein the aurora A kinase inhibitor is alisertib.

38. A method of modulating the activity of a Master Regulator for MycN in a subject having MycN-amplified neuroblastoma (MycN^{AMP} NBL), comprising administering to the subject a therapeutically effective amount of a compound selected from the group consisting of mycophenolate, NSC 80997, podofilox, cloxyquin, NSC 305798, NSC 255109, narasin, methylene blue, azure A, azure B, rapamycin, NSC 3905, and combinations thereof, or an N-oxide, crystalline form, hydrate thereof, or a pharmaceutically acceptable salt thereof.

39. The method of claim 38, wherein the modulation comprises reversing the NBL master regulatory activity for cMyc in the subject.

40. A compound having the structure of: 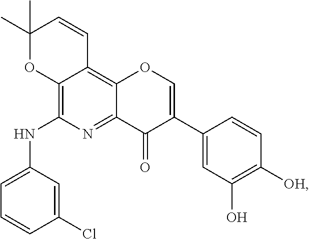 [SMILES: CC1(C)C=Cc2c(c(Nc3cccc(Cl)c3)nc3c(=O)c(-c4ccc(O)c(O)c4)coc23)O1]
